(19) United States
(12) Patent Application Publication
(10) Pub. No.: US 2026/0317787 A1
(43) Pub. Date: Oct. 15, 2026

(54) METHODS COMPRISING ONCOLYTIC VIRUSES EXPRESSING CD19T AND BISPECIFIC T CELL ENGAGERS

(71) Applicants: City of Hope, Duarte, CA (US); Imugene Limited, Sydney (AU)

(72) Inventors: Saul J. Priceman, Valley Village, CA (US); Anthony K. Park, Pomona, CA (US); Yuman Fong, Pasadena, CA (US); Monil Shah, Sydney (AU)

(21) Appl. No.: 19/688,290

(22) Filed: May 26, 2026

Related U.S. Application Data
(63) Continuation of application No. 18/939,460, filed on Nov. 6, 2024, now Pat. No. 12,698,512.
(60) Provisional application No. 63/596,582, filed on Nov. 6, 2023.

(51) Int. Cl. C12N 15/86; A61K 39/00; A61K 39/395; A61P 35/00; C07K 14/705; C07K 16/28

(52) U.S. Cl. CPC C12N 15/86; A61K 39/001112; A61K 39/39541; A61K 39/39558; A61P 35/00; C07K 14/70503; C07K 16/2803; C07K 16/2809; A61K 2039/505; A61K 2039/5256; C07K 2317/31; C12N 2710/24143; C12N 2710/24171

(57) ABSTRACT

Described herein, inter alia, are oncolytic viruses expressing a truncated human CD19 (CD19t) and methods for treating a patient suffering from a solid tumor by administering an oncolytic virus expressing truncated human CD19 (OV19t), and optionally a T cell engager targeted to CD19.

### CLAIM OF PRIORITY

This application is a continuation of U.S. application Ser. No. 18/939,460, filed on Nov. 6, 2024, which claims the benefit of U.S. Provisional Application Ser. No. 63/596,582, filed on Nov. 6, 2023. The entire contents of the foregoing are incorporated herein by reference.

### SEQUENCE LISTING

This application contains a Sequence Listing that has been submitted electronically as an XML file named 40056-0103002_SL_ST26.xml. The XML file, created on May 26, 2026, is 1,638,782 bytes in size. The material in the XML file is hereby incorporated by reference in its entirety.

### TECHNICAL FIELD

This disclosure relates to methods of treating a subject having a solid tumor comprising administering an oncolytic virus expressing a truncated CD19 (CD19t), and a CD19-targeted therapy that binds to the CD19t (e.g., a bispecific T cell engager (TCE)).

### BACKGROUND

A universal tumor-agnostic cancer therapy remains elusive in the field of immunotherapy. There have been impressive patient specific immunotherapies with recent FDA approvals including CAR T cell therapies. Limitations include the autologous nature of these cells and the length of time required to manufacturing. In contrast to autologous CAR T cells, the benefits of bispecific T cell engagers (called BiTEs and TCEs interchangeably herein) are its off-the-shelf availability and the ability to acutely tune targeting with dosing regimens [1,2]. TCEs have emerged as a promising immunotherapy strategy for the treatment of B-cell hematological malignancies. Blinatumomab, an FDA approved TCE carrying CD19 and CD3 single chain variable fragments (scFvs) that drives endogenous T-cell mediated immune responses against malignant cells, has shown durable clinical responses for the treatment of B-cell acute lymphoblastic leukemia (B-ALL) and non-Hodgkin's lymphoma [3, 4].

Challenges facing solid tumor therapies using CAR T cells and TCE therapies are driven by a lack amenable and targetable tumor antigens [5,6]. The shared expression of solid tumor antigens on normal tissue and their heterogeneous, and nonuniform, expression patterns on tumors limits the potential for effective and durable anti-tumor responses [7,8]. Many solid tumors are also immunologically “cold” and limit T cell trafficking and anti-tumor functionality, a phenomenon uncommonly observed in hematological malignancies [9-11]. Further, the majority of solid tumors have a more complex microenvironment that represents a greater challenge for cancer therapies [24]. Thus, improved and more accessible immunotherapies remain to be explored.

### SUMMARY

This application is based, at least in part, on the discovery that use of oncolytic viruses (OV) to redirect CD19-targeted bispecific T cell engagers (called BiTEs or TCEs interchangeably throughout) drives anti-tumor responses of endogenous T cells against multiple solid tumor types.

Chimeric antigen receptor (CAR)-engineered T cell therapies targeting CD19 demonstrate remarkable clinical successes for patients with hematological malignancies.

However, limited and heterogeneously-expressed tumor targets impede the field in similarly treating solid tumors. This application is describes, inter alia, a combinatorial platform using OV and CD19-targeting BiTEs to target particularly difficult to treat solid tumors. Furthermore, this approach overcame the concerning issue of antigen escape following treatment with HER2-CAR T cells. Combining OV with clinically-approved TCEs as described herein provides a readily translatable, tumor-agnostic, off-the-shelf strategy to effectively target solid tumors.

OV have recently emerged as a promising off-the-shelf treatment modality for various tumor types. OV are tumor-specific viruses that have desirable immunogenic properties with the capacity for transgene delivery to tumors [12]. An OV (OV19t) expressing a truncated non-signaling variant of CD19 (CD19t) has been shown to redirect of CD19-CAR T cells to solid tumors by exploiting the transgene delivery potential of OV19t to selectively infect and drive tumor-specific expression of the truncated nonsignaling variant of CD19 [13]. The parent version of the chimeric poxvirus-based OV used here has shown safety and antitumor activity in several preclinical models [14,15]. Herein, we expand the therapy by making it fully off-the-shelf with the addition of a BiTEs. Here, the data demonstrate robust cell surface CD19t expression on multiple tumor types infected with an OV carrying the CD19t-encoding gene (OV19t) as described herein, which promoted activation and tumor killing by T cells when treated with a CD19-TCE.

Described herein, inter alia, are methods of administering an oncolytic virus (OV) expressing a truncated variant of CD19 (CD19t) and an agent that binds both CD19 and CD3 (e.g., a bispecific T cell engager) to a subject in need thereof. Described herein are methods of killing solid tumor cancer cells comprising: administering to the subject an effective amount of an oncolytic virus expressing CD19 or CD19t (OV19 or OV19t); and administering to the subject a bispecific T cell engager (TCE) that binds to the CD19 or CD19t encoded by the OV19 or OV19t. Also described herein are methods of treating a subject having a solid tumor comprising: administering to the subject an effective amount of an oncolytic virus expressing CD19 or CD19t (OV19 or OV19t); and administering to the subject a bispecific T cell engager (TCE) that binds to the CD19 or CD19t encoded by the OV19 or OV19t.

Described herein is a method of treating a subject having a solid tumor comprising: administering to the subject an effective amount of an oncolytic virus expressing a truncated CD19 (OV19t), the nucleotide sequence of OV19t comprising: (a) an oncolytic virus nucleotide sequence; and (b) a nucleotide sequence encoding a truncated human CD19; and administering to the subject an effective amount of a bispecific T cell engager (TCE) that binds to CD19.

In various embodiments: OV19t does not encode functional thymidine kinase; the OV19t comprises a nucleotide sequence that is at least 90%, 95%, 96%, 97%, 98%, 99%, 99.5%, 99.9% or 100% identical to SEQ ID NO: 1 over the entire length of SEQ ID NO: 1; the oncolytic virus nucleotide sequence comprises a nucleotide sequence that is at 90%, 95%, 96%, 97%, 98%, 99%, 99.5%, 99.9% or 100% identical to: a) SEQ ID NO: 2 over the entire length of SEQ ID NO: 2, but lacks the JR2 gene sequence of SEQ ID NO: 2; or b) SEQ ID NO: 2 over the entire length of SEQ ID NO: 2, but lacks JR2 gene sequence, the 5′ ITR sequence and the 3′ ITR sequence of SEQ ID NO: 2; the oncolytic virus nucleotide sequence has no modifications in the coding regions comprising SEQ ID NOs: 131-452, or wherein any modifications within the coding regions (SEQ ID NOs: 131-452) do not change the amino acid sequence of the encoded proteins; the oncolytic virus nucleotide sequence comprises a nucleotide sequence that is at least 90%, 95%, 96%, 97%, 98%, 99%, 99.5%, 99.9% or 100% identical to: SEQ ID NO: 2 over the entire length of SEQ ID NO: 2, but lacks the JR2 gene sequence and comprises nucleotide sequences that encode the proteins having SEQ ID NOs: 453-653, or a variant of each thereof having 1, 2, 3, 4, or 5 single amino acid substitutions.

In various embodiments: the OV19t comprises a nucleotide sequence that is at least 90%, 95%, 96%, 97%, 98%, 99%, 99.5%, 99.9% or 100% identical to: SEQ ID NO: 1 over the entire length of SEQ ID NO: 1, and encodes proteins having the amino acid sequences of SEQ ID NOs: 453-653, or a variant of each thereof having 1, 2, 3, 4, or 5 amino acid substitutions; the OV19t comprises a nucleotide sequence: a) that is at least 90%, 95%, 96%, 97%, 98%, 99%, 99.5%, 99.9% or 100% identical to: SEQ ID NO: 1 over the entire length of SEQ ID NO: 1, but lacks the 5′ ITR sequence and the 3′ ITR sequence of SEQ ID NO: 1; and b) encodes proteins having the amino acid sequences of SEQ ID NOs: 453-653, or a variant of each thereof having 1, 2, 3, 4, or 5 amino acid substitutions; the oncolytic virus sequence is at least 90%, 95%, 96%, 97%, 98%, 99%, 99.5%, 99.9% or 100% identical to: SEQ ID NO: 2 over the entire length of SEQ ID NO: 2, but lacks the 5′ ITR sequence, the 3′ ITR sequence and the J2R gene sequence of SEQ ID NO: 2; OV19t is at least 90%, 95%, 96%, 97%, 98%, 99%, 99.5%, 99.9% or 100% identical to: SEQ ID NO: 2 over the entire length of SEQ ID NO: 2 except that the nucleotide sequence encoding CD19t and a promoter sequence for expressing CD19t replaces at least 10 contiguous nucleotides of the JR2 gene sequence; and at least about 10, 20, 30, 40, 50, 60, 70, 80, 90, 100, 125, 150, 175, 200, 225, 250, 275, 300, 325, 250, 275, 400, 425, 450, 475, or 500 nucleotides (nts) of the J2R gene sequence have been deleted (e.g., the deletion comprises nucleotides 77682-78084 of SEQ ID NO:2).

In various embodiments: the nucleotide sequence encoding CD19t is inserted into a noncoding region of SEQ ID NO:2; the nucleotide sequence encoding CD19t encodes the extracellular domain and the transmembrane domain of CD19; the nucleotide sequence encoding CD19t does not encode the entirety of the cytoplasmic domain of CD19; the CD19t comprises an amino acid sequence that comprises or consists of SEQ ID NOs: 3-4; the nucleotide sequence encoding CD19t is operably linked to a synthetic early promoter; the TCE comprises a domain that binds CD19 and a domain that binds CD3; the domain that binds CD19 is a CD19-targeted scFv and the domain that binds CD3 is a CD3-targeted scFv; the CD19 targeted scFv comprises any one of the following:
a variable light chain region having CDR1, CDR2, and CDR3 regions comprising SEQ ID NOs: 66-68 and a variable light chain region having CDR1, CDR2, and CDR3 regions comprising SEQ ID NOs: 70-72;
a variable light chain region having CDR1, CDR2, and CDR3 regions comprising SEQ ID NOs: 74-76 and a variable light chain region having CDR1, CDR2, and CDR3 regions comprising SEQ ID NOs: 78-80;
a variable light chain region having CDR1, CDR2, and CDR3 regions comprising SEQ ID NOs: 82-84 and a variable light chain region having CDR1, CDR2, and CDR3 regions comprising SEQ ID NOs: 86-88;
a variable light chain region comprising SEQ ID NO: 69 and a variable heavy region comprising SEQ ID NO: 73;
a variable light chain region comprising SEQ ID NO: 77 and a variable heavy region comprising SEQ ID NO: 81; and
a variable light chain region comprising SEQ ID NO: 85 and a variable heavy region comprising SEQ ID NO: 89.

In various embodiments: the CD3 targeted scFv comprises any one of the following:
a variable light chain region having CDR1, CDR2, and CDR3 regions comprising SEQ ID NOs: 90-92 and a variable light chain region having CDR1, CDR2, and CDR3 regions comprising SEQ ID NOs: 94-96;
a variable light chain region having CDR1, CDR2, and CDR3 regions comprising SEQ ID NOs: 98-100 and a variable light chain region having CDR1, CDR2, and CDR3 regions comprising SEQ ID NOs: 102-104;
a variable light chain region having CDR1, CDR2, and CDR3 regions comprising SEQ ID NOs: 106-108 and a variable light chain region having CDR1, CDR2, and CDR3 regions comprising SEQ ID NOs: 110-112;
a variable light chain region comprising SEQ ID NO: 93 and a variable heavy region comprising SEQ ID NO: 97;
a variable light chain region comprising SEQ ID NO: 101 and a variable heavy region comprising SEQ ID NO: 105; and
a variable light chain region comprising SEQ ID NO: 109 and a variable heavy region comprising SEQ ID NO: 113.

In various embodiments: the TCE comprises any one of SEQ ID NOs: 931-934.

In some embodiments, the methods comprise administering to the subject, any of the oncolytic viruses described herein; and, simultaneously or subsequently, administering to the subject a TCE described herein (e.g., TCE-CD19). In some embodiments, the TCE is administered at least or about 1, 2, 3, 4, 5, 6, 7, 8, 9, 10, 11, 12, 13, 14, 15, 26, 17, 18, 19, or 20 days after administration of the oncolytic virus. In some embodiments, the TCE is administered at least or about 1, 2, 3, 4, 5, 6, 7, 8, 9, or 10 weeks after administration of the oncolytic virus. In some embodiments, the TCE is administered at least or about 1, 2, 3, 4, 5, 6, 7, 8, 9, 10, 11, 12, or 13 months after administration of the oncolytic virus.

In some embodiments, the solid tumor includes any one or more of: a sarcoma (e.g., tumors in a blood vessel, bone, fat tissue, ligament, lymph vessel, muscle or tendon), a carcinoma (e.g., tumors that form in epithelial cells), adrenocortical carcinoma, non-small cell lung carcinoma, gall bladder cancer, pancreatic cancer, prostate cancer, and urinary bladder cancer, gastric cancer, bone cancer, breast cancer, cervical cancer, colon cancer, rectal cancer, endometrial cancer, esophageal cancer, skin cancer, lung cancer, ovarian cancer, testicular cancer, thyroid cancer, etc., a metastases of one or more of these cancers, or a subpopulation of one or more of these or other cancers. In some embodiments, the solid tumor includes any cancer cell expressing CD19, HER2,

The 5′ ITR can comprise about 1000, 1500, 2000, 2500, 3000, 3500, 4000, 4500, 5000, 5500, 6000, 6500, 7000, 7500, 8000, 8500, 9000, 9500, 10000, 10050, or 11,000 nucleotides (nts) on the 5′ end of SEQ ID NO:1 or 2, and the 3′ ITR can comprise about 1000, 1500, 2000, 2500, 3000, 3500, 4000, 4500, 5000, 5500, 6000, 6500, 7000, 7500, 8000, 8500, 9000, 9500, 10000, 10050, or 110000 nucleotides (nts) on the 3′ end of SEQ ID NO: 1 or 2. In some embodiments, the 5′ ITR comprises or consists of nucleotides (nt) 1-5,013 of SEQ ID NO:1, and the 3′ ITR comprises or consists of nucleotides (nt) 178,074-182,474 of SEQ ID NO:1. In some embodiments, the 5′ ITR comprises or consists of nucleotides (nt) 1-4,054 of SEQ ID NO:2, and the 3′ ITR comprises or consists of nucleotides (nt) 185,351-189,404 of SEQ ID NO:2. In some embodiments, the 5′ ITR comprises or consists of nucleotides (nt) 1-5,013 of SEQ ID NO:5, and the 3′ ITR comprises or consists of nucleotides (nt) 189,901-190,949 of SEQ ID NO:5.

The chimeric oncolytic poxviruses as described herein include transgene (e.g., encoding human a truncated human CD19 (CD19t) that lacks a functional signaling domain, but includes the extracellular domain and transmembrane domain). In some embodiments, the truncated human CD19 comprises the amino acid sequence (or a sequence at least 95%, 97%, 98% or 99% identical to) MPPPRLLFFLLFLTPMEVRPEEPLVVKVEEGDNAVLQCLKGTSDGPTQQLTWSRE SPLKPFLKLSLGLPGLGIHMRPLAIWLFIFNVSQQMGGFYLCQPGPPSEKAWQPG WTVNVEGSGELFRWNVSDLGGLGCGLKNRSSEGPSSPSGKLMSPKLYVWAKDR PEIWEGEPPCVPPRDSLNQSLSQDLTMAPGSTLWLSCGVPPDSVSRGPLSWTHVH PKGPKSLLSLELKDDRPARDMWVMETGLLLPRATAQDAGKYYCHRGNLTMSFH LEITARPVLWHWLLRTGGWKVSAVTLAYLIFCLCSLVGILHLQRALVLRRKR (SEQ ID NO: 3). In some cases, the CD19t comprises or consists of amino acids 22-323 of SEQ ID NO: 3 (e.g., SEQ ID NO:4).

Amino acids 1-21 of SEQ ID NO: 3 are a signaling domain and can be replaced with a different signaling domain. Thus, the oncolytic virus comprises a sequence comprising a nucleotide sequence encoding a truncated human CD19 operably linked to an expression control sequence (e.g., an early promoter).

In some embodiments, the truncated human CD19 comprises or consists of the amino acid sequence (or a sequence at least 95%, 97%, 98% or 99% identical to)

(SEQ ID NO: 4)
EPLVVKVEEGDNAVLQCLKGTSDGPTQQLTWSRESPLKPFLKLSLGLPG
LGIHMRPLAIWLFIFNVSQQMGGFYLCQPGPPSEKAWQPGWTVNVEGSG
ELFRWNVSDLGGLGCGLKNRSSEGPSSPSGKLMSPKLYVWAKDRPEIWE
GEPPCVPPRDSLNQSLSQDLTMAPGSTLWLSCGVPPDSVSRGPLSWTHV
HPKGPKSLLSLELKDDRPARDMWVMETGLLLPRATAQDAGKYYCHRGNL
TMSFHLEITARPVLWHWLLRTGGWKVSAVTLAYLIFCLCSLVGILHLQR
ALVLRRKR.

In some embodiments, the CD19t comprises an amino acid sequence that comprises or consists of SEQ ID NOs: 3-4. In some embodiments, the CD19t operably linked to a promoter, optionally wherein the promoter is a synthetic early promoter.

Sequence identity for nucleotide and amino acid sequences are calculated using BLAST 2.0 with the default parameters. The percent sequence identity refers to a global alignment between the sequences.

In some embodiments, the recombinant oncolytic virus that includes a transgene, e.g., a transgene in an expression cassette wherein the transgene encodes all or a portion of human CD19 (UniProt ID: P15391). The expressed portion of CD19 a portion that can be expressed on the cell surface and can be recognized by an anti-CD19 antibody.

Also described herein are methods of treating a subject having a solid tumor comprising:
administering to the subject an effective amount of an oncolytic virus expressing a human truncated CD19 (CD19t); and
administering to the subject an effective amount of a CD19-targeting therapy that binds to CD19t;
wherein the nucleotide sequence of the oncolytic virus expressing CD19t (OV19t) comprises:
(a) an oncolytic virus nucleotide sequence encoding at least 25, 50, 60, 70, 80, 90, 100, 110, 120, or 121 of SEQ ID NOs: 654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and 928; and
(b) a transgene comprising a nucleotide sequence encoding the CD19t.

Also described herein are methods of treating a subject having a solid tumor comprising:
administering to the subject an effective amount of an oncolytic virus expressing a truncated CD19 (CD19t), wherein the nucleotide sequence of oncolytic virus (OV19t) comprises:
(a) an oncolytic virus nucleotide sequence; and
(b) a nucleotide sequence encoding a truncated human CD19 (CD19t); and
administering to the subject an effective amount of a therapy that binds to the CD19t;
   wherein the oncolytic virus nucleotide sequence comprises a nucleotide sequence that is at 90%, 95%, 96%, 97%, 98%, 99%, 99.5%, 99.9%, or 100% identical to or has no more than 300 single nucleotide changes compared to:
   a) SEQ ID NO: 2 over the entire length of SEQ ID NO: 2, but lacks a functional JR2 gene sequence of SEQ ID NO: 2;
   b) SEQ ID NO: 2 over the entire length of SEQ ID NO: 2, but lacks a functioning JR2 gene sequence, the 5′ ITR sequence and the 3′ ITR sequence of SEQ ID NO: 2 (e.g., a nucleotide sequence that is at 90%, 95%, 96%, 97%, 98%, 99%, 99.5%, 99.9%, or 100% identical to nt 4,055-77,602 of SEQ ID NO:2 and a nucleotide sequence that is at 90%, 95%, 96%, 97%, 98%, 99%, 99.5%, 99.9%, or 100% identical to nt 78,137-158,350 of SEQ ID NO:2); or
   c) nucleotides 4,055-185,350 of SEQ ID NO:2 over the entire length of nucleotides 4,055-185,350 SEQ ID NO: 2, but all or a portion of the J2R sequence;
optionally, further comprising administering to the subject an effective amount of a CD19-targeting therapy that binds to the CD19t.

In various embodiments of any of the methods described herein: the OV19t does not encode functional thymidine kinase; and/or the OV19t does not encode any of: an AFP, a C125, a BCMA, a BCMAt, a CD20, a CD33, a CD22, a CD123, a CD30, a CD38, a GPC-3, a CEA, a HER2, a GD2, a PSMA, a Claudin 18.2, a EpCAM, a GD2, a MSLN, an EGFR, an EGFRVIII, a Trop-2, a c-MET, a Nectin-4, a CD79b, a CCK4, a GPA33, a HLA-2, a CLEC12A, a p-cadherin, a TDO2, a MART-I, a MUCI, a Pmel 17, a MAGE-I, a TRP-1, a TRP-2, a NY-ESQ, a PSA, a CDK4, a BC225, a CA 125, a MG7-Ag, a NY—CO—I, a RCAS 1, a SDCCAG16, a TAAL6, and a TAG72, and optionally functional variants of one or more thereof; the oncolytic virus nucleotide sequence has no modifications in the coding regions comprising SEQ ID NOs: XX1-XX275, or wherein any modifications within the coding regions (SEQ ID NOs: XX1-XX275) do not change the amino acid sequence of the encoded protein; the oncolytic virus nucleotide sequence comprises a nucleotide sequence that is at least 90%, 95%, 96%, 97%, 98%, 99%, 99.5%, 99.9%, 99.99%, or 100% identical to or has no more than 100 single nucleotide changes compared to: SEQ ID NO: 2 over the entire length of SEQ ID NO: 2, but lacks all or a portion of the JR2 gene sequence and comprises nucleotide sequences that encode the proteins having SEQ ID NOs: 654-928, or a variant thereof having 1, 2, 3, 4, or 5 single amino acid substitutions; the OV19t comprises a nucleotide sequence has no more than 100 single nucleotide changes compared to: SEQ ID NO: 1 excepting the 5′ ITR and the 3′ ITR over the entire length of SEQ ID NO: 1 excepting the 5′ ITR and the 3′ ITR, and encodes proteins having the amino acid sequences of SEQ ID NOs: SEQ ID NO:654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and 928, or a variant thereof having 1, 2, 3, 4, or 5 single amino acid substitutions;
the OV19t comprises a nucleotide sequence has no more than 100 single nucleotide changes compared to: SEQ ID NO: 5 excepting the 5′ ITR and the 3′ ITR over the entire length of SEQ ID NO: 5 excepting the 5′ ITR and the 3′ ITR, and encodes proteins having the amino acid sequences of SEQ ID NOs: SEQ ID NO: 654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and928, or a variant thereof having 1, 2, 3, 4, or 5 single amino acid substitutions;
the OV19t comprises a nucleotide sequence:
   a) that is at least 90%, 95%, 96%, 97%, 98%, 99%, 99.5%, 99.9% or 100% identical to or has no more than 100 single nucleotide changes compared to: SEQ ID NO: 1 over the entire length of SEQ ID NO: 1, but lacks the 5′ ITR sequence and the 3′ ITR sequence of SEQ ID NO: 1; and
   b) encodes proteins having the amino acid sequences of SEQ ID NOs: 654-928, or a variant of thereof having 1, 2, 3, 4, or 5 single amino acid substitutions;
   optionally, wherein the 5′ ITR comprises or consists of nucleotides (nt) 1-5,013 of SEQ ID NO:1, and the 3′ ITR comprises or consists of nucleotides (nt) 178,074-182,474 of SEQ ID NO:1;
   the OV19t comprises a nucleotide sequence:
   a) that is at least 90%, 95%, 96%, 97%, 98%, 99%, 99.5%, 99.9% or 100% identical to or has no more than 100 single nucleotide changes compared to: SEQ ID NO: 5 over the entire length of SEQ ID NO: 5, but lacks the 5′ ITR sequence and the 3′ ITR sequence of SEQ ID NO: 5; and
   b) encodes proteins having the amino acid sequences of SEQ ID NOs: 654-928, or a variant of thereof having 1, 2, 3, 4, or 5 single amino acid substitutions;
   optionally, wherein the 5′ ITR comprises or consists of nucleotides (nt) 1-5,013 of SEQ ID NO:5, and the 3′ ITR comprises or consists of nucleotides (nt) 189,901-190,949 of SEQ ID NO:5.
the oncolytic virus sequence is at least 90%, 95%, 96%, 97%, 98%, 99%, 99.5%, 99.9% or 100% identical to or has no more than 300 single nucleotide changes compared to nt 4,055-77,602 of SEQ ID NO:2 and nt 78, 137-158,350 SEQ ID NO: 2 over the entire length of nt 4,055-77,602 of SEQ ID NO:2 and nt 78,137-158,350 SEQ ID NO: 2;
the OV19t is at least 90%, 95%, 96%, 97%, 98%, 99%, 99.5%, 99.9% or 100% identical to or has no more than 300 single nucleotide changes compared to: SEQ ID NO: 2 over the entire length of SEQ ID NO: 2 except that the nucleotide sequence encoding CD19t and a promoter sequence for expressing CD19t replaces at least 10 contiguous nucleotides of the JR2 gene sequence;
at least about 10, 20, 30, 40, 50, 60, 70, 80, 90, 100, 125, 150, 175, 200, 225, 250, 275, 300, 325, 250, 275, 400, 425, 450, 475, or 500 contiguous nucleotides of the J2R gene sequence (nt 77,603-78,136 of SEQ ID NO:2) have been deleted;
the deletion comprises nucleotides 77,682-78,084 of SEQ ID NO:2;
the nucleotide sequence encoding CD19t is inserted into a noncoding region of SEQ ID NO:2;
the nucleotide sequence encoding CD19t encodes the extracellular domain and the transmembrane domain of CD19;
the nucleotide sequence encoding CD19t does not encode the entirety of the cytoplasmic domain of CD19;
the CD19t comprises an amino acid sequence that comprises or consists of SEQ ID NO: 3 or 4;
the nucleotide sequence encoding CD19t is operably linked to a promoter;
the therapy that binds to the CD19t is a TCE comprising a domain that binds CD19 and a domain that binds CD3;
the domain that binds CD19 is a CD19-targeted scFv and the domain that binds CD3 is a CD3-targeted scFv;
the CD19-targeting therapy comprises:
   (a) a CD19 target domain comprising:
   a variable heavy (VH) chain as set forth in Table 5, or a variant thereof having 1, 2, 3, 4, or 5 single amino acid modifications (e.g., conservative substitutions), wherein the modifications are not in a CDR region, and
   a variable light (VL) chain as set forth in Table 5, or a variant thereof having 1, 2, 3, 4, or 5 single amino acid modifications (e.g., conservative substitutions), wherein the modifications are not in a CDR region; and
   (b) a CD3 target domain comprising:
   a variable heavy (VH) chain as set forth in Table 6, or a variant thereof having 1, 2, 3, 4, or 5 single amino acid modifications (e.g., conservative substitutions), wherein the modifications are not in a CDR region, and
   a variable light (VL) chain as set forth in Table 6, or a variant thereof having 1, 2, 3, 4, or 5 single amino acid modifications (e.g., conservative substitutions), wherein the modifications are not in a CDR region;
the TCE comprises any one of the constructs set forth in Table 4;
the CD19-targeting therapy comprises a population of autologous or allogeneic human immune cells expressing a chimeric antigen receptor (CAR) or harboring a nucleic acid encoding a CAR, wherein the CAR comprises a CD19-targeting domain comprising a CD19-targeted scFv (e.g., as set forth in Tables 4-6), a spacer (e.g., as set forth in Table 2), a transmembrane domain (e.g., as set forth in Table 1), a costimulatory domain (e.g., as set forth in Table 3), and a CD3z signaling domain (e.g., as set forth in Table 3).

In some embodiments of any of the methods described herein, the solid tumor comprises HER2-positive cells. In some embodiments of any of the methods described herein, the method further comprises administering an effective amount of a population immune cells expressing a HER2 CAR (e.g., a CAR comprising SEQ ID NO: 57, or a variant thereof having 1, 2, 3, 4, or 5 amino acid substitutions) or harboring a nucleic acid encoding a CAR (e.g., HER2 CAR T cells).

Also described herein are oncolytic viruses expressing a truncated CD19 (OV19t), the nucleotide sequence of OV19t comprising:
(a) an oncolytic virus nucleotide sequence; and
(b) a nucleotide sequence encoding a truncated human CD19 (CD19t);
   wherein the oncolytic virus nucleotide sequence encodes at least 25, 50, 60, 70, 80, 90, 100, 110, 120, or 121 of SEQ ID NO: 654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and928; and/or
   wherein the OV19t nucleotide sequence is at least 90%, 95%, 96%, 97%, 98%, 99%, 99.5%, 99.9% identical to or has no more than 300 single nucleotide changes compared to:
      i) nucleotides 5,014-178,073 of SEQ ID NO: 1 over the entire length of nucleotides 5,014-178,073 SEQ ID NO: 1;
      ii) nucleotides 7,000-178,000 of SEQ ID NO:1 over the entire length of nucleotides 7,000-178,000 SEQ ID NO: 1;
      iii) nucleotides 8,000-177,000 of SEQ ID NO: 1 over the entire length of nucleotides 8,000-177,000 SEQ ID NO: 1;
      iv) nucleotides 10,000-175,000 of SEQ ID NO: 1 over the entire length of nucleotides 10,000-175,000 SEQ ID NO: 1;
      v) nucleotides 5,014-186,900 of SEQ ID NO:5 over the entire length of nucleotides 5,014-186,900 SEQ ID NO: 5;
      vi) nucleotides 7,000-186,000 of SEQ ID NO:5 over the entire length of nucleotides 7,000-186,000 SEQ ID NO: 5;
      vii) nucleotides 8,000-185,000 of SEQ ID NO: 1 over the entire length of nucleotides 8,000-185,000 SEQ ID NO: 5; or
      viii) nucleotides 10,000-184,000 of SEQ ID NO:1 over the entire length of nucleotides 10,000-184,000 SEQ ID NO: 5.

In some embodiments, the OV19t nucleotide sequence is at least 95%, 96%, 97%, 98%, 99%, 99.5%, 99.6%, 99.7%, 99.8%, 99.9%, 99.99%, or 100% identical to or has no more than 100 single nucleotide changes compared to:
i) SEQ ID NO: 1 over the entire length of SEQ ID NO: 1;
ii) SEQ ID NO: 1 over the entire length of SEQ ID NO: 1, but lacks the 5′ ITR sequence and the 3′ ITR sequence of SEQ ID NO: 1;
iii) nucleotides 5,014-178,073 of SEQ ID NO: 1 over the entire length of nucleotides 5,014-178,073 SEQ ID NO: 1;
iv) nucleotides 7,000-178,000 of SEQ ID NO:1 over the entire length of nucleotides 7,000-178,000 SEQ ID NO: 1;
v) nucleotides 8,000-177,000 of SEQ ID NO: 1 over the entire length of nucleotides 8,000-177,000 SEQ ID NO: 1; or
vi) nucleotides 10,000-175,000 of SEQ ID NO:1 over the entire length of nucleotides 10,000-175,000 SEQ ID NO: 1.

In some embodiments, any nucleotide modifications in the oncolytic virus nucleotide sequence do not change the amino acid sequence of encoded proteins having an amino acid sequences SEQ ID NO: 654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and928.

In some embodiments, any nucleotide modifications in the oncolytic virus nucleotide sequence do not change the amino acid sequence of encoded proteins having an amino acid sequences SEQ ID NO: 654-928.

In some embodiments, the OV19t does not encode any of an AFP, a C125, a CD19, a CD19t, a CD20, a CD33, a CD22, a CD123, a CD30, a CD38, a GPC-3, a CEA, a HER2, a GD2, a PSMA, a Claudin 18.2, a EpCAM, a GD2, a MSLN, an EGFR, an EGFRVIII, a Trop-2, a c-MET, a Nectin-4, a CD79b, a CCK4, a GPA33, a HLA-2, a CLEC12A, a p-cadherin, a TDO2, a MART-I, a MUCI, a Pmel 17, a MAGE-I, a TRP-1, a TRP-2, a NY-ESQ, a PSA, a CDK4, a BC225, a CA 125, a MG7-Ag, a NY—CO—I, a RCAS 1, a SDCCAG16, a TAAL6, and a TAG72, and optionally functional variants of one or more thereof.

In some embodiments, the nucleotide sequence encoding CD19t encodes the extracellular domain and the transmembrane domain of CD19.

In some embodiments, the nucleotide sequence encoding CD19t does not encode the entirety of the cytoplasmic domain of CD19.

In some embodiments, the CD19t comprises an amino acid sequence that comprises or consists of SEQ ID NO: 3 or 4.

In some embodiments, the nucleotide sequence encoding CD19t is operably linked to a promoter.

Described herein, inter alia, are oncolytic viruses expressing a truncated CD19 (OV19t) comprising a nucleotide sequence that is at least 90%, 95%, 96%, 97%, 98%, 99%, 99.5%, 99.6%, 99.7%, 99.8%, 99.9%, 99.99%, or 100% identical to or has no more than 100 single nucleotide changes compared to nucleotides 6,000-176,000 of SEQ ID NO: 1 over the entire length of nucleotides 6,000-176,000 of SEQ ID NO: 1. Described herein, inter alia, are oncolytic viruses comprising a nucleotide sequence that has no more than 300, 200, or 100 single nucleotide changes compared to nucleotides 6,000-176,000 of SEQ ID NO: 1.

Described herein, inter alia, are oncolytic viruses expressing a truncated CD19 (OV19t) comprising a nucleotide sequence that is at least 90%, 95%, 96%, 97%, 98%, 99%, 99.5%, 99.6%, 99.7%, 99.8%, 99.9%, 99.99%, or 100% identical to or has no more than 100 single nucleotide changes compared to nucleotides 5,014-186,900 of SEQ ID NO: 5 over the entire length of nucleotides 5,014-186,900 of SEQ ID NO: 5. Described herein, inter alia, are oncolytic viruses comprising a nucleotide sequence that has no more than 300, 200, or 100 single nucleotide changes compared to nucleotides 5,014-186,900 of SEQ ID NO: 5.

Described herein, inter alia, are oncolytic viruses expressing a truncated CD19 (OV19t) comprising a nucleotide sequence that is at least 90%, 95%, 96%, 97%, 98%, 99%, 99.5%, 99.6%, 99.7%, 99.8%, 99.9%, 99.99%, or 100% identical to or has no more than 500, 450, 400, 350, 300, 250, 200, 150, 100, 50, 25, 10, 5, or 1 single nucleotide changes compared to nucleotides 5,000-175,000 of SEQ ID NO: 1 over the entire length of nucleotides 5,000-175,000 of SEQ ID NO: 1. Described herein, inter alia, are oncolytic viruses expressing a truncated CD19 (OV19t) comprising a nucleotide sequence that has no more than 500, 450, 400, 350, 300, 250, 200, 150, 100, 50, 25, 10, 5, or 1 single nucleotide changes compared to nucleotides 5,000-175,000 of SEQ ID NO: 1. Described herein, inter alia, are oncolytic viruses expressing a truncated CD19 (OV19t) comprising a nucleotide sequence that has no more than 500, 450, 400, 350, 300, 250, 200, 150, 100, 50, 25, 10, 5, or 1 single nucleotide changes compared to nucleotides 5,000-175,000 of SEQ ID NO: 5.

Also described herein are oncolytic viruses expressing a truncated CD19 (OV19t), the nucleotide sequence of OV19t comprising:
(a) an oncolytic virus nucleotide sequence; and
(b) a nucleotide sequence encoding a truncated human CD19 (CD19t);
   wherein the oncolytic virus nucleotide sequence encodes at least 25, 50, 60, 70, 80, 90, 100, 110, 120, or 121 of SEQ ID NO: 654, 670, 679, 680, 681, 685, 687, 689, 694, 705, 706, 708, 716, 718, 719, 729, 730, 732, 734, 735, 740, 743, 744, 745, 746, 757, 761, 763, 764, 769, 771, 773, 776, 781, 782, 783, 791, 793, 795, 796, 798, 799, 800, 802, 803, 804, 805, 806, 807, 808, 809, 810, 811, 813, 814, 818, 821, 822, 823, 825, 826, 827, 828, 829, 830, 831, 834, 835, 839, 845, 846, 847, 848, 850, 851, 852, 856, 859, 864, 865, 869, 872, 873, 875, 876, 877, 878, 879, 880, 881, 882, 884, 885, 886, 887, 889, 891, 893, 894, 896, 897, 905, 906, 907, 910, 911, 912, 913, 914, 916, 917, 918, 919, 920, 921, 922, 923, 924, 925, 927, and928; and/or
   wherein the OV19t nucleotide sequence comprises nucleotides 6,000-176,000 of SEQ ID NO: 1 or a variant thereof with up to 100, 200, or 300 nucleotide substitutions.

In some embodiments, the OV19t nucleotide sequence comprises nucleotides 5,014-178,073 of SEQ ID NO:1 or a variant thereof with up to 100 nucleotide substitutions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

In some embodiments, the OV19t nucleotide sequence comprises nucleotides 6,000-176,000 of SEQ ID NO:1 or a variant thereof with up to 100 nucleotide substitutions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

In some embodiments, the oncolytic virus comprises at least 25, 50, 75, 100, 125, 150, 175, 200, 225, or 250 of SEQ ID NO: XX1-XX275.

In some embodiments, the OV19t does not encode any of an AFP, a C125, a CD19, a CD19t, a CD20, a CD33, a CD22, a CD123, a CD30, a CD38, a GPC-3, a CEA, a HER2, a GD2, a PSMA, a Claudin 18.2, a EpCAM, a GD2, a MSLN, an EGFR, an EGFRVIII, a Trop-2, a c-MET, a Nectin-4, a CD79b, a CCK4, a GPA33, a HLA-2, a CLEC12A, a p-cadherin, a TDO2, a MART-I, a MUCI, a Pmel 17, a MAGE-I, a TRP-1, a TRP-2, a NY-ESQ, a PSA, a CDK4, a BC225, a CA 125, a MG7-Ag, a NY—CO—I, a RCAS 1, a SDCCAG16, a TAAL6, and a TAG72, and optionally functional variants of one or more thereof.

In some embodiments, the nucleotide sequence encoding CD19t encodes the extracellular domain and the transmembrane domain of CD19. In some embodiments, the nucleotide sequence encoding CD19t does not encode the entirety of the cytoplasmic domain of CD19. In some embodiments, the CD19t comprises an amino acid sequence that comprises or consists of SEQ ID NO: 3 or 4.

In some embodiments, the nucleotide sequence encoding CD19t is operably linked to a promoter.

Also described herein are oncolytic viruses expressing a truncated CD19 (OV19t) comprising nucleotides 5,014-178,073 of SEQ ID NO: 1 or a variant thereof with up to 100 nucleotide modifications (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

Also described herein are oncolytic viruses expressing a truncated CD19 (OV19t) comprising nucleotides 5,014-186,900 of SEQ ID NO: 5 or a variant thereof with up to 100 nucleotide modifications (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

Also described herein are oncolytic viruses expressing a truncated CD19 (OV19t) comprising nucleotides 6,000-177,000 of SEQ ID NO: 1 or a variant thereof with up to 100 nucleotide modifications (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

Also described herein are oncolytic viruses expressing a truncated CD19 (OV19t) comprising nucleotides 6,000-184,000 of SEQ ID NO: 5 or a variant thereof with up to 100 nucleotide modifications (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotide substitutions).

An oncolytic virus described herein (e.g., OV19t) can be administered in single or repeated doses. An OV19t can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, preservatives and antioxidants can also be present in the compositions.

Pharmaceutical formulations can be prepared according to any method known to the art for the manufacture of pharmaceuticals. Formulations of the compositions of the invention include those suitable for intradermal, subcutaneous, intravenous, transdermal, intraperitoneal, intramuscular, pulmonary, and/or parenteral administration. The formulations may conveniently be presented in unit dosage form and may be prepared by any methods well known in the art of pharmacy. The amount of active ingredient (e.g., an OV19t) which can be combined with a carrier to produce a single dosage form will vary depending upon the host being treated, the particular mode of administration, e.g., subcutaneous or intravenous. The amount of active ingredient which can be combined with a carrier to produce a single dosage form will generally be that amount of the compound which produces a therapeutic effect, e.g., expression of a CD19 or CD19t molecule on the surface of an infected cell, e.g., an infected solid tumor cell. Useful carriers are well known in the art and can include phosphate buffered saline solutions, water, liposomes, various types of wetting agents, sterile solutions, etc. Preparations for parenteral administration include sterile aqueous or non-aqueous solutions, or suspensions. Examples of non-aqueous solvents are propylene glycol, polyethylene glycol, and injectable organic esters such as ethyl oleate. Aqueous carriers include water, aqueous solutions, or suspensions, including saline and buffered media. Parenteral vehicles include sodium chloride solution, Ringer's dextrose, dextrose and sodium chloride, or lactated Ringer's. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the composition might comprise proteinaceous carriers, like, e.g., serum albumine or immunoglobuline, preferably of human origin.

Generally, an “effective amount” means a dose that produces the effects for which it is administered, e.g., an amount of an OV19t described herein sufficient to infect at least one solid tumor cell or cancer cell or sufficient to express CD19t on the surface of at least one solid tumor cell or cancer cell. The exact dose will depend on the purpose of the treatment, and will be ascertainable by one skilled in the art using known techniques. The attending physician and clinical factors will determine the dosage regimen. As is well known in the medical arts, dosages for any one pediatric patient depends upon many factors, including the pediatric patient's size, body surface area, age, body weight, the particular compound to be administered, sex, time and route of administration, general health status, and other drugs being administered concurrently.

Bispecific T cell Engagers (TCEs)

In some embodiments, the methods described herein comprise the use of TCEs (e.g., agents that binds to CD3 and to CD19 or CD19t).

TCEs have emerged as a technology that is capable of re-directing cytotoxic T-cells, independent of their natural T-cell receptor (TCR) specificity, to tumor antigens [24]. However, a fundamental challenge for designing effective TCE therapies for solid tumors is the identification of tumor selective targeting antigens [24]. The combination of treating a subject with an OV19t and a CD19-TCE, as described herein, overcomes this challenge and leads to the efficient killing of tumor cells.

There are a number of CD19-TCEs known in the art that can be used in any of the methods described herein. For example, a useful CD19 TCE can include BLINCYTO® (blinatumomab). Blinatumomab, a BiTE composed of two scFv domains (one targeting CD19 on malignant B-cells and the other targeting CD3 on T-cells) connected by a linker, to induce a cytolytic synapse between a T-cell and a CD19-positive tumor cell, is FDA approved for treatment of cancers, such as relapsed and refractory acute lymphoblastic leukemia [24].

A TCE described herein can be administered in single or repeated doses.

A TCE can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, preservatives and antioxidants can also be present in the compositions.

Pharmaceutical formulations can be prepared according to any method known to the art for the manufacture of pharmaceuticals. Formulations of the compositions of the invention include those suitable for intradermal, subcutaneous, intravenous, transdermal, intraperitoneal, intramuscular, pulmonary, and/or parenteral administration. The formulations may conveniently be presented in unit dosage form and may be prepared by any methods well known in the art of pharmacy. The amount of active ingredient (e.g., a CD19 TCE) which can be combined with a carrier to produce a single dosage form will vary depending upon the host being treated, the particular mode of administration, e.g., subcutaneous or intravenous. The amount of active ingredient which can be combined with a carrier to produce a single dosage form will generally be that amount of the compound which produces a therapeutic effect, e.g., an antigen specific T cell response. Useful carriers are well known in the art and can include phosphate buffered saline solutions, water, liposomes, various types of wetting agents, sterile solutions, etc. Preparations for parenteral administration include sterile aqueous or non-aqueous solutions, or suspensions. Examples of non-aqueous solvents are propylene glycol, polyethylene glycol, and injectable organic esters such as ethyl oleate. Aqueous carriers include water, aqueous solutions, or suspensions, including saline and buffered media. Parenteral vehicles include sodium chloride solution, Ringer's dextrose, dextrose and sodium chloride, or lactated Ringer's. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the composition might comprise proteinaceous carriers, like, e.g., serum albumine or immunoglobuline, preferably of human origin.

Generally, an “effective amount” means a dose that produces the effects for which it is administered, e.g., an amount of a CD19-TCE described herein sufficient to kill at least one cell expressing CD19 (e.g., a solid tumor cell successful infected by OV19t and expressing CD19t) or sufficient to illicit an immune response (e.g., an antigen specific T cell response). The exact dose will depend on the purpose of the treatment, and will be ascertainable by one skilled in the art using known techniques. The attending physician and clinical factors will determine the dosage regimen. As is well known in the medical arts, dosages for any one pediatric patient depends upon many factors, including the pediatric patient's size, body surface area, age, body weight, the particular compound to be administered, sex, time and route of administration, general health status, and other drugs being administered concurrently.

More information on formulations, dosage, and administration of TCEs is known in the art, for example, in US 2023/0235053 A1, WO 2018/204907, WO 2023/199235, and WO 2023/062188.

Examples of additional CD19 TCE constructs can include any one the following:

| Name | Organization/Company | Clinical Stage |
|---|---|---|
| AMG-562 | Amgen | Phase I |
| | | (NCT03571828) |
| A-319 | Generon (Shanghai) | Phase I |
| | | (NCT04056975) |
| AFM-11 | Affirmed | Suspended |
| CD3 × CD19 | Avacta | Preclinical |
| GNR-047 | IBC Generium | Preclinical |
| Next-generation | MacroGenics | Preclinical |
| CD19 × CD3 DART | | |
| ZW-38 | Merck; Zymeworks | Preclinical |
| B-193 | Shandong Danhong; | Preclinical |
| | Shanghai Yanyi | |
| 19-3-19 | SpectraMab | Preclinical |
| SV-202 | SYSVAX | Preclinical |
| TNB-486 | TeneoBio | Phase I |
| | | (NCT04594642) |
| CD3 × CD19 | Tianjin Chase Sun Jinboda | Preclinical |
| CD3 × CD19 | Wuhan YZY | Preclinical |

Adapted from Table 4 of Nie, S., et al. (2020) “Biology drives the discovery of bispecific antibodies as innovative therapeutics” Antibody Therapeutics, 3 (1): 18-62.

A TCE as described herein includes bispecific antibody constructs, which are recombinant protein constructs made from two flexibly linked antibody derived binding domains. One binding domain of a CD19-TCE is specific for CD19, CD19t, or a variant thereof; the second binding domain is specific for CD3 or a variant thereof (e.g., a functional variant). The TCEs and bispecific antibody constructs disclosed herein can be prepared by methods known in the art, for example, by methods disclosed in WO 2008/119657 and WO 2017/134140. TCE constructs are uniquely suited to transiently 10 connect T cells with target cells and, at the same time, potently activate the inherent cytolytic potential of T cells against target cells. First generation TCE constructs (see WO 99/54440 and WO 2005/040220) developed into the clinic as AMG 103 and AMG 110 were then modified by the provision of bispecific antibody constructs binding to a context independent epitope at the N-terminus of the CD3s chain (WO 2008/119567). TCE constructs binding to this CD3 epitope displayed cross-species specificity for human and Callithrix jacchus, Saguinus oedipus or Saimiri sciureus CD3e chain. Furthermore, binding this specific epitope instead of previously described epitopes for CD3 binders, reduces or eliminates non-specific activation of T cells compared to what was observed for the previous generation of T cell engaging antibodies. This reduction in T cell activation was connected with less or reduced T cell redistribution in patients, which was identified as a risk for side effects. Thus, in some embodiments, the portion of the TCE that targets CD3 can bind to CD3e (e.g., via a scFv targets to CD3e).

An increased half-life is generally useful in in vivo applications of TCEs and immunoglobulins in general, especially antibodies and antibody fragments of small size. Some approaches described in the art to achieve such effect comprise the fusion of the TCE or bispecific antibody construct to larger proteins, which preferably do not interfere with the therapeutic effect of the TCE or bispecific antibody construct. Examples for such further developments of bispecific T cell engagers comprise bispecific Fc-molecules e.g. described in US 2014/0302037, US 2014/0308285, WO 2014/144722, WO 2014/151910, WO 2015/048272, WO 2018/204907, and WO 2020/072306. Antibody constructs as described in WO 2008/119567 are likely to suffer from rapid clearance from the body; thus, whilst they are able to reach most parts of the body rapidly, and are quick to produce and easier to handle, their in vivo applications may be limited by their brief persistence in vivo. Prolonged administration by continuous intravenous infusion is used to achieve therapeutic effects because of the short in vivo half-life of this small, single chain molecule.

In some embodiments, a useful TCE can be a bispecific antibody construct that further comprises a half-life extending (HLE) moiety (e.g., a scFc domain, a heteroFc domain, or an albumin binding domain). In some embodiments, the N-terminus or the C-terminus of the HLE domain is connected to the TCE (e.g., the portion that binds CD3, e.g., the VH or VL of a CD3-targeted scFv). In some embodiments, the HLE domain is connected to the bispecific antibody construct via a linker.

In some embodiments, the TCE is a BiTE, wherein the BiTE further comprises a third domain comprising two polypeptide monomers, each comprises a hinge, a CH2 and a CH3 domain, wherein the two polypeptide monomers are linked to each other via a peptide linker. In another embodiment, the third domain comprises in an amino to carboxyl order hinge-CH2-CH3-linker-hinge-CH2-CH3. In one embodiment, the third domain is a half-life extended (HLE) domain.

A useful TCE can comprise an amino acid sequence having 90, 95, 96, 97, 98, 99, or 100% identity to any one of SEQ ID NOs: 931-934. A TCE can comprise or consist of an amino acid sequence of any one of SEQ ID NOs: 931-934 or a variant thereof having 1, 2, 3, 4, 5, 6, 7, 8, 9, 10, 15, 20, 25, or 30 amino acid modifications (e.g., substitutions), wherein the modifications are not in the CDR regions; preferably wherein the modifications are conservative.

TABLE 4
Examples of CD19-TCE Sequences

| | |
|---|---|
| CD19xCD3 scFv | DIQLTQSPASLAVSLGQRATISCKASQSVDYDGDSYLNWY |
| BLINCYTO | QQIPGQPPKLLIYDASNLVSGIPPRFSGSGSGTDFTLNIHPV |
| (with linker) | EKVDAATYHCQQSTEDPWTFGGGTKLEIKGGGGSGGGG |
| | SGGGGSQVQLQQSGAELVRPGSSVKISCKASGYAFSSYW |
| | MNWVKQRPGQGLEWIGQIWPGDGDTNYNGKFKGKATLT |
| | ADESSSTAYMQLSSLASEDSAVYFCARRETTTVGRYYYA |
| | MDYWGQGTTVTVSSGGGGSDIKLQQSGAELARPGASVK |
| | MSCKTSGYTFTRYTMHWVKQRPGQGLEWIGYINPSRGY |
| | TNYNQKFKDKATLTTDKSSSTAYMQLSSLTSEDSAVYYCA |
| | RYYDDHYCLDYWGQGTTLTVSSVEGGSGGSGGSGGSGG |
| | VDDIQLTQSPAIMSASPGEKVTMTCRASSSVSYMNWYQQ |
| | KSGTSPKRWIYDTSKVASGVPYRFSGSGSGTSYSLTISSME |
| | AEDAATYYCQQWSSNPLTFGAGTKLELK (SEQ ID NO: |
| | 931) |
| CD19xCD3 scFv | DIQLTQSPASLAVSLGQRATISCKASQSVDYDGDSYLNWY |
| BLINCYTO | QQIPGQPPKLLIYDASNLVSGIPPRFSGSGSGTDFTLNIHPV |
| (with linker and his- | EKVDAATYHCQQSTEDPWTFGGGTKLEIKGGGGSGGGG |
| tag) | SGGGGSQVQLQQSGAELVRPGSSVKISCKASGYAFSSYW |
| | MNWVKQRPGQGLEWIGQIWPGDGDTNYNGKFKGKATLT |
| | ADESSSTAYMQLSSLASEDSAVYFCARRETTTVGRYYYA |
| | MDYWGQGTTVTVSSGGGGSDIKLQQSGAELARPGASVK |
| | MSCKTSGYTFTRYTMHWVKQRPGQGLEWIGYINPSRGY |
| | TNYNQKFKDKATLTTDKSSSTAYMQLSSLTSEDSAVYYCA |
| | RYYDDHYCLDYWGQGTTLTVSSVEGGSGGSGGSGGSGG |
| | VDDIQLTQSPAIMSASPGEKVTMTCRASSSVSYMNWYQQ |
| | KSGTSPKRWIYDTSKVASGVPYRFSGSGSGTSYSLTISSME |
| | AEDAATYYCQQWSSNPLTFGAGTKLELKHHHHHH (SEQ |
| | ID NO: 932) |
| CD19 TCE + I2C0 | MDMRVPAQLLGLLLLWLRGARCDIVMTQSPLSLPVISGEP |
| | ASISCRSSQSLLHKNAFNYLDWYLQKPGQSPQLLIYLGSN |
| | RASGVPDRFSGSGSGTDFTLKISRVEAEDVGVYYCMQAL |
| | QTPFTFGCGTKVDIKGGGGSGGGGSGGGGSQVQLVESGG |
| | GVVQPGRSLRLSCAASGFTFSSYGMHWVRQAPGKCLEW |
| | VAVISYEGSNKYYAESVKGRFTISRDNSKNTLYLQMNSLR |
| | DEDTAVYYCARDRGTIFGNYGLEVWGQGTTVTVSSGGG |
| | GSEVQLVESGGGLVQPGGSLKLSCAASGFTFNKYAMNWV |
| | RQAPGKGLEWVARIRSKYNNYATYYADSVKDRFTISRDD |
| | SKNTAYLQMNNLKTEDTAVYYCVRHGNFGNSYISYWAY |
| | WGQGTLVTVSSGGGGSGGGGSGGGGSQTVVTQEPSLTVS |
| | PGGTVTLTCGSSTGAVTSGNYPNWVQQKPGQAPRGLIGG |
| | TKFLAPGTPARFSGSLLGGKAALTLSGVQPEDEAEYYCVL |
| | WYSNRWVFGGGTKLTVL (SEQ ID NO: 933) |
| CD19 TCE + I2C0 | MDMRVPAQLLGLLLLWLRGARCDIVMTQSPLSLPVISGEP |
| scFc | ASISCRSSQSLLHKNAFNYLDWYLQKPGQSPQLLIYLGSN |
| | RASGVPDRFSGSGSGTDFTLKISRVEAEDVGVYYCMQAL |
| | QTPFTFGCGTKVDIKGGGGSGGGGSGGGGSQVQLVESGG |
| | GVVQPGRSLRLSCAASGFTFSSYGMHWVRQAPGKCLEW |
| | VAVISYEGSNKYYAESVKGRFTISRDNSKNTLYLQMNSLR |
| | DEDTAVYYCARDRGTIFGNYGLEVWGQGTTVTVSSGGG |
| | GSEVQLVESGGGLVQPGGSLKLSCAASGFTFNKYAMNWV |
| | RQAPGKGLEWVARIRSKYNNYATYYADSVKDRFTISRDD |
| | SKNTAYLQMNNLKTEDTAVYYCVRHGNFGNSYISYWAY |
| | WGQGTLVTVSSGGGGSGGGGSGGGGSQTVVTQEPSLTVS |
| | PGGTVTLTCGSSTGAVTSGNYPNWVQQKPGQAPRGLIGG |
| | TKFLAPGTPARFSGSLLGGKAALTLSGVQPEDEAEYYCVL |
| | WYSNRWVFGGGTKLTVLGGGGDKTHTCPPCPAPELLGGP |
| | SVFLFPPKPKDTLMISRTPEVTCVVVDVSHEDPEVKFNWY |
| | VDGVEVHNAKTKPCEEQYGSTYRCVSVLTVLHQDWLNG |
| | KEYKCKVSNKALPAPIEKTISKAKGQPREPQVYTLPPSREE |
| | MTKNQVSLTCLVKGFYPSDIAVEWESNGQPENNYKTTPP |
| | VLDSDGSFFLYSKLTVDKSRWQQGNVFSCSVMHEALHNH |
| | YTQKSLSLSPGKGGGGSGGGGSGGGGSGGGGSGGGGSG |
| | GGGSDKTHTCPPCPAPELLGGPSVFLFPPKPKDTLMISRTP |
| | EVTCVVVDVSHEDPEVKFNWYVDGVEVHNAKTKPCEEQ |
| | YGSTYRCVSVLTVLHQDWLNGKEYKCKVSNKALPAPIEK |
| | TISKAKGQPREPQVYTLPPSREEMTKNQVSLTCLVKGFYP |
| | SDIAVEWESNGQPENNYKTTPPVLDSDGSFFLYSKLTVDK |
| | SRWQQGNVFSCSVMHEALHNHYTQKSLSLSPGK (SEQ ID |
| | NO: 934) |

BLINCYTO linker GSGGGGSQVQLQQSGAELVRPGSSVKISCKASGYAFSSY (SEQ ID NO: 935)

A useful TCE comprises a domain that binds CD19 and a domain that binds CD3. In some embodiments, the domain that binds CD19 is a CD19-targeted scFv and the domain that binds CD3 is a CD3-targeted scFv.

In some embodiments, a CD19-targeted scFv comprises:
(a) a heavy chain variable region (VH) comprising a VH complementarity determining region (CDR) 1 comprising a sequence that is at least 95% identical to a VH CDR1 amino acid sequence set forth in Table 5; a VH CDR2 comprising a sequence that is at least 95% identical to a VH CDR2 amino acid sequence set forth in Table 5, and a VH CDR3 comprising a sequence that is at least 95% identical to a VH CDR3 amino acid sequence set forth in Table 5; and
(b) a light chain variable region (VL) comprising a VL CDR1 comprising a sequence that is at least 95% identical to a VL CDR1 amino acid sequence set forth in Table 5, a VL CDR2 comprising a sequence that is at least 95% identical to a VL CDR2 amino acid sequence set forth in Table 5, and a VL CDR3 comprising a sequence that is at least 95% identical to a VL CDR3 amino acid sequence set forth in Table 5.

In some embodiments, a CD19-targeted scFv comprises a VH comprising or consisting of an amino acid sequence having at least 95% sequence identity to a VH amino acid sequence set forth in Table 5, and a VL comprising or consisting of an amino acid sequence having at least 95% sequence identity to a VL amino acid sequence set forth in Table 5.

A useful CD19-targeted scFv comprises any one of the following:
a variable light chain region having CDR1, CDR2, and CDR3 regions comprising SEQ ID NOs: 66-68 and a variable light chain region having CDR1, CDR2, and CDR3 regions comprising SEQ ID NOs: 70-72;
a variable light chain region having CDR1, CDR2, and CDR3 regions comprising SEQ ID NOs: 74-76 and a variable light chain region having CDR1, CDR2, and CDR3 regions comprising SEQ ID NOs: 78-80;
a variable light chain region having CDR1, CDR2, and CDR3 regions comprising SEQ ID NOs: 82-84 and a variable light chain region having CDR1, CDR2, and CDR3 regions comprising SEQ ID NOs: 86-88;
a variable light chain region comprising SEQ ID NO: 69 and a variable heavy region comprising SEQ ID NO: 73;
a variable light chain region comprising SEQ ID NO: 77 and a variable heavy region comprising SEQ ID NO: 81; and
a variable light chain region comprising SEQ ID NO: 85 and a variable heavy region comprising SEQ ID NO: 89.

TABLE 5
CD19-Binding TCE Sequences

| | |
|---|---|
| CD19 VL1 CDR1 | KASQSVDYDGDSYLN (SEQ ID NO: 66) |
| CD19 VL1 CDR2 | DASNLVS (SEQ ID NO: 67) |
| CD19 VL1 CDR3 | QQSTEDPWT (SEQ ID NO: 68) |
| CD19 VL1 | DIQLTQSPASLAVSLGQRATISCKASQSVDYDGDSYLNWY |
| | QQIPGQPPKLLIYDASNLVSGIPPRFSGSGSGTDFTLNIHPV |
| | EKVDAATYHCQQSTEDPWTFGGGTKLEIK (SEQ ID |
| | NO: 69) |
| CD19 VH1 CDR1 | SYWMN (SEQ ID NO: 70) |
| CD19 VH1 CDR2 | QJWPGDGDTNYNGKFKG (SEQ ID NO: 71) |
| CD19 VH1 CDR3 | RETTTVGRYYYAMDY (SEQ ID NO: 72) |
| CD19 VH1 | QVQLQQSGAELVRPGSSVKISCKASGYAFSSYWMNWVK |
| | QRPGQGLEWIGQIWPGDGDTNYNGKFKGKATLTADESSS |
| | TAYMQLSSLASEDSAVYFCARRETTTVGRYYYAMDYWG |
| | QGTTVTVSS (SEQ ID NO: 73) |
| CD19 VL2 CDR1 | RSSQSLLHKNAFNYLD (SEQ ID NO: 74) |
| CD19 VL2 CDR2 | LGSNRAS (SEQ ID NO: 75) |
| CD19 VL2 CDR3 | MQALQTPFT (SEQ ID NO: 76) |
| CD19 VL2 | DIVMTQSPLSLPVISGEPASISCRSSQSLLHKNAFNYLDWY |
| | LQKPGQSPQLLIYLGSNRASGVPDRFSGSGSGTDFTLKISR |
| | VEAEDVGVYYCMQALQTPFTFGCGTKVDIK (SEQ ID NO: |
| | 77) |
| CD19 VH2 CDR1 | SYGMH (SEQ ID NO: 78) |
| CD19 VH2 CDR2 | VISYEGSNKYYAESVKG (SEQ ID NO: 79) |
| CD19 VH2 CDR3 | DRGTIFGNYGLEV (SEQ ID NO: 80) |
| CD19 VH2 | QVQLVESGGGVVQPGRSLRLSCAASGFTFSSYGMHWVR |
| | QAPGKCLEWVAVISYEGSNKYYAESVKGRFTISRDNSKNT |
| | LYLQMNSLRDEDTAVYYCARDRGTIFGNYGLEVWGQGT |
| | TVTVSS (SEQ ID NO: 81) |
| CD19 VL3 CDR1 | RASQDVGTAV A (SEQ ID NO: 82) |
| CD19 VL3 CDR2 | WASTRHT (SEQ ID NO: 83) |
| CD19 VL3 CDR3 | QQYANFPLYT (SEQ ID NO: 84) |
| CD19 VL3 | EIVMTQSPAT LSVSPGERAT LSCRASQDVG |
| | TAVAWYQQKP GQAPRLLIYW ASTRHTGIPA |
| | RFSGSGSGTE FTLTISSLQS EDFAVYFCQQ YANFPLYTFG |
| | QGTKLEIK (SEQ ID NO: 85) |
| CD19 VH3 CDR1 | TYWIQ (SEQ ID NO: 86) |
| CD19 VH3 CDR2 | AVYPGDADTR YTQKFQG (SEQ ID NO: 87) |
| CD19 VH3 CDR3 | DAGLEYYALD Y (SEQ ID NO: 88) |
| CD19 VH3 | QVQLVQSGAE VKKPGASVKV SCKASGYTFT |
| | TYWIQWVRQA PGQRLEWMGA VYPGDADTRY |
| | TQKFQGRVTL TADRSASTAY MELSSLRSED |
| | TAVYYCGRDA GLEYYALDYW GQGTLVTVSS (SEQ ID |
| | NO: 89) |

In some embodiments, a CD3-targeted scFv comprises
(a) a heavy chain variable region (VH) comprising a VH complementarity determining region (CDR) 1 comprising a sequence that is at least 95% identical to a VH CDR1 amino acid sequence set forth in Table 6; a VH CDR2 comprising a sequence that is at least 95% identical to a VH CDR2 amino acid sequence set forth in Table 6, and a VH CDR3 comprising a sequence that is at least 95% identical to a VH CDR3 amino acid sequence set forth in Table 6; and
(b) a light chain variable region (VL) comprising a VL CDR1 comprising a sequence that is at least 95% identical to a VL CDR1 amino acid sequence set forth in Table 6, a VL CDR2 comprising a sequence that is at least 95% identical to a VL CDR2 amino acid sequence set forth in Table 6, and a VL CDR3 comprising a sequence that is at least 95% identical to a VL CDR3 amino acid sequence set forth in Table 6.

In some embodiments, a CD3-targeted scFv comprises a VH comprising or consisting of an amino acid sequence having at least 95% sequence identity to a VH amino acid sequence set forth in Table 6, and a VL comprising or consisting of an amino acid sequence having at least 95% sequence identity to a VL amino acid sequence set forth in Table 6.

In some embodiments, the CD3-targeted scFv comprises any one of the following:
a variable light chain region having CDR1, CDR2, and CDR3 regions comprising SEQ ID NOs: 90-92 and a variable light chain region having CDR1, CDR2, and CDR3 regions comprising SEQ ID NOs: 94-96;
a variable light chain region having CDR1, CDR2, and CDR3 regions comprising SEQ ID NOs: 98-100 and a variable light chain region having CDR1, CDR2, and CDR3 regions comprising SEQ ID NOs: 102-104;
a variable light chain region having CDR1, CDR2, and CDR3 regions comprising SEQ ID NOs: 106-108 and a variable light chain region having CDR1, CDR2, and CDR3 regions comprising SEQ ID NOs: 110-112;
a variable light chain region comprising SEQ ID NO: 93 and a variable heavy region comprising SEQ ID NO: 97;
a variable light chain region comprising SEQ ID NO: 101 and a variable heavy region comprising SEQ ID NO: 105; and
a variable light chain region comprising SEQ ID NO: 109 and a variable heavy region comprising SEQ ID NO: 113.

TABLE 6
CD3-Binding Portion TCE Sequences

| | |
|---|---|
| CD3 VL1 CDR1 | RASSSVSYMN (SEQ ID NO: 90) |
| CD3 VL1 CDR2 | DTSKVAS (SEQ ID NO: 91) |
| CD3 VL1 CDR3 | QQWSSNPLT (SEQ ID NO: 92) |
| CD3 VL1 | VDDIQLTQSPAIMSASPGEKVTMTCRASSSVSYMNWYQQ |
| | KSGTSPKRWIYDTSKVASGVPYRFSGSGSGTSYSLTISSME |
| | AEDAATYYCQQWSSNPLTFGAGTKLELK (SEQ ID NO: 93) |
| CD3 VH1 CDR1 | RYTMH (SEQ ID NO: 94) |
| CD3 VH1 CDR2 | YINPSRGYTNYNQKFKD (SEQ ID NO: 95) |
| CD3 VH1 CDR3 | YYDDHYCLDY (SEQ ID NO: 96) |
| CD3 VH1 | DIKLQQSGAELARPGASVKMSCKTSGYTFTRYTMHWVK |
| | QRPGQGLEWIGYINPSRGYTNYNQKFKDKATLTTDKSSST |
| | AYMQLSSLTSEDSAVYYCARYYDDHYCLDYWGQGTTLT |
| | VSS (SEQ ID NO: 97) |
| CD3 VL2 CDR1 | RSSTGAVTTSNYAN (SEQ ID NO: 98) |
| CD3 VL2 CDR2 | GTNKRAP (SEQ ID NO: 99) |
| CD3 VL2 CDR3 | ALWYSNLWV (SEQ ID NO: 100) |
| CD3 LC2 (VL2) | QTVVTQEPSL TVSPGGTVTL TCRSSTGAVT |
| | TSNYANWVQQ KPGQAPRGLI GGTNKRAPGT |
| | PARFSGSLLG GKAALTLSGV QPEDEAEYYC |
| | ALWYSNLWVF GGGTKLTVLG QPKAAPSVTL |
| | FPPSSEELQA NKATLVCLIS DFYPGAVTVA |
| | WKADSSPVKA GVETTTPSKQ SNNKYAASSY |
| | LSLTPEQWKS HRSYSCQVTH EGSTVEKTVA PTECS (SEQ |
| | ID NO: 101) |
| CD3 VH2 CDR1 | TYAMN (SEQ ID NO: 102) |
| CD3 VH2 CDR2 | RIRSKYNNYATYYAASVKG (SEQ ID NO: 103) |
| CD3 VH2 CDR3 | HGNFGNSYVSWFAY (SEQ ID NO: 104) |
| CD3 HC2 (VH2) | EVQLVESGGG LVQPGGSLRL SCAASGFTFN |
| | TYAMNWVRQA PGKGLEWVAR IRSKYNNYAT |
| | YYAASVKGRF TISRDDSKNS LYLQMNSLKT |
| | EDTAVYYCAR HGNFGNSYVS WFAYWGQGTL |
| | VTVSSASTKG PSVFPLAPCS RSTSESTAAL GCLVKDYFPE |
| | PVTVSWNSGA LTSGVHTFPA VLQSSGLYSL SSVVTVPSSS |
| | LGTKTYTCNV DHKPSNTKVD KRVESKYGPP |
| | CPPCPAPEAA GGPSVFLFPP KPKDTLMISR TPEVTCVVVD |
| | VSQEDPEVQF NWYVDGVEVH NAKTKPREEQ |
| | FNSTYRVVSV LTVLHQDWLN GKEYKCKVSN |
| | KGLPSSIEKT ISKAKGQPRE PQVYTLPPSQEEMTKNQVSL |
| | TCLVKGFYPSDIAVEWESNGQPENNYKTTPPVLDSDGSFL |
| | LYSKLTVDKSRWQEGNVFSCSVMHEALHNHYTQKSLSLS |
| | L GK (SEQ ID NO: 105) |
| CD3 VL3 CDR1 | RSSTGAVTTS NYAN (SEQ ID NO: 106) |
| CD3 VL3 CDR2 | GTNKRAP (SEQ ID NO: 107) |
| CD3 VL3 CDR3 | ALWYSNLWV (SEQ ID NO: 108) |
| CD3 VL3 | QAVVTQEPSL TVSPGGTVTL TCRSSTGAVT |
| | TSNYANWVQQ KPGQAPRGLI GGTNKRAPWT |
| | PARFSGSLLG DKAALTLSGA QPEDEAEYFC |
| | ALWYSNLWVF GGGTKLTVL (SEQ ID NO: 109) |
| CD3 VH3 CDR1 | TYAMN (SEQ ID NO: 110) |
| CD3 VH3 CDR2 | RIRSKYNNYA TYYADSVKD (SEQ ID NO: 111) |
| CD3 VH3 CDR3 | HGNFGNSYVS WFAY (SEQ ID NO: 112) |
| CD3 VH3 | EVQLVESGGG LVQPGGSLKL SCAASGFTFN TYAMNWV |
| | RQA SGKGLEWVGR IRSKYNNYATYYADSVKDRF TISRD |
| | DSKST LYLQMNSLKT EDTAVYYCVR HGNFGNSYVS WF |
| | AYWGQGTL VTVSS (SEQ ID NO: 113) |
| CD3 scFv 3 | EVQLVESGGG LVQPGGSLKL SCAASGFTFN TYAMNWV |
| | RQA SGKGLEWVGR IRSKYNNYAT |
| | YYADSVKDRF TISRDDSKST LYLQMNSLKT EDTAVYYC |
| | VR HGNFGNSYVS WFAYWGQGTL |
| | VTVSSGGGGS GGGGSGGGGS GGGGSQAVVT QEPSLTVS |
| | PG GTVTLTCRSS TGAVTTSNYA |
| | NWVQQKPGQA PRGLIGGTNK RAPWTPARFS GSLLGDK |
| | AAL TLSGAQPEDE AEYFCALWYS |
| | NLWVFGGGTK LTVL (SEQ ID NO: 114) |
| CD3 VL4 CDR1 | RSSTGAVTTS NYAN (SEQ ID NO: 115) |
| CD3 VL4 CDR2 | GTNKRAP (SEQ ID NO: 116) |
| CD3 VL4 CDR3 | ALWYSNLWV (SEQ ID NO: 117) |
| CD3 VL4 | QTVVTQEPSLTVSPGGTVTLTCRSSTGAVTTSNYANWFQQ |
| | KPGQAPRGLIGGTNKRAPGVPARFSGSLLGGKAALTLSGV |
| | QPEDEAEYYCALWYSNLWVFGGGTKVEIK (SEQ ID NO: |
| | 118) |
| CD3 VH4 CDR1 | TYAMN (SEQ ID NO: 119) |
| CD3 VH4 CDR2 | RIRSKYNNYA TYYADSVKD (SEQ ID NO: 120) |
| CD3 VH4 CDR3 | HGNFGNSYVS WAAY (SEQ ID NO: 121) |
| CD3 VH4 | QVQLVESGGGVVQPGRSLRLSCAASGFTFSTYAMNWVR |
| | QAPGKGLEWVARIRSKYNNYATYYADSVKDRFTISRDDS |
| | KNTLYLQMNSLRAEDTAVYYCARHGNFGNSYVSWAAYW |
| | GQGTLVTVSS (SEQ ID NO: 122) |
| CD3 VL5 CDR1 | GSSTGAVTSG NYPN (SEQ ID NO: 123) |
| CD3 VL5 CDR2 | GTKFLAP (SEQ ID NO: 124) |
| CD3 VL5 CDR3 | VLWYSNRWV (SEQ ID NO: 125) |
| CD3 VL5 | QTVVTQEPSLTVSPGGTVTLTCGSSTGAVTSGNYPNWVQ |
| | QKPGQAPRGLIGGTKFLAPGTPARFSGSLLGGKAALTLSG |
| | VQPEDEAEYYCVLWYSNRWVFGGGTKLTVL (SEQ ID |
| | NO: 126) |
| CD3 VH5 CDR1 | KYAMN (SEQ ID NO: 127) |
| CD3 VH5 CDR2 | RIRSKYNNYA TYYADSVKD (SEQ ID NO: 128) |
| CD3 VH5 CDR3 | HGNFGNSYIS YWAY (SEQ ID NO: 129) |
| CD3 VH5 | EVQLVESGGGLVQPGGSLKLSCAASGFTFNKYAMNWVR |
| | QAPGKGLEWVARIRSKYNNYATYYADSVKDRFTISRDDS |
| | KNTAYLQMNNLKTEDTAVYYCVRHGNFGNSYISYWAYW |
| | GQGTLVTVSS (SEQ ID NO: 130) |

### Combination Therapies (e.g., Chimeric Antigen Receptors (CAR) and CAR Immunotherapy)

In some embodiments, any of the methods described herein may comprise additional therapies (e.g., cancer treatments). In some embodiments, the methods herein further comprise administering to the subject an effective amount of a population of immune cells harboring a nucleic acid molecule encoding a chimeric antigen receptor targeted to HER2. In various embodiments, the methods described here include other methods known in the art for treating cancer; for example, the methods can further comprise administering an effective amount of one or more checkpoint inhibitors, such as an anti-PD-1 antibody (e.g., nivolumab, lambrolizumab, or pembrolizumab) or an anti-CTLA-4 antibody (e.g., ipilimumab). In some embodiments, the methods described here can further comprise administering a subject an effective amount of a chemotherapy.

Examples of clinical trials that include therapies combined with blinatumomab (a CD19-TCE) include:
ClinicalTrials.gov Identifier: NCT02879695, blinatumomab can be given with nivolumab alone or nivolumab and ipilimumab in treating patients (e.g., subjects with poor-risk CD19+ precursor B-lymphoblastic leukemia that has come back after a period of improvement (relapsed) or has not responded to treatment (refractory))
ClinicalTrials.gov Identifier: NCT03512405, Pembrolizumab and Blinatumomab in Treating Participants With Recurrent or Refractory Acute Lymphoblastic Leukemia;
ClinicalTrials.gov Identifier: NCT02997761, Ibrutinib and Blinatumomab in Treating Patients With Relapsed or Refractory B Acute Lymphoblastic Leukemia;
ClinicalTrials.gov Identifier: NCT02568553, Lenalidomide and Blinatumomab for the Treatment of Relapsed Non-Hodgkin Lymphoma;
ClinicalTrials.gov Identifier: NCT03263572, Blinatumomab, Methotrexate, Cytarabine, and Ponatinib in Treating Patients with Philadelphia Chromosome-Positive, or BCR-ABL Positive, or Relapsed/Refractory, Acute Lymphoblastic Leukemia;
ClinicalTrials.gov Identifier: NCT02143414, Blinatumomab and Combination Chemotherapy or Dasatinib, Prednisone, and Blinatumomab in Treating Older Patients with Acute Lymphoblastic Leukemia;
ClinicalTrials.gov Identifier: NCT03914625 A Study to Investigate Blinatumomab in Combination With Chemotherapy in Patients With Newly Diagnosed B-Lymphoblastic Leukemia; etc.

In some embodiments, any of the methods described herein further comprise administering to the subject an effective amount of a population of immune cells expressing a CAR or harboring a nucleic acid encoding a CAR. In some embodiments, the CAR is targeted to HER2. In some embodiments, a HER2 CAR comprises a scFv targeted to HER2. In some embodiments, the solid tumor is a HER2-expressing cancer. In some embodiment, the population of immune cells expressing a CAR or harboring a nucleic acid encoding a CAR is a population of HER2 CAR T cells. In some embodiments, the population of immune cells expressing a CAR or harboring a nucleic acid encoding a CAR (e.g., HER2 CAR T cells) are autologous or allogeneic. In some embodiments, the population of immune cells expressing a CAR or harboring a nucleic acid encoding a CAR (e.g., HER2 CAR T cells) are administered simultaneously or subsequently to administering to the subject a TCE described herein (e.g., TCE-CD19). In some embodiments, the HER2 CAR T cells are administered at least or about 0.1, 0.25, 0.5, 0.75, 1, 2, 3, 4, 5, 6, 7, 8, 9, 10, 11, 12, 13, 14, 15, 26, 17, 18, 19, or 20 days after administration of the TCE. In some embodiments, the HEER2 CAR T cells are administered at least or about 1, 2, 3, 4, 5, 6, 7, 8, 9, or 10 weeks after administration of the TCE.

Useful HER2 CAR constructs are described in WO 2017/079694.

In general, HER2 CAR include a HER2 scFv, a spacer domain, a transmembrane domain, one or more co-stimulatory domains, and a CD3zeta cytoplasmic domain. For example, a HER2 CAR can comprise or consist of the amino acid sequence: DIQMTQSPSSLSASVGDRVTITCRASQDVNTAVAWYQQKPGKAPKLLIYSA SFLYSGVPSRFSGSRSGTDFTLTISSLQPEDFATYYCQQHYTTPPTFGQGTKVEIKG STSGGGSGGGSGGGGSSEVQLVESGGGLVQPGGSLRLSCAASGFNIKDTYIHWVR QAPGKGLEWVARIYPTNGYTRYADSVKGRFTISADTSKNTAYLQMNSLRAEDTA VYYCSRWGGDGFYAMDYWGQGTLVTVSSESKYGPPCPPCPAPEFEGGPSVFLFPP KPKDTLMISRTPEVTCVVVDVSQEDPEVQFNWYVDGVEVHNAKTKPREEQFQST YRVVSVLTVLHQDWLNGKEYKCKVSNKGLPSSIEKTISKAKGQPREPQVYTLPPS QEEMTKNQVSLTCLVKGFYPSDIAVEWESNGQPENNYKTTPPVLDSDGSFFLYSR LTVDKSRWQEGNVFSCSVMHEALHNHYTQKSLSLSLGKMFWVLVVVGGVLAC YSLLVTVAFIIFWVRSKRSRGGHSDYMNMTPRRPGPTRKHYQPYAPPRDFAAYRS GGGRVKFSRSADAPAYQQGQNQLYNELNLGRREEYDVLDKRRGRDPEMGGKPR RKNPQEGLYNELQKDKMAEAYSEIGMKGERRRGKGHDGLYQGLSTATKDTYDA LHMQALPPR (SEQ ID NO:57), or a variant thereof have 1, 2, 3, 4, 5, 6, 7, 8, 9, or 10 amino acid modifications (e.g., substitutions), wherein the amino acid modifications are not in the HER2 targeting domain (e.g., the CDRs of the HER2 scFv). A useful HER2 CAR can comprise or consist of the amino acid sequence: DIQMTQSPSSLSASVGDRVTITCRASQDVNTAVAWYQQKPGKAPKLLIYSA SFLYSGVPSRFSGSRSGTDFTLTISSLQPEDFATYYCQQHYTTPPTFGQGTKVEIKG STSGGGSGGGSGGGGSSEVQLVESGGGLVQPGGSLRLSCAASGFNIKDTYIHWVR QAPGKGLEWVARIYPTNGYTRYADSVKGRFTISADTSKNTAYLQMNSLRAEDTA VYYCSRWGGDGFYAMDYWGQGTLVTVSSESKYGPPCPPCPAPEFEGGPSVFLFPP KPKDTLMISRTPEVTCVVVDVSQEDPEVQFNWYVDGVEVHNAKTKPREEQFQST YRVVSVLTVLHQDWLNGKEYKCKVSNKGLPSSIEKTISKAKGQPREPQVYTLPPS QEEMTKNQVSLTCLVKGFYPSDIAVEWESNGQPENNYKTTPPVLDSDGSFFLYSR LTVDKSRWQEGNVFSCSVMHEALHNHYTQKSLSLSLGKIYIWAPLAGTCGVLLL SLVITKRGRKKLLYIFKQPFMRPVQTTQEEDGCSCRFPEEEEGGCELGGGRVKFSR SADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO:49), or a variant thereof have 1, 2, 3, 4, 5, 6, 7, 8, 9, or 10 amino acid modifications (e.g., substitutions), wherein the amino acid modifications are not in the HER2 targeting domain (e.g., the CDRs of the HER2 scFv).

(a) HER2 scFv

A useful HER2 scFv can comprise or consist of the amino acid sequence: DIQMTQSPSSLSASVGDRVTITCRASQDVNTAVAWYQQKPGKAPKLLIYSASFLYS GVPSRFSGSRSGTDFTLTISSLQPEDFATYYCQQHYTTPPTFGQGTKVEIKGGGGS GGGGSGGGGSEVQLVESGGGLVQPGGSLRLSCAASGFNIKDTYIHWVRQAPGKG LEWVARIYPTNGYTRYADSVKGRFTISADTSKNTAYLQMNSLRAEDTAVYYCSR WGGDGFYAMDYWGQGTLVTVSS (SEQ ID NO: 60) or is least 90%, 95%, 98%, 99% identical to SEQ ID NO: 60. A HER2 scFv can comprise: a light chain variable domain that is at least 90%, 95%, 98%, 99% or 100% identical to: DIQMTQSPSSLSASVGDRVTITCRASQDVNTAVAWYQQKPGKAPKLLIYSASFLYS GVPSRFSGSRSGTDFTLTISSLQPEDFATYYCQQHYTTPPTFGQGTKVEIK (SEQ ID NO: 61) and comprises light chain CDRs 1-3 (underlined) and a heavy chain variable domain that is at least 90%, 95%, 98%, 99% or 100% identical to: EVOLVESGGGLVQPGGSLRLSCAASGFNIKDTYIHWVRQAPGKGLEWVARIYPT NGYTRYADSVKGRFTISADTSKNTAYLQMNSLRAEDTAVYYCSRWGGDGFYAM DYWGQGTLVTVSS (SEQ ID NO: 62) and comprises heavy chain CDRs 1-3 (underlined). In some embodiments, the light chain variable domain can precede the heavy chain variable domain and they can be joined by a linker that includes 5-20 amino acids, preferably G and S. In some embodiments, the heavy chain variable domain can precede the light chain variable domain and they can be joined by a linker that includes 5-20 amino acids, preferably G and S. Such linkers can comprise the sequence: GGGGSGGGGSGGGGS (SEQ ID NO: 58) or GGGGSGGGGS (SEQ ID NO: 59) and can be located between the VH and VL domains.

A useful scFv can target HER2. A useful HER2 scFv can comprise or consist of the amino acid sequence: DIQMTQSPSSLSASVGDRVTITCKASQDVSIGVAWYQQKPGKAPKLLIYSASYRYT GVPSRFSGSGSGTDFTLTISSLQPEDFATYYCQQYYIYPYTFGQGTKVEIKGGGGS GGGGSGGGGSEVQLVESGGGLVQPGGSLRLSCAASGFTFTDYTMDWVRQAPGK GLEWVADVNPNSGGSIYNQRFKGRFTLSVDRSKNTLYLQMNSLRAEDTAVYYCA RNLGPSFYFDYWGQGTLVTVSS (SEQ ID NO:63) or is least 90%, 95%, 98%, 99% identical to SEQ ID NO:63. A HER2 scFv can comprise: a light chain variable domain that is at least 90%, 95%, 98%, 99% or 100% identical to: DIQMTQSPSSLSASVGDRVTITCKASQDVSIGVAWYQQKPGKAPKLLIYSASYRYT GVPSRFSGSGSGTDFTLTISSLQPEDFATYYCQQYYIYPYTFGQGTKVEIK (SEQ ID NO: 64) and comprises light chain CDRs 1-3 (underlined) and a heavy chain variable domain that is at least 90%, 95%, 98%, 99% or 100% identical to: EVOLVESGGGLVQPGGSLRLSCAASGFTFTDYTMDWVRQAPGKGLEWVADVNP NSGGSIYNQRFKGRFTLSVDRSKNTLYLQMNSLRAEDTAVYYCARNLGPSFYFD YWGQGTLVTVSS (SEQ ID NO: 65) and comprises heavy chain CDRs 1-3 (underlined). In some embodiments, the light chain variable domain can precede the heavy chain variable domain and they can be joined by a linker that includes 5-20 amino acids, preferably G and S. In some embodiments, the heavy chain variable domain can precede the light chain variable domain and they can be joined by a linker that includes 5-20 amino acids, preferably G and S. Such linkers can comprise the sequence: GGGGSGGGGSGGGGS (SEQ ID NO: 58) or GGGGSGGGGS (SEQ ID NO: 59) and can be located between the VH and VL domains.

A CAR can comprise any scFv described herein or a variant thereof having 1-5 (e.g., 1 or 2) amino acid modifications (e.g., substitutions); a transmembrane domain selected from: a CD4 transmembrane domain or variant thereof having 1-5 (e.g., 1 or 2) amino acid modifications (e.g., substitutions), a CD8 transmembrane domain or variant thereof having 1-5 (e.g., 1 or 2) amino acid modifications (e.g., substitutions), a CD28 transmembrane domain or a variant thereof having 1-5 (e.g., 1 or 2) amino acid modifications (e.g., substitutions), and a CD3ξ transmembrane domain or a variant thereof having 1-5 (e.g., 1 or 2) amino acid modifications (e.g., substitutions); a costimulatory domain (e.g., a CD28 co-stimulatory domain or a variant thereof having 1-5 (e.g., 1 or 2) amino acid modifications (e.g., substitutions); or a 4-1 BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions); and a CD3ζ signaling domain or a variant thereof having 1-5 (e.g., 1 or 2) amino acid modifications.

### (b) Transmembrane Domain

A CAR disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of a CAR as provided herein can be a CD28 (M) transmembrane domain having the sequence: MFWVLVVVGGVLACYSLLVTVAFIIFWV (SEQ ID NO: 17). Other transmembrane domains can be used including those shown below in Table 1.

TABLE 1
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 15) |
| CD28 | NM_006139 | 27 aa | FWVLVVVGGVLACYSLLVTVA |
| | | | FIIFWV |
| | | | (SEQ ID NO: 16) |
| CD28(M) | NM_006139 | 28 aa | MFWVLVVVGGVLACYSLLVTV |
| | | | AFIIFWV |
| | | | (SEQ ID NO: 17) |
| CD4 | M35160 | 22 aa | MALIVLGGVAGLLLFIGLGIF |
| | | | F |
| | | | (SEQ ID NO: 18) |
| CD8tm | NM_001768 | 21 aa | IYIWAPLAGTCGVLLLSLVIT |
| | | | (SEQ ID NO: 19) |
| CD8tm2 | NM_001768 | 23 aa | IYIWAPLAGTCGVLLLSLVIT |
| | | | LY |
| | | | (SEQ ID NO: 20) |
| CD8tm3 | NM_001768 | 24 aa | IYIWAPLAGTCGVLLLSLVIT |
| | | | LYC |
| | | | (SEQ ID NO: 21) |
| 4-1BB | NM_001561 | 27 aa | IISFFLALTSTALLFLLFFLT |
| | | | LRFSVV |
| | | | (SEQ ID NO: 22) |
| NKG2D | NM_007360 | 21 aa | PFFFCCFIAVAMGIRFIIMVA |
| | | | (SEQ ID NO: 23) |

### (c) Spacer Domain

A HER2 CAR described herein can include a spacer located between the HER2 targeting domain (i.e., a HER2 targeted scFv or functional variant thereof) and the transmembrane domain. Without being bound by theory, the spacer can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain, or variants thereof. Table 2 below provides various spacers that can be used in the CARs or polypeptides described herein.

TABLE 2
Examples of Spacer Domains

| Name | Length | Sequence |
|---|---|---|
| a3 | 3 aa | AAA |
| linker | 10 aa | GGGSSGGGSG (SEQ ID NO: 24) |
| IgG4 hinge (S→P) | 12 aa | ESKYGPPCPPCP (SEQ ID NO: 25) |
| (S228P) | | |
| IgG4 hinge | 12 aa | ESKYGPPCPSCP (SEQ ID NO: 26) |
| IgG4 hinge (S228P) + | 22 aa | ESKYGPPCPPCPGGGSSGGGSG (SEQ ID NO: 27) |
| linker | | |
| Also called HL | | |
| CD28 hinge | 39 aa | IEVMYPPPYLDNEKSNGTIIHVKGKHLCPSPLFPGPSKP |
| | | (SEQ ID NO: 28) |
| CD8 hinge-48aa | 48 aa | AKPTTTPAPRPPTPAPTIASQPLSLRPEACRPAAGGAV |
| | | HTRGLDFACD (SEQ ID NO: 29) |
| CD8 hinge-45aa | 45 aa | TTTPAPRPPTPAPTIASQPLSLRPEACRPAAGGAVHTR |
| | | GLDFACD (SEQ ID NO: 30) |
| IgG4 (HL-CH3) | 129 aa | ESKYGPPCPPCPGGGSSGGGSGGQPREPQVYTLPPSQ |
| Also called: IgG4(HL- | | EEMTKNQVSLTCLVKGFYPSDIAVEWESNGQPENNYK |
| ΔCH2) | | TTPPVLDSDGSFFLYSRLTVDKSRWQEGNVFSCSVMH |
| (includes S228P in hinge) | | EALHNHYTQKSLSLSLGK (SEQ ID NO: 31) |
| IgG4 | 229 aa | ESKYGPPCPSCPAPEFEGGPSVFLFPPKPKDTLMISRT |
| (L235E, N297Q) | | PEVTCVVVDVSQEDPEVQFNWYVDGVEVHNAKTKPRE |
| | | EQFQSTYRVVSVLTVLHQDWLNGKEYKCKVSNKGLPS |
| | | SIEKTISKAKGQPREPQVYTLPPSQEEMTKNQVSLTC |
| | | LVKGFYPSDIAVEWESNGQPENNYKTTPPVLDSDGSF |
| | | FLYSRLTVDKSRWQEGNVFSCSVMHEALHNHYTQKSL |
| | | SLSLGK (SEQ ID NO: 32) |
| IgG4(S228P, | 229 aa | ESKYGPPCPPCPAPEFEGGPSVFLFPPKPKDTLMISRT |
| L235E, N297Q) | | PEVTCVVVDVSQEDPEVQFNWYVDGVEVHNAKTKPRE |
| | | EQFQSTYRVVSVLTVLHQDWLNGKEYKCKVSNKGLPS |
| | | SIEKTISKAKGQPREPQVYTLPPSQEEMTKNQVSLTC |
| | | LVKGFYPSDIAVEWESNGQPENNYKTTPPVLDSDGSF |
| | | FLYSRLTVDKSRWQEGNVFSCSVMHEALHNHYTQKSL |
| | | SLSLGK (SEQ ID NO: 33) |
| IgG4(CH3) | 107 aa | GQPREPQVYTLPPSQEEMTKNQVSLTCLVKGFYPSDI |
| Also called IgG4 | | AVEWESNGQPENNYKTTPPVLDSDGSFFLYSRLTVDK |
| (ΔCH2) | | SRWQEGNVFSCSVMHEALHNHYTQKSLSLSLGK (SEQ |
| | | ID NO: 34) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ACH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO: 26) or ESKYGPPCPPCP (SEQ ID NO: 25). The hinge/linger region can also comprise the sequence ESKYGPPCPPCP (SEQ ID NO: 25) followed by the linker sequence GGGSSGGGSG (SEQ ID NO: 24) followed by IgG4 CH3 (HL-CH3) sequence GQPREPQVYTLPPSQEEMTKNQVSLTCLVKGFYPSDIAVEWESNGQPENNYKTT PPVLDSDGSFFLYSRLTVDKSRWQEGNVFSCSVMHEALHNHYTQKSLSLSLGK (SEQ ID NO: 34). Thus, the entire linker/spacer region can comprise the sequence: ESKYGPPCPPCPGGGSSGGGSGGQPREPQVYTLPPSQEEMTKNQVSLTCLVKGFY PSDIAVEWESNGQPENNYKTTPPVLDSDGSFFLYSRLTVDKSRWQEGNVFSCSV MHEALHNHYTQKSLSLSLGK (SEQ ID NO: 31). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (L235E; N297Q) in a manner that reduces binding by Fc receptors (FcRs).

### (d) Intracellular Signaling Domains

A CAR construct described herein contains one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD33 contains three immunoreceptor tyrosine-based activation motifs (ITAMs), which transmit an activation signal to the T cell after the T cell is engaged with a cognate antigen. In some cases, CD3ζ provides a primary T cell activation signal but not a fully competent activation signal, which requires a co-stimulatory signal.

Accordingly, in some examples, the CAR constructs disclosed herein may further comprise one or more co-stimulatory signaling domains in addition to CD32. For example, the co-stimulatory domain CD28 and/or 4-1BB can be used to transmit a proliferative/survival signal together with the primary signaling mediated by CD32.

The co-stimulatory domain(s) are located between the transmembrane domain and the CD3ζ signaling domain. Table 3 includes examples of suitable co-stimulatory domains together with the sequence of the CD33 signaling domain.

TABLE 3
CD3ζ Domain and Examples of Co-stimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR |
| | | | (SEQ ID NO: 35) |
| | | | ITAMS 1-3 underlined |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLFQGLSTATKDTFDALHMQALPPR |
| | | | (SEQ ID NO: 50) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLYQGLSTATKDTYDALHMQALPPR |
| | | | (SEQ ID NO: 51) |
| CD3ζ | | 113 aa | RVKFSRSADAPAYQQGQNQLYNELNLGRREEYDVLDKRRGR |
| variant | | | DPEMGGKPRRKNPQEGLYNELQKDKMAEAYSEIGMKGERR |
| | | | RGKGHDGLFQGLSTATKDTFDALHMQALPPR |
| | | | (SEQ ID NO: 52) |
| CD3ζ | | 113 aa | RVKFSRSADAPAYQQGQNQLYNELNLGRREEYDVLDKRRGR |
| variant | | | DPEMGGKPRRKNPQEGLYNELQKDKMAEAFSEIGMKGERR |
| | | | RGKGHDGLFQGLSTATKDTFDALHMQALPPR |
| | | | (SEQ ID NO: 53) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAYSEIGMKGERR |
| | | | RGKGHDGLFQGLSTATKDTFDALHMQALPPR |
| | | | (SEQ ID NO: 54) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLYQGLSTATKDTFDALHMQALPPR |
| | | | (SEQ ID NO: 55) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLFQGLSTATKDTYDALHMQALPPR |
| | | | (SEQ ID NO: 56) |
| CD28 | NM_006139 | 42 aa | RSKRSRLLHSDYMNMTPRRPGPTRKHYQPYAPPRDFAAYRS |
| | | | (SEQ ID NO: 36) |
| CD28gg* | NM_006139 | 42 aa | RSKRSRGGHSDYMNMTPRRPGPTRKHYQPYAPPRDFAAYR |
| | | | S (SEQ ID NO: 37) |
| 41BB | NM_001561 | 42 aa | KRGRKKLLYIFKQPFMRPVQTTQEEDGCSCRFPEEEEGGCEL |
| | | | (SEQ ID NO: 38) |
| OX40 | NM_003327 | 42 aa | ALYLLRRDQRLPPDAHKPPGGGSFRTPIQEEQADAHSTLAKI |
| | | | (SEQ ID NO: 39) |
| 2B4 | NM_016382 | 120 aa | WRRKRKEKQSETSPKEFLTIYEDVKDLKTRRNHEQEQTFPGG |
| | | | GSTIYSMIQSQSSAPTSQEPAYTLYSLIQPSRKSGSRKRNHS |
| | | | PSFNSTIYEVIGKSQPKAQNPARLSRKELENFDVYS |
| | | | (SEQ ID NO: 40) |

In some examples, the CD3 signaling domain comprises an amino acid sequence that is at least 90%, at least 95%, at least 98% identical to SEQ ID NO: 35. In such instances, the CD3ξ signaling domain has 1, 2, 3, 4, or 5 amino acid changes (preferably conservative substitutions) compared to SEQ ID NO: 35. In other examples, the CD32 signaling domain is SEQ ID NO: 35.

In various embodiments: the co-stimulatory domain is selected from the group consisting of: a co-stimulatory domain depicted in Table 3 or a variant thereof having 1-5 (e.g., 1 or 2) amino acid modifications, a CD28 co-stimulatory domain or a variant thereof having 1-5 (e.g., 1 or 2) amino acid modifications, a 4-1BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3 signaling domain and a short linker consisting of 2-10, e.g., 3 amino acids (e.g., GGG) can be positioned between the co-stimulatory domain and the CD35 signaling domain.

In various embodiments: the costimulatory domain is selected from the group consisting of: a CD28 costimulatory domain or a variant thereof having 1-5 (e.g., 1 or 2 acid modifications, a 4-1 BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions). In various embodiments the 1-5 (e.g., 1 or 2) amino acid modification are substitutions.

In some cases there is a short sequence of 1-6 amino acids (e.g. GGG) between the co-stimulatory domains and the CD3& signaling domain and/or between the two co-stimulatory domains.

Also disclosed is a population of human T cells transduced by a vector comprising an expression cassette encoding a chimeric antigen receptor, wherein chimeric antigen receptor comprises: an scFv targeted to HER2; a transmembrane domain selected from: a CD4 transmembrane domain or variant thereof having 1-5 amino acid modifications (e.g., 1 or 2) amino acid modifications (e.g., substitutions), a CD8 transmembrane domain or variant thereof having 1-5 amino acid modifications (e.g., 1 or 2) amino acid modifications (e.g., substitutions), a CD28 transmembrane domain or a variant thereof having 1-5 amino acid modifications (e.g., 1 or 2) amino acid modifications (e.g., substitutions), and a CD3ξ transmembrane domain or a variant thereof having 1-5 amino acid modifications (e.g., 1 or 2) amino acid modifications (e.g., substitutions); a costimulatory domain (e.g., a CD28 co-stimulatory domain or a variant thereof having 1-5 (e.g., 1 or 2) amino acid modifications (e.g., substitutions); or a 4-1 BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions); and CD3ζ signaling domain of a variant thereof having 1-5 amino acid modifications (e.g., 1 or 2) amino acid modifications (e.g., substitutions).

In various embodiments: the population of human T cells comprises central memory T cells (TCM cells) e.g., at least 20%, 30%, 40%, 50% 60%, 70%, 80% of the cells are TCM cells, or the population of T cells comprises a combination of central memory T cells, naive T cells and stem central memory cells (TCM/SCM/N cells) e.g., at least 20%, 30%, 40%, 50% 60%, 70%, 80% of the cells are TCM/SCM/N cells. In either case, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells).

The CAR can include a spacer region located between the tumor targeting domain (e.g., a scFv; e.g., a HER2 scFv) and the transmembrane domain. A variety of different spacers can be used. Some of them include at least portion of a human Fc region, for example a hinge portion of a human Fc region or a CH3 domain or variants thereof.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

For amino acid positions in immunoglobulin discussed herein, numbering is according to the EU index or EU numbering scheme (Kabat et al. 1991 Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al. 1969 Proc Natl Acad Sci USA 63:78-85).

The methods described herein can further comprise administering a population of autologous or allogeneic human immune cells (e.g., macrophages, NK cells, NKT cells, T cells, subpopulations of each thereof, and combinations thereof). In some embodiments, useful autologous or allogenic T cells comprise central memory T cells (TCM cells) or a combination of central memory T cells, naive T cells, and stem central memory cells (i.e., the T cells are TCM/SCM/N cells). In some embodiments, the population of T cells includes both CD4+ cells and CD8+ cells. In some embodiments, the immune cells express a CAR or are transduced by a nucleic acid encoding a CAR (e.g., a HER2 CAR). Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G. CD19-TCE redirects in vitro activation and cytotoxicity of human T cells against tumor cells infected with OV19t. FIG. 1A) Schematic depicting combination therapy concept utilizing OV19t to introduce CD19t to solid tumors cells allowing for targeting of tumor cells by peripheral blood mononuclear cells (PBMC) with the addition of CD19-TCE. FIGS. 1B-1C) Quantification of (FIG. 1B) CD69 and (FIG. 1C) CD137 expression on human T cells following 24 hours co-culture with tumor cells at an effector: target (E:T) ratio of 1:1 with or without CD19-TCE and the indicated multiplicity of infection (MOI) of OV19t. FIG. 1D) IFNy (left) and IL-2 (right) production measured by enzyme-linked immunosorbent assay (ELISA) in supernatants collected from co-cultures with or without CD19-TCE with indicated MOI of OV19t after 24 hours. FIG. 1E) Tumor cell killing assay of MDA-MB-468 tumor cells visualized by phase-contrast microscopy with or without CD19-TCE in the presence of human T cells or CD19-CAR T cells and indicated MOI of OV19t. MDA-MB-468-CD19t cells were used as positive control. Images are shown. FIG. 1F) Quantification of MDA-MB-468 cell killing assessed by flow cytometry. Tumor cells were co-cultured with human T cells with or without CD19-TCE and indicated MOI of OV19t after 24 (left), 48 (middle), and 72 (right) hours. FIG. 1G) CD19t percentage on MDA-MB-468 tumor cells from FIG. 1F.

FIGS. 2A-2C. Comparing efficacy of FDA-approved BLINCYTO® to research grade CD19-TCE. FIG. 2A) Quantification of CD137 on human T cells following 24 (left), 48 (middle), or 72 (right) hours following coculture with MDA-MB-468 tumor cells at an effector: target (E:T) ratio of 1:1 with or without BLINCYTOR or CD19-TCE and the indicated MOI of OV19t. FIG. 2B) Percent of CD19t expression on MDA-MB-468 tumor cells in killing assay described in FIG. 2A. FIG. 2C) Quantification of MDA-MB-468 cell killing assessed by flow cytometry.

FIGS. 3A-3E. In vivo efficacy of combination therapy of CD19-TCE and OV19t in a human xenograft MDA-MB-468 tumor model. FIG. 3A) Schematic depicting subcutaneous MDA-MB-468 tumor-bearing NSG mice (5×10⁶ cells on day 0) treated with IT OV19t (1×10⁷ pfu) on day 32, engrafted with IV PBMC expressing firefly luciferase (PBMC-ffluc) (1×10⁷ cells) on day 34, and treated with IV CD19-TCE (8 ug/dose, 8 times) on days 38-42 and 45-47. FIG. 3B) Flux imaging of mice on indicated days after engraftment with PBMC-ffluc only (left), OV19t with PBMC-ffluc (middle), and OV19t with PBMC-ffluc and CD19-TCE (right). FIG. 3C) Quantification of T cell flux at the tumor site. FIG. 3D) Schematic depicting subcutaneous MDA-MB-468 tumor-bearing NSG mice (5×10⁶ cells on day 0) treated with intratumoral (IT) OV19t (1×106 pfu) on day 32, engrafted with intravenous (IV) PBMC (1×10⁷ cells) on day 34, and treated with IV CD19-TCE (100 μg/dose, 8 times) on days 38-42 and 45-47. FIG. 3E) Tumor volumes are shown as mean±SEM (n≥5 per group).

FIGS. 4A-4E. HER2-CAR T cells combined with CD19-TCE directs activation and cytotoxicity against HER2-positive and-negative tumor cells infected with OV19t. FIG. 4A) Protein L and CD3 expression on T cells preloaded with CD19-TCE (10 μg/mL) for 1 hour. FIG. 4B) Protein L and CD137 expression on T cells at 0.5, 4, 24, 48, and 72 hours following CD19-TCE preloading (10 ug/mL). FIG. 4C) Quantification of CD137 expression (left), tumor cell killing (middle), and CD19t expression (right) on CD19-TCE preloaded human T cells following 24 hours co-culture with MDA-MB-468 tumor cells at an E:T ratio of 1:1 with or without CD19-TCE and indicated MOI of OV19t. FIG. 4D) Schematic of tumor cell rechallenge killing assay (top). MDA-MB-468 tumor cells with or without HER2 expression (1:4 ratio) were plated and infected with OV19t at indicated MOI. human T cells or HER2-CAR T cells were added with or without CD19-TCE, and rechallenged with non-HER2 expressing MDA-MB-468 cells. CD137 expression, tumor cell killing, CD19t expression, and T cell counts were quantified using flow cytometry. FIG. 4E) Assay was performed as demonstrated in FIG. 4D with an additional rechallenge 48 hours after the first rechallenge, and CD137 expression, tumor cell killing, CD19t expression, and T cell counts at the lowest MOI (0.003125) of OV19t were quantified using flow cytometry.

FIGS. 5A-5B. CD19-TCE redirected human T cell cytotoxicity against positive control cell line and OV90 tumor cell killing visualized by phase-contrast microscopy. (FIG. 5A) Quantification of MDA-MB-468 cells stably expressing CD19t co-cultured with T cells with or without CD19-TCE. (FIG. 5B) OV90 tumor cells were co-cultured with human T cells with or without CD19-TCE (0, 20, or 100 ng/ml) or CD19-CAR T cells and the indicated MOI of OV19t. Images were taken using phase-contrast microscopy.

FIGS. 6A-6C. OV19t induces CD19t expression on various solid tumor cell lines, which directs activation and cytotoxicity of human T cells in the presence of CD19-TCE in vitro. (FIG. 6A) PC3, (FIG. 6B) U87, or (FIG. 6C) SNU-16 tumor cells were co-cultured with human T cells with or without CD19-TCE or CD19-CAR T cells and the indicated MOI of OV19t for 24 hours. CD137 expression was quantified by flow cytometry. Supernatants were collected and ELISA was used to detect levels of IFN-γ and IL-2. Tumor cell killing and CD19t expression were quantified using flow cytometry.

FIG. 7. Anti-tumor efficacy of combination therapy of CD19-TCE and OV19t with PBMC-ffluc engraftment in human xenograft MDA-MB-468 tumor model.

NSG mice were subcutaneously injected with MDA-MB-468 (5×10⁶ cells) on day 0. Tumor-bearing mice were treated with IT OV19t (1×106 pfu) on day 32, engrafted with IV PBMC-ffluc (1×10⁷ cells) on day 34, and treated with IV CD19-TCE (100 μg/dose, 8 times) on days 38-42 and 45-47. Tumor volumes are shown as means±SEM (n=5).

FIGS. 8A-8C. Expression of exhaustion markers on human T cells preloaded with CD19-TCE. Expression of (FIG. 8A) Tim3, (FIG. 8B) Lag3, and (FIG. 8C) PD-1 on human T cells following preloading with CD19-TCE (10 μg/mL) at 24, 48, and 72 hours.

FIGS. 9A-9C. CD19t expression on tumor cells following OV19t infection directs activation and cytotoxicity of human T cells in the presence of CD19-TCE in vitro at 48 hours. (FIG. 9A) Quantification of CD137 expression on CD19-TCE preloaded human T cells or T cells alone following 48 hours coculture with MDA-MB-468 tumor cells at an E:T ratio of 1:1 with or without CD19-TCE with the addition of indicated MOI of OV19t. (FIG. 9B) Tumor killing and (FIG. 9C) CD19t expression of MDA-MB-468 tumor cells in killing assay described in FIG. 9A.

FIGS. 10A-10B. OV19t induces CD19t expression on MDA-MB-468, which directs activation and cytotoxicity of human T cells and HER2-CAR T cells in the presence of CD19-TCE in vitro. Human T cells or HER2-CAR T cells with or without CD19-TCE were added to wild-type and HER2 expressing MDA-MB-468 tumor cells (1:4 ratio) infected with indicated MOIs of OV19t. CD137 expression (left), tumor cell killing (middle), and CD19t expression (right) were quantified at (FIG. 10A) 24 and (FIG. 10B) 48 hours using flow cytometry.

FIGS. 11A-11BE provides the nucleotide sequence of an OV19t (SEQ ID NO:1). The sequence for the synthetic early promoter (pSE) and the sequence encoding the transgene, human truncated CD19 (CD19t also hCD19t), are underlined and identified on FIG. 11Y.

FIGS. 12A-12AU provides the nucleotide sequence for the parent strain OV (CF33; SEQ ID NO: 2). The J2R gene encoding thymidine kinase (tk) is underlined and identified. The nucleotides deleted for CD19t transgene insertion for OV19t are double underlined and identified (“[del]”).

FIGS. 13A-13BL provides the nucleotide sequences for coding regions of OV19t. Genes 1-322 coincide with SEQ ID NOs: 131-322, respectively.

FIGS. 14A-14AB provides the amino acid sequences for proteins encoded by OV19t. Protein 1-201 coincide with SEQ ID NOs: 453-653, respectively.

FIGS. 15A-15BH provides the nucleotide sequence of an OV19t (SEQ ID NO:5). The 5′ ITR and the 3′ ITR are underlined.

### DETAILED DESCRIPTION

We previously developed a novel combination therapy for solid tumors by delivering a truncated CD19 antigen with an oncolytic virus (OV) followed by CD19-CAR T cells to mitigate antigen heterogeneity and elicit an immunologically warm TME [13]. The present application is directed to, inter alia, the ability of OV to deliver truncated CD19 to solid tumors to be targeted with bispecific T cell engagers (TCE) to induce endogenous T cell activation and anti-tumor responses. The current findings support a fully off-the-shelf therapeutic combination with immediate clinical applications. Further, we demonstrate that this paradigm can be advanced with two clinically active TCEs, with the prospect of additional tumor antigens that may be exploited for this combinatorial immunotherapy strategy.

Using a TCE rather than a CAR T cell carries several key advantages. Given the length of time required to generate patient-specific CD19-CAR T cells, we reasoned that the availability of off-the-shelf immunotherapies overcomes time restraints for patients undergoing treatment. With current manufacturing practices and capacities, the demand for CAR T cells exceeds supply and is still viewed as a niche technology that not all sites can perform [17]. Moreover, TCEs such as blinatumomab provide a safety advantage over CAR T cells due to the nature of their short in vivo half-life and dosing strategies [18, 19]. This treatment can then be suspended or delayed if needed, with the potential for reversing unfavorable immune-related adverse effects. Importantly, the proof-of-concept trial for the combination therapy using OV19t and blinatumomab is underway with recent FDA clearance.

TABLE 4
OV19t Open Read Frames (ORFs) and Corresponding Sequences
(the nucleotide sequences for the identified coding
regions of OV19t, ORF1-ORF275, respectively;
the amino acid sequences below coincide with
SEQ ID NOs: 654-928, respectively)

>ORF1:
MRDVRPEYWMGLDVAVFLFFATSDVDRHLTVFPSADSEMDLI
>ORF2:
MDSLRPVVVVRVHDVSLTGYRLISTSI
>ORF3:
MTTGDIILTLEGKSSSRIKAYLIQANNGSGYR
>ORF4:
MAVLIIERDVVTLAPYSNNRFANCILLTSTA
>ORF5:
MRRYIVMKRVLTHITVVHTGTKYVTGAVTF
>ORF6:
MVYVDLRTIYHRKVSHKYSCNGRCFCILLINIYKRRM
>ORF7:
MYILQVILGTHHHINTGNYILRNIKLVCICKMFRKWIFPHCFKMDGANNCHTSING
>ORF8:
MTRSLNRLCDARLLRLSSLRIIFMLVILL
>ORF9:
MYLSNSDFFSHVCVDTSLVIGDMCSASSTSDTLSIL
>ORF10:
MDFPIQSVHRQSIMTLTQQCMLYWQVLLSLR
>ORF11:
MIITYFEIRIKSQQHFSHMNIITHENTKIQMEWCITTII
>ORF12:
MLYKQRNNSLGLFYRYRFSTLTWFINVCETQIIIM
>ORF13:
MISVQSLTSSHGIFFTSSRMSMNTDSASSLKERSVEYNCPAHCLLRYA
>ORF14:
MDSNPSILSGRMTIILVSPTYGSSMIEKLPNDSFTDA
>ORF15:
MNTFSPFIEDAISLKISIVIDIGRVAR
>ORF16:
MSSFSILLTTIVLLELFMNNPPVTKIVLLTETADTLLFL
>ORF17:
MNTAISSFGIEIFGNYTQPYFQSPCFLFSLLTSSNRHGVDDRGEQKDYNQGPHLENG
>ORF18:
MQFYFVFVSLIKISYEYLVKLSMLRKVVTSFDVLPRNLVK
>ORF19:
MYKLIWSSYSVNVTVGGPIFTSTHSPVQYSSTL
>ORF20:
MESNPTSHDSVIIEIVYSLKFNTNSLLSSVYEFSILSTPSI
>ORF21:
MAYHIIQRHNQKLCTFSCMDFRISIYLFRRQHLDPKNDIPDHM
>ORF22:
MILRTRLPYTSRLVHFTKEGCLPYLIKVLMSLIQKTNWNTVLFFI
>ORF23:
MISISSKHFKIYHLYCCSDCTSIRVS
>ORF24:
MDDDNLRLCINDPFLLIFNTLSRKKIIIFVLASSIE
>ORF25:
MNRHVYLMSSFFSQKCTTQSLLICFSISFSISLR
>ORF26:
MNKYPPFVLVKDESIKGSCCRIRVLSCLAETDVCLTINRITKFD
>ORF27:
MRMHITAFFTIHNIKMIIYNRPHMAYSFQTFHEYIIHAVFLRGGYLDGAADIVEE
RFSVSFSPSLTFTRFGICSTAPFLNLILIISFPCTSTAVT
>ORF28:
MAVFPEPDGPLNMIDFTIPDIYVSQKSRLCSLFCIYVNVYYIYNM
>ORF29:
MTYINPFNIATPISHFGSRILAYFCQYGFDGFHLSILDVNDES
>ORF30:
MYSSSASPPQLERGYFSSGISYIQEFVHVLYAITFELLYGD
>ORF31:
MEILSCELLIPIDINSMLWYLGNDSSKNAILFKNVIHS
>ORF32:
MRISGAKSSFFIFNISSLKIFIIDSSVKYLKFSSLMSLKIFRNIVLNDADSIFLL
LYLSAYM
>ORF33:
MYFSHSVMADPICKKSAVERIYFTLSLVRLKGIP
>ORF34:
MTSIRIIGDCGICMRWAAFTFCVIVTITSNLGKFSRWTPITTLIMPLIKIFSLVT
>ORF35:
MNLTPRMHTICLMRLIVHRHLYPFL
>ORF36:
MSVISNPSKYKQSNTNKHMLEFLYSFFLCIPKLPNGSGTLCSSTRTISESNTIDV
AFLNAFGMYSLR
>ORF37:
MGLIGVITEIDIESRFCLDAFSIISFKLLDFLNTS
>ORF38:
MLLSRGRISVFSLKKVLIVSPIYPYPKSCSISS
>ORF39:
MIKYLSSLSVIKSLINLDFSLYPDA
>ORF40:
MRVRYAYRTRIRVLIPSCQSNMLHPFITLY
>ORF41:
MLSVCPRRYLHRLYRSLDSSDIGILSDICSANAIHLYFLFVNSQCLDTRLDRATT
ILESQHIISFTKLTKSNSPESESTLSFSLNNRSMSPYCLIAKSSSVINAE
>ORF42:
MDWFKVNNSSDIIVVYYTNSHYLVK
>ORF43:
MSGSNGRVRGYWWCRISRQVYSKTVN
>ORF44:
MEYADYILYLLHYRSYQNTNAHNIIPHPLNEI
>ORF45:
MSCMPIPALLTSSIWILSISSSSFKIDTTYEYYFLF
>ORF46:
MRGIISPGLILANVLLGIAARTFFVVL
>ORF47:
MAASSNLLFFEISSIILCHSAIVIYYICFLIRHLLE
>ORF48:
MDTFIIIAYPVSRTIFSNICIFYRFIY
>ORF49:
MLLVNHLLELRYPPCINHNHMVRQIFSIV
>ORF50:
MRSIFVLSVNVYSPAYYWIVDDDVTIYISYNY
>ORF51:
MRCTPHYLLSIGRIHFNGFGFGIEFKGMC
>ORF52:
MCIIPRASRRGRGTRNYCYFIIILDLILILFIRFFASSFISRYTTEFHKKRRE
>ORF53:
MLIFILSILPYNICSLIGTELYDCIFLYTKSNGCSRLQTCIKVSCSMLEFMYLAE
DIPIQFDGIGNCLLSMVIMEYHLLFANI
>ORF54:
MSFVNDVTNRLLGISALTTNMVVGPLLTTTHLLSGDMITA
>ORF55:
MGKVRIFLPLTSLSIEITHPPTELIQSIIVDNL
>ORF56:
MCSVLGAITLDSLSHIKLPAKLSSSNISFLE
>ORF57:
MSEVSSVAPSDADPSTYLTSSSDDSLHMSPIRTLNSRFLTHFVSTIVEPMISS
>ORF58:
MLDPSTGWVLMSLSFSSSLHLMSIDTSQSFHQRILRWI
>ORF59:
MIVSFFAFVNCLEEESLLLHLISKFRVHTFLL
>ORF60:
MTRPLIGSRLVSFLRSNRRRERHLVEQRFNHR
>ORF61:
MRQHCCYNFERRYYSIRLSECRTYLVDTVVY
>ORF62:
MMGVQSRLLRILVSAPRDKRAYTTRGLCLVYC
>ORF63:
MRRCIHIKERKIHMTNIVDRNVTFILTVVHKYVRYVPHTVANDAYNLVHLIMIYF
FIIKDVKEKQNNIFF
>ORF64:
MGKDPPSSLPWMETLLLIKHLITSLDRD
>ORF65:
MMKAIIPHKMIKSVVREHEGDLVFSSADMIQEGEIVVLVQNLSQHK
>ORF66:
MGPSMRNPQDVFSQTMSDVGHGIQGVLSSVAMTGSELGTRRGHCKGRPVAVYANR
GL
>ORF67:
MLYIPSMSSGSIITILEFPSYGISIIENPPNNSLICTYCKLFSVPTIISTLVTIF
LEI
>ORF68:
MDSGLVNKELILPFVIVLTHSLISSFVWLALLKFTV
>ORF69:
MSLLSSFLSSGVFSFIFSIYFDSCSAVEPAAP
>ORF70:
MSHTTYISSLCETTPGFIVPVQKLGNVFHILLTVLYLITLFEIFVSSSISMLFTV
CTDV
>ORF71:
MEFAFVYLHLVRLYHFRYTLITSCN
>ORF72:
MRSNYTLNTVTILCSYGTQIPTVTEFRCIVGYP
>ORF73:
MYHHFHRHLYYTALKYLYNISFFTHIA
>ORF74:
MYNISYIISEGSNVSRQFLSVKTGNSLSYTFDTYISL
>ORF75:
MVYGEFSLLSIMSCVACLLLSVNAFISPLIRIR
>ORF76:
MSRKFMQVYEYDREQYLDEFIEDRYNDSFITSPEYYSAEKYMCRYTTLNHNCINV
RRCALDSKLLHDIITNCKIYNNIELVRATKFVYYLDLIKCNWVSKVGDSVLYPVI
FITHTSTRNLDKVSVKTYKGVKVKKLNRCADHAIVINPFVKFKLTLPNKTSHAKV
LVTFCKLRTDITQIEAPLSGNVLVYTFPDINKRIPGYIHVNIEGCIDGMIYINSS
KFACVLKLHRSMYRIPPFPIDICSCCSQYTNDDIEIPIHDLIKDVAIFKNKETVY
YLKLNNKTIARFTYFNNIDTAITQEHEYVKIALGIVCKLMINNMHSIVGVNHSNT
FVNCLLEDNV
>ORF77:
MSAPYNICVYVTSNKCINIGTYHFASRSLIYYLWIRDKIYI
>ORF78:
MFLLFSISTFFSISSFSLSISFMISSTFVTSVSMLVVSNKTIG
>ORF79:
MIEGNRRTIIRDLILKMVRAYKGVNOCIICSFISFLYH
>ORF80:
MLQSVVQFLLRRIVPYNTLPLYNRLVQI
>ORF81:
MYIPSSLKALLYLSLARIIDTLLIISYIS
>ORF82:
MNSIPVPTLTETSRSFIIIPLSVSITHCLTRDSFGIVRTISSNGITAHLVNSLYI
LCNMALLLPYTINLLL
>ORF83:
MILILISYSDIYIYVIMYNRNCLLHLYKIHDLLKYIYRY
>ORF84:
MFERDYNPVLYGMWKNWNTRVLYNEEINIHSSQVFPF
>ORF85:
MNLIIRFSVSTTKLGLNHFKRGRSYIHRVRH
>ORF86:
MVIDTFSFHRTRKSSGMGHHKSKYIPIVFWLWLSFLNQMVRSI
>ORF87:
MIFYIISNWFRSRDFEYRRAIKIIYILWFLRTSFW
>ORF88:
MSTMNPDRVCRLYNKCIRHQPVPHN
>ORF89:
MFFISCLKKLSRGYTVNVVAIASSPGNVVSISY
>ORF90:
MMFIHCVVFPDLSNPSKTINAPRDIFVNLTYFLQFMRIIKLKINIWSSETIRSTF
IDHG
>ORF91:
MAVYAVTGGAGFLGRYIVKLLISADDVQEIRVIDIVEDPQPITSKVKVINYIQCD
INDFDKVREALDGVNLIIHTAALVDVFGKYTDNEIMKVNYYGTQTILAACVDLGI
KYLIYTSSMEAIGPNKHGDPFIGHEHTLYDISPGHVYAKSKRMAEQLVMKANNSV
IMNGAKLYTCCLRPTGIYGEGDKLTKVFYEQCKQHGNIMYRTVDDDAVHSRVYVG
NAAWMHVLAAKYIQYPGSKIKGNAYFCYDYSPSCSYDMFNLLLMKPLGIEQGSRI
PRWMLKMYACKNDMKRILFRKPSLLNNYTLKISNTTFEVRTNNAELDFNYSPIFN
VDVAFERTRKWLEESE
>ORF92:
MYSLLFIILMCIPFSFQTVYDDKSVCDSDNKEYMGIEVYVEATLDEHLRQTTCES
EIHKYGASVSNGGLNISVDLLNCFLNFHTVGVYTNRDTVYAKFASLDPWTTEPIN
SMTHDDLVKLTEECIVDIYLKCEVDKTKDFMKTNGNRLKPRDFKTVPPSDVGSMI
ELQSDYCVNDVTAYVKIYDECGNIKQHSIPTLRDYFTTKNGQPRKILKKKFDNC
>ORF93:
MSRVRISLIYLYTLVVITTTKTIEYTACNDTIIIPCTIDNPTKYIRWKLDNHDIL
TYNKTSKTTILSKWHTSARLHSLSDSDVSLIMEYKDILPGTYTCGDNTGIKSTVK
LVQLHTNWFNDYQTMLMFIFTGITLFLLFLEITYTSISVVFSTNLGILQVFGCVI
AMIELCGAFLFYPSMFTLRHIIGLLMMTLPSIFLIITKVFSFWLLCKLSCAVHLI
IYYQLAGYILTVLGLGLSLKECVDGTLLLSGLGTIMVSEHFSLLFLVCFPSTQRD
YY
>ORF94:
MTPLKRERENENENKNILVTPSERGLIFFMRPSKRERE
>ORF95:
MMSFTTSALYMMFLFSNTKSNTVFSRKVDVVSDV
>ORF96:
MGNFEIKLSSVLMLSTMESILSLFFTVSVMMDVSSFLYHLMSDSLRISICGVCFV
>ORF97:
MLLAELILRCFYKVSRLRERVTVEQS
>ORF98:
MIARSSSAVFMESFLMKHLMISTQLSILSHGSESFNSPPKSSVASVLKEMRSL
>ORF99:
MLIEGFNFHFFNGLWMRNESDIILRYVVM
>ORF100:
MTFSHKWTYRCRSNEEKYYLYHLHN
>ORF101:
MIQILHSLDNSVTESYLLLVCLYHSILQTV
>ORF102:
MLQLNVMIQRSFSYACFITPRTCRSKIYSQR
>ORF103:
MDEVTNIRHAINPYIIILILLIITICLIFSLYY
>ORF104:
MLNFSLCLYPVFILNKLVLRTQSIILHTINNASIKNR
>ORF105:
MLYSAMKWCTSFKVTIDIESIQRSNNI
>ORF106:
MVVFVYIIYALLSYTVY AIIMFLPYIPPSYVRLSSIYYFIN
>ORF107:
MNMTESNISMSSLKTDITIVLSLVQNTIVRKNTCVDILH
>ORF108:
MLGIVNAIHVNVVASSSLTGLMFNRDVITRLLSVI
>ORF109:
MPSGNCIVEGIILSRTSNFTILVTLYVLPLYIYSKTHV
>ORF110:
MVIIPGVRCLSLLFLRRRCPLHIISAFTLLAINALILGHTISPVDLSFTICGYEI
RSIFDSETDTIVKFNDIMSQ
>ORF111:
MYNSSIHTPEYDVIIHVIEHLKHHKQCVQTVTSGMVFTSPVSSSICTKSDDGRNL
SDGFLLIRYITTDDFCTIFDIIPRHIFYQLANVDEH
>ORF112:
MEVCHRTILSCNIPTFLNLLVLKLLLQDDCLHTYN
>ORF113:
MLDVQIDMVVVFSQCYSRWSNFYFDA
>ORF114:
MGFHLPSIHVDDGDPVSIKPLLQLRCITPPIENVDPEINPLDRDGISHFC
>ORF115:
MYEAPITSYPTSQLIVIYSPNEYFSLTG
>ORF116:
MHNTKSFDMLFYIILYQSEVVVTHRESKYFVCIIINSNEQISTLCVGKR
>ORF117:
MNAYAHNSILYHSQYQNDHIQQTTYGV
>ORF118:
MLNFSIYSVSCFKYVMMNPYSFQTQV
>ORF119:
MKILVVPTESVCVKHRHDFGCTHEINNKLSYFLIFIDKNFTSTKHHT
>ORF120:
MVIYCSNSLGAYCKLVINKVAVLLTYFCFVALTNAQFFVSDILVHGKIPPNHLII
QYPSVSRSPRLDTTYPLCESIGVNSRVPLSILFGFLINGTCSPLDMNLETNP
>ORF121:
MVFKISIPINITDLFGNLLLLALITASRSAPNLLITKNRYSTLMDLYRPNALHVI
RPTCFIL
>ORF122:
MIVAVYGNLIMRITYTDRYQLDAMVSRK
>ORF123:
MTRWWRSRYICRSQCSNVKNSTCSQQILYIHLLILSYTTITKSFFKTLNHHQKSM
FKVIRLKYIFEINSCISYPSILDLYIFEINSCISHPSILDLYVLKNTILLIL
>ORF124:
MYFFNYYRKNNKYGIQPYIFLLSLTFYDISTYTSLFCSIDFILSSM
>ORF125:
MSGSYFPNILDPNSPTSGKFIIIQCLFISHDDPLSISSLSRILICGSK
>ORF126:
MSDEINSSQTSLSFPIDMITGITSLGLK
>ORF127:
MSRFFPRFILSYPSKSRKSNGYSICGMGLGRCKLCS
>ORF128:
MFVYKNNFRIQYDRRSFFKCFRYVFFEIIHFMREYWFHVSTKEGKLVIIFMNLYS
FTIVFDFRKHS
>ORF129:
MGNKNIKPSKENRLSILSKDKMDSFKRGSWATSSFKEKSRATIQRFSSLRREHIK
VDHPDKFLELKRGIYEIIQKSSSIDVDKRTKLMSNIKTMMINPFMIEGLMTSLEN
LDPDNKMSYSSVMILGEFDIINISDNEAAFEFINSLLKSLLLLNTRQLKLLEYSI
SNDLLYAHINALEYIIKNTFNVPERQLILRGQYLTPIFSDLLKYAGLTIKSNILM
WNKQFIKPVSDLYTSIRLLYCVTV
>ORF130:
MSIPFMIYHQDMYTQKVNVWPSNWL
>ORF131:
MLHGCTCWLQNISSIRDLRLKEMLTFATITLHRVRTICLIFY
>ORF132:
MSLYLSFISLYHIIESYPIIVIHVDRCRE
>ORF133:
MRHDTFTEHCISLYFKFPDNETIKLGLSEPYLCFKK
>ORF134:
MSSRYNDRRYRGVPSKQLHSIRVLIH
>ORF135:
MPFSHCYLLYMFFNKTSIGINLTLAFYSYYKI
>ORF136:
MQFGSIRCTTNIFPFFSSILRGIFSYKIIWGCLV
>ORF137:
MHLLSFYLLMVYELIYVLQLLIVSL
>ORF138:
MKYILYSRCSFSFSFISNHLTPDGPSKIGWLDKGFIISSSTLYFSMTSNVVCGTT
FPAGKHIPGAGDGNCFVYPSNFDVEKDCLMVFIE
>ORF139:
MINDPHTRNLLIHRNTNAHIYIINSTVQYLFLDRYRIVRLHFFVY
>ORF140:
MFCIYIISTVIISNKIANFRNAEYREYLHLYNVTAAIILNITSIL
>ORF141:
MILFLLIKIIIAMCSSKRIILTKFIVEFCNERI
>ORF142:
MAPSTEHIYDSVAGSTLLINNDRNEQTIYQNTTVVLNEDTKQNPNYSSNPFVNYN
KTSICSKSNPFITELNNKFSENNPFRRAHSDDYLNKQEQDHEHDDIESLV
>ORF143:
MEIFPVFGISKISNFIANNDCRYYIDTEHQKIISDEINRQMDETVLLTNILSVEV
VNDNEMYHLIPHRLSTIILCISSVGGCVISIDNDVNGKNILTFPIDHAVIISPLS
KCVVVSKGPTTILVVKADIPSKRLVTSFTNDILYVNNLSLINYLPLSVFIIRRVT
DYLDRHICDQIFANNKWYSIITIDNKQFPIPSNCIGMSSAKYINSSIEQDTLIHV
CNLEHPFDLVYKKMQSYNSVPIKEQILYGRIDNINMSISISVD
>ORF144:
MQSHRLRNSQTVVLSCFLHQKEEQF
>ORF145:
MAEWHKIIEDISKNNKFEDAAIVDYKTTKNVLAAIPNRTFAKINPGEIIPLITNR
NILKPLIGQKYCIVYTNSLMDENTYAMELLTGYAPVSPIVIARTHTALIFLMGKP
TTSRRDVYRTCRDHATRVRATGN
>ORF146:
MAFDISVNASKTINALVYFSTQQNKLVIRNEVNDTHYTVEFDRDKVVDTFISYNR
HNDTIEIRGVLPEETNIGCAVNTPVSMTYLYNKYSFKLILAEYIRHRNTISGNIY
SALMTLDDLAIKQYGDIDLLFNEKLKVDSDSGLFDFVNFVKDMICCDSRIVVALS
SLVSKHWELTNKKYRCMALAEHISDSIPISELSRLRYNLCKYLRGHTESIEDEFD
YFEDDDSSTCSAVTDRETDV
>ORF147:
MTSLREFRKLCCDIYHASGYKEKSKLIRDFITDRDDKYLIIKLLLPGLDDRIYNM
NDKQIIKLYSIIFKQSQEDMLQDLGYGYIGDTIRTFFKENTEIRPRDKSILTLEE
VDSFLTTLSSVTKESHQIKLLTDVASVCTCNDLKCVVMLIDKDLKIKAGPRYVLN
AISPHAYDVFRKSNNLKEIIENASKQNLDSISISVMTPINPMLAESCDSVNKAFK
KFPSGMFAEVKYDGERVQVHKNNNEFAFFSRNMKPVLSHKVDYLKEYIPKAFKKA
TSIVLDSEIVLVDEHNVPLPFGSLGIHKKKEYKNSNMCLFVFDCLYFDGFDMTDI
PLYERRSFLKDVMVEIPNRIVFSELTNISNESQLTDVLDDALTRKLEGLVLKDIN
GVYEPGKRRWLKIKRDYLNEGSMADSADLVVLGAYYGKGAKGGIMAVFLMGCYDD
ESGKWKTVTKCSGHDDNTLRELQDQLKMIKINKDPKKIPEWLVVNKIYIPDFVVE
DPKQSQIWEISGAEFTSSKSHTANGISIRFPRFTRIREDKTWKESTHLNDLVNLT
KS
>ORF148:
MRGVKFISRSHSSHECFLFYQRKCLVDYSPV
>ORF149:
MDIKNLLTACTIFYITTLATADIPTPPPTGHVTRENILIRGIINVVIGVHLENLP
RLDVMVTITQNVNAAHLIHIPQSPIILMDVINVENAQPDHLIR
>ORF150:
MNKEILFVNRAVLVNIATTYVIIDLIHFLHANYLNVINYDFDDNVTIHYIATWLV
YYSV
>ORF151:
MNSSSKLIAVINGFRNSGRFCDINIVINDERINAHRLILSGASEYFSILFSNNFI
DSNEYEVNLSHLDYQSVNDLIDYIYGIPLSLTNDNVKYILSTADFLQIGSAITEC
EKYILKNLCSRNCIDFYIYADKYNNKKIESASFNTILRNILRLINDENFKYLTEE
SMIKILSDDMLNIKNEDFAPLILIKWLESTQQSCTVELLRCLRISLLSPQVIKSL
YSHQLVSSIYECITFLNNIAFLDESFPRYHSIELISIGISNSHDKISINCYNHKK
NTWEMISSRRYRCSFAVAVLDNIIYMMGGYDQSPYRSSKVIAYNTCTNSWIYDIP
ELKYPRSNCGGLADDEYIYCIGGIRDQDSSLTSSIDRWKPSKPYWQKYAKMREPK
CDMGVAMLNGLIYVMGGIVKGDTCTDALESLSEDGWMKHQRLPIKMSNMSTIVHD
GKIYISGGYNNSSVVNVISNLVLSYNPIYDEWTKLSSLNIPRINPALWSAHNKLY
VGGGISDDVRTNTSETYDKEKDCWTLDNGHVLPRNYIMYKCEPIKHKYPLEKTQY
TNDFLKYLESFIGS
>ORF152:
MSGIVKSIILSGPSGSGKTAIAKRLWEYIWICGVPYH
>ORF153:
MAMFYAHALGGYDENLHAFPGISSTVANDVRKYSVVSVYNNKYDIVKDKYMWCYS
QVNKRYIGALLPMFECNEYLQIGDPIHDQEGNQISIITYRHKNYYALSGIGYESL
DLCLEGVGIHHHVLETGNAVYGKVQHDYSTIKEKAKEMNALSSGPIIDYHVWIGD
CICQVTAVDVHGKEIMRMRFKKGAVLQIPNLVKVKLGENDTENLSSTISAAPSR
>ORF154:
MDFFKKEILDWSVYLSLHYIARVCSNSSTSHIIQDYNLVRTYEKVDKTIVDFLSR
LPNLFHILEYGENILHIYSMDDANTNIIIFFLDRVLNINKNGSFIHNLRLSSSIN
IKEYVYQLVNNDHPDNRIRLMLENGRRTRHFLSYISDTVNIYICILINHGFYIDA
EDSYGCTLLHRCIYHYKKSESESYNELIKILLNNGSDVDKKDTYGNTPFILLCKH
DINNVELFEICLENANIDSVDFNRYTPLHYVSCRNKYDFVKLLISKGANVNARNK
FGTTPFYCGIIHGISLIKLYLESDTELEIDNEHIVRHLIIFDAVESLDYLLSRGV
IDINYRTIYNETSIYDAVSYNAYNTLVYLLNRNGDFETITTSGCTCISEAVANNN
KIIMEVLLSKRPSLKIMIQSMIAITKNKQHNADLLKMCIKYTACMTDYDTLIDVQ
SLQQYKWYILKCFDEIDIMKRCYIKNKTVFQLVFCIKDINTLMRYGKHPSFVKCT
SLDVYGSRVRNIIASIRYRQRLISLLSKKLDAGDKWSCFPNEIKYKILENFNDNE
LSTYLKIL
>ORF155:
MLISTMPLEHFYSGTIITRDLLCMEEVTPYYIIYTGYLQDAPLYRSMI
>ORF156:
MYKKLITFLFVIGALASYSNNEYTPFNKLSVKLYIDGVDNIENSYTDDNNELVLN
FKEYTISIITESCDVGFDSIDIDVINDYKIIDMYTIDSSTIQRRGHTCRISTKLS
CHYDKYPYIHKYDGDEQQYSITAEGKCYKGIKYEISMINDDTLLRKHTLKIGSTY
IFDRHGHSNTYYSKYDF
>ORF157:
MRYIIILAVLFINSIHAKITSYKFESVNFDSKIEWTGDGLYNISLKNYGIKTWQT
MYTNVPEGTYDISAFPKNDFVSFWVKFEQGDYKVEEYCTGLCVEVKIGPPTVTLT
EYDDHINLYIEHPYATRGSKKIPIYKRGDMCDIYLLYTANFTFGDSKEPVPYDID
DYDCTSTGCSIDFVTTEKVCVTAQGATEGFLEKITPWSSKVCLTPKKSVYTCAIR
SKEDVPNFKDKMARVIKRKFNKQSQSYLTKFLGSTSNDVTTFLSMLNLTKYS
>ORF158:
MDSGIYETPINYKKSNVSAVSVNNTIFVTGGLFINNSNSTIVVNNMEKLDIYKDK
QWSIIEMPMARVYHGIDSTFGMLYFAGGLSVTEQYGNLEKNNEISCYNPRTNKWF
DISYTIYKISISSLCKLNNVFYVFSKDIGYVEKYDGAWKLVHDRLPAIKALSTSP
Y
>ORF159:
MARNGLSEILYILVIQYYIRSEKISLVRSTIT
>ORF160:
MDIFREIASSMKGENVFISPASISSVLTILYYGANGSTAEQLSKYVETEENTDKV
SAQNISFKSMNKVYGRYSAVFKDSFLRKIGDKFQTVDFTDCRTIDAINKCVDIFT
EGKINPLLDEPLSPDTCLLAISAVYFKAKWLMPFEKEFTSDYPFYVSPTEMVDVS
MMSMYGKAFNHASVKESFGNFSIIELPYVGDTSMMVILPDKIDGLESIEQNLTDT
NFKKWCNSLEATFIDVHIPKFKVTGSYNLVDTLVKSGLTEVFGSTGDYSNMCNSD
VSVDAMIHKTYIDVNEEYTEAAAATCALVSDCASTITNEFCVDHPFIYVIRHVDG
KILFVGRYCSPTTNC
>ORF161:
MTANFSTHVFSPQHCGCDRLTSIDDVRQCLTEYIYWSSYAYRNRQCAGQLYSTLL
SFRDDAESVFIDIRELVKNMPWDDVKDCTEIIRCYIPDEQKTIREISAIIGLCAY
AATYWGGEDHPTSNSLNALFVMLEMLNYVDYNIIFRRMN
>ORF162:
MYRQRAIFCIIHGVRKRASNLTMSSNKYAIIRI
>ORF163:
MTHFTIGTLRIAILKRIDTHFQNTCRLINTMV
>ORF164:
MRDTLMYIIKICTCQRYMINYFLFSQIKIVY
>ORF165:
MEMYPRHRYSKHSVFKGFSDKVRKNDLDMNVVKELLSNGASLTIKDSSNKDPITV
YFRRTIMNLEMIDIINKHTTIDERKYIVHSYLKNYKNFDYPFFRKLVLTNKHCLN
NYYNISDSKYGTPLHILASNKKLITPNYMKLLVYNGNDINARGEDTQMRTPLHKY
LCKFVYHNIEYGIRYYNEKIIDAFIELGADLTIPNNDGMIPVVYCIHSNAEYGYN
NITNIKIIRKLLNLSRRASHNLFRDRVMHDYISNTYIDLECLDIIRSLDGFDING
YFEGRTPLHCAIQHNFTQIAKYLLDRGADIVVPNTLIIHQYIQ
>ORF166:
MIYMLYATSKMKKMLRLVYLDITRIRITDGNWLRRQKADYQLCILFFMTIP
>ORF167:
MNRICYKIHLMCTLANGIICVINIRIVISCTIYYPSTKYNRIK
>ORF168:
MNLQKLSLAIYLTATCSWCYETCIRKTALYHDIQLEHVEDNKDSVASLPYK
>ORF169:
MIEVILFFITSASICPLMRKRHLIRCIVKRNLLKSW
>ORF170:
MKPKVNNIGNTPLHNYVSQYDITLIPHPQPIKKMEIKALY
>ORF171:
MRYIVTSSINAIQTLRLFDCYSLAESRDFVETTKD
>ORF172:
MAVNHVSNNGRLCMYGLILSRFNNCGYHCYETILIDVFDILSKYMDNIDMIDNEN
KTLLYYAVDVNNIQFAKRLLEYGASVTTSRSIINTAIQKSSYRRENKTRIVDLLL
SYHPTLETMIDAFNRDIRYLYPEPLFACIRYALILDDDFPSKVSMISPVVIRN
>ORF173:
MLFYLEEPIRGYVIILIVHPSWNDCATGHILIMLLNWHEQKEEGQHLLYLFIKHN
QGYTLNILRYLLDRFDIQKDEYYNTAFQNCNNNVASYIGYDINLPTKDGIRLGV
>ORF174:
MYDDLKPVPRNTFNYIDVEINLYPVNDTSCTRTTTTGLSESISTSELTITMNHKD
CNPVFRDGYFSVLNKVATSGFFTGERCAL
>ORF175:
MHVPASLQQSSSSCTEEENKHHMGIDVIIKVTKQDQTPTDDKICQSVTEITESES
DPDPEVESVEDVDPPTTYYSIIGGGLRMNFGFTKCPQIKSISESADGKTVRCLST
SDVAKKRKTATSRPIQYSGLTSLIRK
>ORF176:
MSTDRHLKVQPMILHSSIQQNSKRVSESITLFI
>ORF177:
MSRVNRTIQAHHELKKCTGHYIPVCKTKSYLSRLLLFLSQ
>ORF178:
MVSLKYFYSHSLFNGVIKYFYSLSLFDGLTKILNLFLMVSLKYFYSHSLFNGVIK
YFYSLSLFDGLIKKVLQKYFYSLSLFDGLIKKVLQKYFYSLSLFDGLIKKFYKNI
FILFLSLMVSQKYFYSLSLFDGLIKKVLQKYFYSLSLFDGLIKNIKPLSDGVTKI
FLFSFSLQWSHKIFLFSFSLRWSHKNIKPLSDGVTKIFLFSFSLQWSHKIFLFSF
SL
>ORF179:
MTMRCTILFLIDYRRLYSVLVLSEDVLSL
>ORF180:
47818:
MVIQFPSNILCRIIYSTRNNDGIITCCILYGLCSCYNNQRIEVYQRYSNS
>ORF181:
MCLNDEGGPSSLSSHRWSTFLKVELECDIDGRSYRQIIHSRTIKTDNDTILYVFF
DSPYSKSALCTYSMNTIKQSFSTSKLEGYTKQLPSPAPGICLPAGKVVPHTTFEV
IEKYNVLDDIIKPLSNQPIFEGPSGVKWFDIKEKENEHREYRIYFIKENSIYSFD
TKSKQTRSSQVDARLFSVMVTSKPLFIADIGIGVGMPQMKKILKM
>ORF182:
MPRRMELKNGNIFVVQRILPNCIRVTLYNNYTTFLS
>ORF183:
MLFNVSALIVYFDVCSHIVYAIVRL
>ORF184:
MGHRIYRFRSPWIQTSKLRVYGIAISVYTNCMKIKKTV
>ORF185:
MCIERVEITLPVYAQLVIKIKSNIHM
>ORF186:
MMMMKWIISILTMSIMPVLAYSSSIFRFHSEDVELCYGHLYFDRIYNVVNIKYNP
HIPYRYNFINRTLTVDELDDNVFFTHGYFLKHKYGSLNPSLIVSLSGNLKYNDIQ
CSVNVSCLIKNLATSTSTILTSKHKTYSLHRSTCITIIGYDSIIWYKDINDKYND
IYDFTAICMLIASTLIVTIYVFKKIKMNS
>ORF187:
MHRVIIDCTIHNVTVLLTLLVKDFRQFVSFSVNSSGS
>ORF188:
MLMADEWITVFIGSYCFHATSIDQILDS
>ORF189:
MYNFFYSVKDMIKNIIVVFIPFQSPYMIL
>ORF190:
MIYSNAFMWAYNKSLLMEYSKSFSCLVFNKRRDFNRLFMNSNAASLSLILMMSNS
PNIITDE
>ORF191:
MNGFIIIVFMLDMSLVRLSTSIDDDF
>ORF192:
MVARDFSLKDDVAQDPLLNESILSLDKMDSLFSLDGLIFLLPMIYKGRPNRLG
>ORF193:
MDEAYYSGNLESVLGYVSDMHTELASISQLVIAKIETIDNDILNKDIVNFIMCRS
NLDNPFISFLDTVYTIIDQEIYQTELINSLDDNEIIDCIVNKFMSFYKDNLENIV
DAIITLKYIMNNPDFKTTYAEVLGSRIADIDIKQVIRKNILQLSNDIRERYL
>ORF194:
MDGVIVYCLNALVKHGEEINHIKNDFMIKPCCERVCEKVKNVHIGGQSKNNTVIA
DLPYMDNAVSDVCNSLYKKNVSRISRFANLIKIDDDDKTPTGVYNYFKPKDVIPV
IISIGKDKDVCELLISSDISCACVELNSYHVAILPMNVSFFTKGNASLIILLFDF
SIDAAPLLRSVTDNNVIISRHQRLHDELPSSNWFKFYISIKSDYCSILYMVVDGS
VMHAIADNRTHAIISKNILDNTTINDECRCCYFEPQIRILDRDEMLNGSSCDMNR
HCIMMNLPDVGEFGSSMLGKYEPDMIKIALSVAGNLIRNRDYIPGRRGYSYYVYG
IASR
>ORF195:
MDIKIDISISGDKFTVTTRRENEERKKYLPLQKEKTTDVIKPDYLEYDDLLDRDE
MFTILEEYFMYRGLLGLRIKYGRLFNEIKKFDNDAEEQFGTIEELKQKLRLNSEE
GADNFIDYIKVQKQDIVKLTVYDCISMIGLCACVVDVWRNEKLFSRWKYCLRAIK
LFINDHMLDKIKSILQNRLVYVEMS
>ORF196:
MMFELIHLKHTIKKKIVGHWIMVTCYHAII
>ORF197:
MTRLPILLLLISLVYATPFPQTSKKIGDDATLSCNRNNTNDYVVMSAWYKEPNSI
ILLAAKSDVLYFDNYTKDKISYDSPYDDLVTTITIKSLTARDAGTYVCAFFMTST
TNDTDKVDYEEYSTELIVNTDSESTIDIILSGSTHSPETSSEKPEDIDNFNCSSV
FEIATPEPITDNVEDHTDTVTYTSDSINTVSASSGESTTDETPEPITDKEEDHTV
TDTVSYTTVSTSSGIVTTKSTTDDADLYDTYNDNDTVPPTTVGGSTTSISNYKTK
DFVEIFGITALIILSAVAIFCITYYIYNKRSRKYKTENKV
>ORF198:
MEREGVDYHYVNREAIWKGIAAGNFLEHTEFLGNIYGTSKTAVNTAAINNRICVM
DLNIDGVRSLKNTYLMPYSVYIRPTSLKMVETKLRCRNTEADDEIHRRVMLAKTD
MDEAGEAGLFDTIIIEDDVNLAYSKLIQILQDRIRMYFNTN
>ORF199:
31420:
MNFQGLVLTDNCKNQWVVGPLIGKGGFGSIYTTNDNNYVVKIEPKANGSLFTEQA
FYTRVLKPSVIEEWKKSHNIKHVGLITCKAFGLYKSINVEYRFLVINRLGADLDA
VIRANNNRLPKRSVMLIGIEILNTIQFMHEQGYSHGDIKASNIVLDQIDKNKLYL
VDYGLVSKFMSNGEHVPFIRNPNKMDNGTLEFTPIDSHKGYVVSRRGDLETLGYC
MIRWLGGILPWTKISETKNCALVSATKQKYVNNTATLLMTSLQYAPRELLQYITM
VNSLTYFEEPNYDEFRHILMQGVYY
>ORF200:
MPFLEYHRLLPMMSGNILLCQFIITSMTL
>ORF201:
MNIYKLEIRSMIKKEIKSLSSHIATKTTML
>ORF202:
MIQKIFLLLYRRHHRGNHLSRRPRE
>ORF203:
MRLPRHTCALSKWKDAVVLCLQIGQNHGIWILMVSRSILQMNGCHI
>ORF204:
MLQNINYIWIFLKRKYLTGVYIYLFII
>ORF205:
MQKTVTVVHYYIDVYITIRNQNQNHTMN
>ORF206:
MKTISVVTLLCVLPAVVYSTCTVPTMNNAKLTSTETSFNDKQKVTFTCDQGYHSL
DPNAVCETDKWKYENPCKKMCTVSDYVSELYDKPLYEVNSTMTLSCNGETKYFRC
EEKNGNTSWNDTVTCPNAECQPLQLEHGSCQPVKEKYSFGEYMTINCDVGYEVIG
ASYISCTANSWNVIPSCQQKCDIPSLSNGLISGSTFSIGGVIHLSCKSGFILTGS
PSSTCIDGKWNPILPTCVRSNEKFDPVDDGPDDETDLSKLSKDVVQYEQEIESLE
ATYHIIIVALTIMGVIFLISVIVLVCSCDKNNDQYKFHKLLP
>ORF207:
MISTLIFTNMMVMSNNILLLQRENAIKE
>ORF208:
MDIVIHIIQNMIFKNLKYIITSVTVVK
>ORF209:
MVYTIYPLKIMASRRGKQCIQMYQKEHTTYPHFQRMISYLSGLNLNKAIIKWKSI
VRDYASK
>ORF210:
MLLEVAKRFLFTNAVTCVISTCCIRLTSHSEILKNQYHMISMTTIARLQVAA
>ORF211:
MIQKRNKTAYYTLVNALIGSNHFQDEVPDYSPSVPLYHLLHVY
>ORF212:
MRHYDKHVRVYTLSRIYPWKNRTEEYTD
>ORF213:
MSMKSIQKQLQQLVHWCQTVHQQLQMSSV
>ORF214:
20587:
MHTATGNALDSCIPHSSLLEMMRNQCSSTFASW
>ORF215:
MMSKIVQKSSVVIYRMSKKPSERFRPSSDFVHMLLLTGEVKTIPLVTV
>ORF216:
MSILPIIFLPIFFYSSFVQTFNAPECIDKGQYFASFMELENEPVILPCPQINTLS
SGYNILDILWEKRGADNDRIIPIDNGSNMLILNPTQSDSGIYICITTNETYCDMM
SLNLTIVSVSESNIDLISYPQIVNERSTGEMVCPNINAFIASNVNADIIWSGHRR
LRNKRLKQRTPGIITIEDVRKNDAGYYTCVLEYIYRGKTYNVTRIVKLEVRDKII
PSTMQLPDGIVTSIGSNLTIACRVSLRPPTTDADVFWISNGMYYEEDDGDGDGRI
SVANKIYMTDKRRVITSRLNINPVKEEDATTFTCMAFTIPSISKTVTVSIT
>ORF217:
MRTFTTKHHKNESSSDLCFKYQPQINS
>ORF218:
MSRRNNVTQTVHCSRHYTICSFKMP
>ORF219:
MTMKMMVHIYFVSLLLLLFHSYAIDIENEITEFFNKMRDTLPAKDSKWLNPACMF
GGTMNDIAALGEPFSAKCPPIEDSLLSHRYKDYVVKWERLEKNRRRQVSNKRVKH
GDLWIANYTSKFSNRRYLCTVTTKNGDCVQGIVRSHIKKPPSCIPKTYELGTHDK
YGIDLYCGILYAKHYNNITWYKDNKEINIDDIKYSQTGKKLIIHNPELEDSGRYN
CYVHYDDVRIKNDIVVSRCKILTVIPSQDHRFKLILDPKINVTIGEPANITCTAV
STSLLIDDVLIEWENPSGWLIGFDFDVYSVLTSRGGITEATLYFENVTEEYIGNT
YKCRGHNYYFEKTLTTTVVLE
>ORF220:
MNSESDNISIKTEYEFYDETQDQSTQLVGYDIKLKTNEDDFMAMIDQWVSMII
>ORF221:
MDSISMVTLKDVHHFIALYNITSLRLLSTY
>ORF222:
MLALLMVCNMIDCVRNTNLLSMNTGYKHKLKFNIIYL
>ORF223:
MMIYGLIACLIFVTSSIASPLYIPVIPPITEDKSFNSVEVLVSLFRDDQKDYTVT
SQFNNYTIDTKDWTIGVLSTPDGLDIPLTNITYWSRFTIGRALFKSESEDIFQKK
MSILGVSIECKKSSTLLTFLTVRKMTRVFNKFPDMAYYRGDCLKAVYVTMTYKNT
KTGETDYTYLSNGGLPAYYRNGVDG
>ORF224:
MIHVLKYFPNKSFKYCINYEKLCYASMMQRCLMIRY
>ORF225:
MISLSFLIHNPLKKWKLKPSISINGYRSTFTMASPCAQFRPCHCHATKDSLNTVA
DVRHCLTEYILWVSHRWTHRESAGSLYRLLISFRTDATELFGGELKDSLPWDNID
NCVEIIKCFIRNDSMKTAEELRAIIGLCTQSAIVSGRVFNDKYIDILLMLRKILN
ENDYLTLLDHIRTAKY
>ORF226:
MRIKLYYITRSMSIIYNLQSGYWNMERVLQHHAR
>ORF227:
MTYYKIDSVQDDKNIYRHHKHGVYFICLA
>ORF228:
MDEIVRIVRDSMWYIPNVFMDDGKNEGHVSVNNVCHMYFTFFDVDTSSHLFKLVI
KHCDLNKRGNSPLHCYTMNTRFNPSVLKILLHHGMRNFDSKDEKGHIPLHHYLIH
SLSIDNKIFDILTDTIDDFSKSSDLLLCYLRYKFNGSLNYYVLYKGSDPNCVDED
GLTSLHYYCKHISTFYKSNYYKLSHTKMRAEKRFIYAIIDYGANINAVTHLPSTV
YQT
>ORF229:
MLPHTSDTTSTFRLKTVFDLVFENRNIIYKADVVNDIIHHRLKVSLPMIKSLFYK
MSLPTTITT
>ORF230:
MWKLICIPSTTHRVLGRPLPVSANPSQRRN
>ORF231:
MKQYIVLACMCLPVFSNHPHRVRKKKTNIIWESMLLSKSQSKTKHRPMIRFANP
>ORF232:
MVSLKYFYSLSLFNGVIKYFYSLSLFDGLTKILNLFLMVSLKYFYSLSLFNGVIK
YFYSLSLFDGLTKIFLFSFSL
>ORF233:
MVSLKYFYSHSLFNGVIKYFYSLSLFDGLTKILNLFLMVSLKYFYSHSLFNGVIK
YFYSLSLFDGLIKKVLQKYFYSLSLFDGLIKKVLQKYFYSLSLFDGLIKKVLQKY
FYSLSLFDGLIKKVLQKYFYSLSLFDGLIKKVLQKYFYSLSLFDGLIKNIKPLSD
GVTKIFLFSFSFSLSLFNGVIKYFYSLSLFDGLTKILNLFLMVSLKYFYSHSLFN
GVIKYFYSLSLFDGLIKKVLQKYFYSLSLFDGLIKKVLQKYFYSLSLFDGLIKKV
LQKYFYSLSLFDGLIKKVLQKYFYSLSLFDGLTKILNLFLMES
>ORF234:
MDSFSSLFMKLCCISTDKTGSKKSDKKNKNKIKDYYKITIVPGSSSTSTSSWYYT
HA
>ORF235:
MRCTAHDNLHSNQNENTLVIIRNIDGNVIINNPIICRRVNIDG
>ORF236:
MIPLLFILFYFANGIEWHKFETSEEIISTYLLDDVLYTGVNGAVYTFSNNKLNKT
GLTNNNYITTSIKVEDAEPITEIPNVGK
>ORF237:
MVYEEHSSIKMVLMTKFTFFSLILSAQRELSKFRI
>ORF238:
MNKHKTDYAGYACCVICGLIVGIIFTATLLKVVERKLVHTPSIDKTIKDAYIRED
CPTDWISYNNKCIHLSTDRKNLGGRT
>ORF239:
MLPTSEGGTVLKSLGFNLLPLVFMKSFVLSTSHFK
>ORF240:
MSTIHSSVNFTRSSWVIEFIGSVVHGSKLANFAYTVSRLVYTPTV
>ORF241:
MLLEMDKIKITVDSKIGNVVTISYNLEKITIDVTPKKKKEKDVLLAQSVAVEEAK
DVKVEEKNIIDIEDDDDMDVESA
>ORF242:
MITELLAFITSCSAIRLLFAYTCPGDIS
>ORF243:
MAVCIIDHDNIRGVIYFEPVHGKDKVLGSVIGLKSGTYSLIIHRYGDISQGCDSI
GSPEIFIGNIFVNRYGVAYVYLDTDVNIFTIIGKALSISKNDQRLACGVIGISYI
NEKIIHFLTINENGV
>ORF244:
MSRGALIVFEGLDKSGKTTQCMNIMESIPANTIKYLNFPQRSTVTGKMIDDYLTR
KKTYNDHIVNLLFCANRWEFASFIQEQLEQGITLIVDRYAFSGVAYAAAKGASMT
LSKSYESGLPKPDLVIFLESGSKEINRNVGEEIYEDVTFQQKVLQEYKKMIEEGD
IHWQIISSEFEEDVKKELIKNIVIEAIHTVTGPVGQLWM
>ORF245:
MLVQCYKWMKHITLATWNQYSDTCPICIPNSHQYLN
>ORF246:
MVKNKARLFERGFHGRFCRFSSTRCLLW
>ORF247:
MTMTRLLLVYIIILNLKMLFLLSYL
>ORF248:
MSVDAVILNHRLGFLIEMRCSMDHRVI
>ORF249:
MKKEKNIYLSKKKKLLMLSNLIILSTMTC
>ORF250:
MFGEMRNCFLDGNIVYERLNCLLMITCLIR
>ORF251:
MWKCHRKLMRAKIYKVVFHICYFFCNS
>ORF252:
MIILKDGYKEFADCMYYFLHYYIGYGRYTYSATNGSCDKGEYLDKRHNQCCNRCP
PGEFAKVRCNGNDNTKCERCPPHTYTTIPNYSNGCHQCRKCPTGSFDKVKCTGTQ
NSKCSCLPGWYCATDSSQTEDC
>ORF253:
MGIYLDLWCPIPLDFLVLWNEKVSITITLTERPSGRE
>ORF254:
MKRLETIRHMWSVVYDHFDIVNGKECCYVHTHSSNQNPIPSTVKTNLYMKTMGSC
IQMDSMEALEYLSELKESGGWSPRPEMQEFEYPDGVEDTESIERLVEEFFNRSEL
QAGKLVKFGNSINC
>ORF255:
MINRKLHLHVIRDIILWIQMLSAKQINGNTKIHARKCAQFLIMSLNYMISHYTK
>ORF256:
MTNISSINCYRKYKSVKIINNLITNKYQKIKDL
>ORF257:
MSSSVDVDIYDAVRAFLLRHYYNKRFIVYGRSNAILHNIYRLFTRCAVIPFDDIV
RTMPNESRVKQWVMDTLNGIMMNERDVSVSVGTGILFMEMFFDYNKNSINNQLMY
DIINSVSIILANERYRSAFNDDGIYIRRNMINKLYGYASLTTIGTIAGGVCYYLL
MHLVSLYK
>ORF258:
MRSLIIVLLFPSIIYSMSIRRCEKTEEETWGLKIGLCIIAKDFYPERTDCSVHLP
TASEGLITEGNGFRDIRNTDKL
>ORF259:
MDTDVTNVEDIINEIDREKEEILKNVEIENNKNINKNHPSGYIREALVINTSSNS
DSIDKEVIECISHDVGI
>ORF260:
MESFKYCFDNDGKKWIIGNTLYSGNSILYKVRKNFTSSFYNYVMKIDHKSHKPLL
SEIRFYISVLDPLTIDNWTRERGIKYLAIPDLYGIGETDDYMFFVIKNSGRVFAP
KDTESVFEACVTMINTLEFIHSRGFTHGKIEPRNILIRNKRLSLIDYSRTNKLYK
SGNSHIDYNEDMITSGNINYMCVDNHLGATVSKRGDLEMLGYCMIEWFGGKLPWK
NESSIKVIKQKKEYKKFIATFFEDCFPEGNEPLELVRYIELVYTLDYSQTPNYDR
LRKLFIQD
>ORF261:
MGISMSSLVYCEQQEQISIGKGGMRYIDLCSFKTHANFEEFI
>ORF262:
MNSSRYCSLSYSYTCINFLDILQSVLF
>ORF263:
MSRRLIYVLNINRKSTHKIQENEIYTYFSHCNIDHTSTELDFVVKNYDLNRRQHV
TGYTALHCYLYNNYFTNDVLKILLNHDVNVTMKTSSGRMPVYILLTRCCNISHDV
VIDMIDKDKNHLLHRDYSNLLLEYIKSRYMLLKEEDIDENIVSTLLDKGIDPNFK
QDGYTALHYYYLCLAHVYKPGECRKPITIKKAKRIISLFIQHGANLNALDNCGNT
PFHLYLSIEMCNNIHMTKMLLTFNPNFKICNNHGLTPILCYITSDYIQHDILVML
IHHYETNVGEMPIDERRMIVFEFIKTYSTRPADSITYLMNRFKNINIYTRYEGKT
LLHVACEYNNTHVIDYLIRINGDINALTDNNKHATQLIIDNKENSPYTIDCLLYI
LRYIVDKNVIRSLVDQLPSLPIFDIKSFEKFISYCILLDDTFYDRHVKNRDSKTY
RYAFSKYMSFDKYDGIITKCHDETMLLKLSTVLDTTLYAVLRCHNSKKLRRYLNE
LKKYNNDKSFKIYSNIMNERYLNVYYKDMYVSKVYDKLFPVFTDKNCLLTLLPSE
IIYEILYMLTINDLYNISYPPTKV
>ORF264:
MSTFIIIDQSTENTSIDTTVTINIIYLAIMKIIMNIIMMIMIELV
>ORF265:
MEEDTNISNKVIRYNTVNNIWKTLPNFWTGTINPGVVSHKDDIYVVCDIKDEKNV
KTCIFRYNTNTYNGWELVTTTESRLSALHTILHDNTIMMLHCYESYMLQDTFNVY
TREWNHMCHQHSNSYIMYNILPIY
>ORF266:
MDIFKELILKHPDENVLISPVSILSTLSILNHGAAGSTAEQLSKYIENMNENTPD
DKKDDNNDMDVDIPYCATLATANKIYGSDSIEFHASFLQKIKDDFQTVNFNNANQ
TKELINEWVKTMTNGKINSLLTSPLSINTRMTVVSAVHFKAMWKYPFSKHLTYTD
KFYISKNIVTSVDMMVGTENNLQYVHINELFGGFSIIDIPYEGNSSMVIILPDDI
EGIYNIEKNITDEKFKKWCGMLSTKSIDLYMPKFKVEMTEPYNLVPILENLGLTN
IFGYYADFSKMCNETITVEKFLHTTFIDVNEEYTEASAVTGVFMTNFSMVYRTKV
YINHPFMYMIKDNTGRILFIGKYCYPQ
>ORF267:
MSLESFIITTFNNNSSTNIDNMCHLYVKVCPSSLLFRLFVECCDINKLVEGTTPL
HCYLMNEGFESSVLKNLLKEYVMNTFNVHDIHYTNI
>ORF268:
MSTYEKTSFNKMHREKKFIKELVKYETESK
>ORF269:
MIAFIIFREIGIISTRIAMDCTCILCRLLDEDVTYKKIKLEIETCHNLSKHIDRR
GNNALHCYVFNKCDTDIKIVRLLLSRGVERLCRNNEGLTPLGAYSKHRYVKSQIV
HLLISSYSNSSNELKSNINDFDLYSYMSSDNIDLRLLKYLIVDKRIRPSKNTNYA
INGLGLVDIYVTTPNPRPEVLLWLLKSECYSTGYVFRTCMYDSDMCKNSLHYYIS
SHRESQSLSKDVIKCLINNNVSIHGRDEGGSLPIQYYWSCSTIDIEIVKLLIKDV
DTCRVYDVSPILEADYLNKRFRVTPYNVDMEIVNLLIERRHTLVDVMRSITSYDS
RDYNHYIIDNILKRFRQQDESIVQAMLINYLHYGDMVVRCMLDNGQQLSSARLLC
>ORF270:
MKNAYISGVSMFDILFKRSKRHRLRYAKNPTSNGTKKN
>ORF271:
MRWTRLYASFATVCGTYLTYLWTTVRMKVTFLSTMFVICILRSLMWIHRLICLS
>ORF272:
MEQTLTRLHTYLQQYTKHSPRVVYALLSRGADTRIRNNLDCTPIMERLCNRSYSH
NVTQLARTKGRRTTSTLSIHKT
>ORF273:
MPHSETVTLASTSIYYIVIPILKTTKLIOSLIMWVM
>ORF274:
MCKESSELEVKYVDGSASEGATDDTSLIDSTKLKACV
>ORF275:
MVSQKYFYSLSLFDGLIKKVLQKYFYSLSLFDGLIKKVLQKYFYSLSLFDGLIKK
VLQKYFYSLSLFDGLIKKVLQKYFYSLSLFDGLIKKVLQKYFYSLSLFDGLIKNI
KPLSDGVTKIFLFSFSLQWSHKIFLFSFSLRWSHKNIKPLSDGVTKIFLFSFSLQ
WSHKIFLFSFSL

TABLE 5
OV19t Open Read Frames (ORFs) and
nr NCBI Database Entry from Blast

| OV19t | Subject.accession |
|---|---|
| ORF1 | P20549.1 |
| ORF17 | ABZ80166.1 |
| ORF26 | DAA80683.1 |
| ORF27 | P20543.1 |
| ORF28 | UJQ44755.1 |
| ORF32 | QGQ59895.1 |
| ORF34 | ABZ80145.1 |
| ORF36 | P68628.1 |
| ORF41 | JQ1823 |
| ORF52 | ABZ80122.1 |
| ORF53 | P20524.1 |
| ORF55 | P20523.1 |
| ORF63 | AAW23389.1 |
| ORF65 | AUL80383.1 |
| ORF66 | AND73835.1 |
| ORF76 | AAQ93296.1 |
| ORF77 | DAA80696.1 |
| ORF79 | AAS49884.1 |
| ORF81 | H36855 |
| ORF82 | P68474.1 |
| ORF87 | P20530.1 |
| ORF90 | ABZ80136.1 |
| ORF91 | AGB75887.1 |
| ORF92 | AAB96478.1 |
| ORF93 | ABD52653.1 |
| ORF104 | YP_910501.1 |
| ORF108 | AAG37700.1 |
| ORF110 | AAW23645.1 |
| ORF111 | P68476.1 |
| ORF116 | ABZ80159.1 |
| ORF118 | AAW23628.1 |
| ORF120 | P68472.1 |
| ORF123 | ALF05177.1 |
| ORF128 | P20527.1 |
| ORF129 | AGB75890.1 |
| ORF130 | WVM33661.1 |
| ORF138 | AAW23599.1 |
| ORF140 | CRL86663.1 |
| ORF142 | AAB96531.1 |
| ORF143 | AGK06629.1 |
| ORF145 | AAX78481.1 |
| ORF146 | AAS49868.1 |
| ORF147 | AGB75893.1 |
| ORF149 | AAW23902.1 |
| ORF150 | AHB35818.1 |
| ORF151 | AGB75897.1 |
| ORF152 | AGB75899.1 |
| ORF153 | AGB75902.1 |
| ORF154 | AGB75904.1 |
| ORF155 | ABG56123.1 |
| ORF156 | AAS49882.1 |
| ORF157 | AAX78486.1 |
| ORF158 | YP_233074.1 |
| ORF160 | AGB75913.1 |
| ORF161 | ADZ29313.1 |
| ORF165 | AGB75921.1 |
| ORF168 | AAQ93098.1 |
| ORF169 | DAD53330.1 |
| ORF170 | ALF04987.1 |
| ORF172 | AIX98921.1 |
| ORF173 | AAX78507.1 |
| ORF174 | AND73828.1 |
| ORF175 | AAT10397.1 |
| ORF176 | WLW36592.1 |
| ORF177 | ALF04974.1 |
| ORF178 | UXO30862.1 |
| ORF181 | AAT10549.1 |
| ORF182 | AIX98861.1 |
| ORF186 | P21065.1 |
| ORF192 | SNB48636.1 |
| ORF193 | AGB75892.1 |
| ORF194 | AGB75894.1 |
| ORF195 | YP_233060.1 |
| ORF197 | P08714.1 |
| ORF198 | AGB75900.1 |
| ORF199 | AAX78484.1 |
| ORF203 | ABD52684.1 |
| ORF206 | ACA50704.1 |
| ORF211 | UZL86921.1 |
| ORF212 | YP_717506.1 |
| ORF216 | AGB75915.1 |
| ORF219 | AAR91035.1 |
| ORF220: | YP_233084.1 |
| ORF222 | AUO38306.1 |
| ORF223 | AAR17846.1 |
| ORF224 | DAD53328.1 |
| ORF225 | AIX98923.1 |
| ORF226 | ABZ79903.1 |
| ORF227 | AGY98963.1 |
| ORF228 | AGB75726.1 |
| ORF229 | YP_232888.1 |
| ORF231 | AXN56046.1 |
| ORF232 | QEM25152.1 |
| ORF233 | UXO30862.1 |
| ORF234 | QMT29602.1 |
| ORF236 | AAT10548.1 |
| ORF238 | AND73998.1 |
| ORF240 | AAG37656.1 |
| ORF241 | YP_233051.1 |
| ORF243 | YP_010509376.1 |
| ORF244 | YP_010509379.1 |
| ORF252 | AGB75896.1 |
| ORF253 | ALF05182.1 |
| ORF254 | P21000.1 |
| ORF257 | AAX78498.1 |
| ORF258 | P21005.1 |
| ORF259 | ABD52693.1 |
| ORF260 | AAR18037.1 |
| ORF261 | QGQ59915.1 |
| ORF263 | AGB75917.1 |
| ORF264 | YP_233083.1 |
| ORF265 | AGB75922.1 |
| ORF266 | AGB75728.1 |
| ORF267 | P21099.1 |
| ORF268 | QMT29643.1 |
| ORF269 | AIX98922.1 |
| ORF270 | UIC71746.1 |
| ORF271 | AUO38295.1 |
| ORF272 | AUO38293.1 |
| ORF274 | AUO38287.1 |
| ORF275 | UXO30862.1 |

### Examples

The invention is further described in the following examples, which do not limit the scope of the invention described in the claims.

### MATERIALS AND METHODS

### Study Design

In this study, we evaluated a combination approach using a novel oncolytic virus to induce de novo cell surface expression of tumor antigen targets on solid tumors, which in combination with off-the-shelf bispecific T cell engagers (TCE), redirects endogenous T cell-mediated anti-tumor immunity. Our oncolytic vaccinia virus, CF33 carrying a truncated CD19 (CD19t) was previously developed in combination with CD19-CAR T cells (13).

All in vitro assays were performed with at least duplicate samples and were repeated in at least three independent experiments. In vivo studies were performed using 6- to 8-week-old NSG, using at least three mice per group for all studies, and four to nine mice were included within each group for all therapeutic and survival studies to ensure statistical power and evenly distributed tumor sizes across groups at treatment initiation. The health condition of mice was monitored daily by the Department of Comparative Medicine at City of Hope with euthanasia applied according to the American Veterinary Medical Association Guidelines. Investigators were not blinded when monitoring mouse survival. All studies were performed under approved protocols of the Institutional Animal Care and Use Committee and the institutional review board.

### Cell Lines and Viruses

Human triple-negative breast cancer cell line MDA-MB-468 [American Type Culture Collection (ATCC); HTB-132] was cultured in Dulbecco's modified Eagle's medium (DMEM) containing 10% fetal bovine serum (FBS; HyClone) and 1× antibiotic/antimycotic (AA; Gibco), supplemented with 25 mM Hepes (Irvine Scientific) and 2 mM L-glutamine (Thermo Fisher Scientific; complete DMEM). Both MDA-MB-468-CD19t and MDA-MB-468-HER2 cell lines were cultured as mentioned above. Human pancreatic cancer cell line Capan-1 (ATCC, HTB-79) was cultured in Iscove's modified Dulbecco's medium containing 20% FBS and 1×AA. Human pancreatic cancer cell line Panc-1 (ATCC, CRL-1469) was cultured in RPMI containing 10% FBS and 1× AA. Human ovarian cancer cell line OV90 (ATCC CRL-11732) was cultured in 1:1 volume of MCDB 105 medium (Sigma-Aldrich) and medium 199 (Gibco) containing 20% FBS and 1× AA. Human head and neck carcinoma line UM-SCC-47 (EMD Millipore) was cultured in DMEM containing 10% FBS, 1× AA, and 1× non-essential amino acids (NEAA). Human prostate cancer cell line DU145 (ATCC, HTB-81) was cultured in RPMI containing 10% FBS and 1× AA. Human glioblastoma cell line U251T (gift from W. Debinski, Wake Forest School of Medicine) was cultured in complete DMEM. Human embryonic kidney cell line 293T (ATCC CRL-3216) and human fibrosarcoma cell line HT1080 (ATCC CCL-121) were cultured in complete DMEM. African green monkey kidney fibroblasts (CV-1; ATCC CCL-70) were cultured in DMEM containing 10% FBS and 1× AA. CV-1 cells were used for both amplification and titration of orthopoxviruses

### Generation of Recombinant Chimeric Orthopoxvirus Expressing Human CD19t

To generate a shuttle vector containing the human (hCD19t) CD19t expression cassette with the VACV PSE, the hCD19t complementary DNAs (cDNAs) were polymerase chain reaction (PCR) amplified from the plasmids hCD19t-2A-IL2-pHIV7 and mCD19t-epHIV7 using Q5 High-Fidelity 2× Master Mix (New England Biolabs Inc., Ipswich, MA) and the following primers: 5′-GCG GTC GAC CAC CAT GCC ACC TCC TCG CCT CCT CTT CTT CCT CCT CTT CCTC-3′ (SEQ ID NO 929) and 5′-GCG GGA TCC ATA AAA ATT AAT TAA TCA TCT TTT CCT CCT CAG GAC CAG GGC TCT TTG AAG ATG-3′ (SEQ ID NO: 930). The PCR fragment was digested with Sal I and Bam HI and cloned into the same-cut p33NCTK-SE-hNIS replacing hNIS to yield p33NCTK-SE-hCD19t and p33NCTK-SE-mCD19t. The hCD19t and mCD19t CDNAs in p33NCTK-SE-hCD19t and p33NCTK-SE-mCD19t were confirmed by sequencing. CV-1 cells were infected with CF33 at an MOI of 0.1 for 1 hour and then transfected with p33NCTK-SE-hCD19t and p33NCTK-SE-mCD19t by using jetPRIME in vitro DNA and small interfering RNA transfection reagent (Polyplus-transfection Inc., New York, NY). Two days after infection, infected and transfected cells were harvested, and recombinant viruses (OV19t) were selected and plaque purified as described previously (13).

### DNA Constructs

MDA-MB-468 cells were engineered to express hCD19t by transduction with epHIV7 lentivirus carrying the human CD19t gene under the control of the EF1 promoter. This same process was used to engineer the expression of HER2 in MDA-MB-468. The human CD19-28z CAR lentiviral construct with hEGFRt separated by a T2A ribosome skip sequence was used as previously described (21). The human HER2-41BBz CAR lentiviral construct previously described (22) was modified to remove the CD19t domain. For T cell trafficking studies, the firefly luciferase (ffluc) gene was cloned into an epHIV7 lentivirus construct and then used to transduce human T cells cultured as previously described (23).

### Human T Cell Enrichment, Lentivirus Production and Transduction, and Ex Vivo Expansion

T cell isolation, lentivirus production and transduction, and ex vivo expansion of untransduced (mock) and CAR T cells were performed as previously described (23). Untransduced human T cells in all studies were processed in parallel with CAR T cells.

### Extracellular Staining and Flow Cytometry

Flow cytometric analysis was performed as previously described [13, 23]. Tumor cells and T cells were discriminated using CD45 (PerCP, BD Biosciences) for all in vitro studies. T cell activation was determined by using antibodies against CD69 and 4-1BB (CD137) (BD Biosciences). HER2 expression on tumor cells was determined using a HER2 antibody (PE, BD Biosciences). Tumor cells were identified using an antibody recognizing Ep-CAM (APC, BioLegend) for all in vivo studies. CD19t expression following virus infection were determined using an antibody against CD19 (PE-Cy7, BD Pharmingen) for all in vitro and in vivo studies, respectively. For the detection of TCE on the surface of T cells, biotinylated Protein-L (brand) and a secondary streptavidin PE (BD Biosciences) antibody were used. Samples were then washed twice, stained with DAPI for viability, and processed on the MACSQuant Analyzer 10 or 16 (Miltenyi Biotec). Data were analyzed with FlowJo software (v10, TreeStar).

### OV Transduction and T Cell Functional Assays

For OV19t transduction and tumor killing assays, tumor targets plated with varying multiplicity of infections (MOIs) of OV19t were co-cultured with untransduced

T cells or PBMCs at varying effector T cell-to-tumor cell ratios along with TCE concentrations of either 0, 20, 100, or 500 ng/mL. Cocultures were maintained in complete X-VIVO (Lonza) and in the absence of exogenous cytokines in round-bottom 96-well tissue culture-treated plates (Corning) for 1 to 3 days and analyzed by using flow cytometry as described above. Tumor cell killing by T cells with TCE was calculated by comparing CD45-cell counts relative to the killing observed by T cells without TCE from the same healthy donor in the absence of OV. For T cell activation assays, T cells and tumor targets were cocultured at an effector T cell-to-tumor cell ratio of 1:1 along with varying MOIs of OV in complete X-VIVO in the absence of exogenous cytokines in 96-well plates for 1 to 3 days and analyzed by using flow cytometry for specific markers of T cell activation.

For the preloading of TCE onto T cells, T cells and PBMCs were thawed and rested overnight in X-VIVO containing 10% FBS and IL-2 (100 U/mL) and IL-15 (0.5 ng/ml) cytokines. T cells and PBMCs (3×10⁶ cells/mL) were then incubated on ice for 30 minutes with 10 μg/mL, 25 μg/mL, 50 μg/mL or 100 μg/mL TCE. Following incubation, T cells were then washed with PBS and co-cultured with tumor cells targets with varying MOIs of OV. T cell activation and killing was determined as previously mentioned.

For OV transduction of MDA-MB-468-HER2 tumor killing assays, tumor targets plated with varying MOIs of OV19t were co-cultured with untransduced T cells or HER2-CAR T cells at an effector to tumor target ratio of 1:5 (5,000 to 20,000) and maintained in X-VIVO containing 10% FBS. Wells were seeded with either 100% MDA-MB-468, 100% MDA-MB-468-HER2, or 80% MDA-MB-468 and 20% MDA-MD-468-HER2 (80/20). TCE was added at a concentration of 100 ng/ml when indicated. Following 24 hours, wells were rechallenged with 3× the number of MDA-MB-468P tumor cells per well (60,000 cells). Cocultures were maintained for 1-2 days as described above and then analyzed using flow cytometry. For wells rechallenged a second time, an additional round of 3× MDA-MB-468 (60,000 cells) were added to all wells. 72 hours after the second rechallenge, cocultures were processed and analyzed using flow cytometry as mentioned above.

### Cytokine Enzyme-Linked Immunosorbent Assay

Tumor cells and T cells were plated into 96-well round-bottom plates (Costar) along with varying MOIs of OV19t in the presence or absence of TCE (CD19-targeting), respectively. After incubations at 37° C. for 24, 48, or 72 hours, supernatants were collected and analyzed according to the human IFN-or IL-2 enzyme-linked immunosorbent assay (ELISA) Ready-SET-Go! (eBioscience) manufacturer's protocol. Plates were read at 450 nm and 570 nm using the Cytation 3 Cell Imaging Multi-Mode Reader and Gen5 Microplate Reader and Imager Software (BioTek).

### In Vivo Studies

All animal experiments were performed under protocols approved by the City of Hope Institutional Animal Care and Use Committee. For human tumor xenograft studies, MDA-MB-468 and MDA-MB-468-CD19t cells (5× 10⁶ cells per mouse) were prepared in PBS and injected subcutaneously into the flank of female NSG mice. Tumor growth was monitored 2-3 times per week by caliper measurement. Once tumor volumes reached about 100 to 300 mm³, OV19t virus was prepared and diluted in PBS (pH 7.4) and intratumorally administered at 106 pfu per MDA-MB-468 tumor-bearing mice. 2 days post OV19t treatment, PBMCs were isolated from leukapheresis products obtained from a consented research participant (healthy donor) under protocols approved by the City of Hope (COH) Internal Review Board (IRB using density gradient centrifugation over Ficoll-Paque (GE Healthcare) followed by multiple washes in PBS/EDTA (Milteny Biotec). PBMCs were then counted, washed, and prepared in PBS (pH 7.4) for intravenous tail vein injection (5×10⁶ cells per mouse). Four days post T cell engraftment, mice were treated with TCE (8 μg/ms in PBS) intravenously for five consecutive days (Monday-Friday) and then again for three consecutive days the following week (Monday-Wednesday) for a total of 8 treatments (64 μg total per mouse). Mice were euthanized two weeks post OV19t treatment in accordance with our animal safety guidelines.

For in vivo studies with mice bearing MDA-MB-468-CD19t tumors as CD19t+ controls, treatment with PBMCs and TCE coincided with the same schedule as mentioned above, without OV treatment.

For in vivo T cell trafficking studies, mice were engrafted subcutaneously with either MDA-MB-468 or MDA-MB-468-CD19t and treated with OV19t. Two days post OV19t treatment, firefly luciferase (ffluc)-expressing T cells were thawed, washed, and prepared in PBS (pH 7.4) for intravenous tail vein injection (5×10⁶ cells per mouse).

TCE treatment followed as previously described. ffluc-T cell trafficking was monitored 2-3 times a week by noninvasive optical imaging (LagoX). Mice were imaged after intraperitoneal injection of 150 to 250 μL of d-luciferin potassium salt (PerkinElmer) suspended in PBS (pH 7.4, 4.29 mg per mouse). Flux was then analyzed with Living Image software (Aura).

### Statistical Analysis

Data are presented as means±SEM, unless otherwise stated. Statistical comparisons between groups were performed using the unpaired two-tailed Student's t test to calculate P value, unless otherwise stated. Statistical comparison of Kaplan-Meier survival data was performed using the log-rank (Mantel-Cox) test.

### Example 1: CD19-CD3 Bispecific T Cell Engagers Redirect Human T Cells to Target Solid Tumors Infected with OV Carrying CD19t (OV19t)

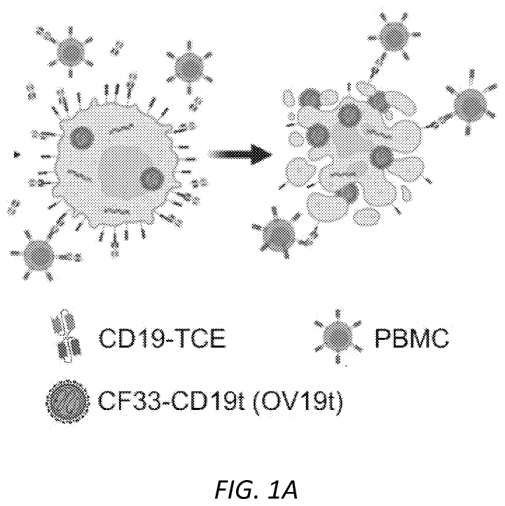

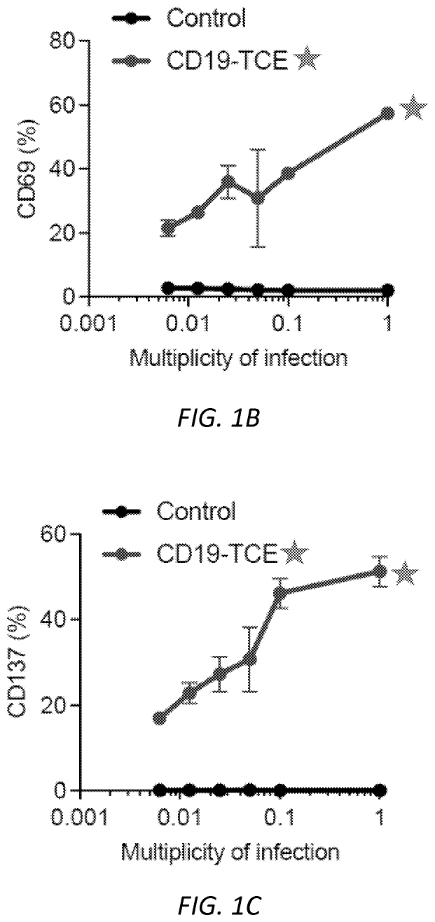

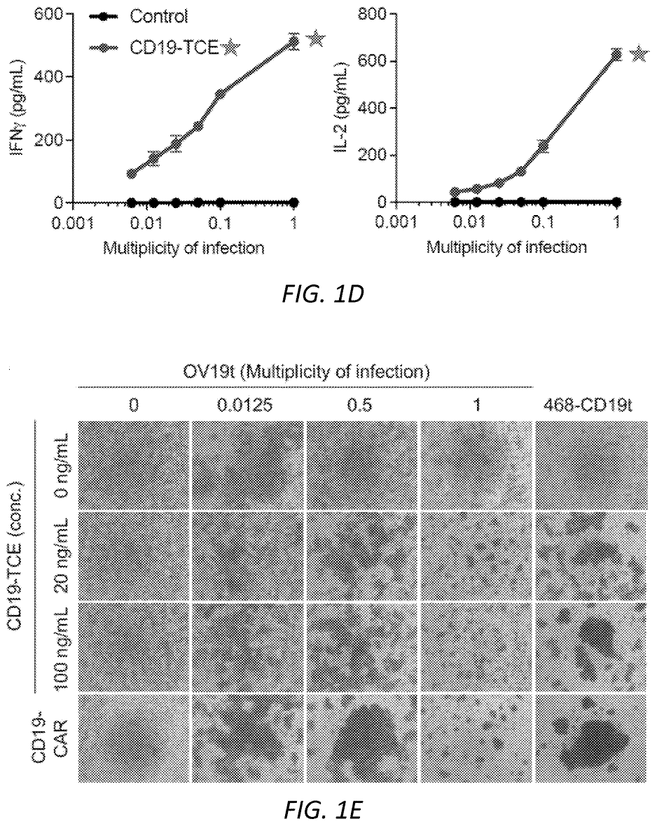

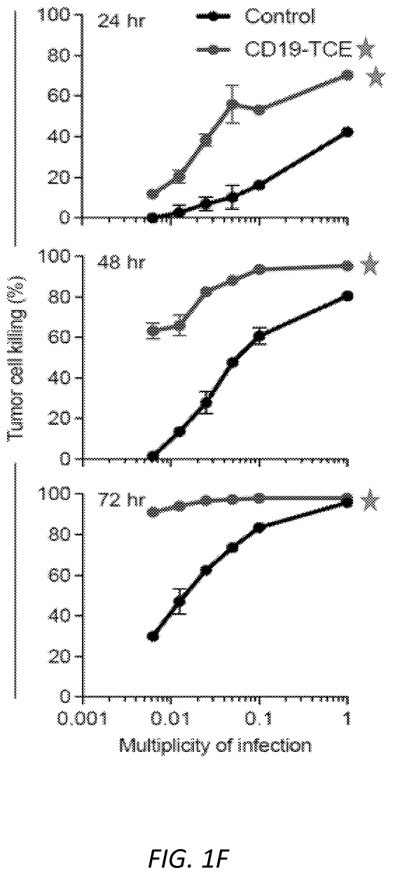

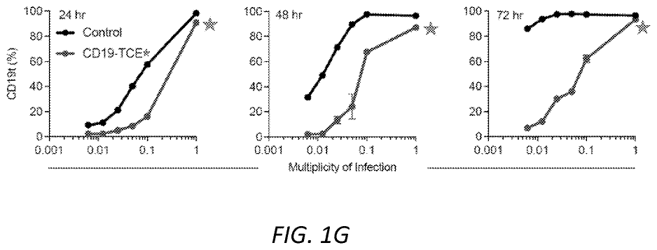

For these studies, we used the oncolytic chimeric orthopoxvirus carrying CD19t (OV19t) as previously described [13]. We first assessed whether the CD19t delivered to tumors via OV could activate non-targeting T cells in the presence of blinatumomab [CD19-CD3 T cell engagers (CD19-TCE)] (FIG. 1A). MDA-MB-468 human triple-negative breast cancer tumor cells were infected with varying multiplicity of infection (MOI: 0, 0.00625, 0.0125, 0.025, 0.05, 0.1, and 1) of OV19t and CD19-TCE (100 ng/mL). Tumor cells were then co-cultured with human peripheral blood mononuclear cell (PBMC)-derived non-targeting T cells at an effector-to-target (E: T) ratio of 1:1. At 48h, T cell activation (CD69, CD137) was induced in the presence of 100 ng/mL CD19-TCE when co-cultured with OV19t-infected tumor cells in an MOI-dependent manner (FIGS. 1B-1C). IFNy and IL-2 cytokines were produced in an MOI dependent manner (FIG. 1D).

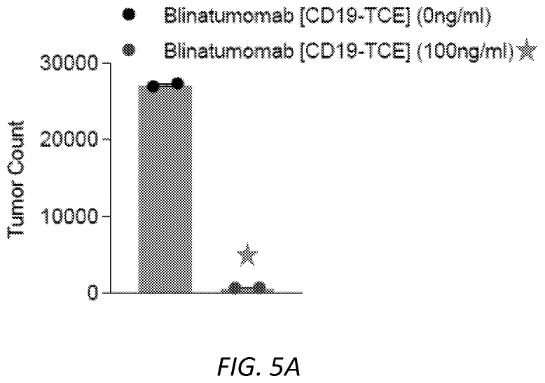

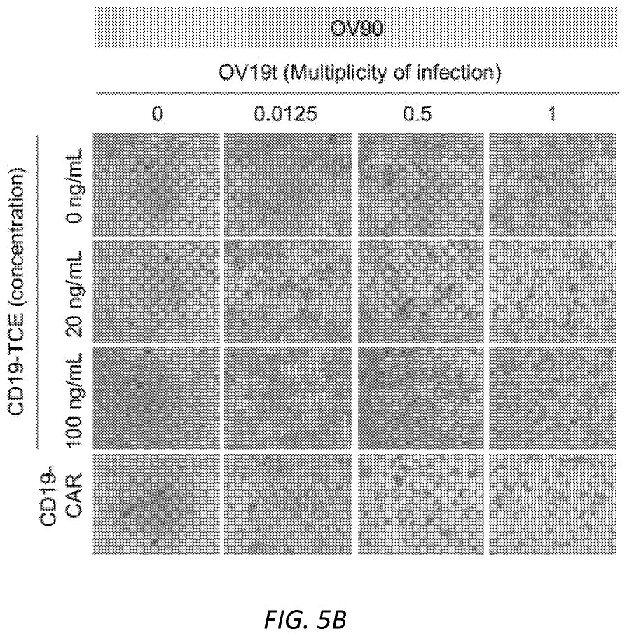

We then performed in vitro cell killing assays with tumor cells infected with OV19t at MOIs of 0, 0.0125, 0.5, and 1 in combination with 0, 20, or 100 ng/ml CD19-TCE. MDA-MB-468 tumor cells stably expressing CD19t via lentiviral transduction (MDA-MB-468-CD19t) were used as a positive control. We observed robust tumor cell killing of MDA-MB-468 tumor cells infected with OV19t and co-cultured with non-targeting T cells in the presence of either 20 or 100 ng/mL CD19-TCE when compared to OV19t-infected tumors alone (FIG. 1E). Tumor cell killing was quantified using flow cytometry. At each time point (24h, 48h, and 72h), the presence of non-targeting T cells in combination with CD19-TCE showed greater killing of tumor cells infected with OV19t compared to tumor cells infected with OV19t co-cultured with non-targeting T cells in the absence of CD19-TCE (FIG. 1F). T cell-mediated tumor cell killing when co-cultured with CD19-TCE was confirmed against MDA-MB-468-CD19t cell lines (FIG. 5A)

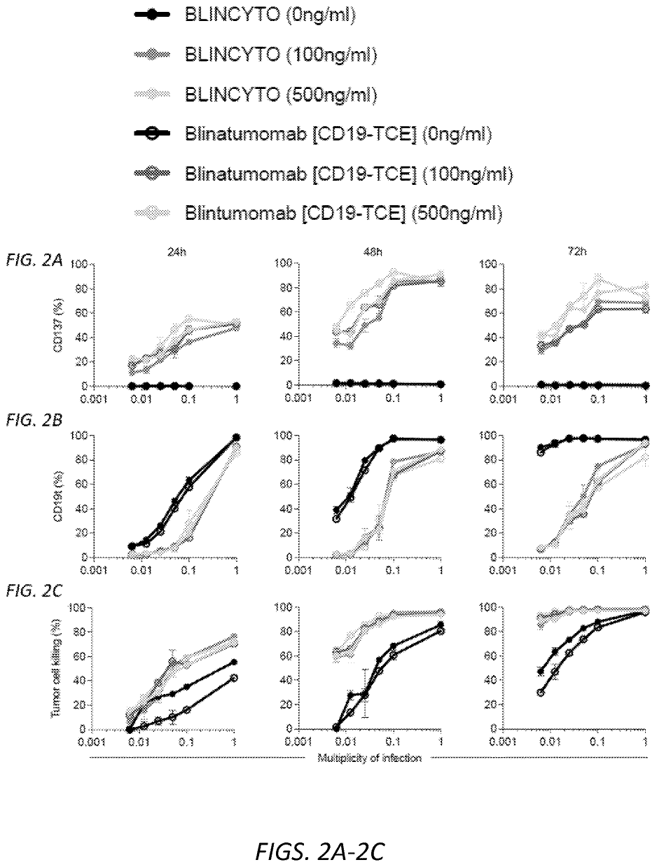

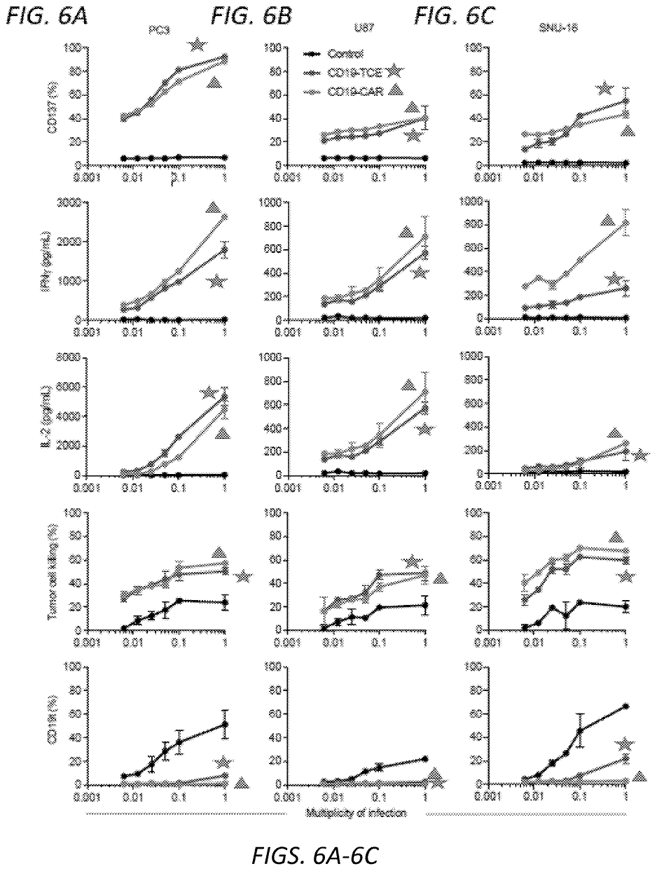

We confirmed activity of this combination comparing the FDA-approved blinatumomab to research grade CD19-TCE (Proteogenix), which demonstrated similar in vitro activity (FIG. 2A-2C). Therefore, research grade CD19-TCE was used for all subsequent studies. Similar tumor cell killing was observed against the OV90 cell line infected with OV19t and co-cultured with non-targeting T cells in the presence of either 20 or 100 ng/mL CD19-TCE when compared to OV19t-infected tumors alone (FIG. 5B). Importantly, CD19t expression was significantly reduced in tumor cells with CD19-TCE, showing on-target activity (FIG. 1G). Additional solid tumor cell lines including PC3 (prostate), U87 (glioma), and SNU-16 (gastric) were tested using this combination therapy and demonstrated similar advantages in activation, tumor cell killing, and targeting of CD19t expressing cells (FIG. 6A-6C). Taken together, our data suggest that CD19-TCE redirects T cell-mediated activation and anti-tumor activity against OV19t-infected tumor cells.

### Example 2: OV19t Promotes Tumor Infiltration of Non-Targeting T Cells Following CD19-TCE Treatment

OV has been previously shown to augment infiltration of T cells and other immune cells into tumors inducing endogenous anti-tumor immunity [16]. Therefore, we assessed the tumor trafficking of engrafted PBMCs lentivirally transduced to stably express firefly luciferase (ffluc) following OV19t infection with or without CD19-TCE treatment in MDA-MB-468 xenograft model.

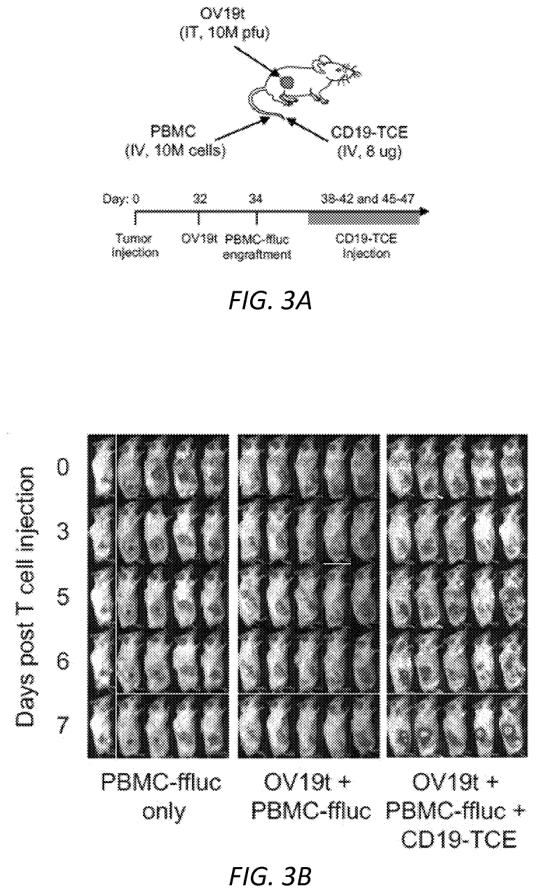

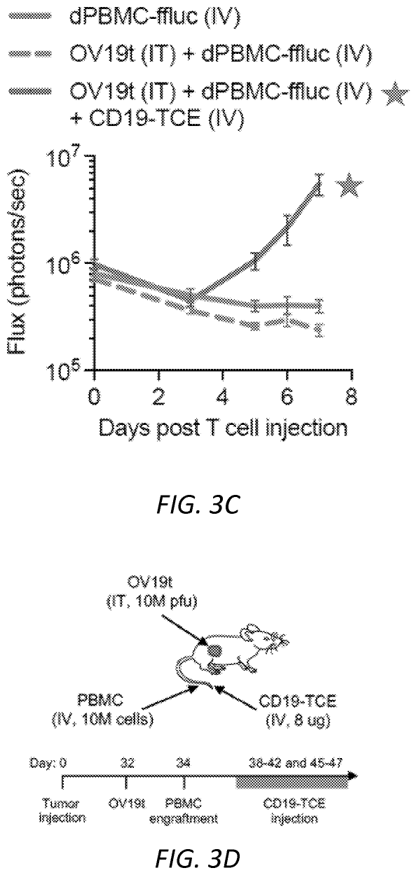

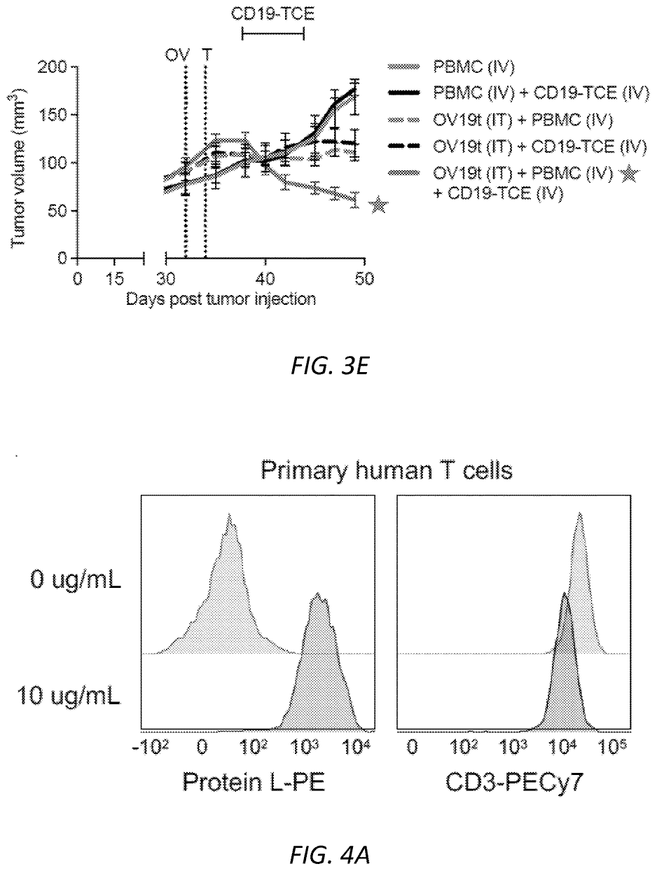

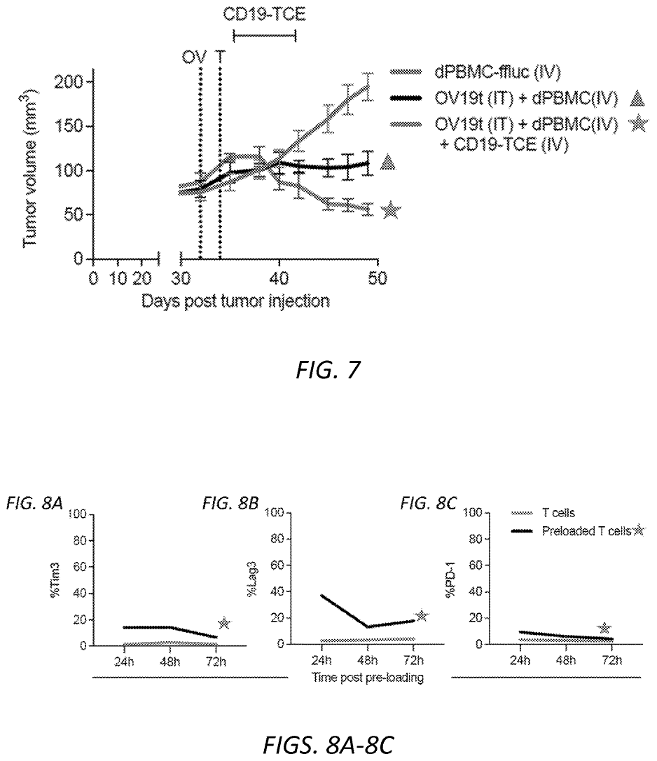

Using ffluc expressing T cells (FIG. 3A), the combination of OV19t, PBMC-ffluc, and CD19-TCE again exhibited significant tumor regression (FIG. 7). Interestingly, OV19t and CD19-TCE combination treatment induced significantly higher influx of non-targeting T cells compared to PBMC-ffluc alone or PBMC-ffluc with OV19t alone (FIGS. 3B-3C). These data suggest that CD19-TCE redirects T cells to target CD19t, enabling tumor-infiltration of endogenous T cells to target and kill tumors expressing CD19t induced by OV19t infection.

### Example 3: Anti-Tumor Efficacy with the Combination of OV and TCE in Human Solid Tumor Xenograft Models

To evaluate the anti-tumor activity of OV19t in combination with CD19-TCE in vivo, we treated NSG mice bearing subcutaneous MDA-MB-468 tumors with a single intratumoral injection of 106 pfu OV19t as previously described [13]. Two days after OV19t treatment, we collected PBMCs from a healthy human donor and engrafted with 5×10⁶ cells per mouse. Four days after PBMC transfer, mice were treated with CD19-TCE (8 μg/dose) or PBS for five consecutive days (Mon-Fri) and then three consecutive days (Mon-Wed) the following week for a total of 64 μg/mouse (FIG. 3D). Over the course of two weeks, MDA-MB-468 tumor-bearing mice treated with PBMCs alone or PBMCs and CD19-TCE showed no tumor control. Notably, mice treated with OV19t and PBMCs or OV19t and CD19-TCE slowed tumor growth as expected with OV19t alone, but we observed marked tumor regression with the combination of OV19t, PBMCs, and CD19-TCE (FIG. 3E). These studies highlight the therapeutic efficacy of combining OV and TCE in preclinical models.

### Example 4: Antitumor Efficacy of CD19-TCE Pre-Loaded T Cells Against OV19t-Infected Solid Tumor Cells In Vitro

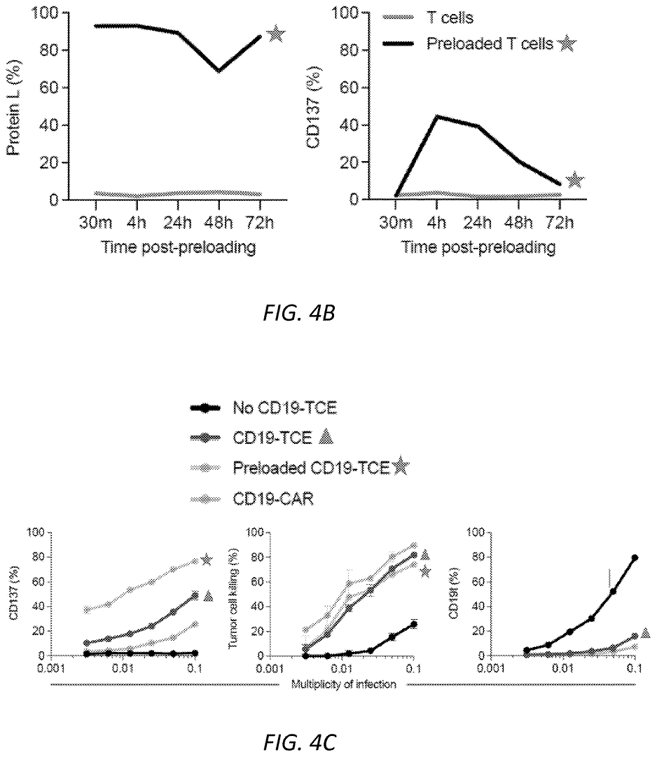

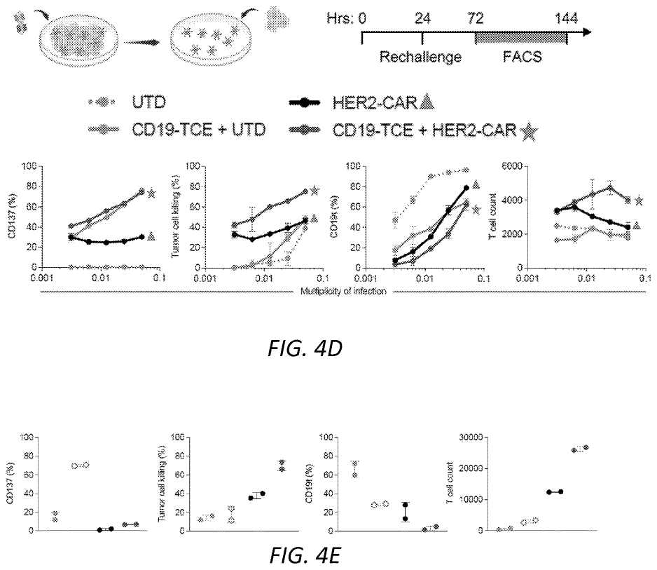

To evaluate if CD19-TCE pre-loaded onto a non-targeting T cell has the same anti-tumor efficacy as T cells exposed to CD19-TCE in culture, we co-cultured MDA-MB-468 tumors cells infected with varying MOIs of OV19t and non-targeting T cells. To appropriate wells, 100 ng/mL CD19-TCE and 1:1 E: T with non-targeting T cells, 1:1 E: T of non-targeting T cells preloaded with 10 μg/mL CD19-TCE, or CD19-CAR T cells were added in a tumor killing assay. CD19-TCE pre-loading was confirmed prior to co-culture with tumor targets by determining shift of CD3 expression and by using biotinylated protein L and secondary streptavidin antibody staining. We demonstrated a shift in CD3 expression while confirming approximately 95-97% protein L staining on T cells preloaded with 10 μg/mL and thus continued all pre-loading experiments with a concentration of 10 μg/mL (FIG. 4A). Additionally, we performed a time course study over 72 h to determine CD19-TCE binding, activation, and exhaustion following pre-loading of T cells. CD3 shift decreased (data not shown) most likely due to proliferation of T cells following activation while protein L expression remained high over 72h (FIG. 4B). After 4 hours of culturing following pre-loading of T cells with CD19-TCE, there was an initial increase in activation marker (CD137) but decreased over 72h (FIG. 4B). There was a slight increase in exhaustion markers (TIM3, LAG3, and PD-1) at 24 h, but decreased over 72h (FIGS. 8A-8C).

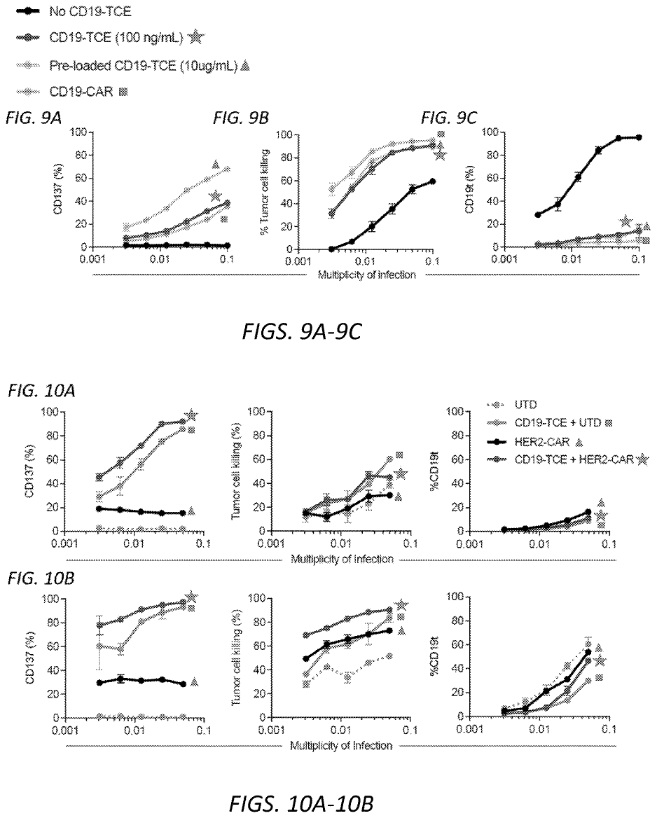

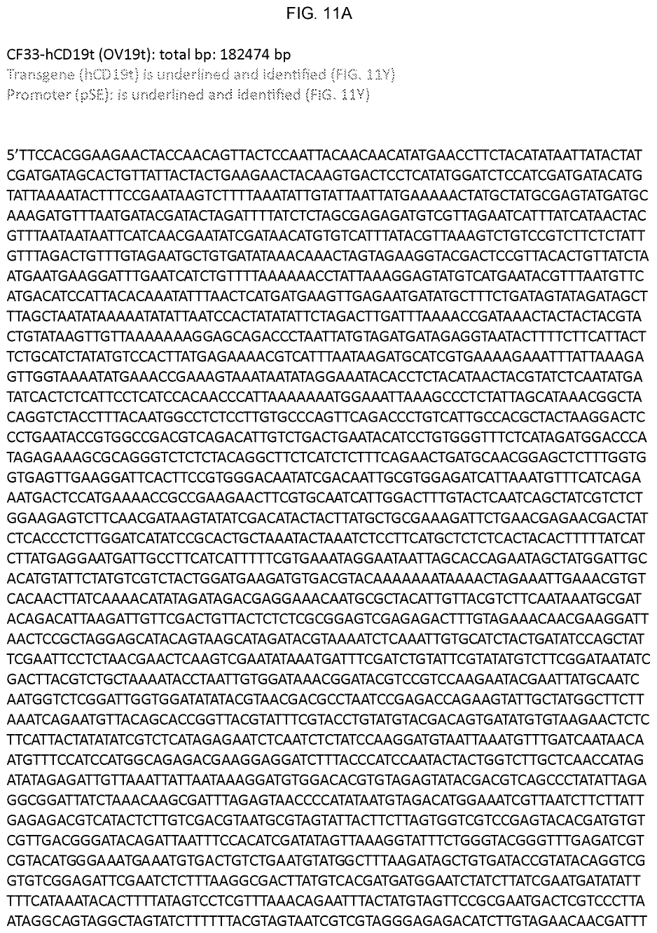

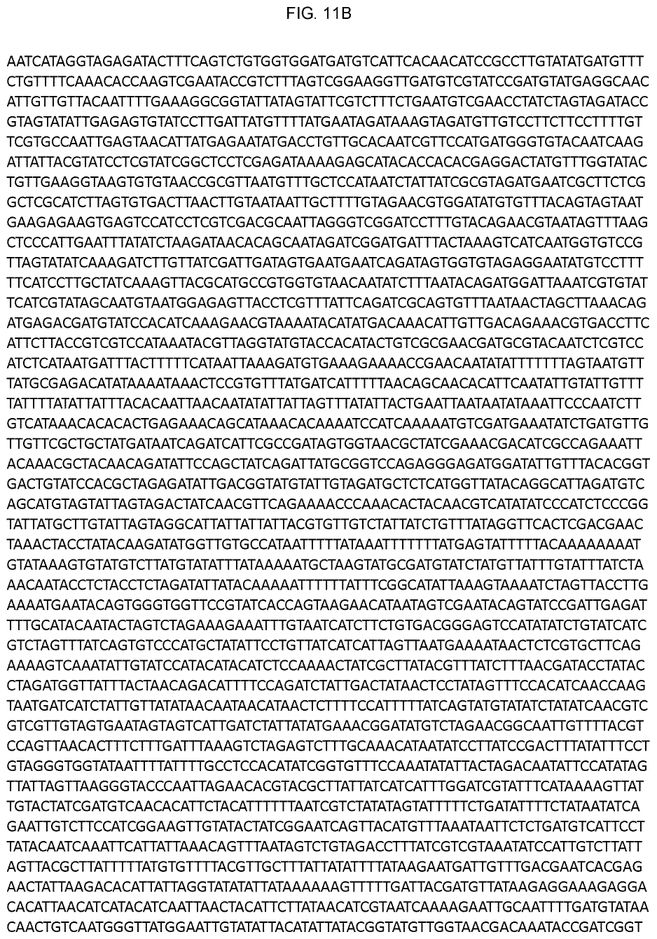

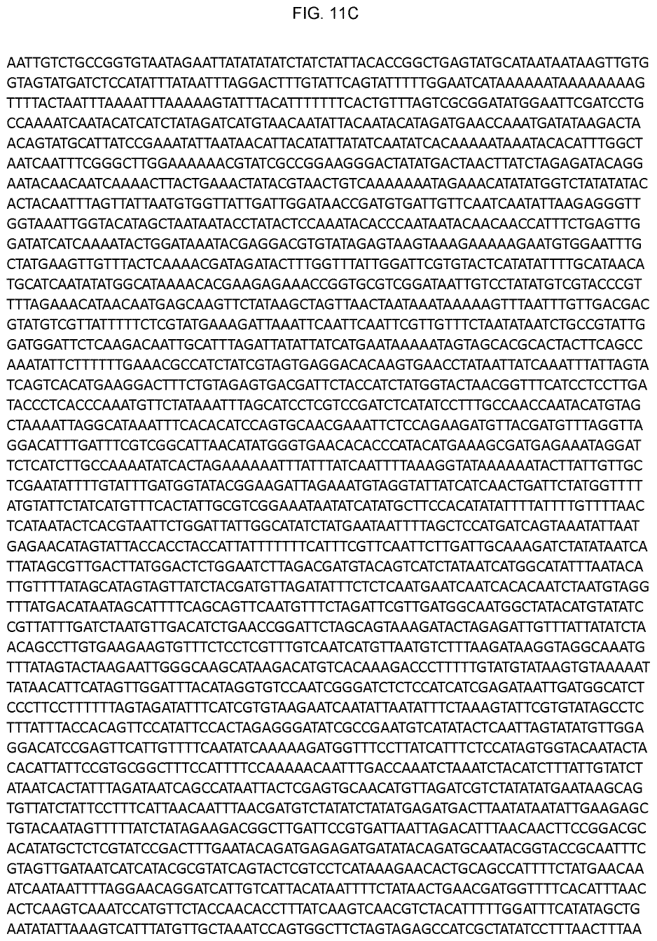

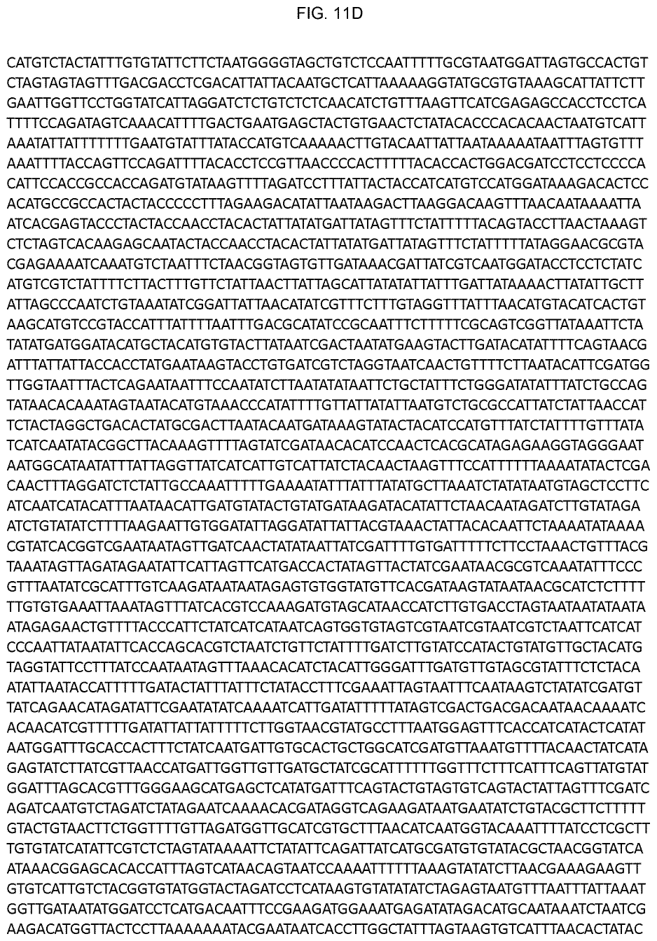

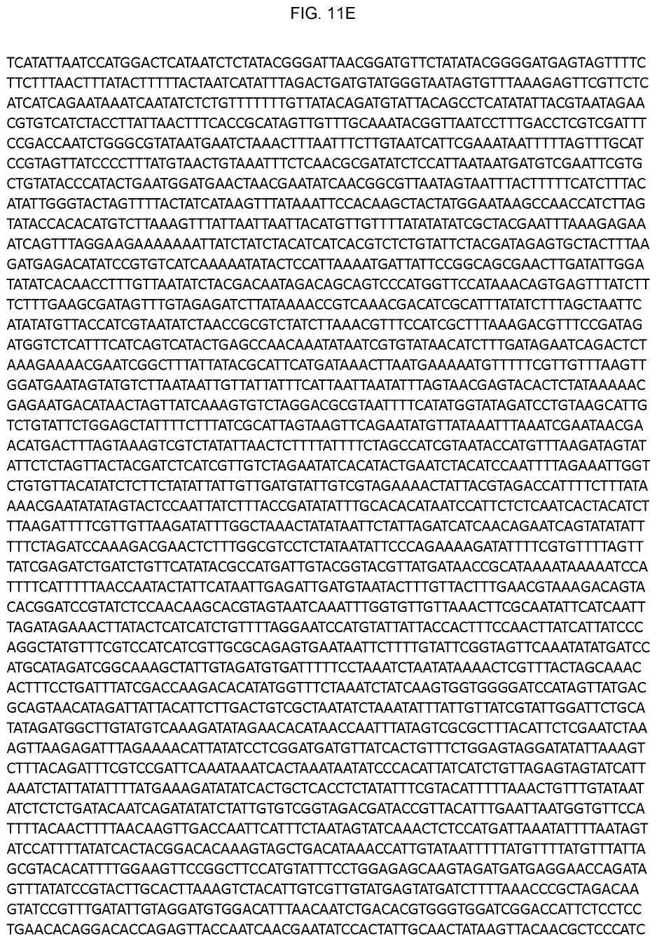

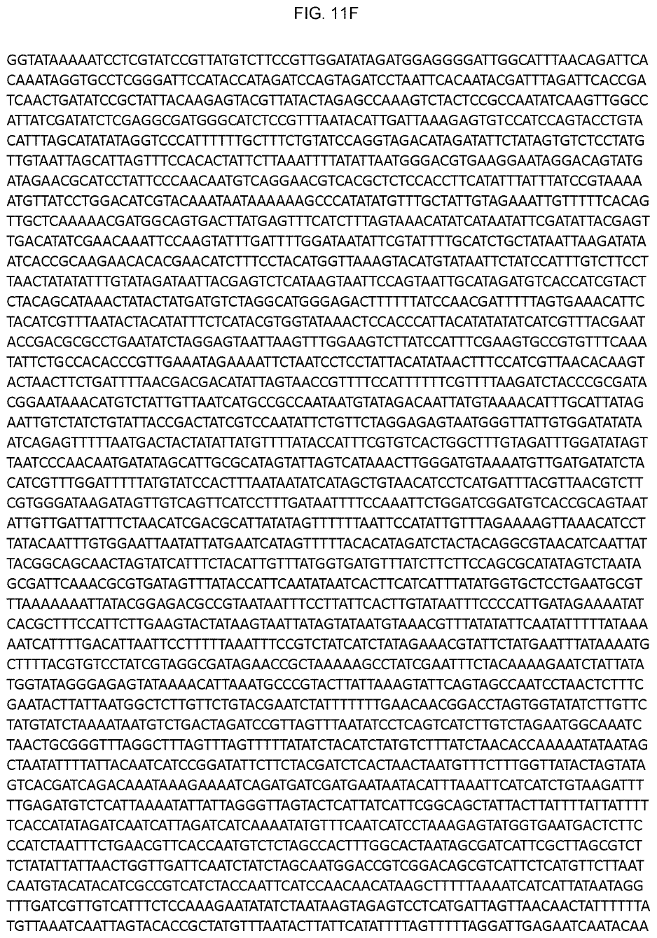

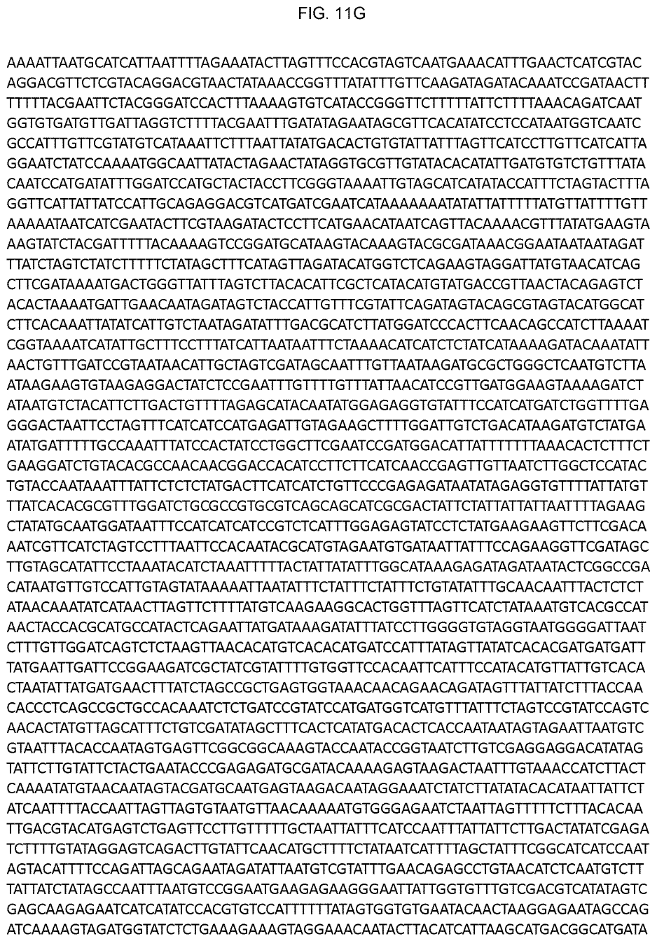

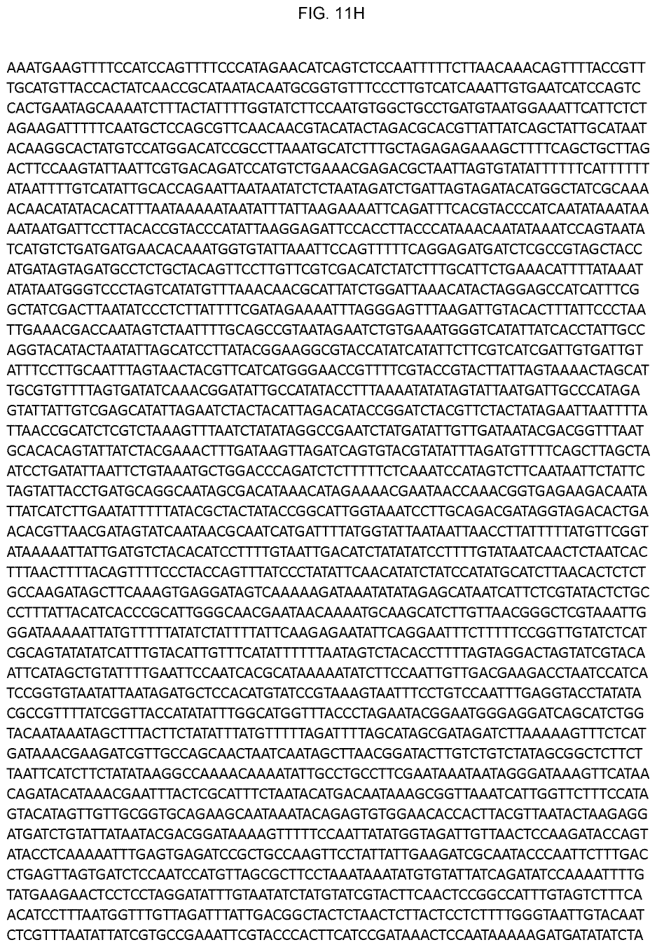

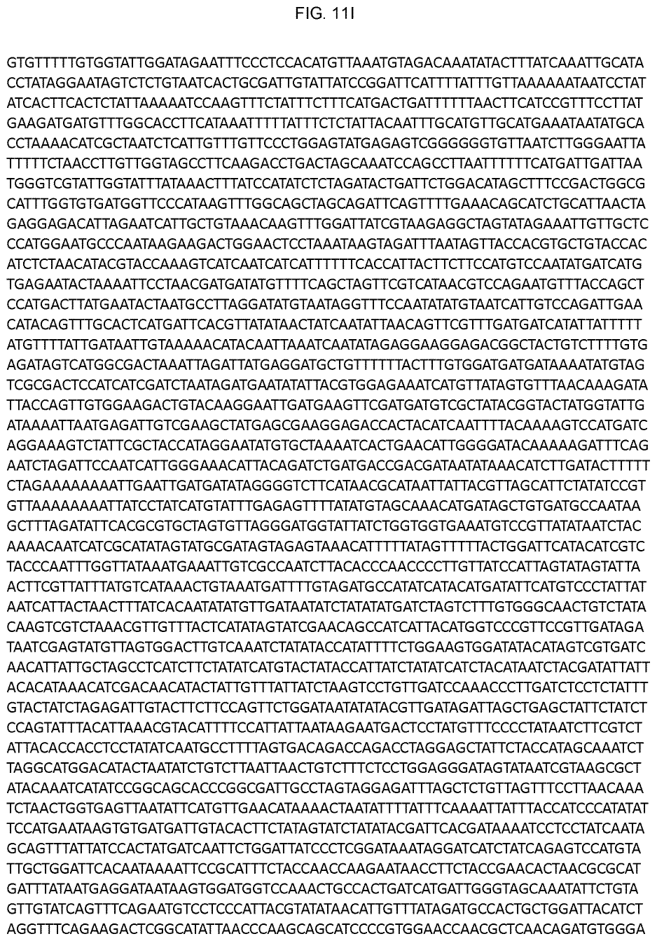

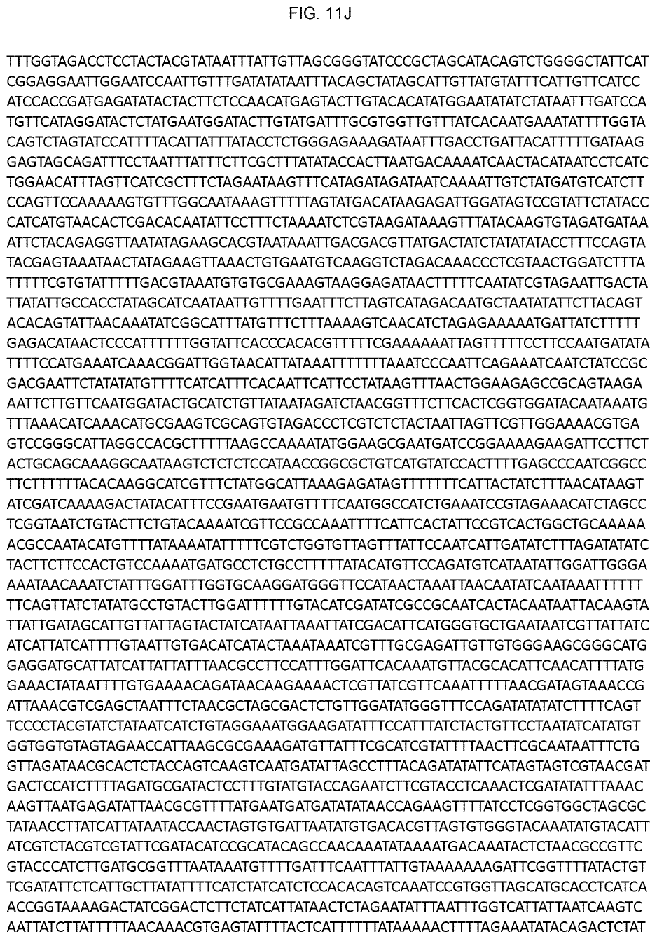

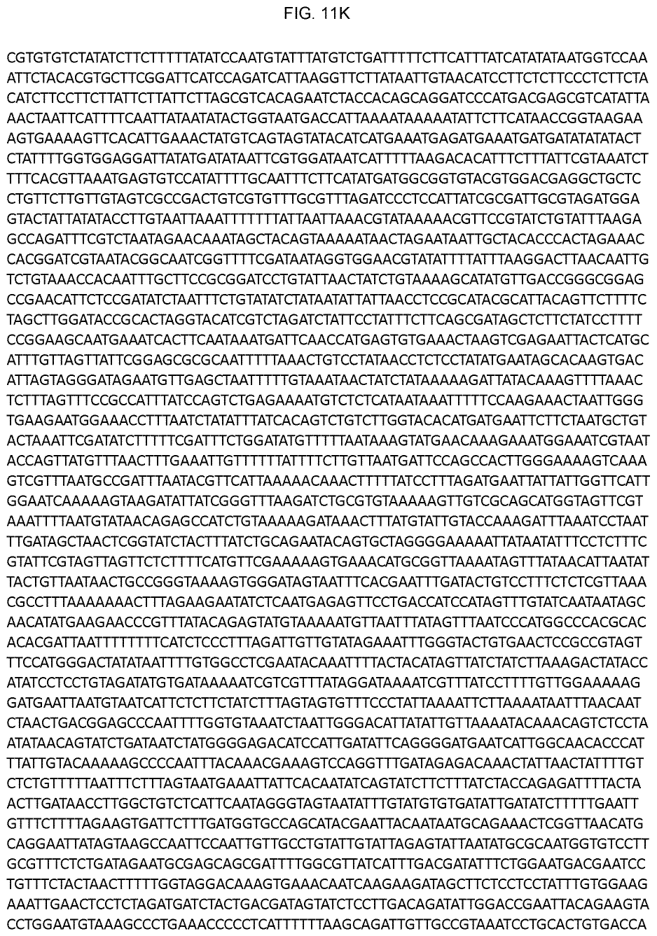

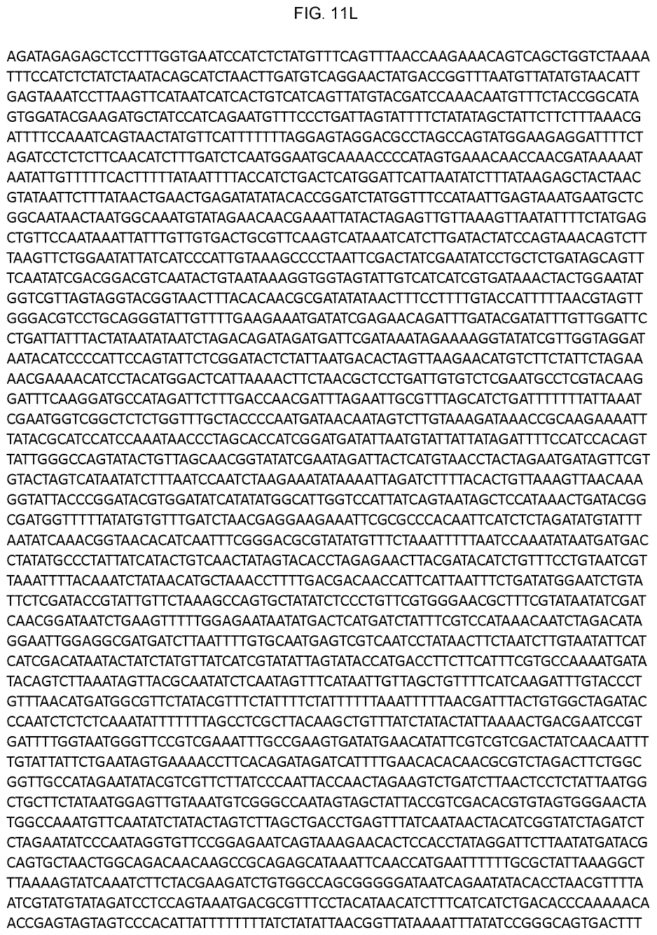

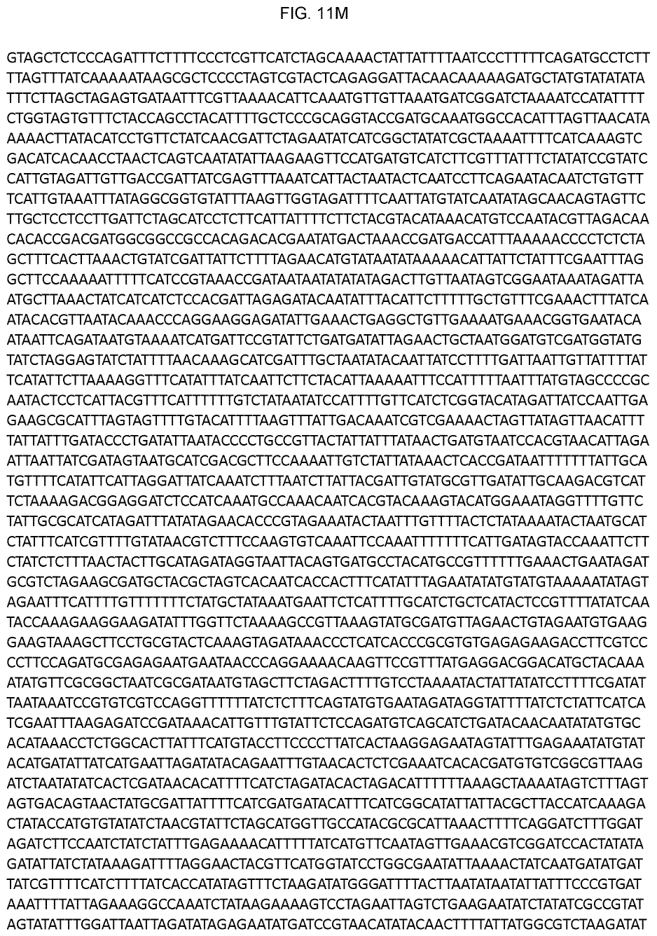

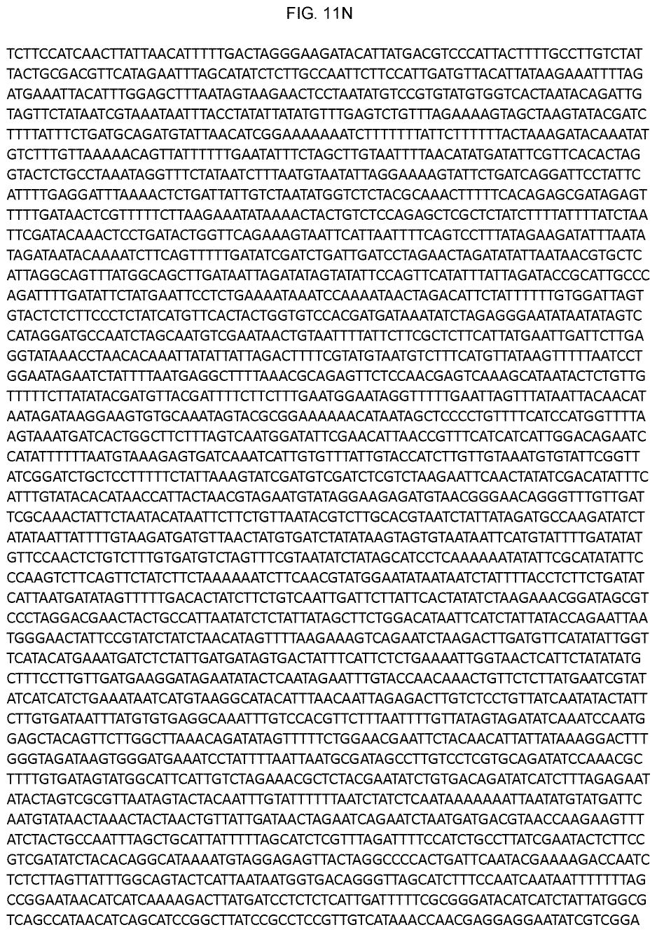

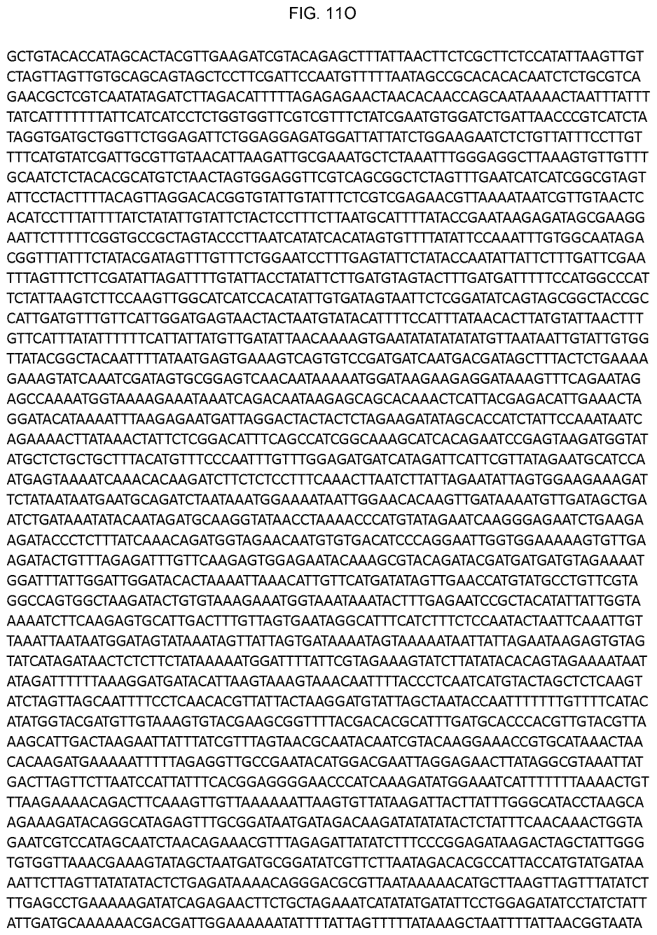

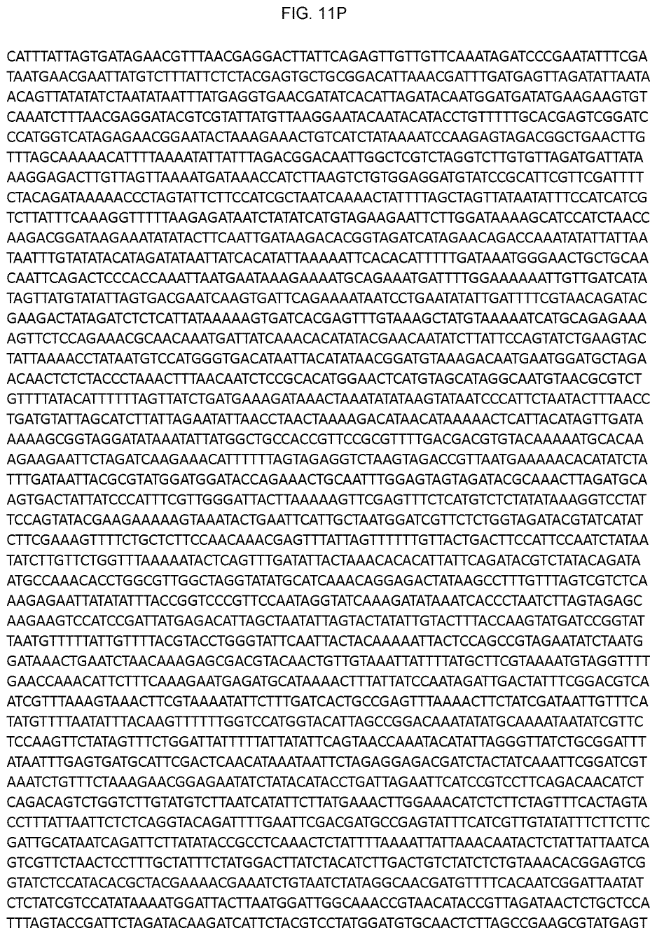

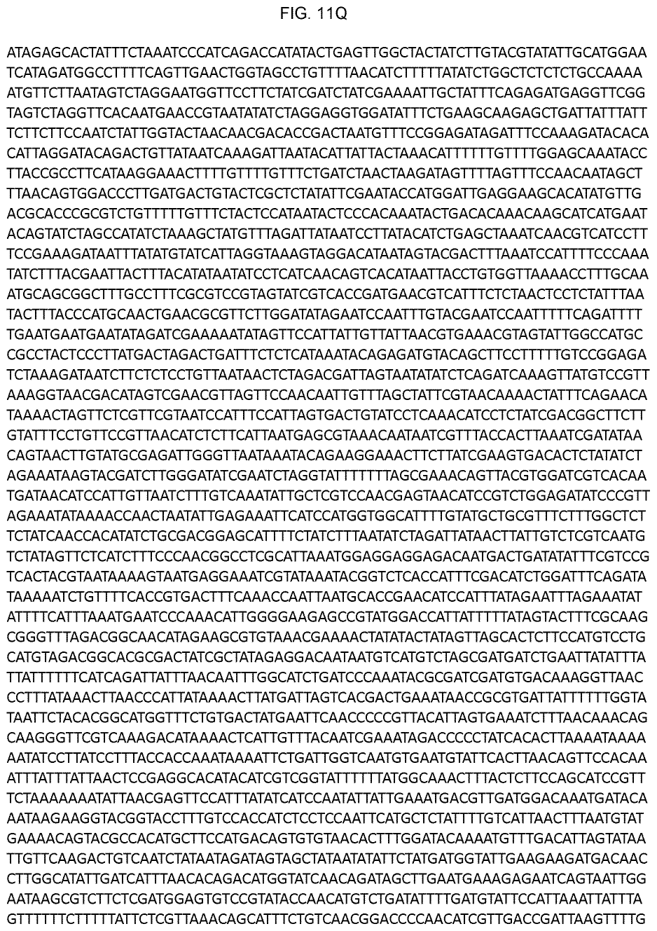

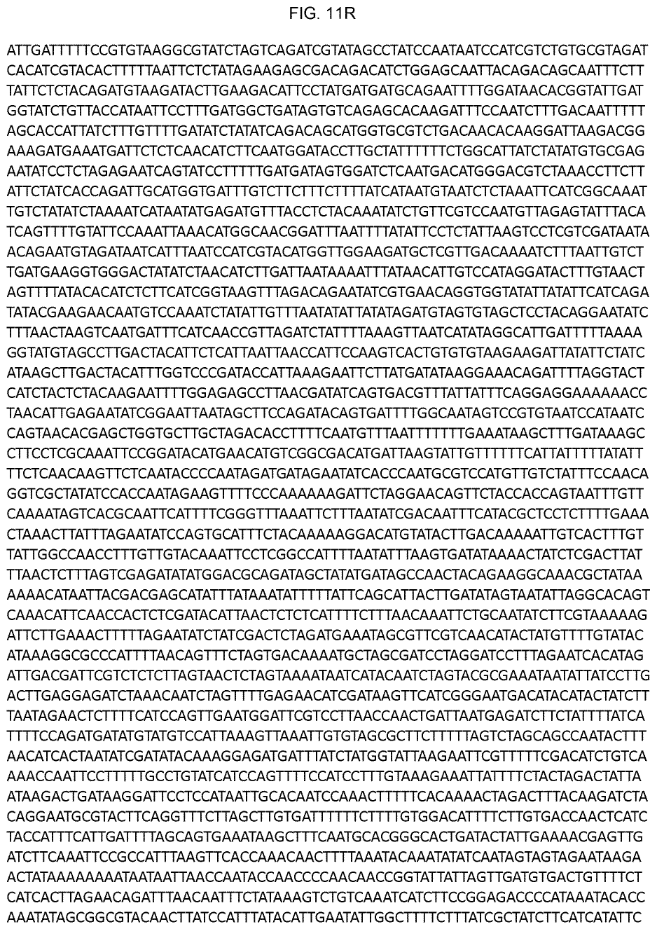

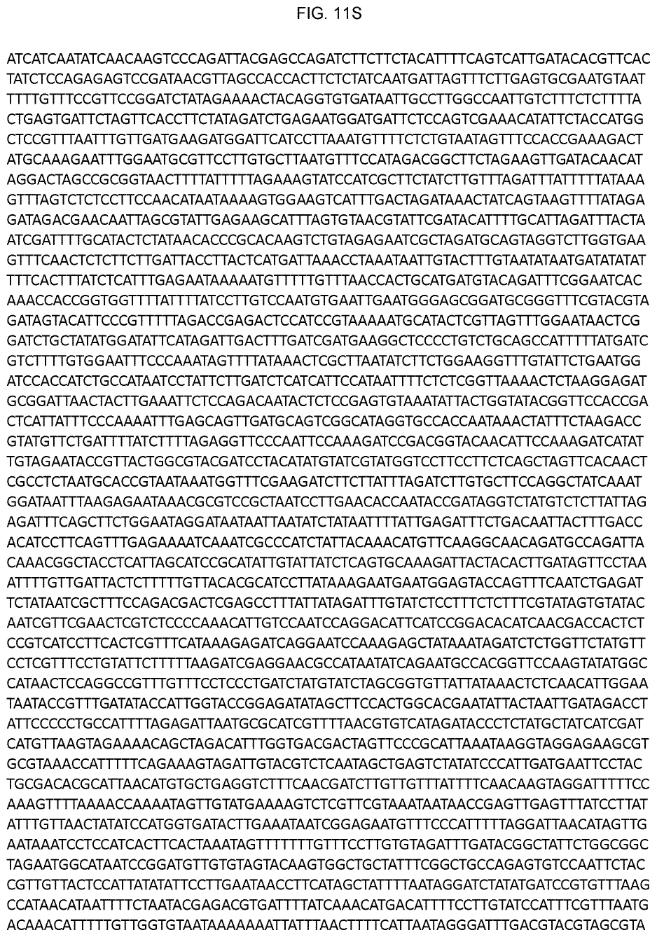

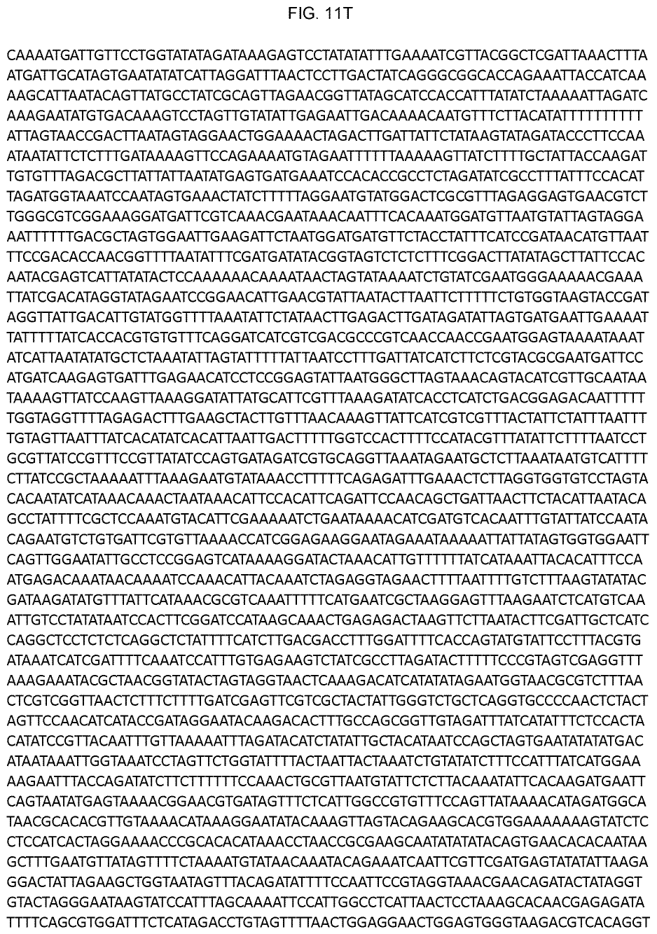

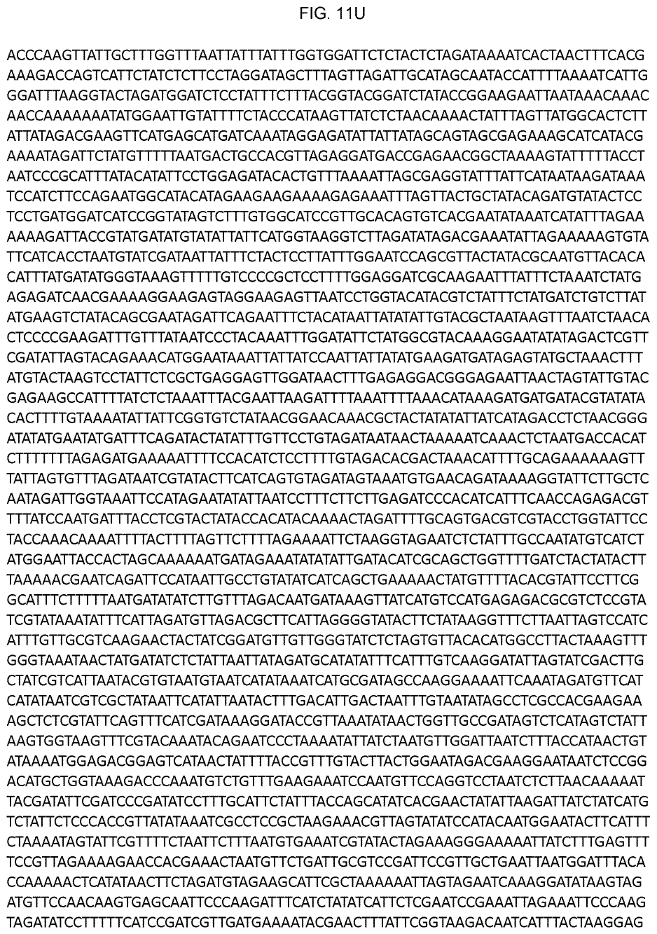

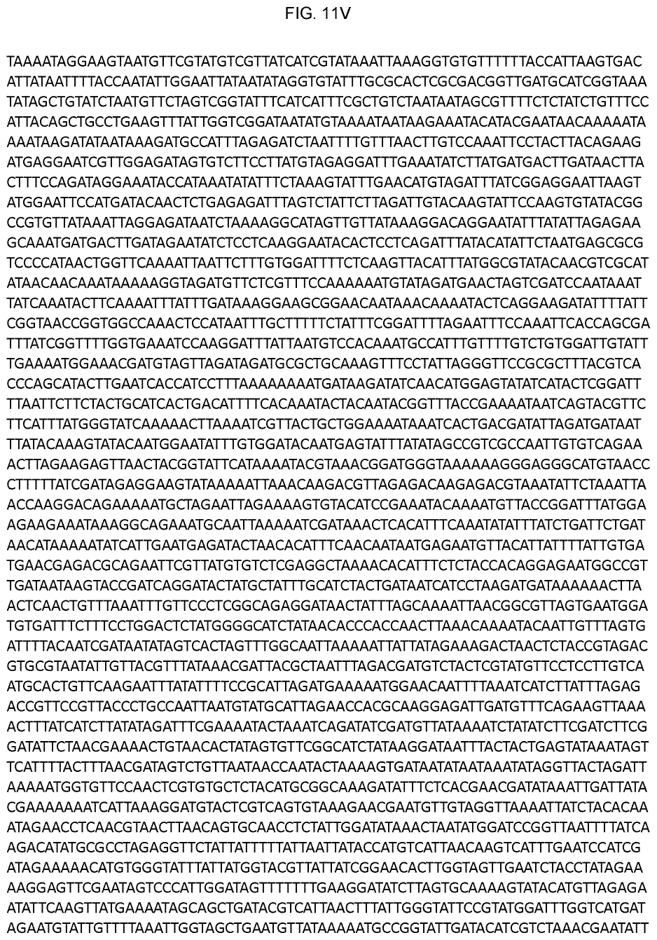

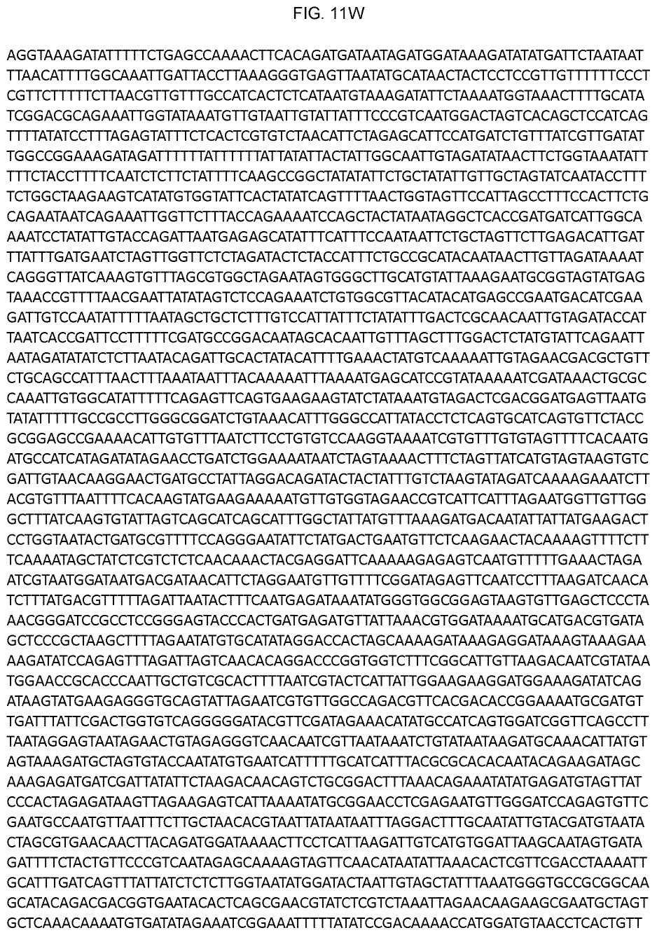

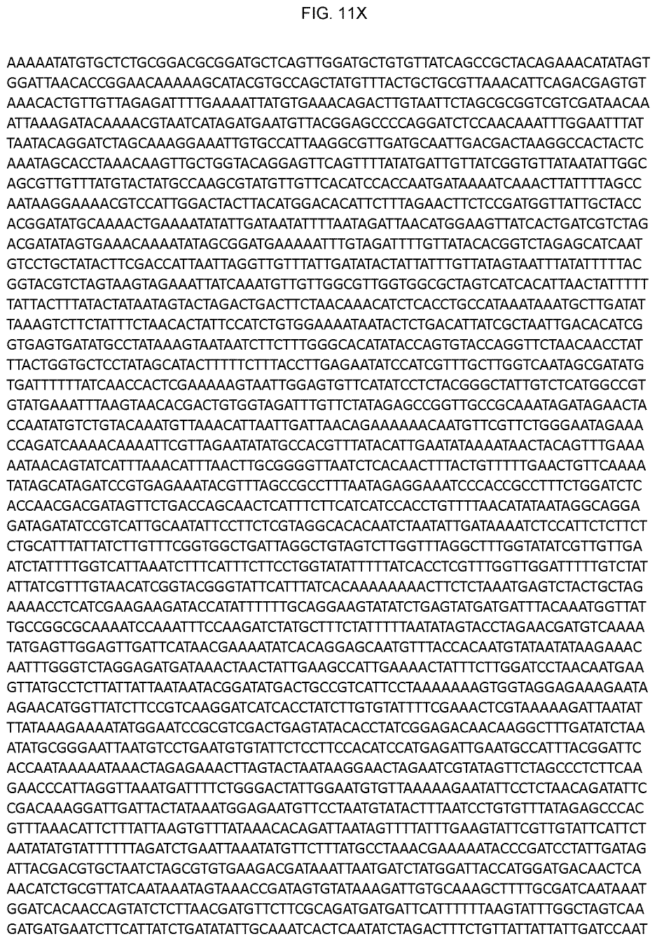

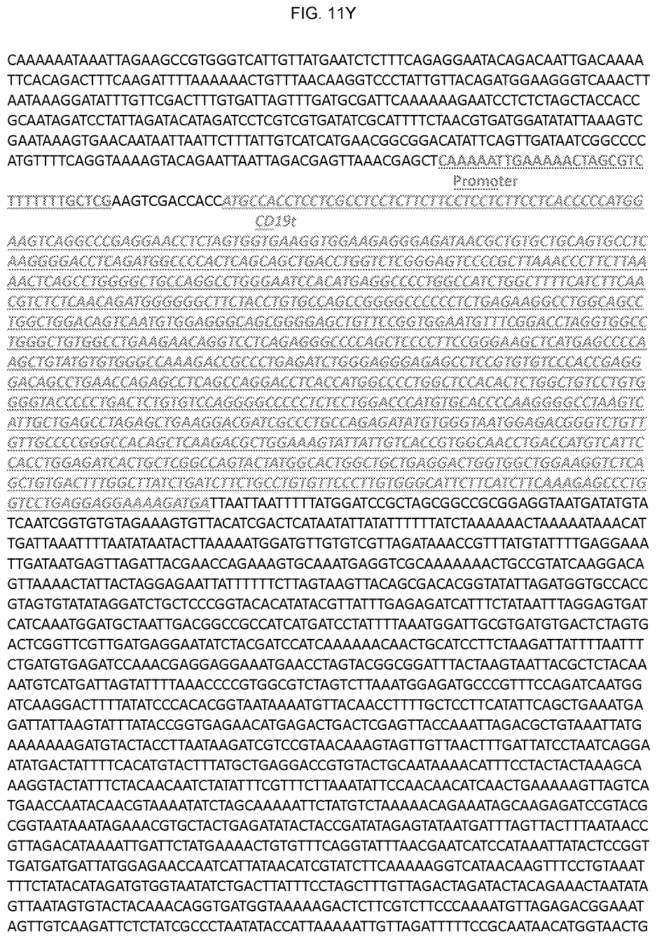

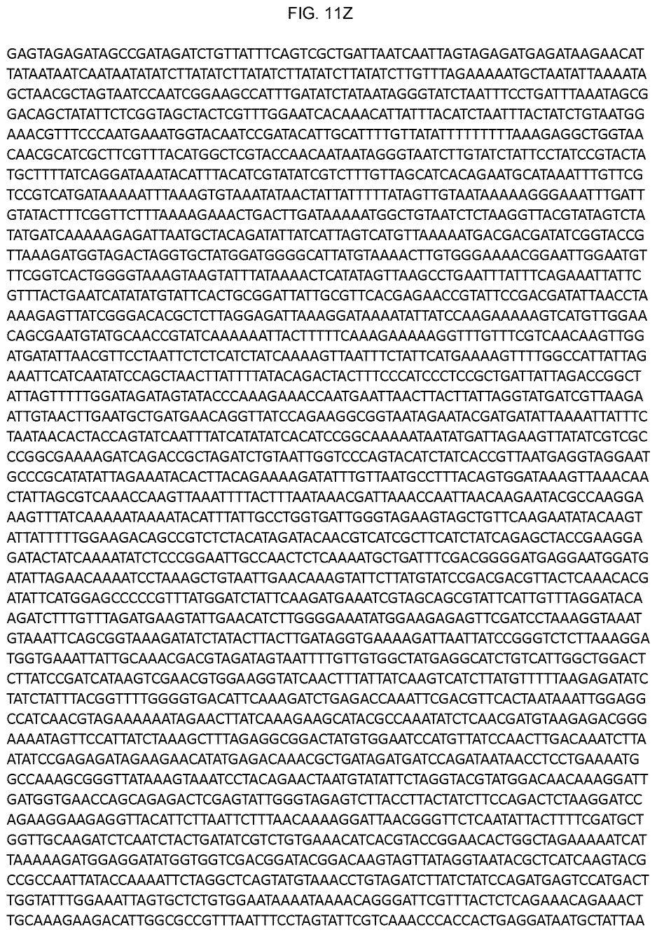

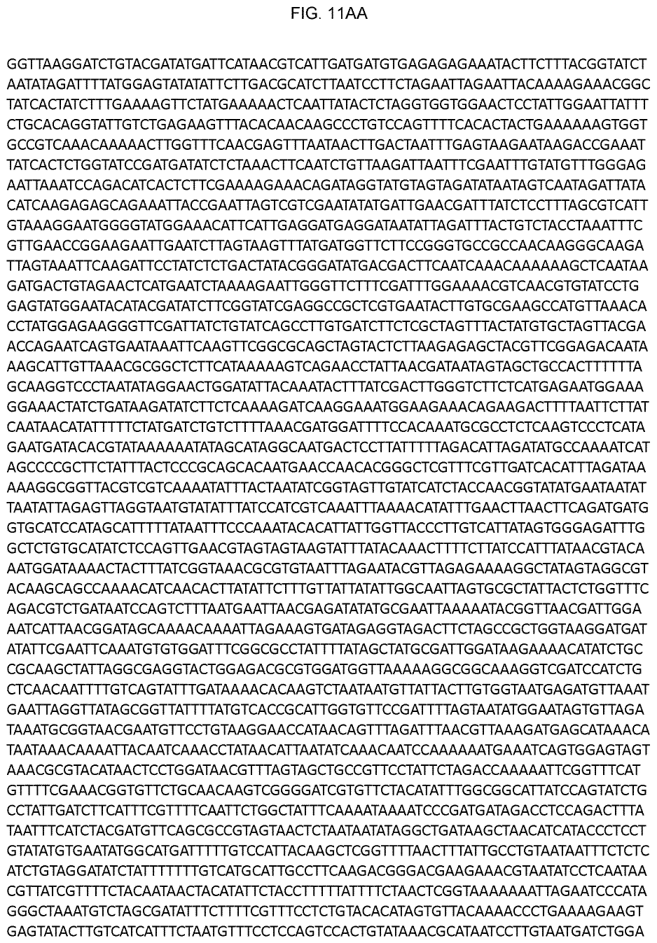

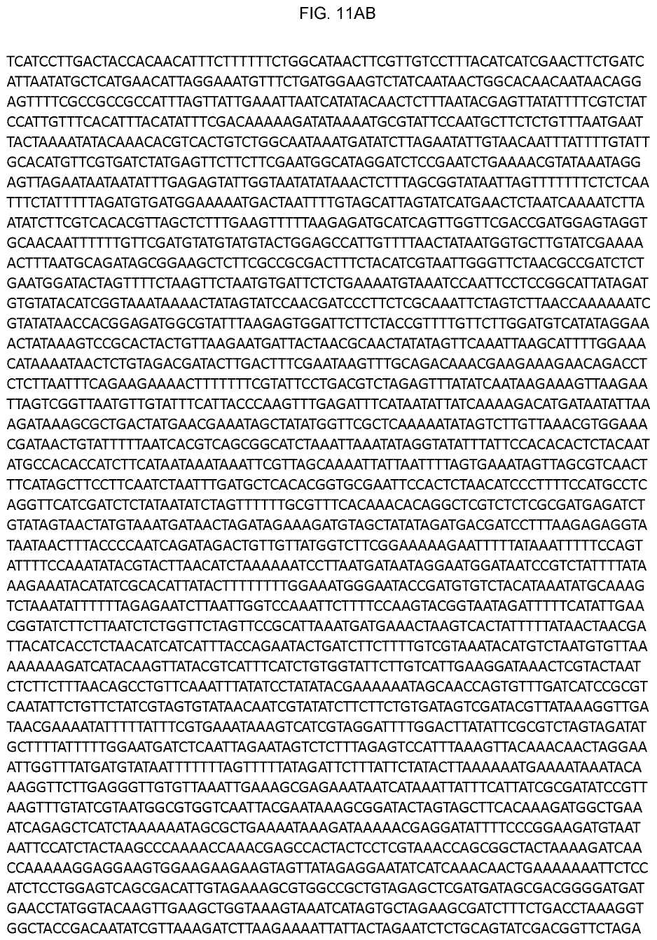

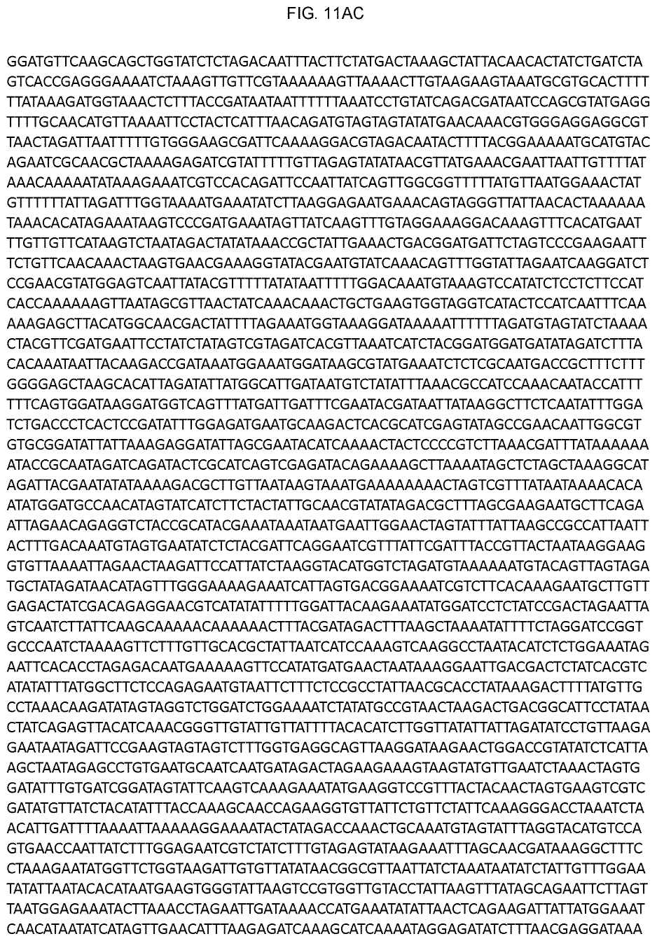

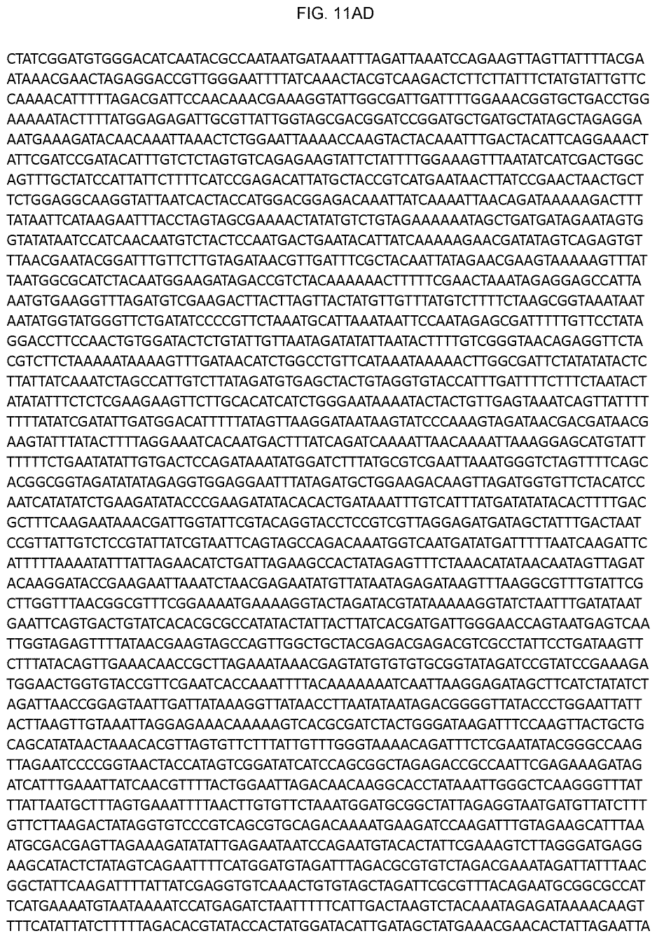

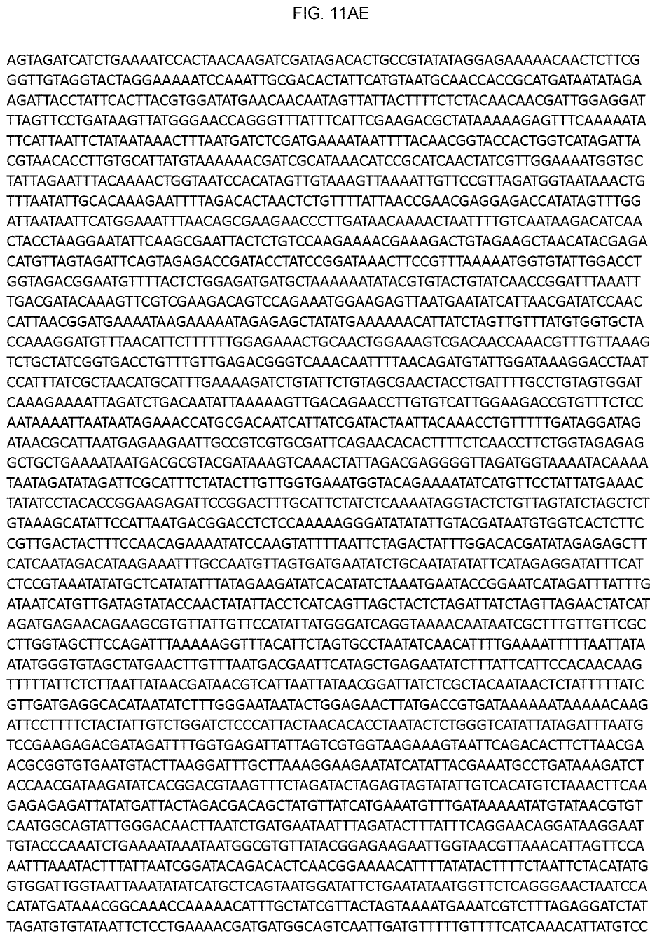

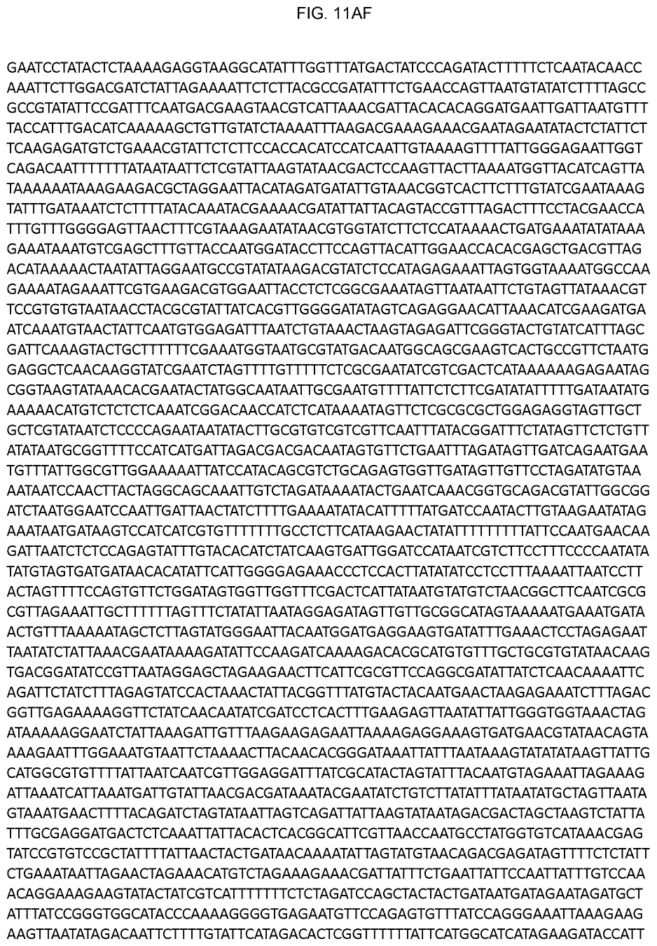

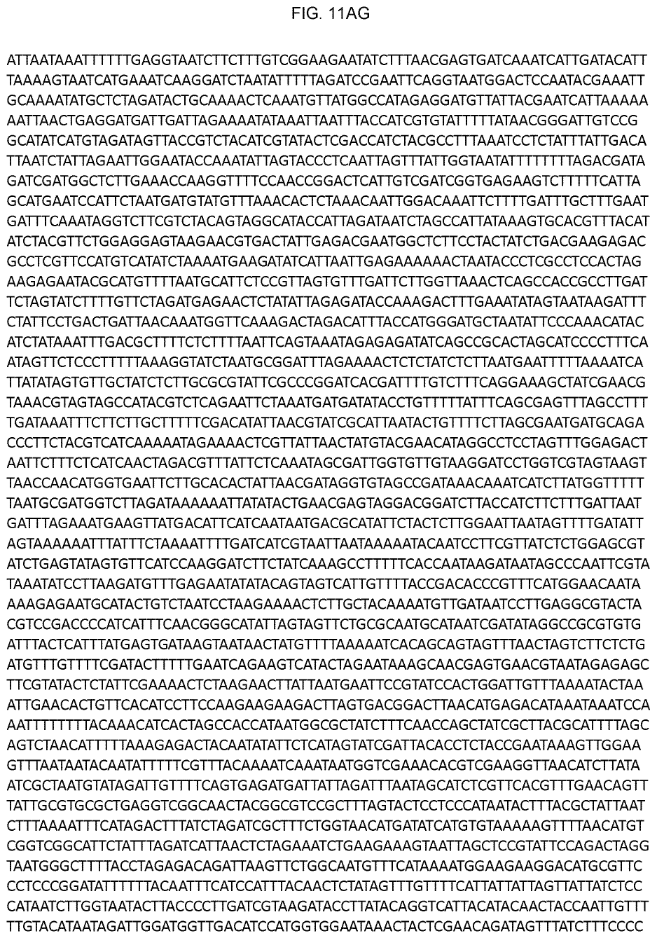

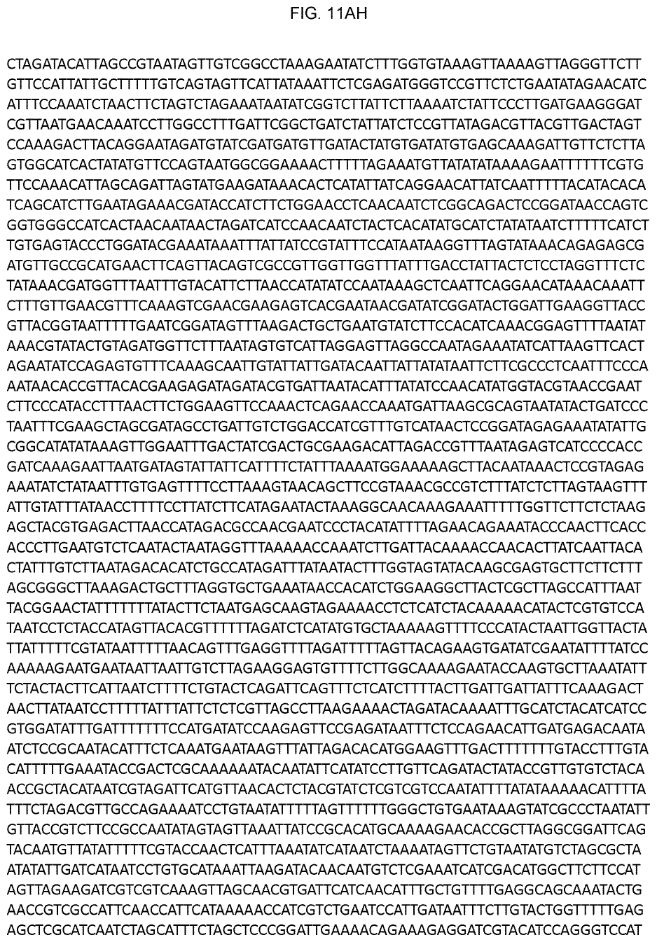

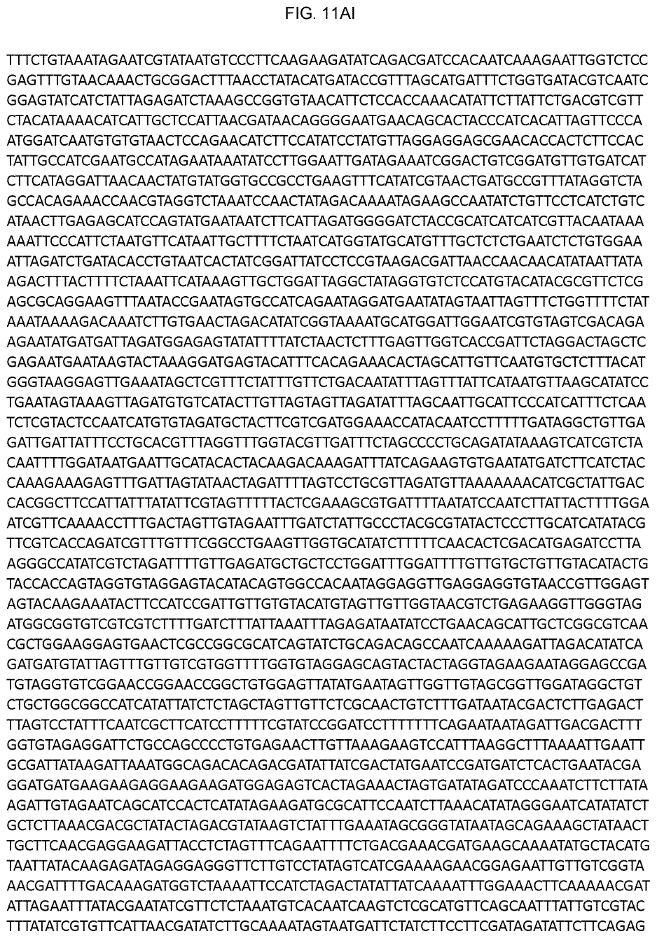

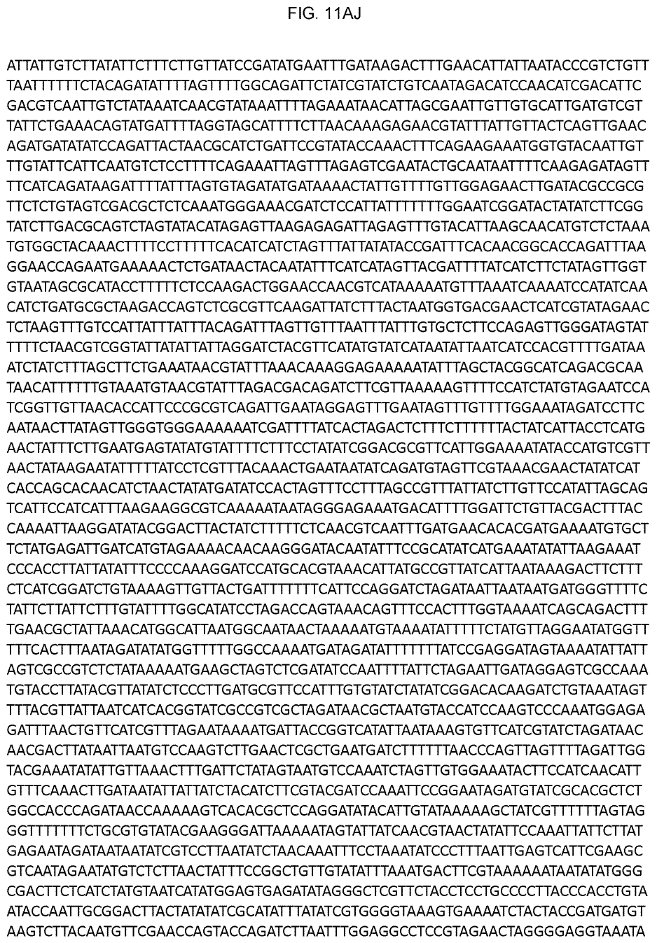

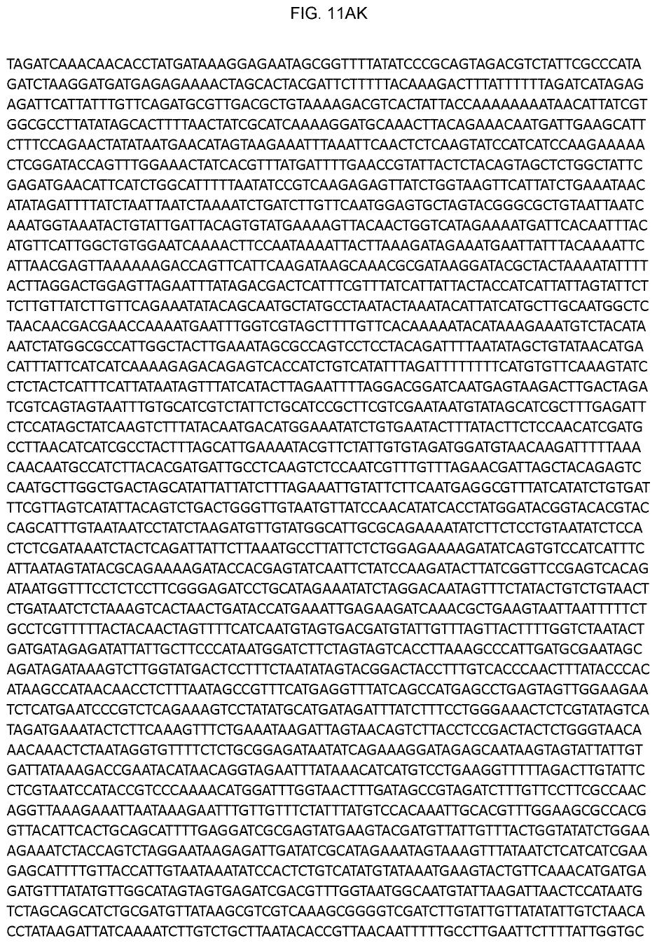

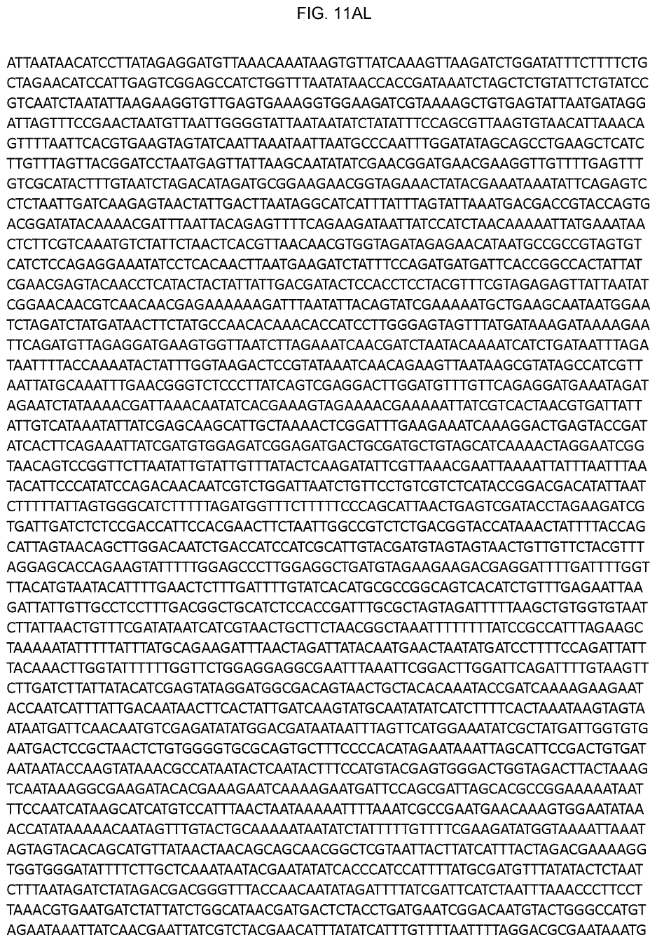

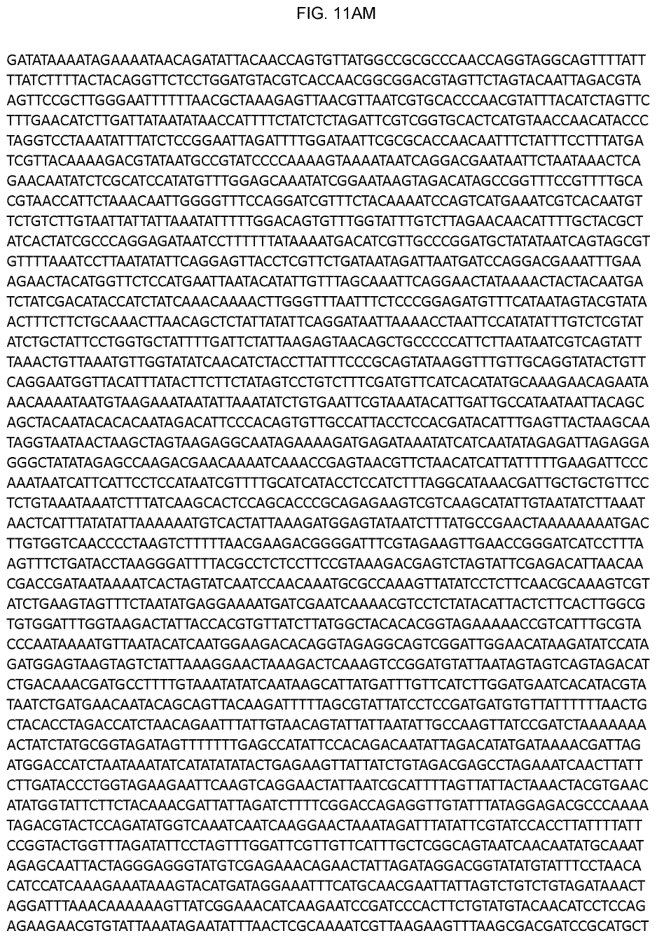

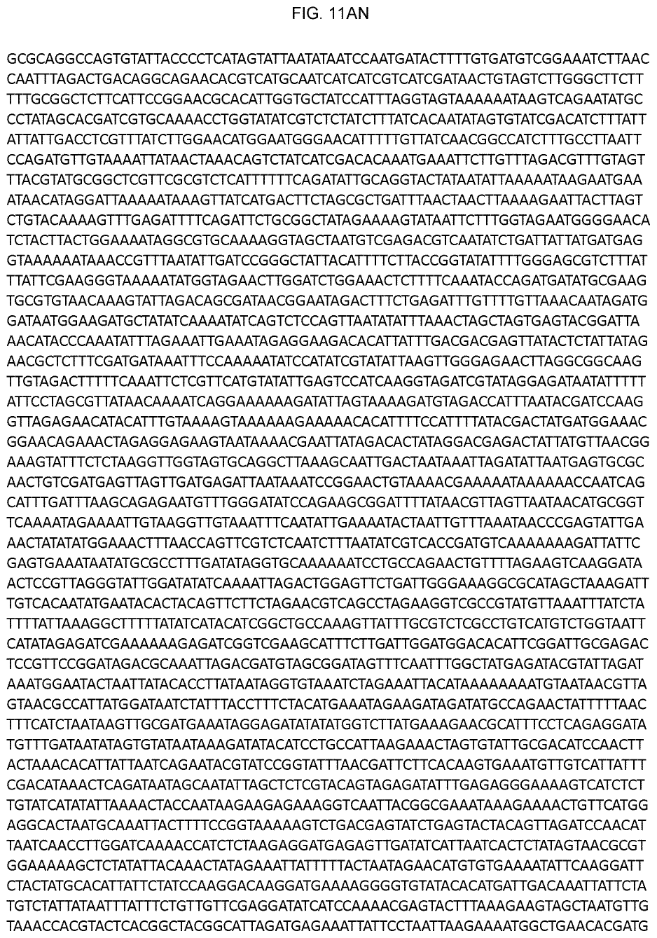

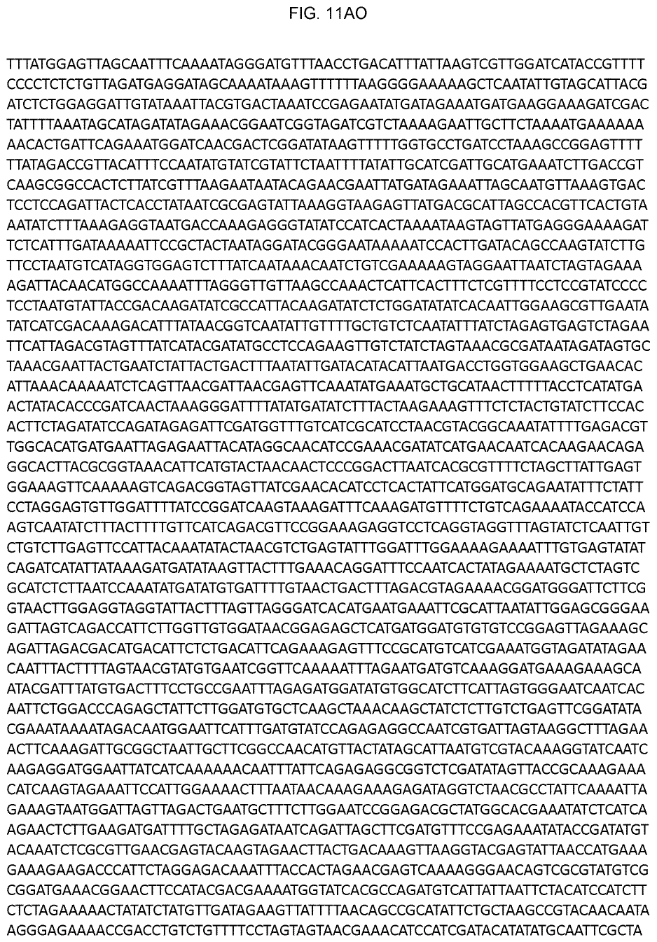

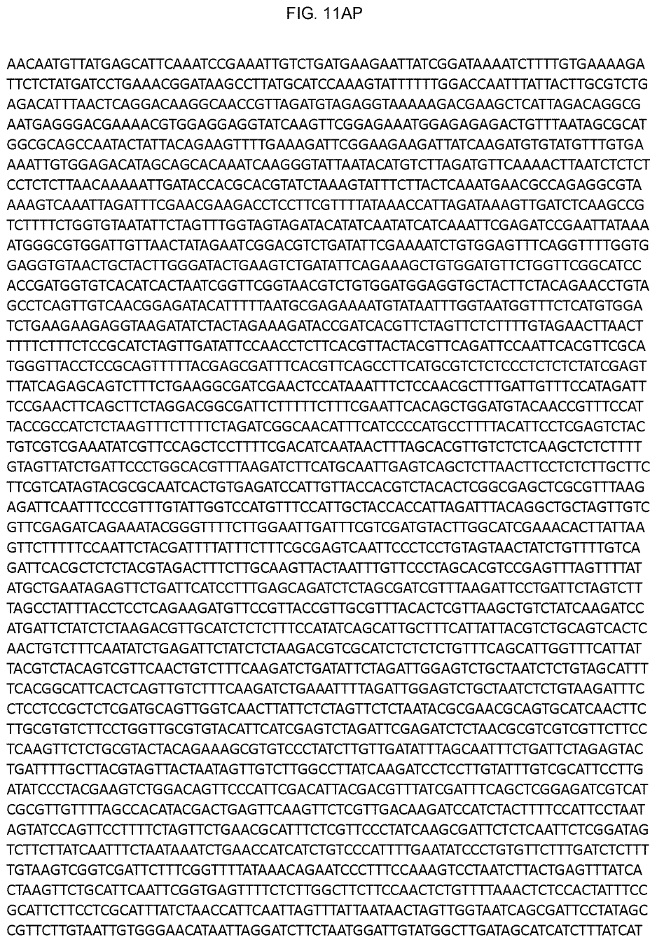

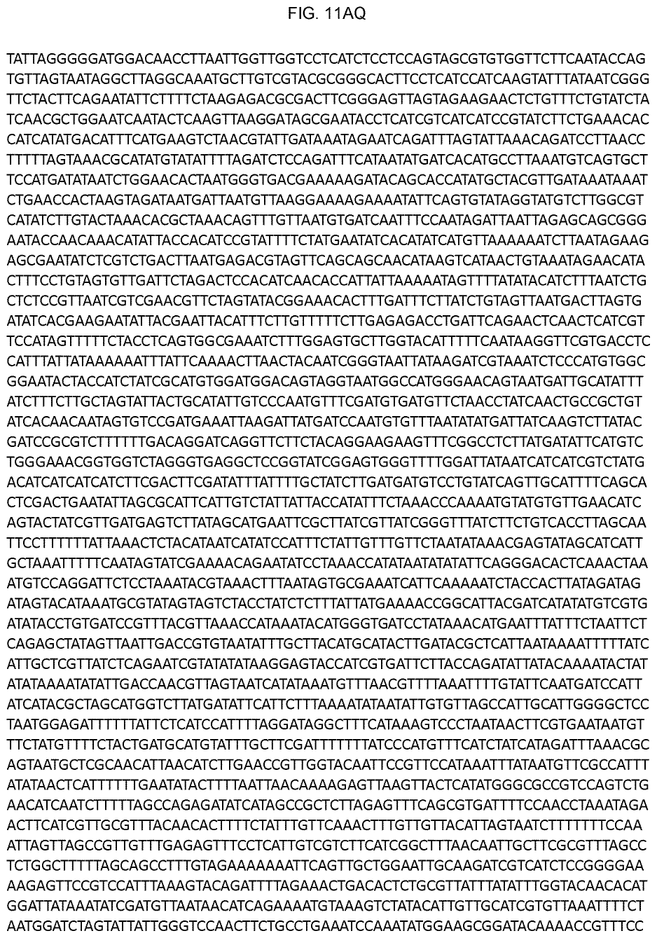

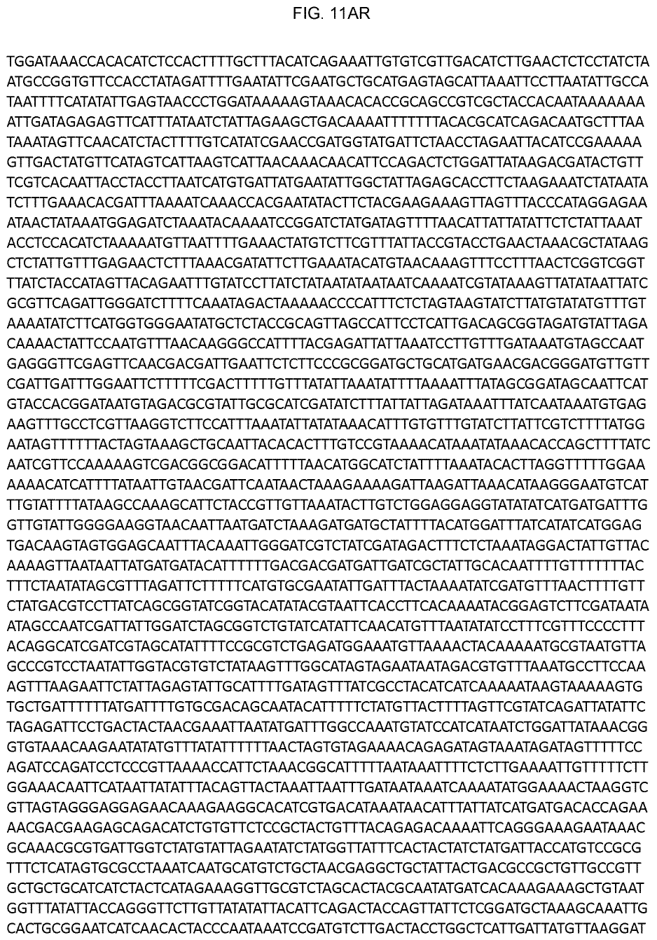

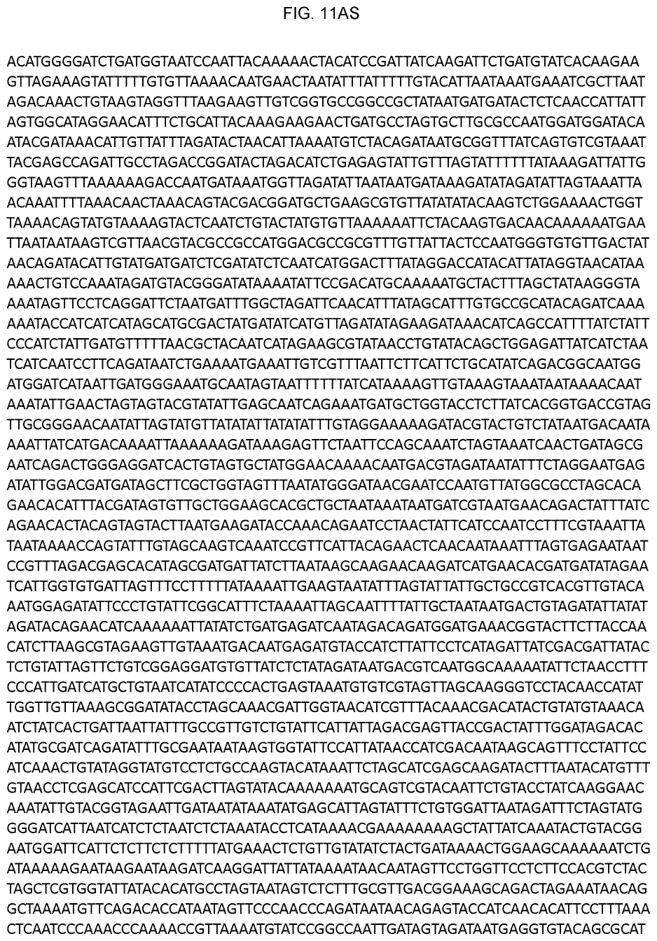

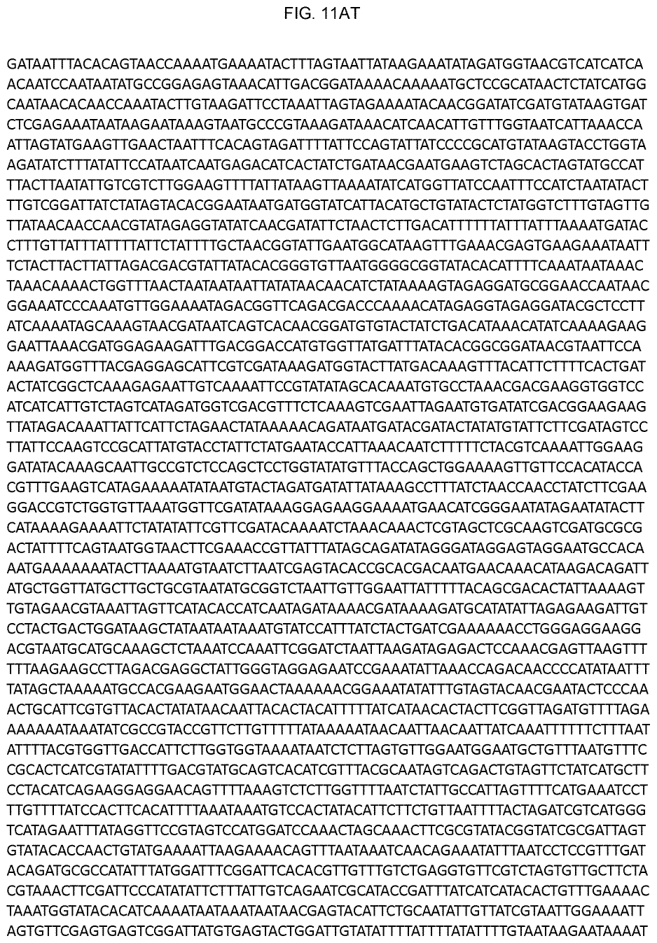

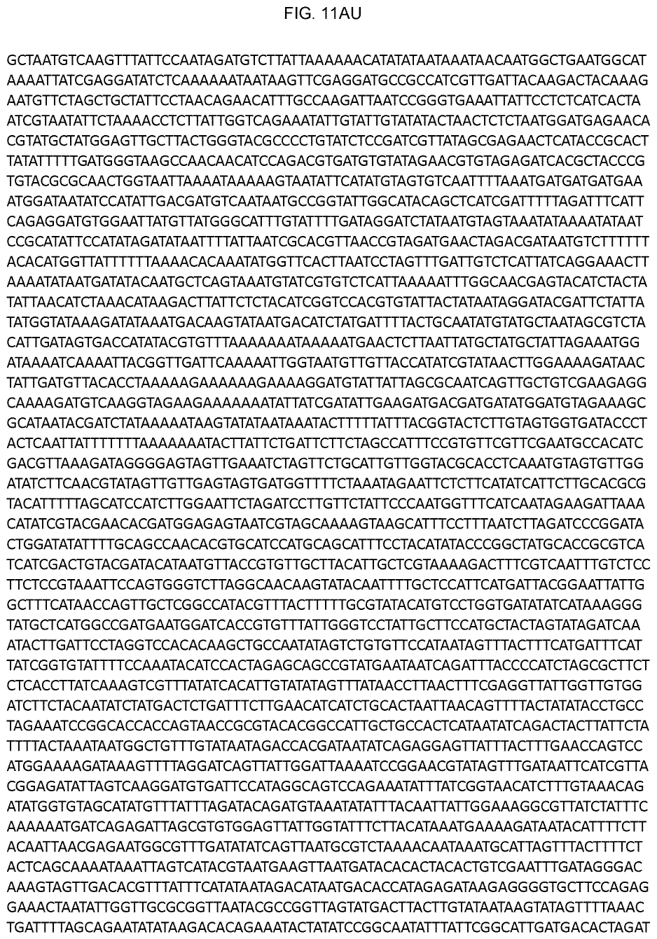

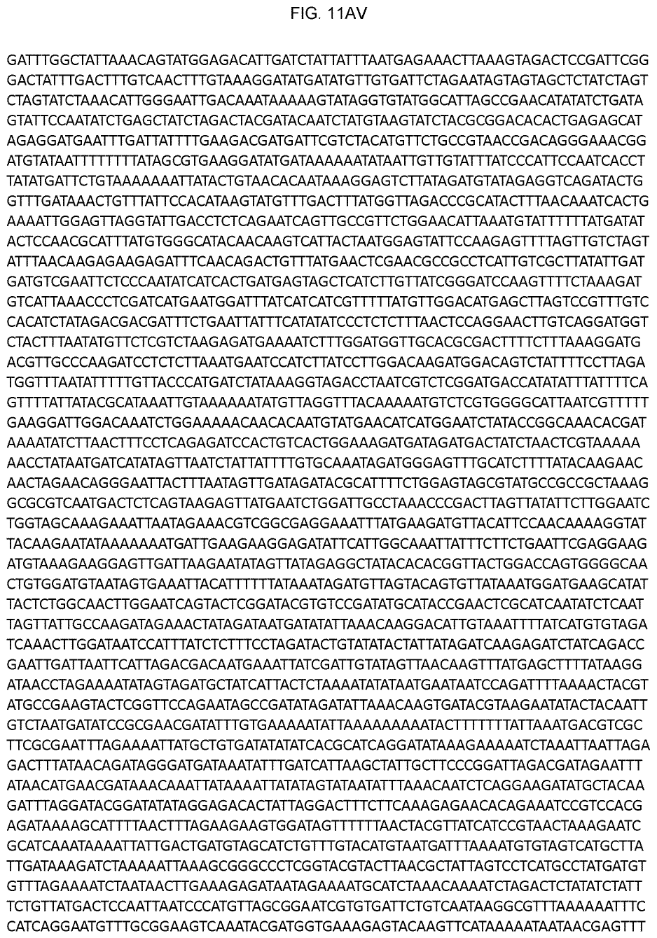

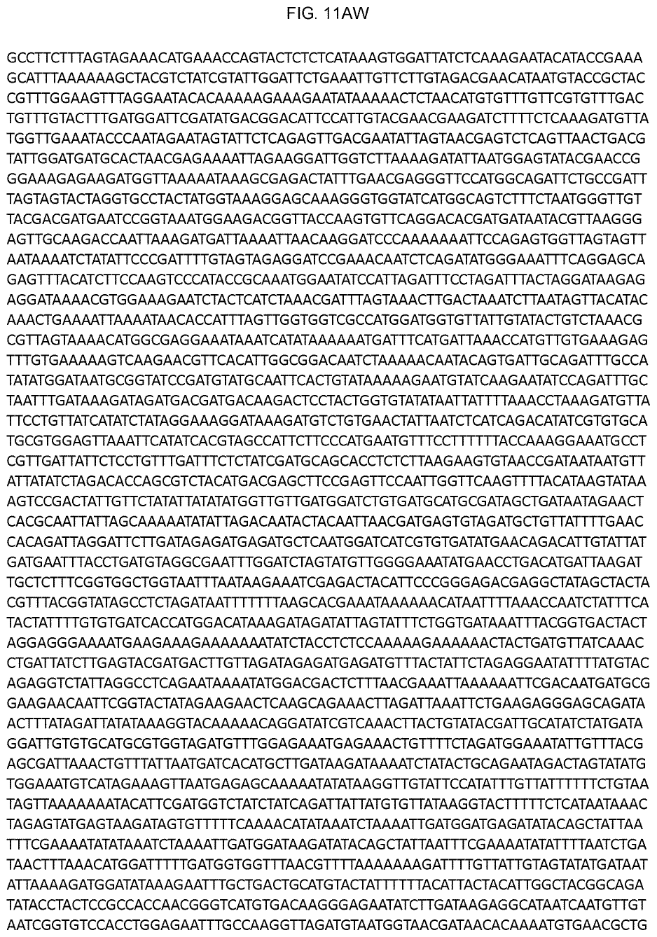

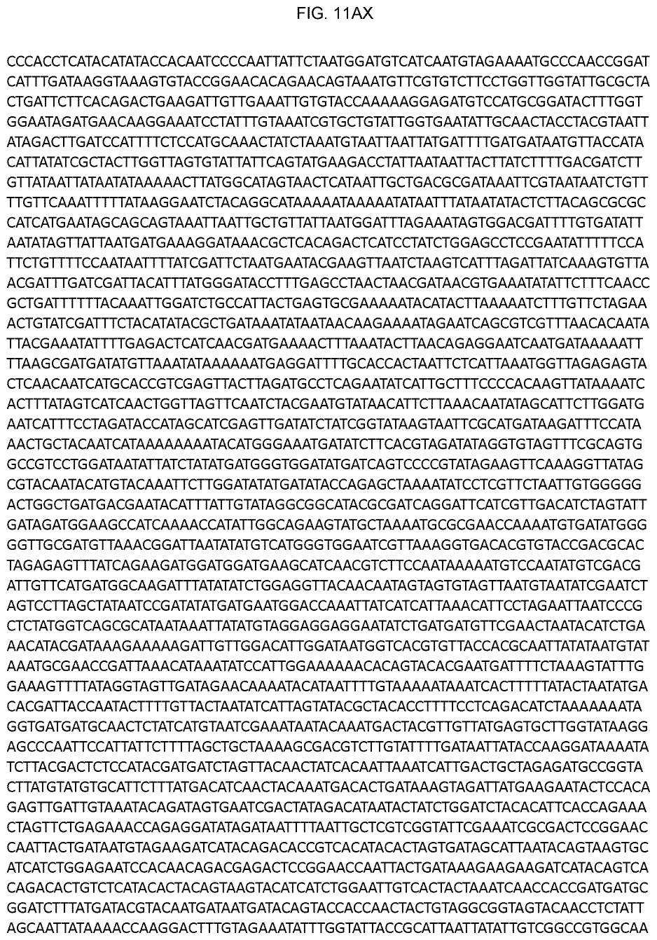

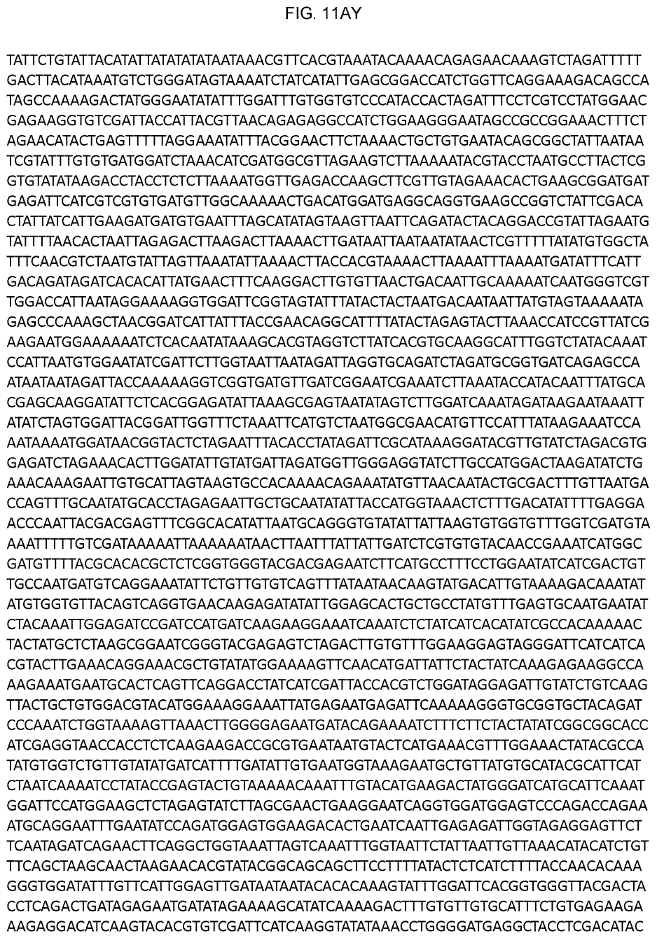

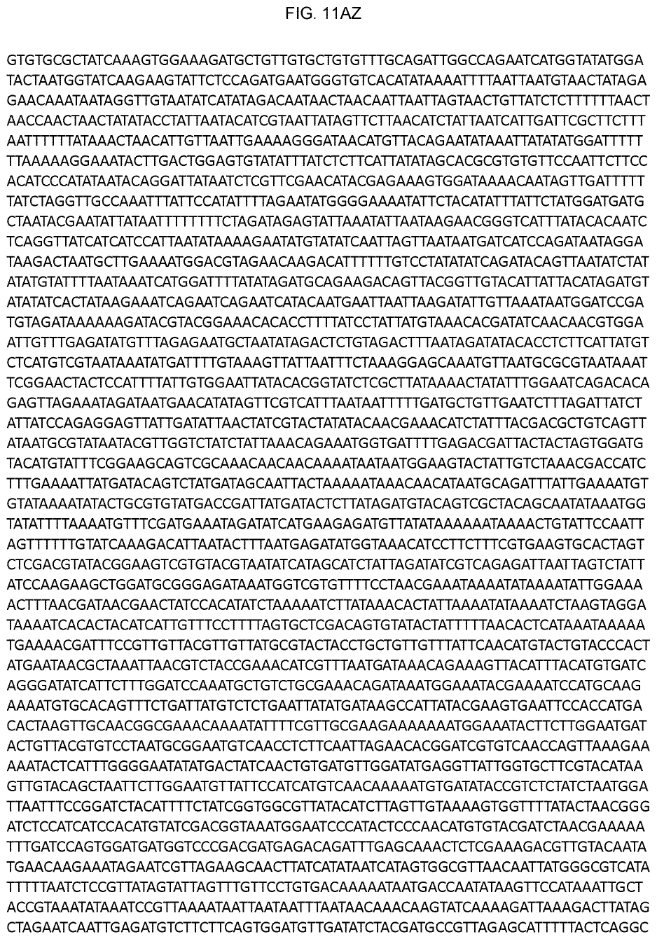

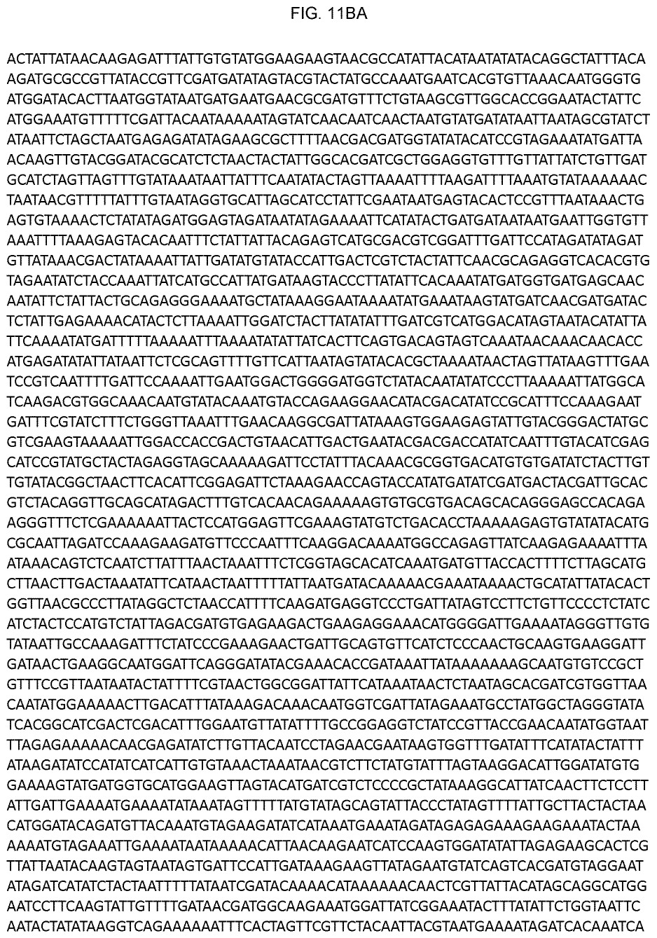

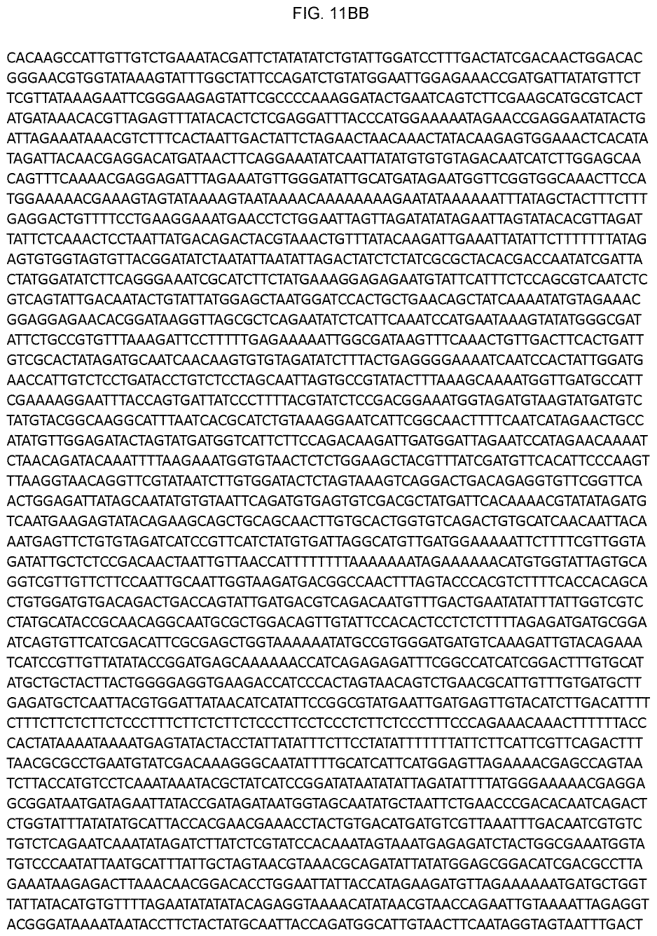

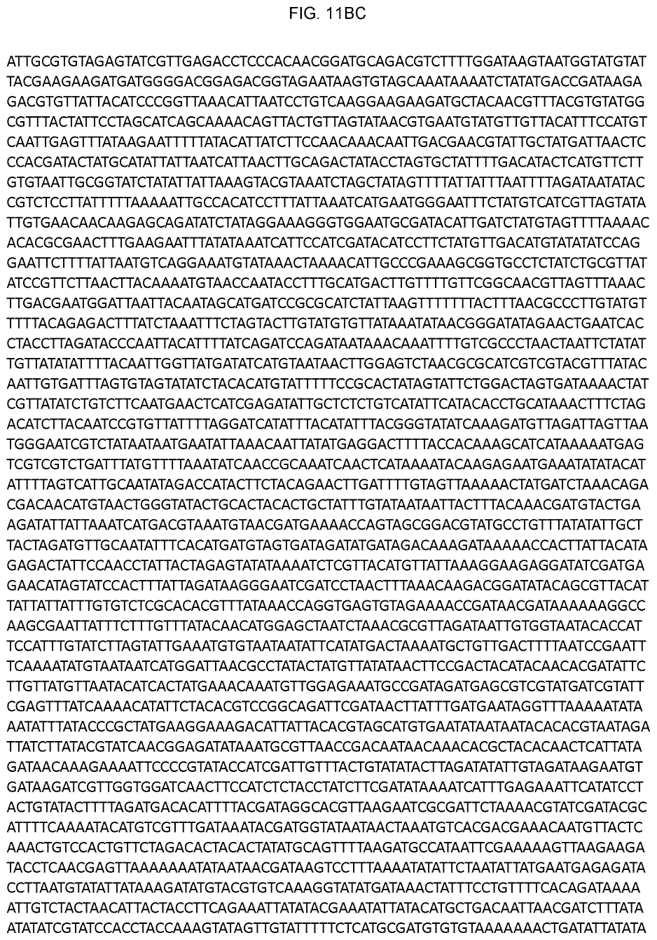

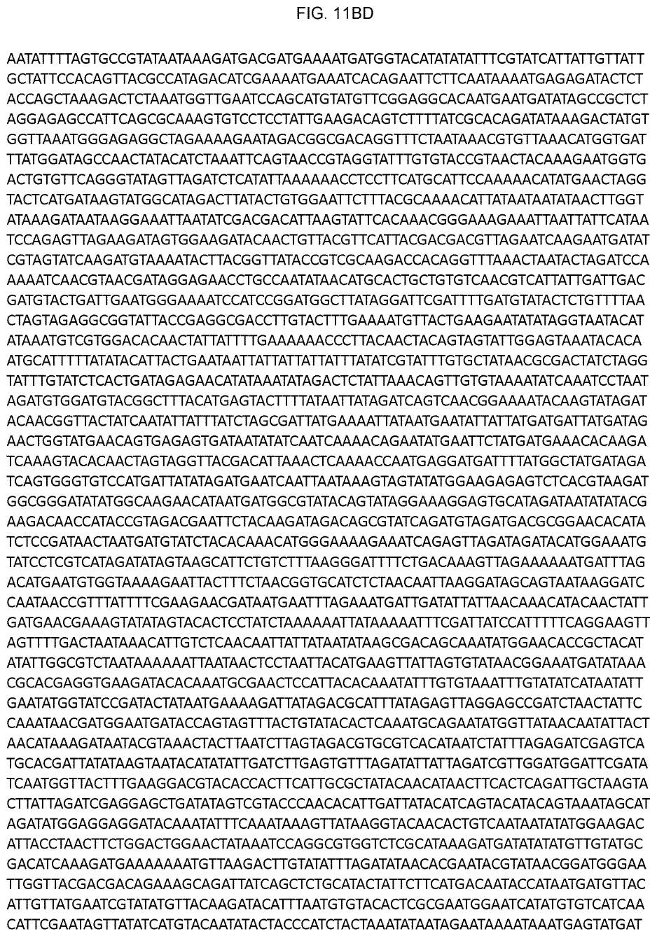

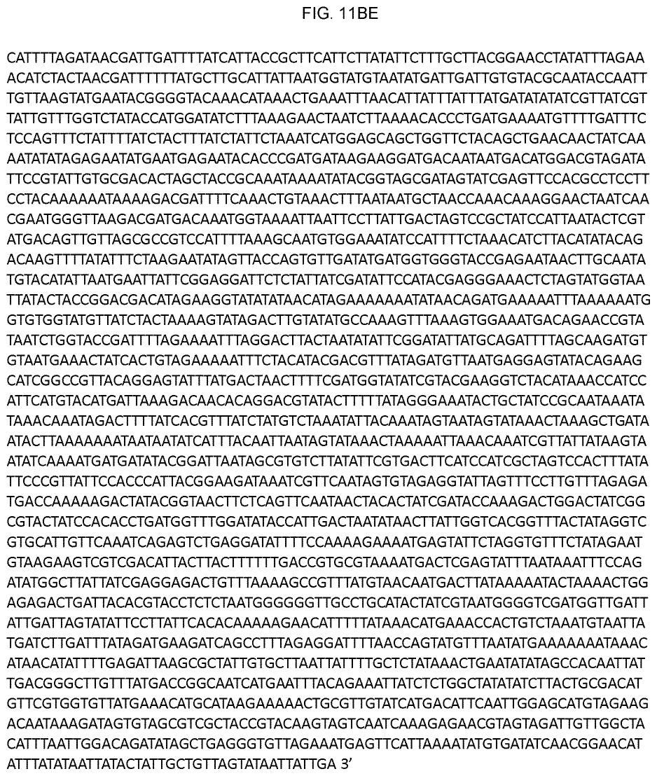

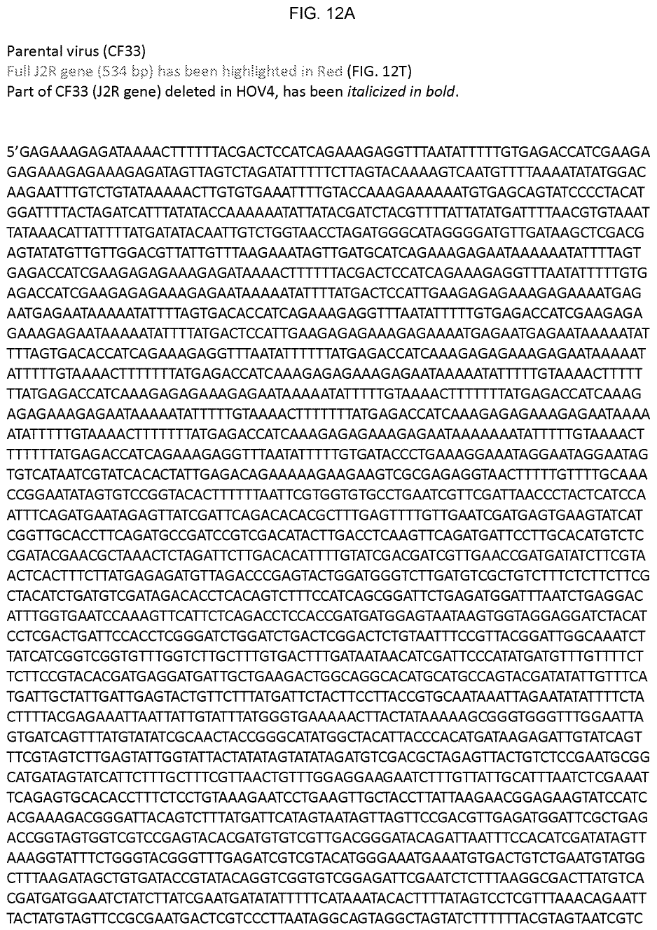

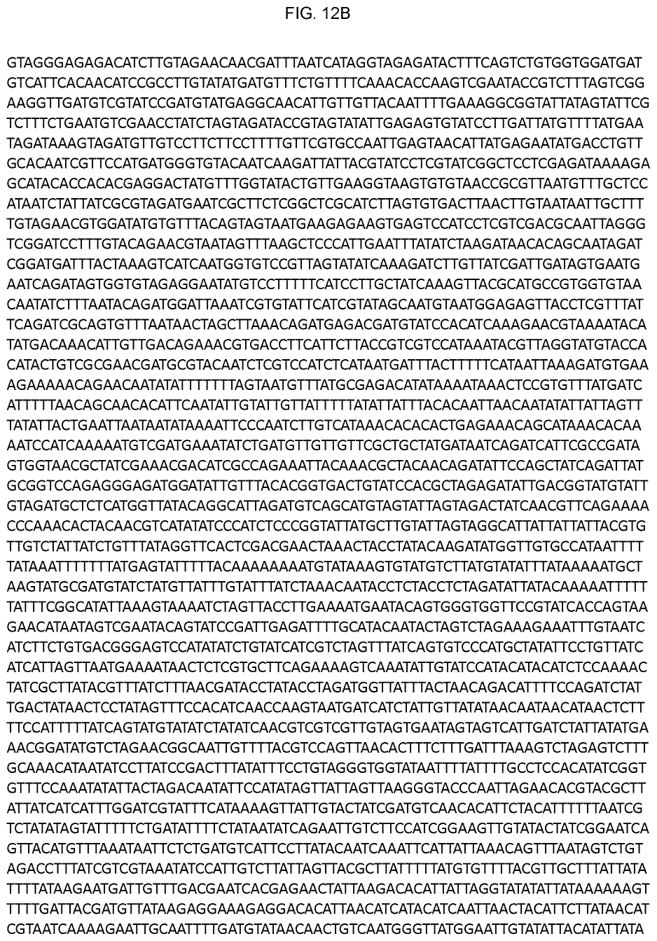

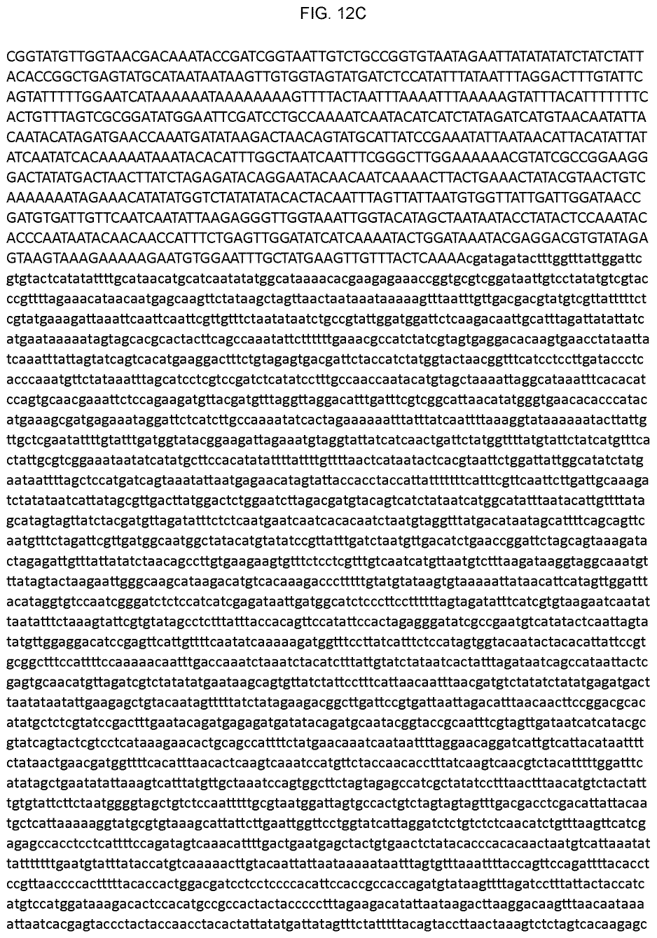

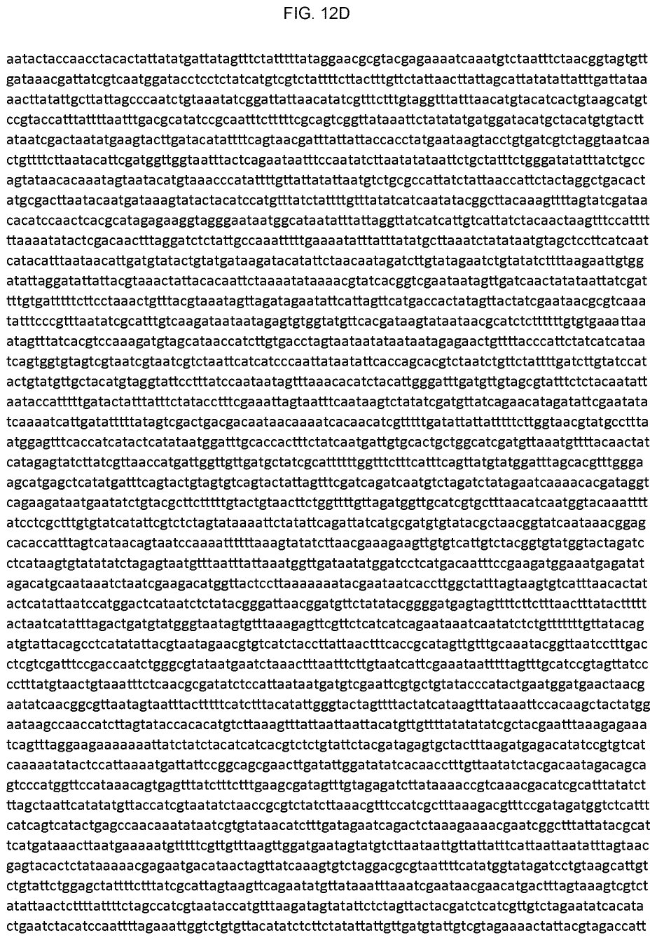

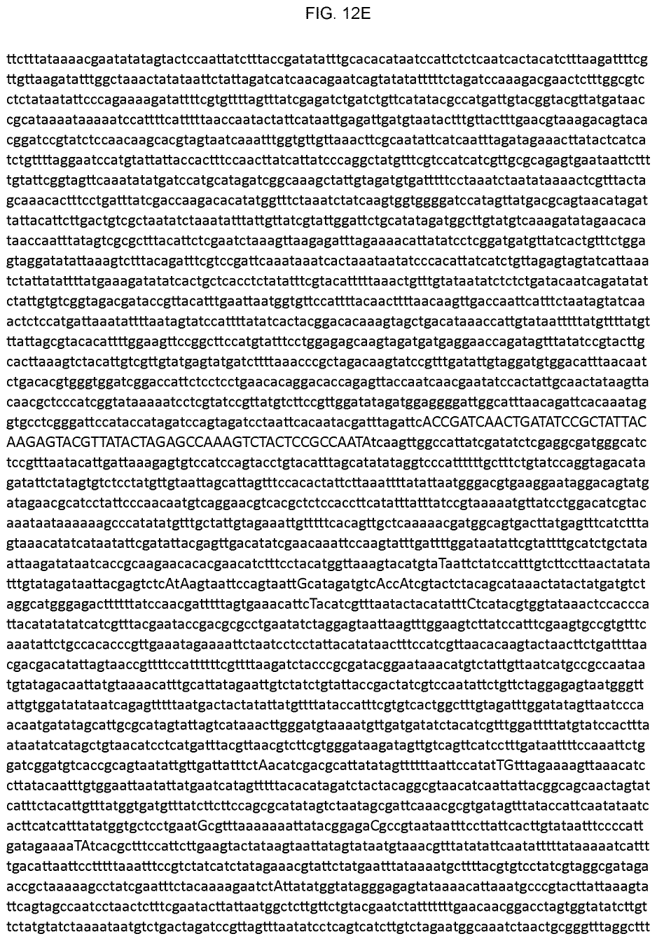

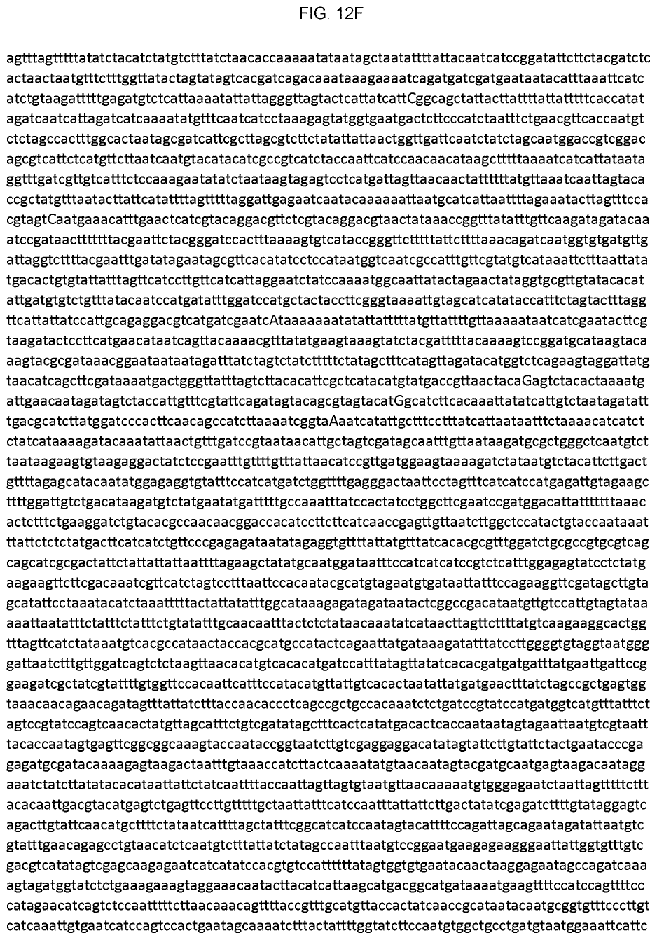

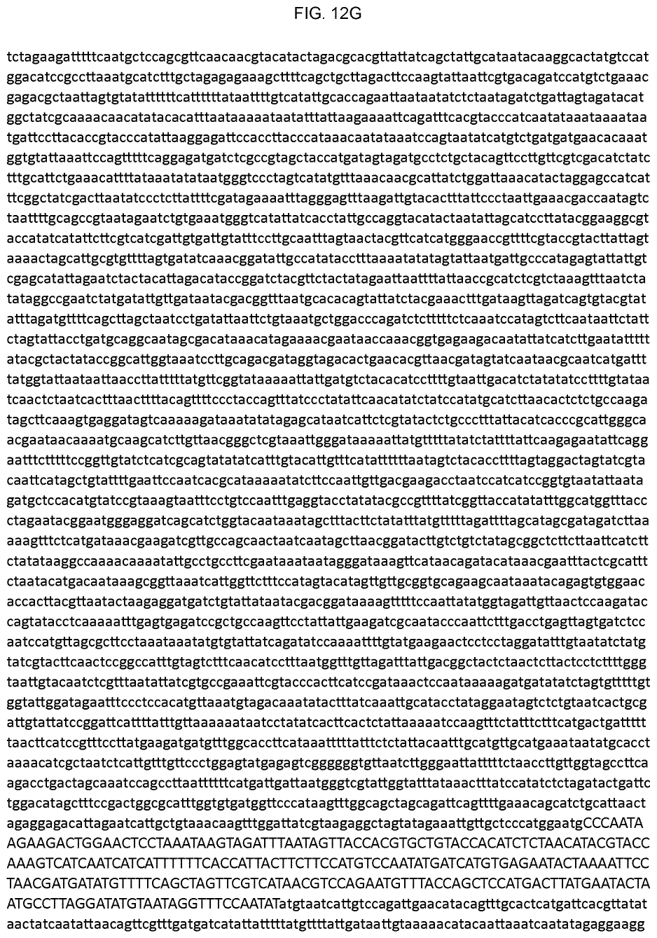

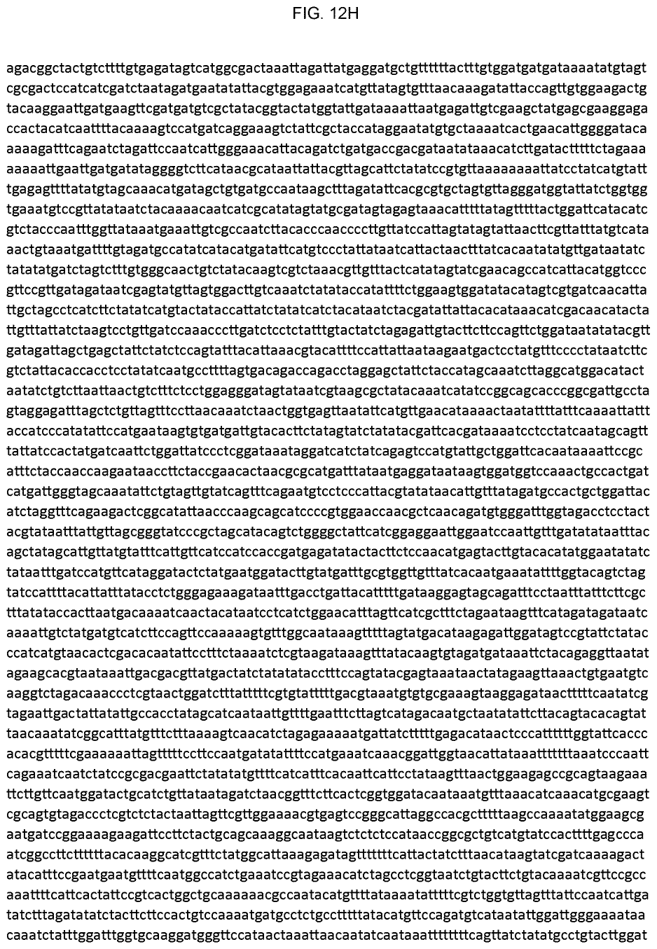

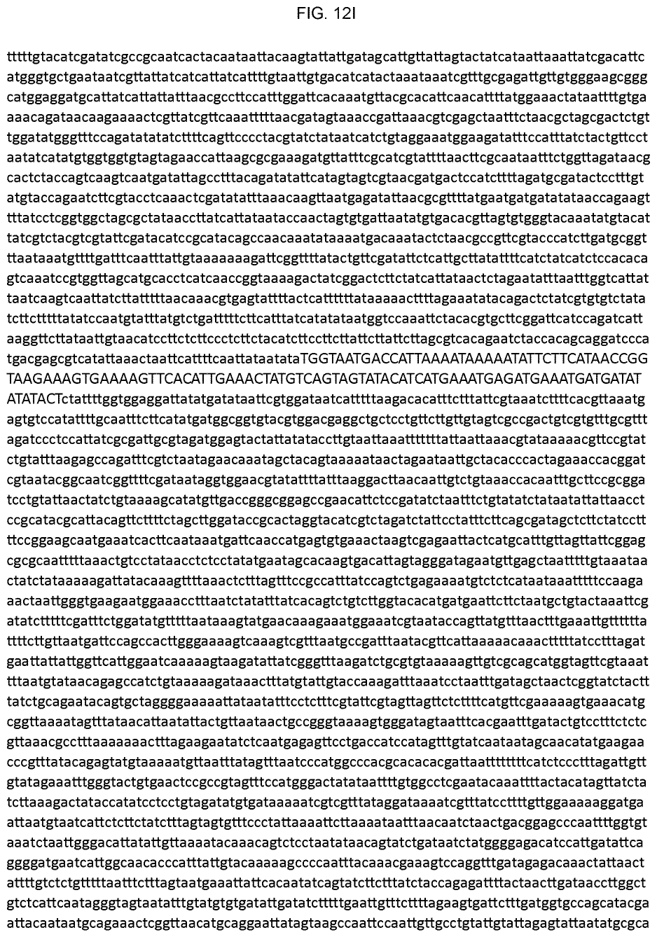

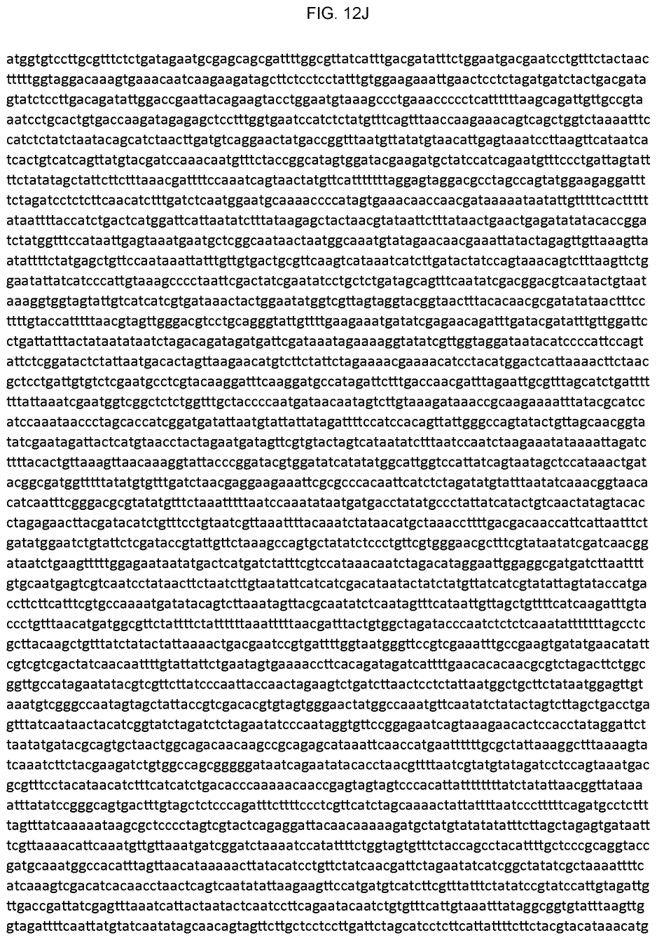

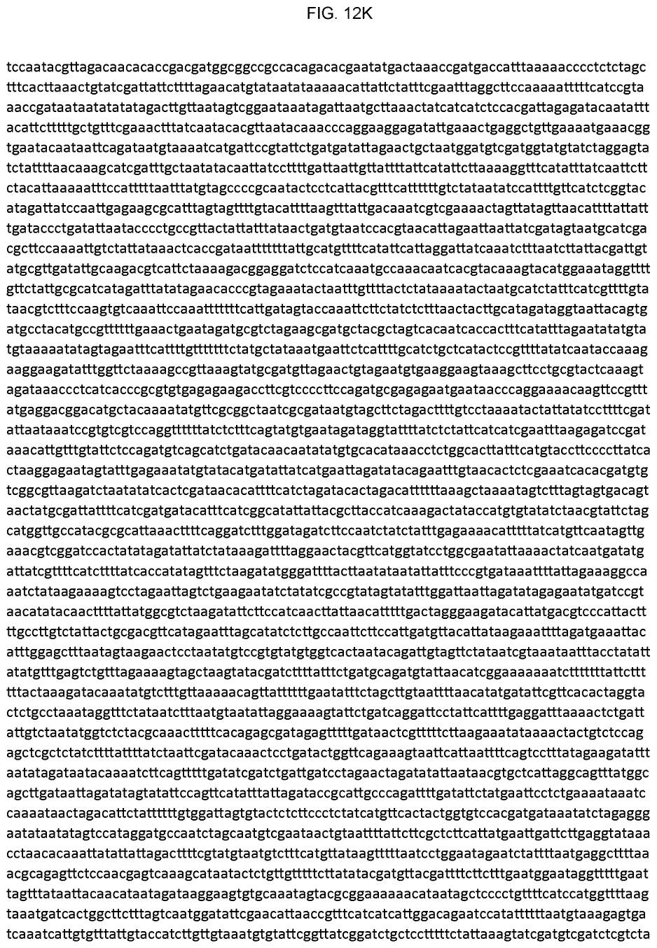

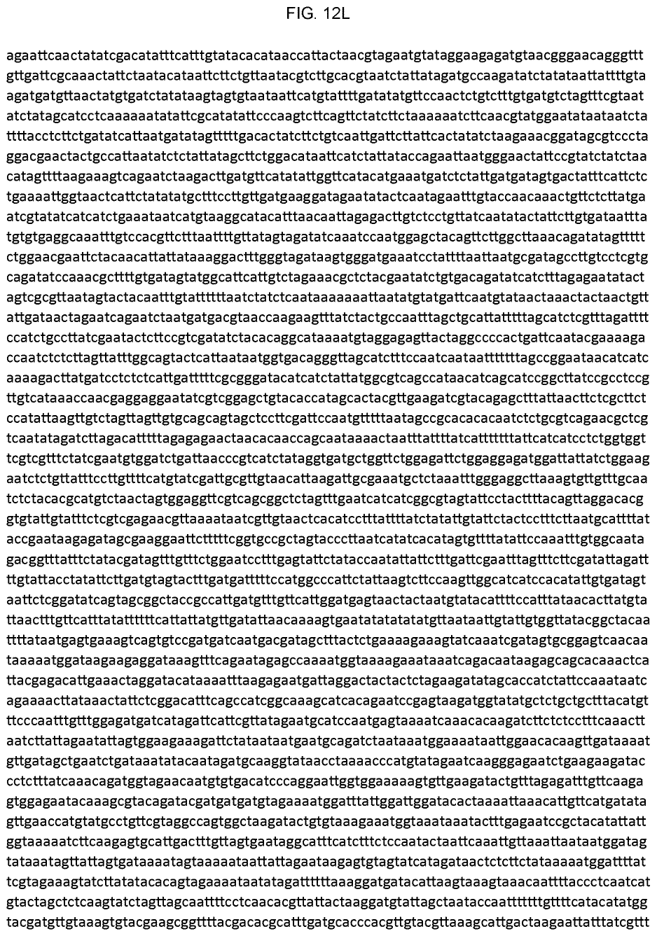

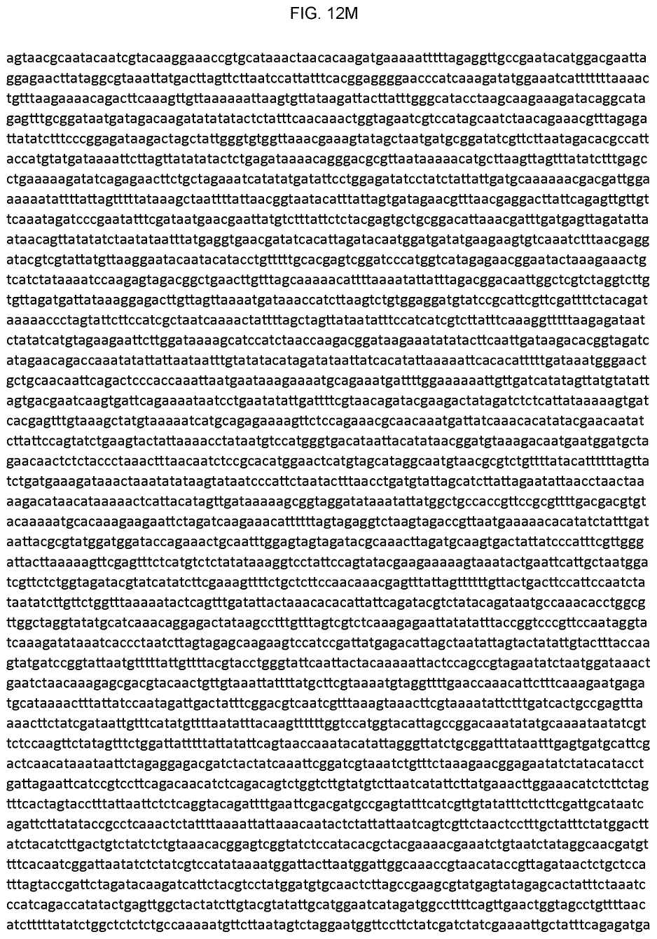

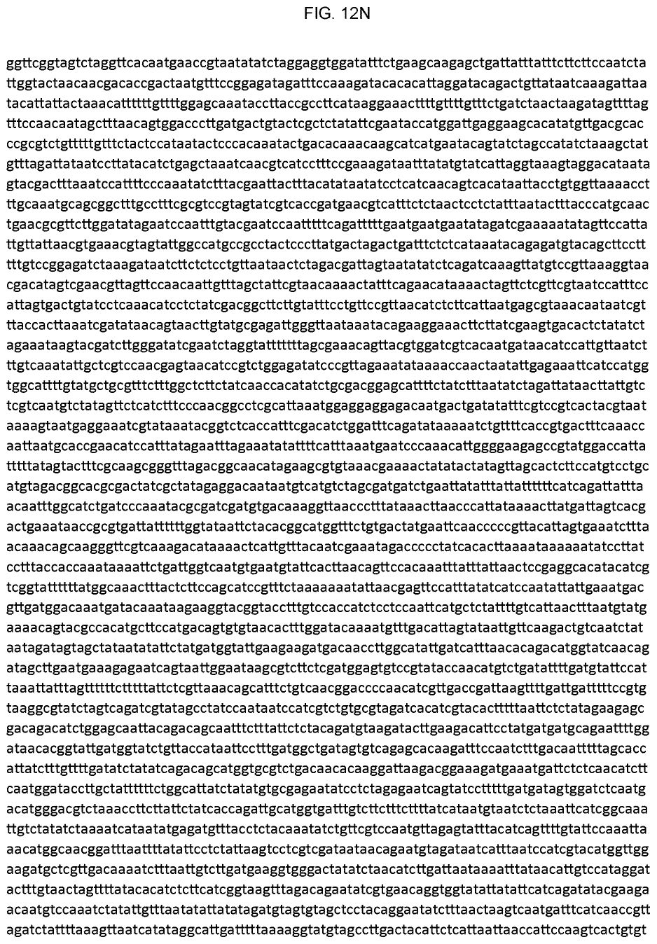

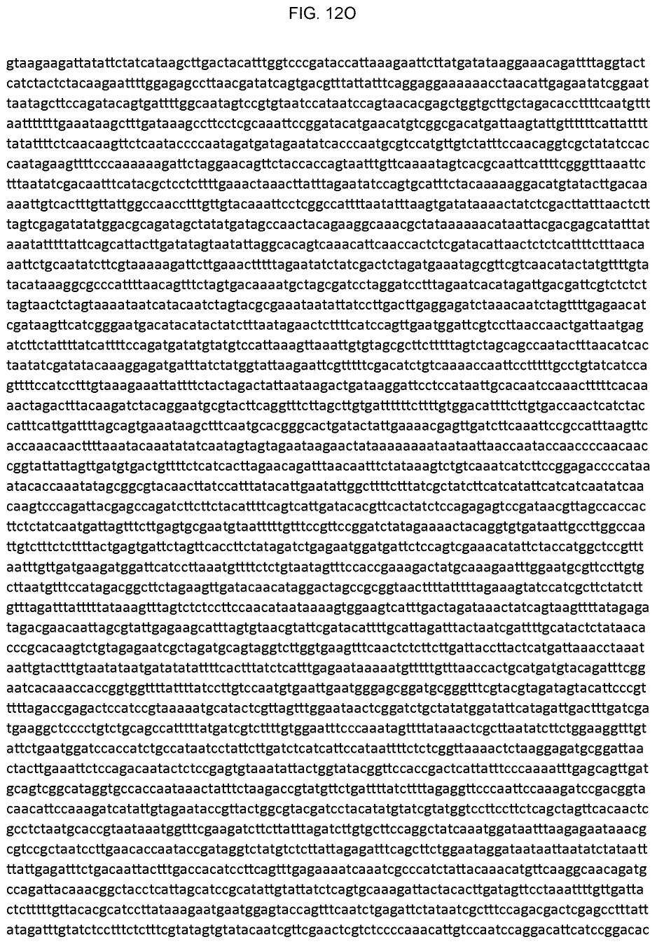

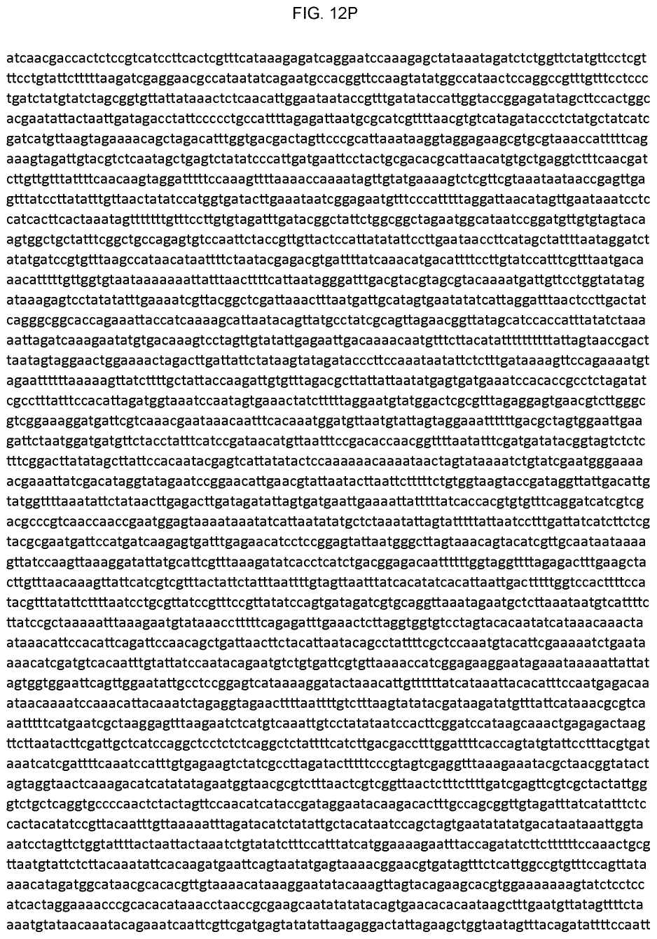

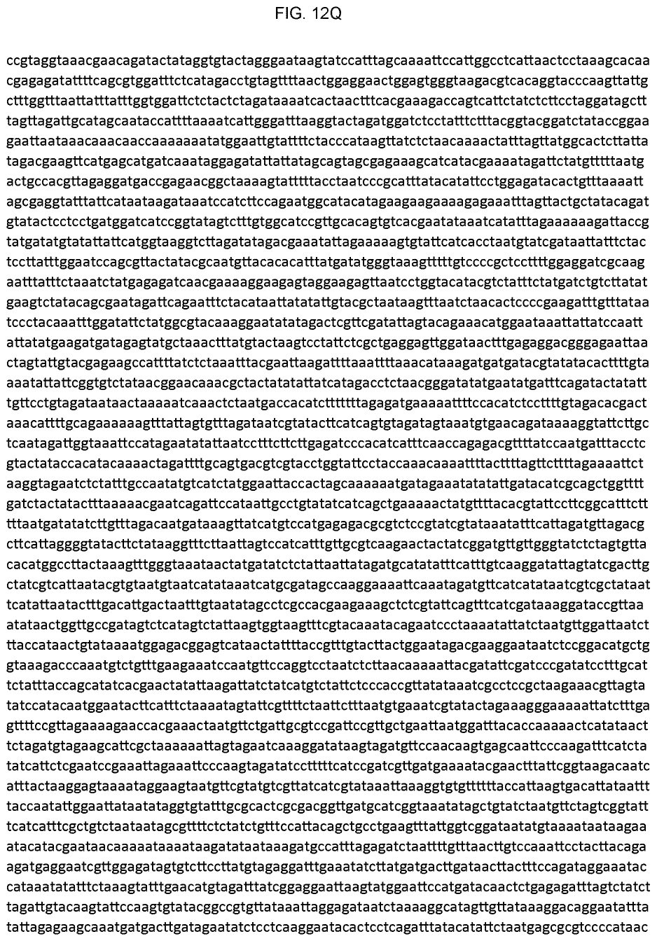

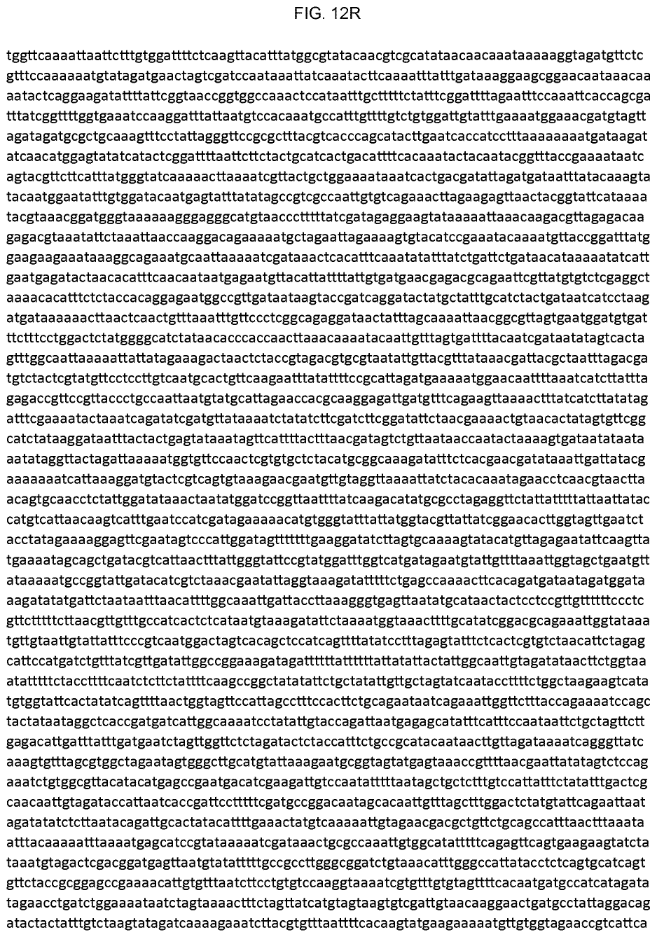

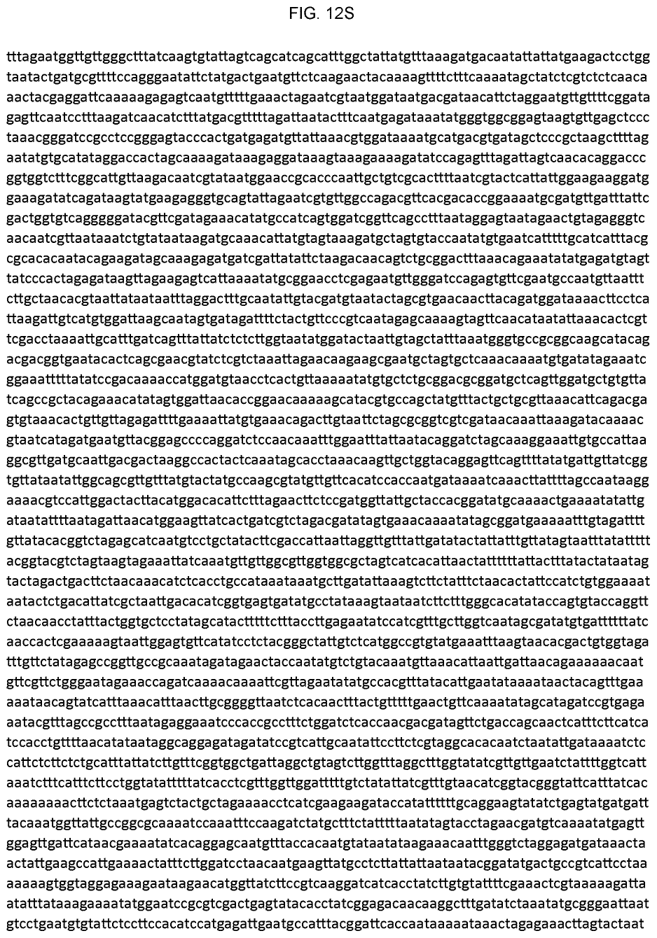

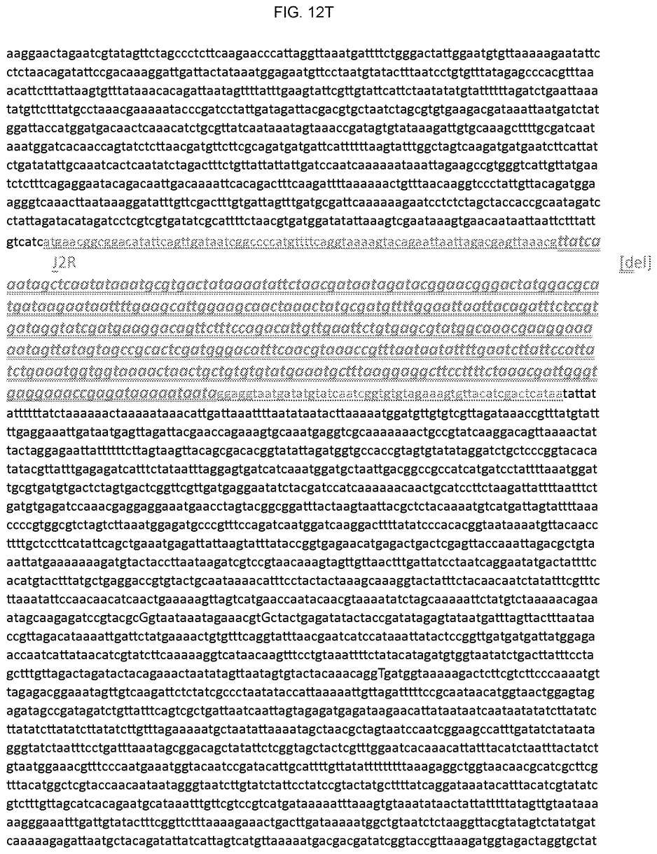

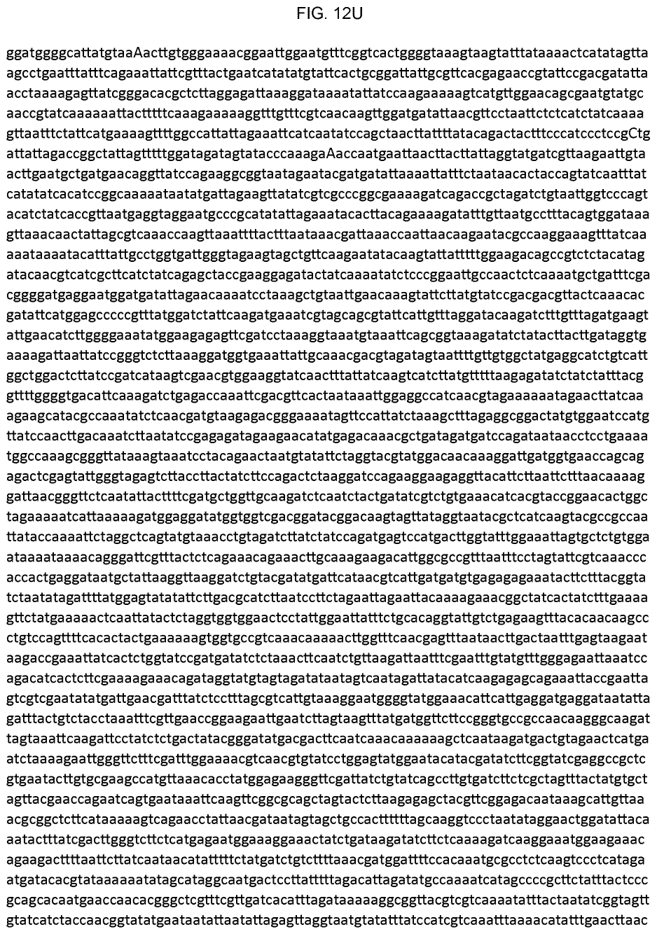

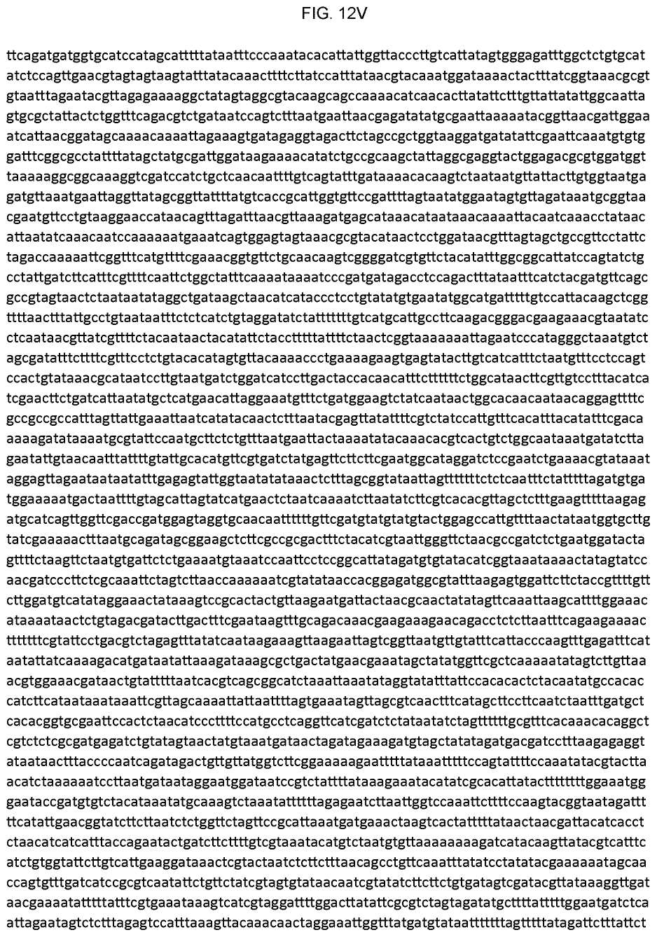

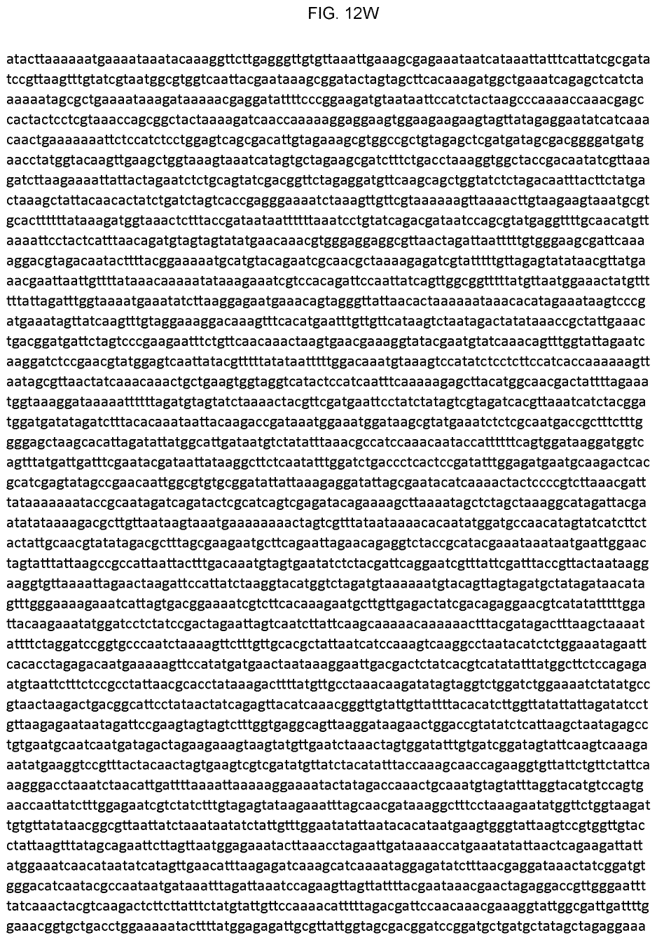

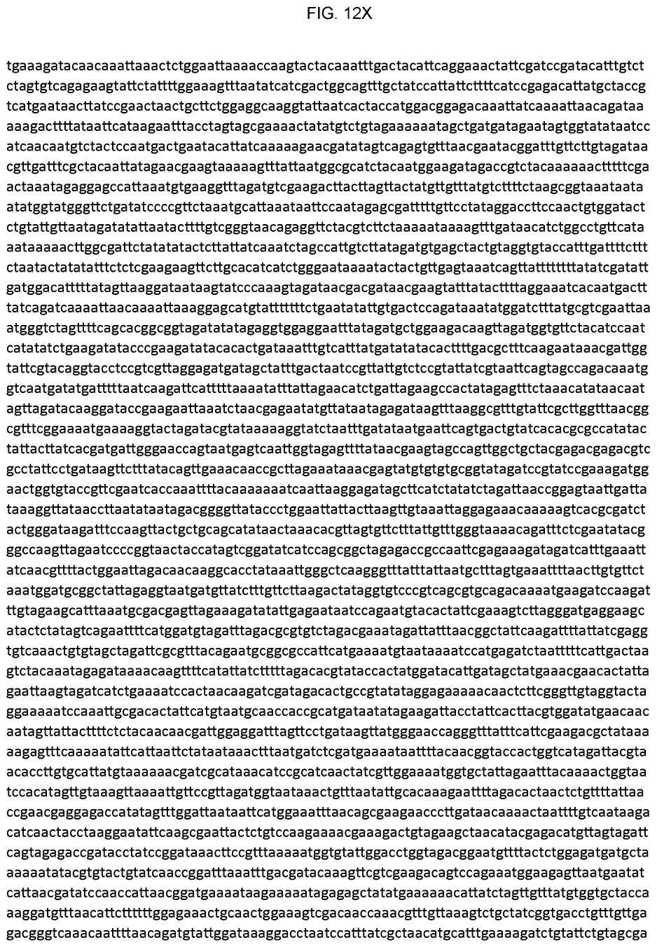

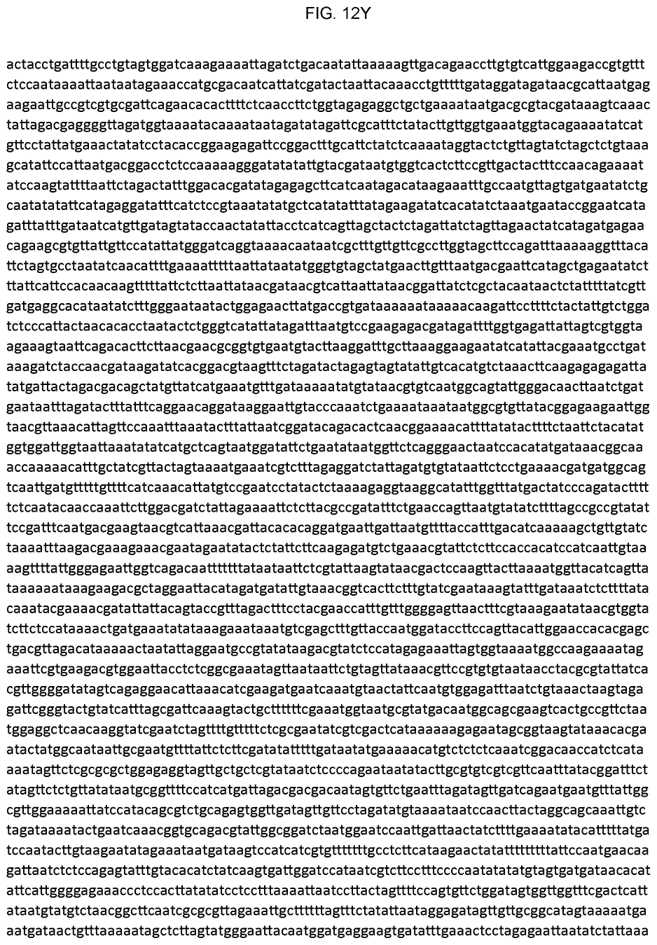

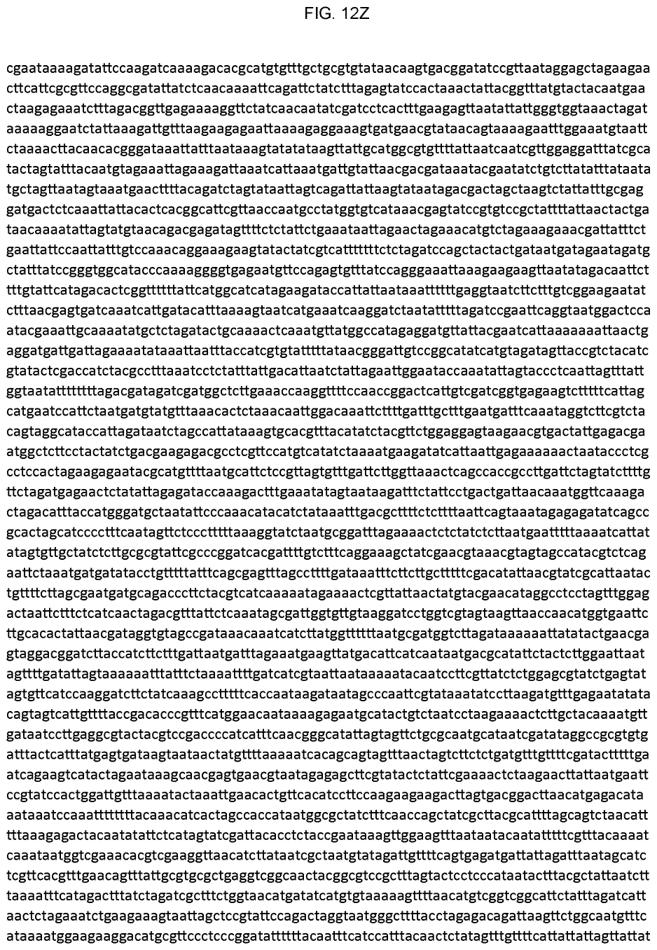

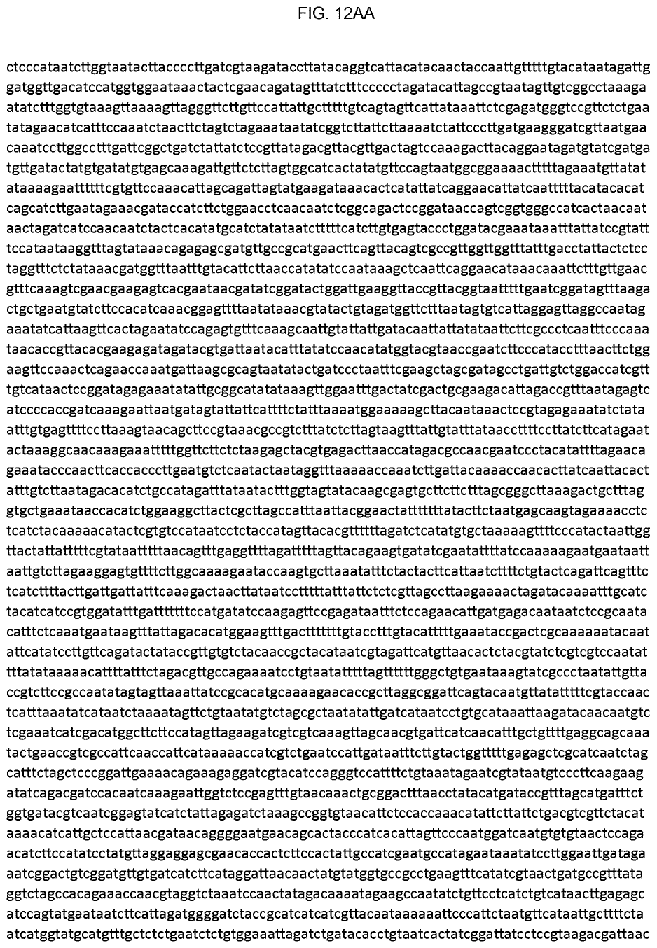

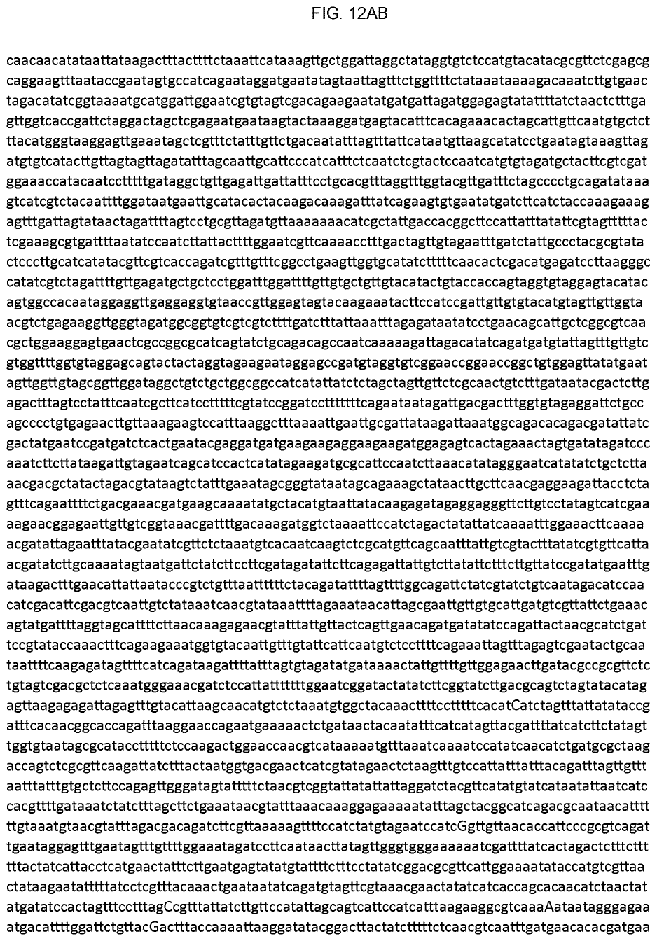

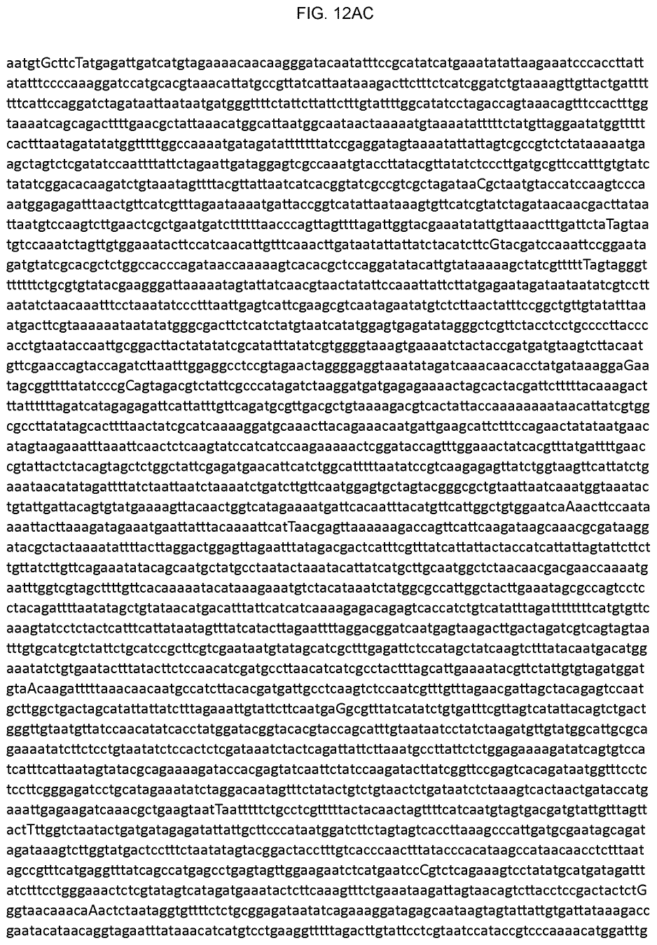

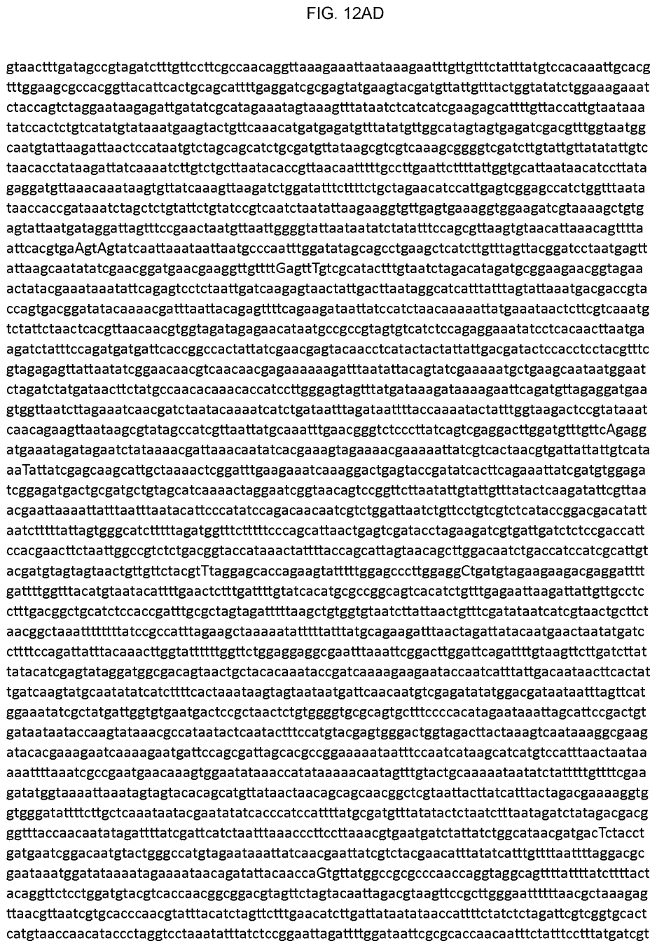

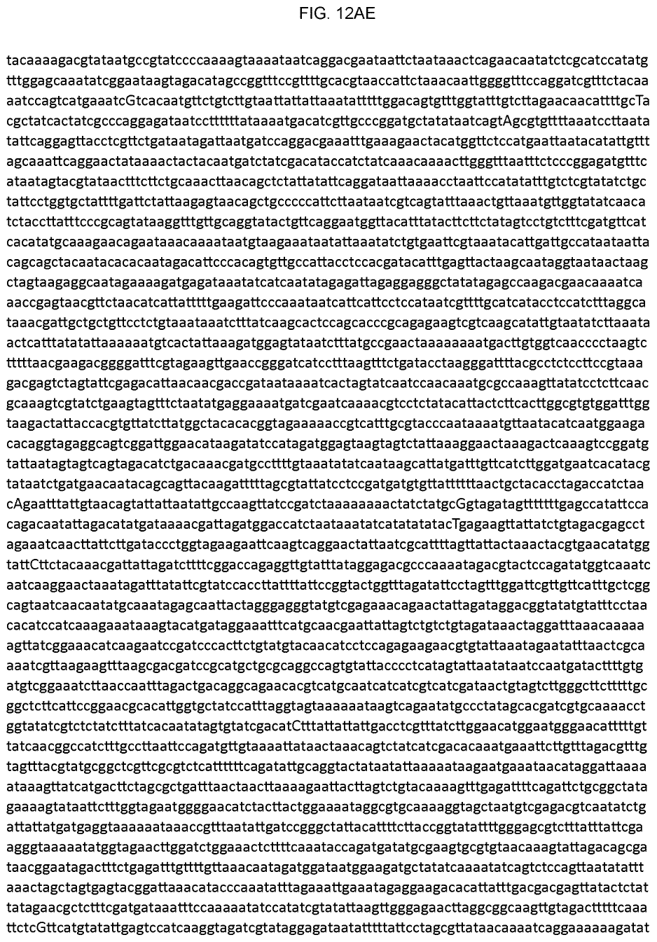

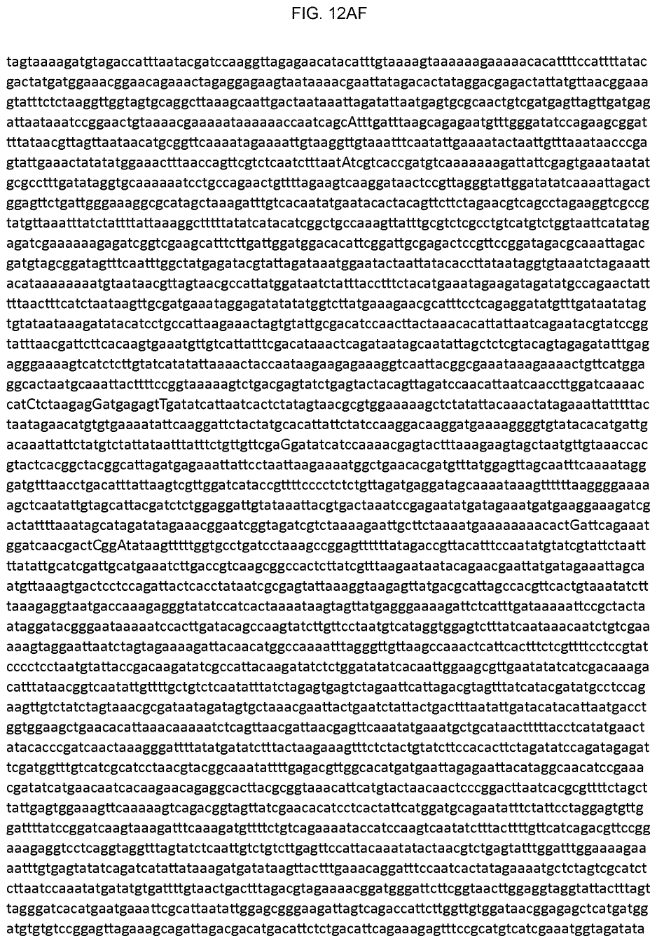

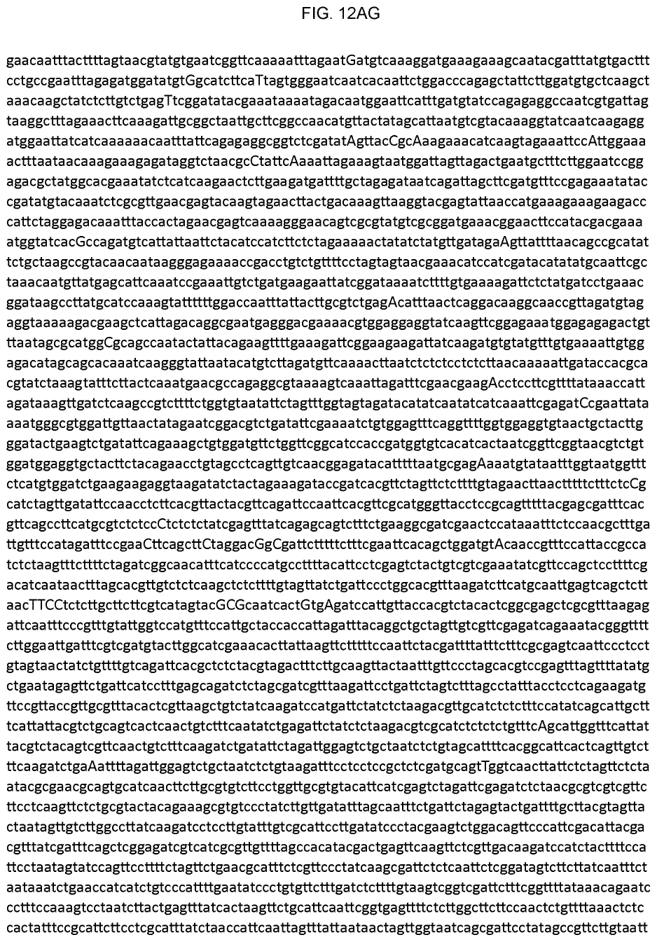

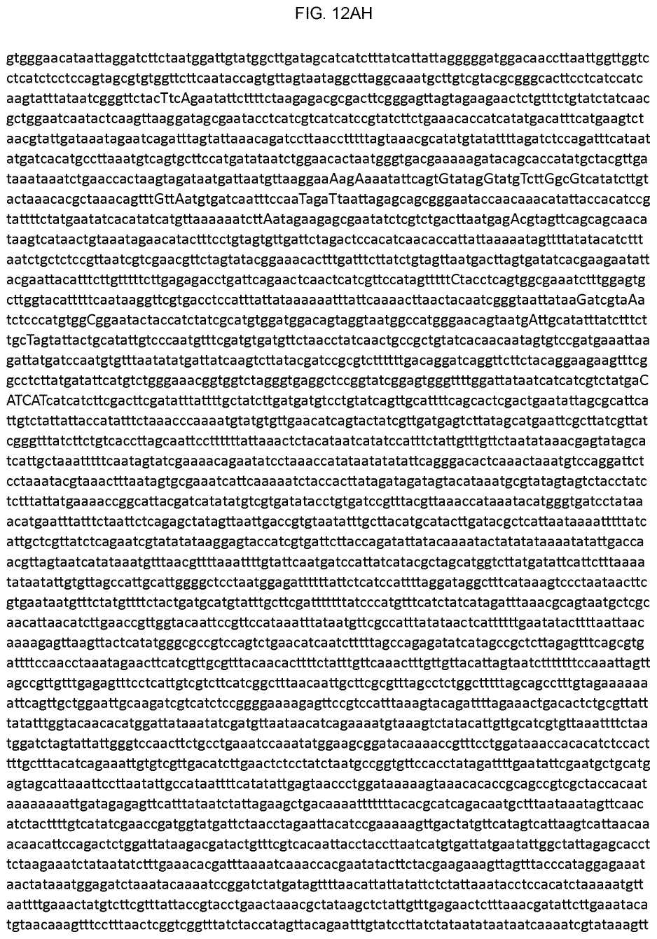

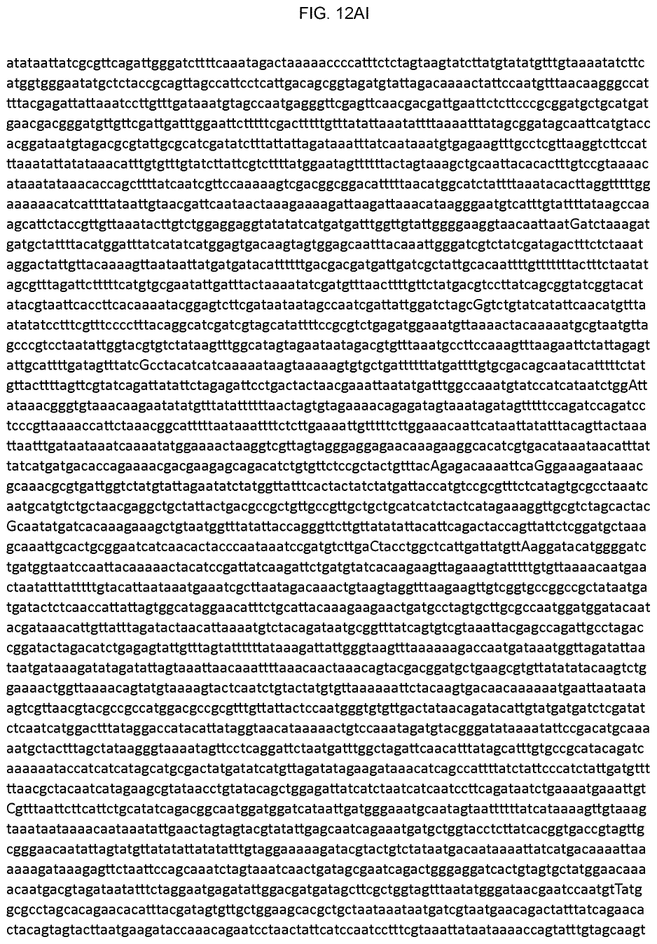

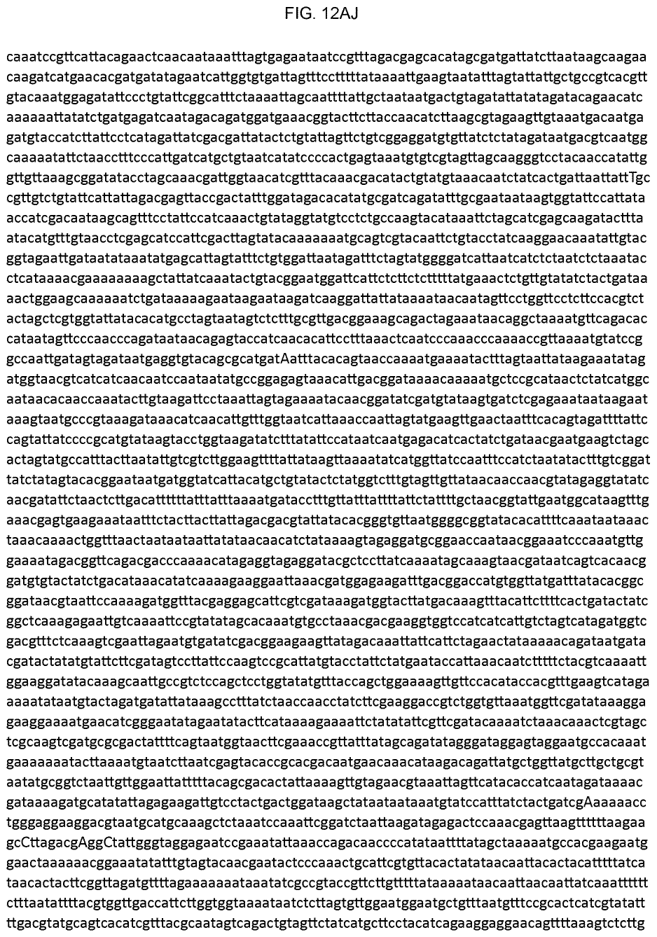

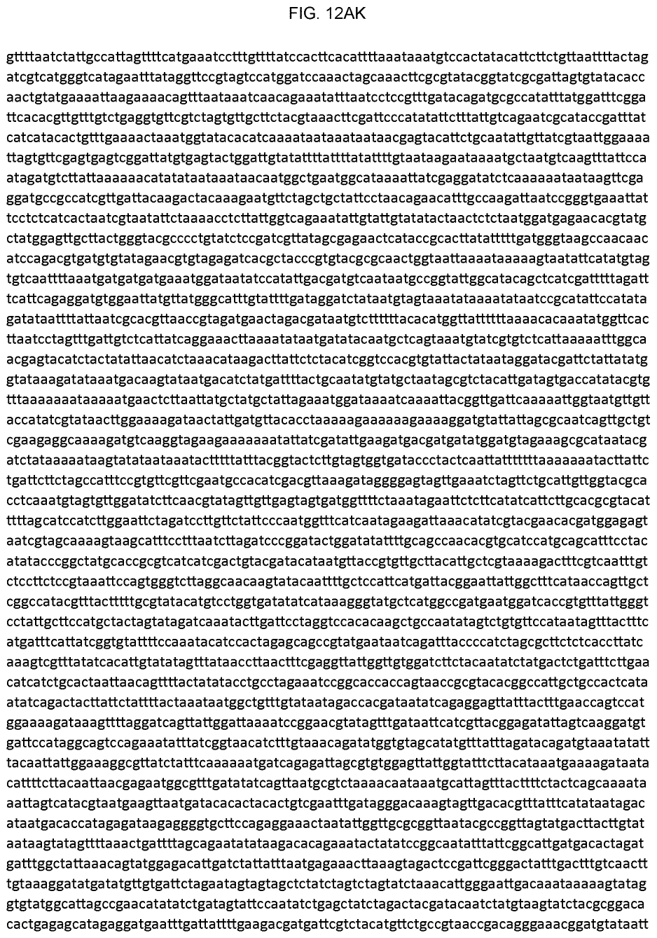

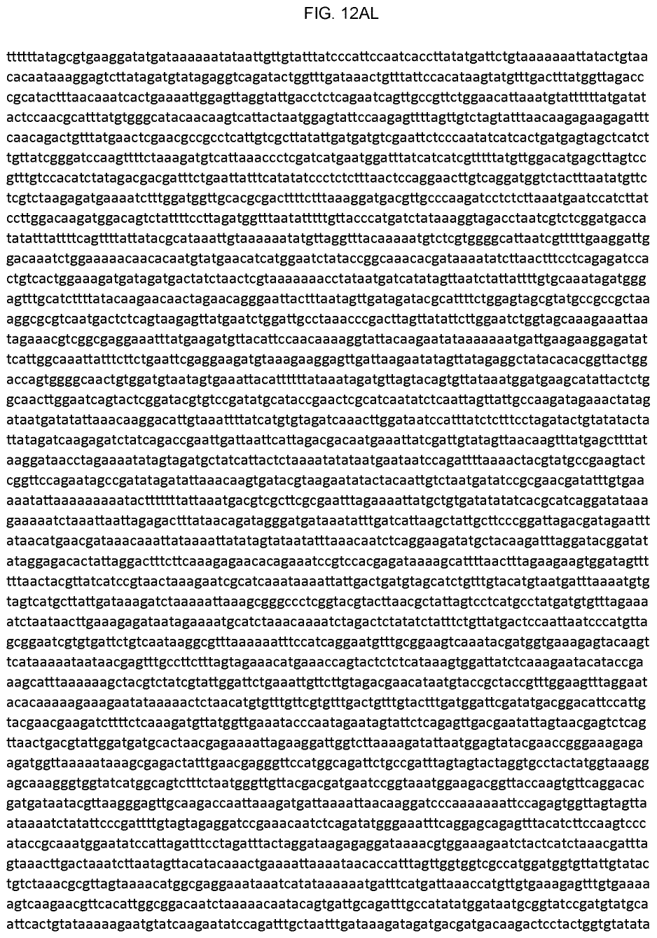

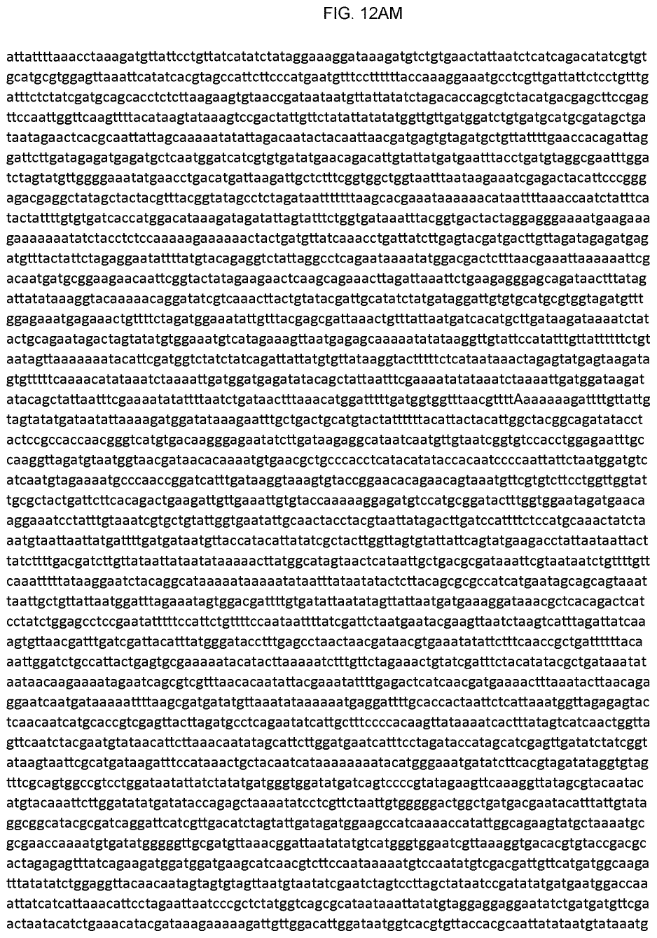

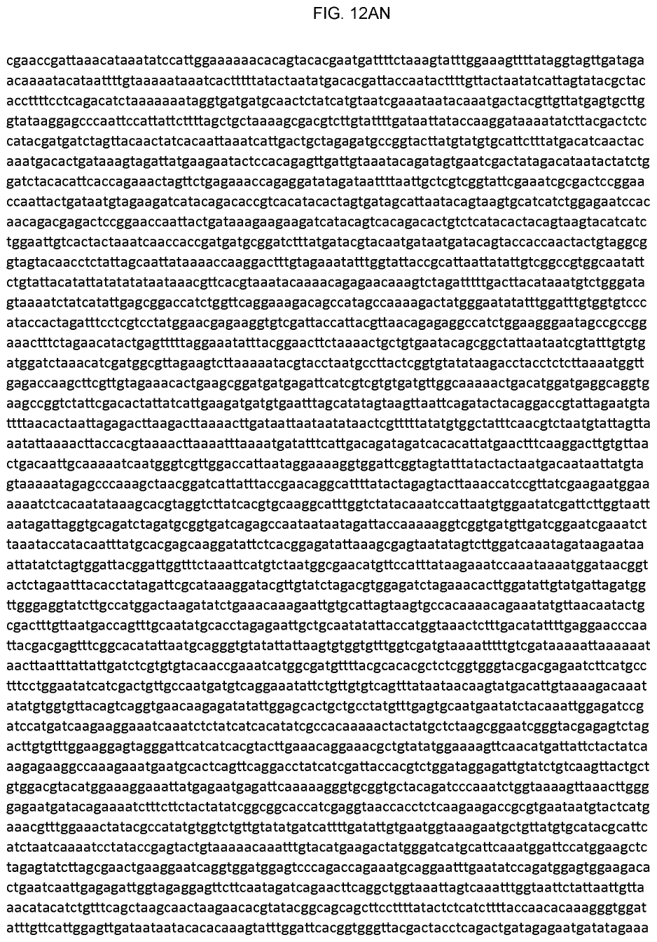

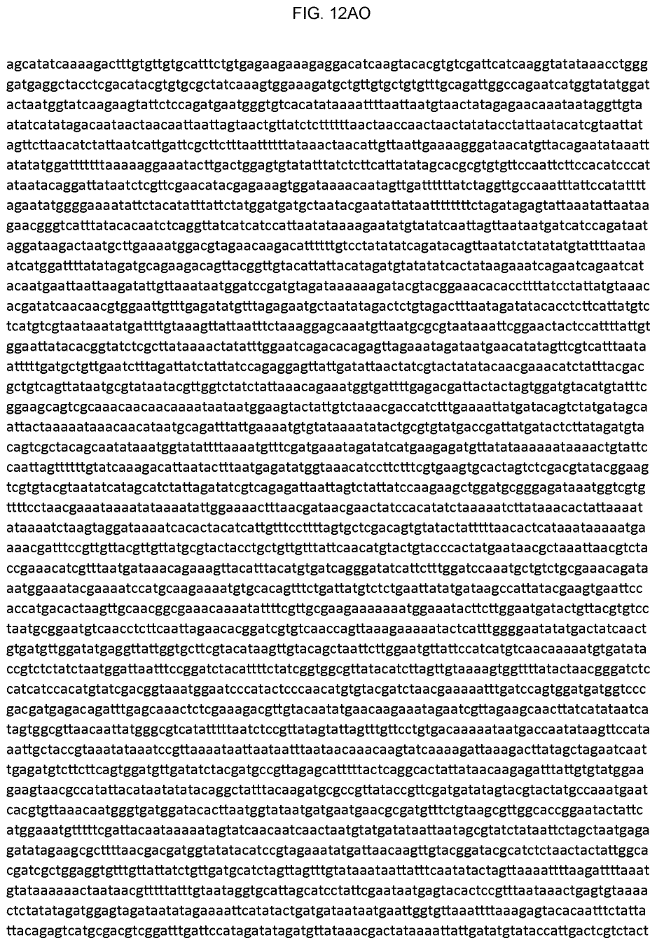

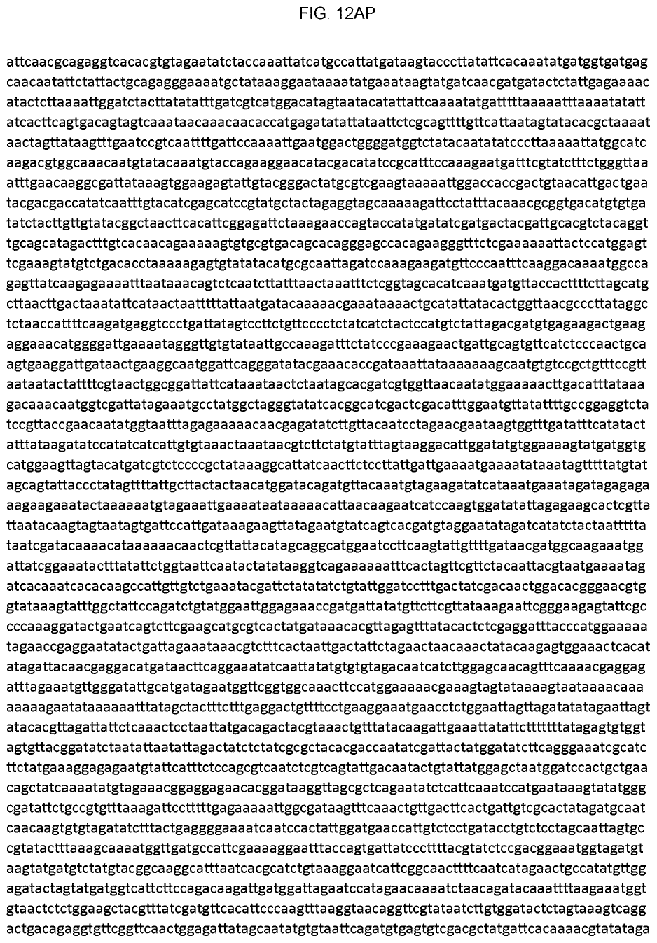

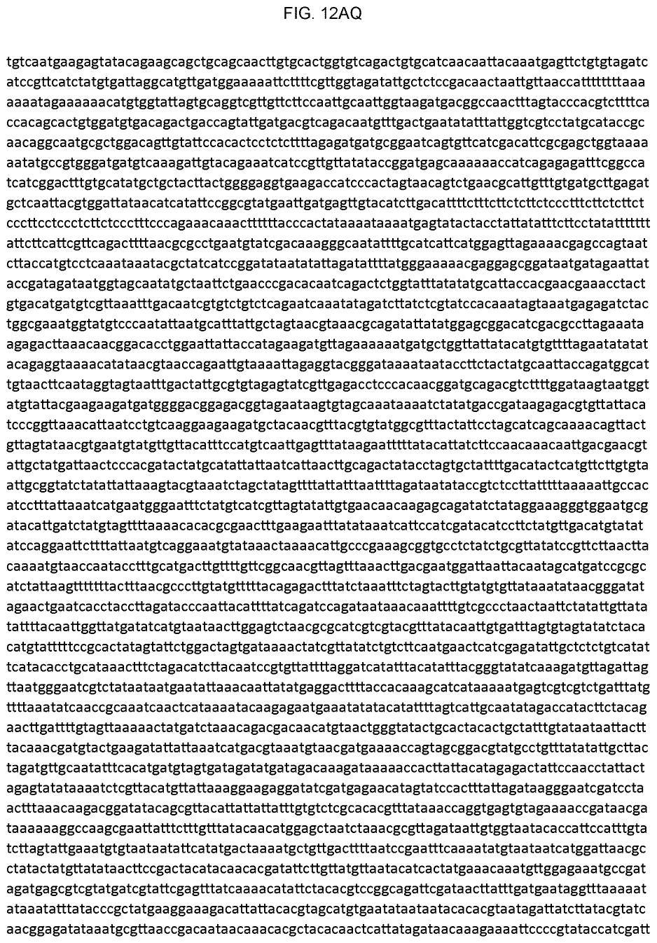

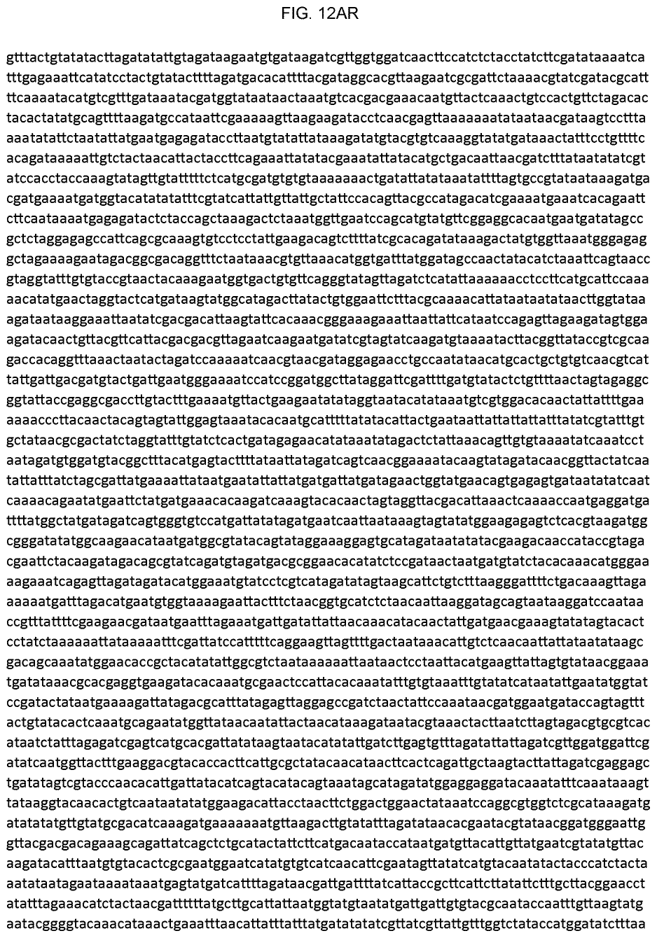

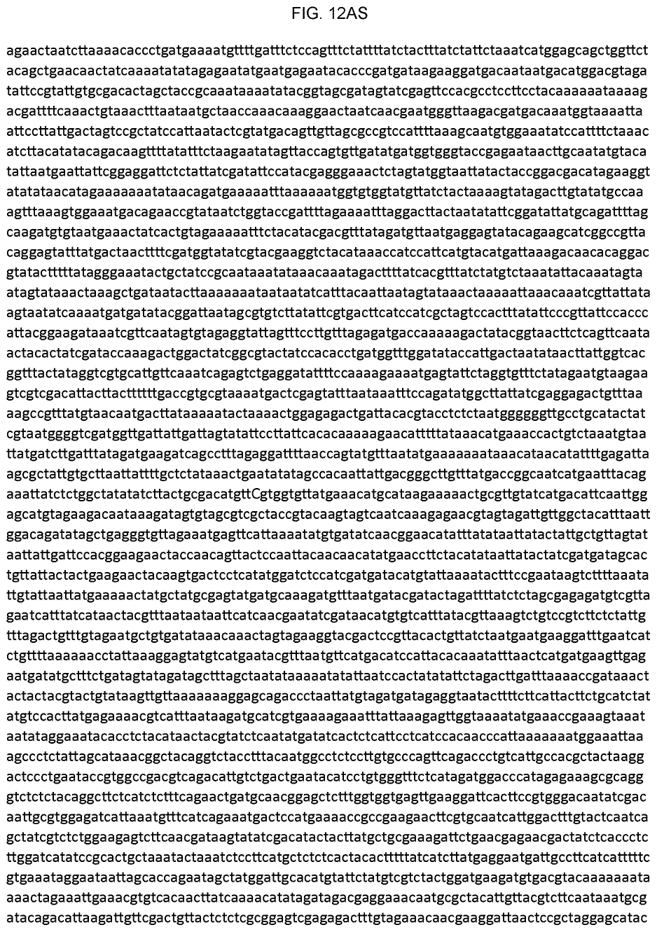

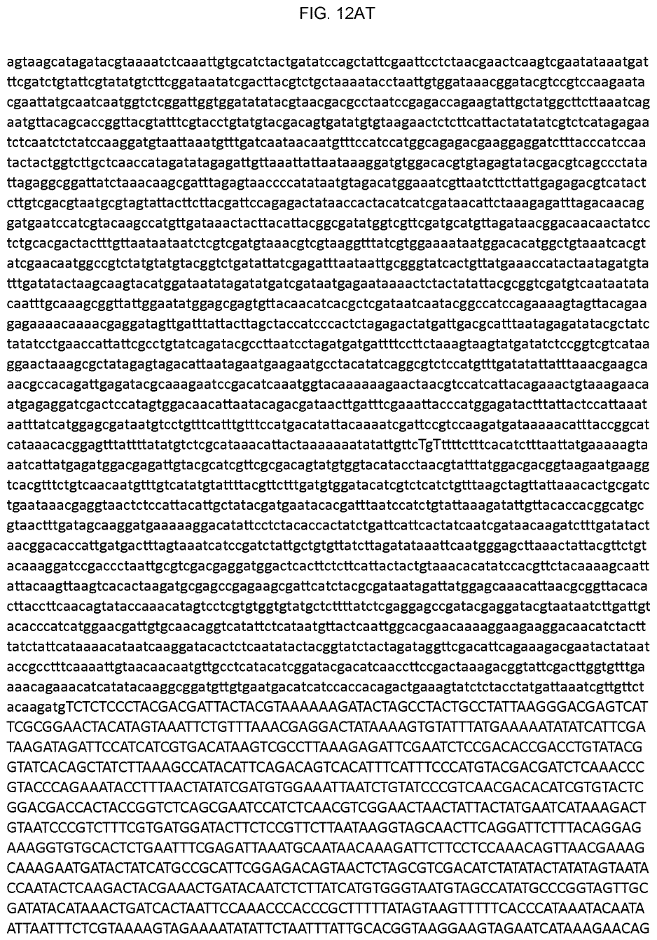

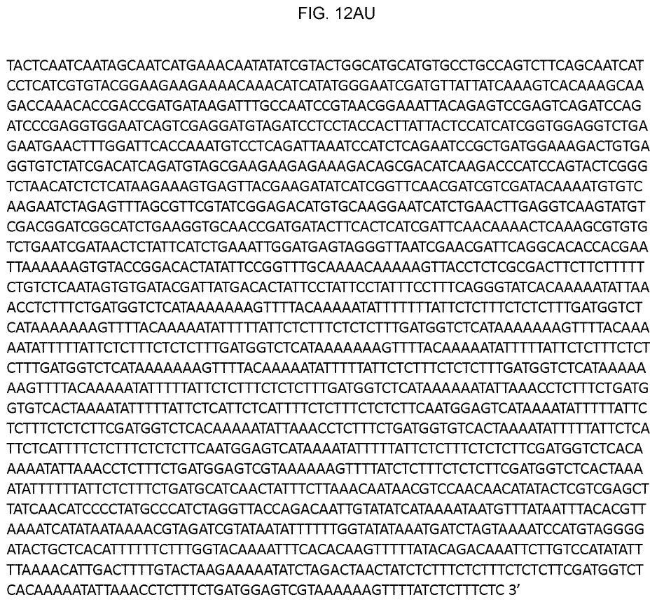

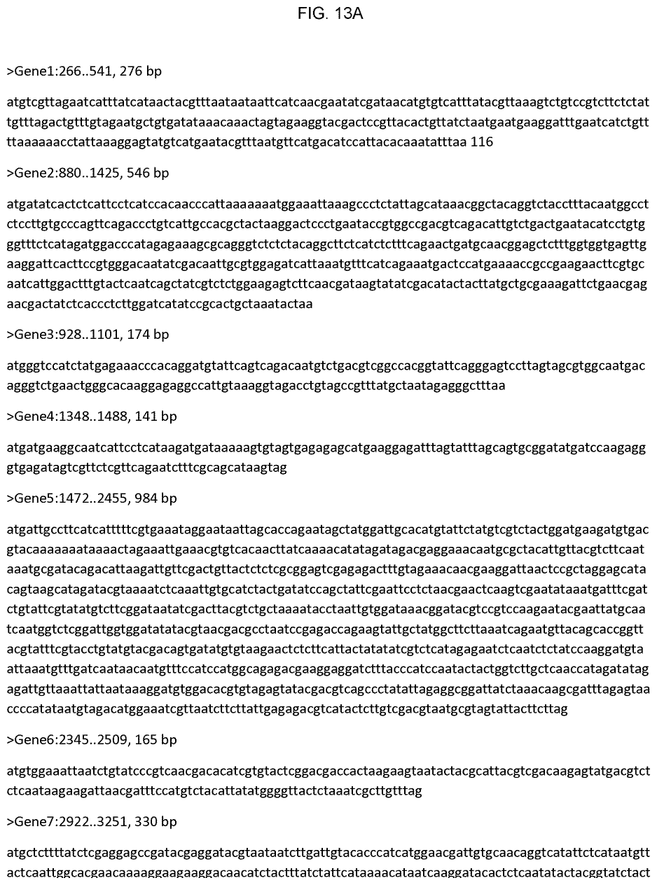

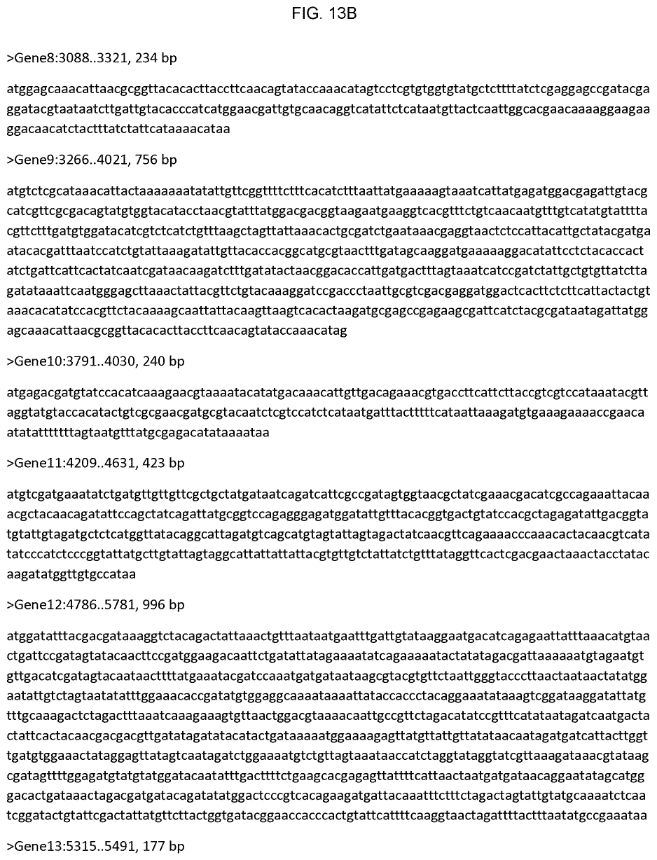

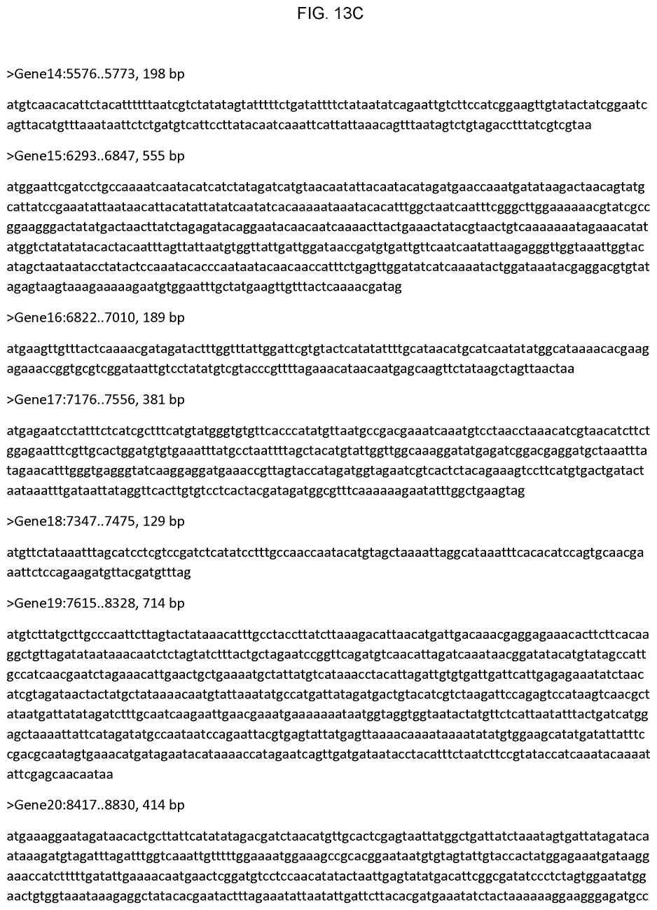

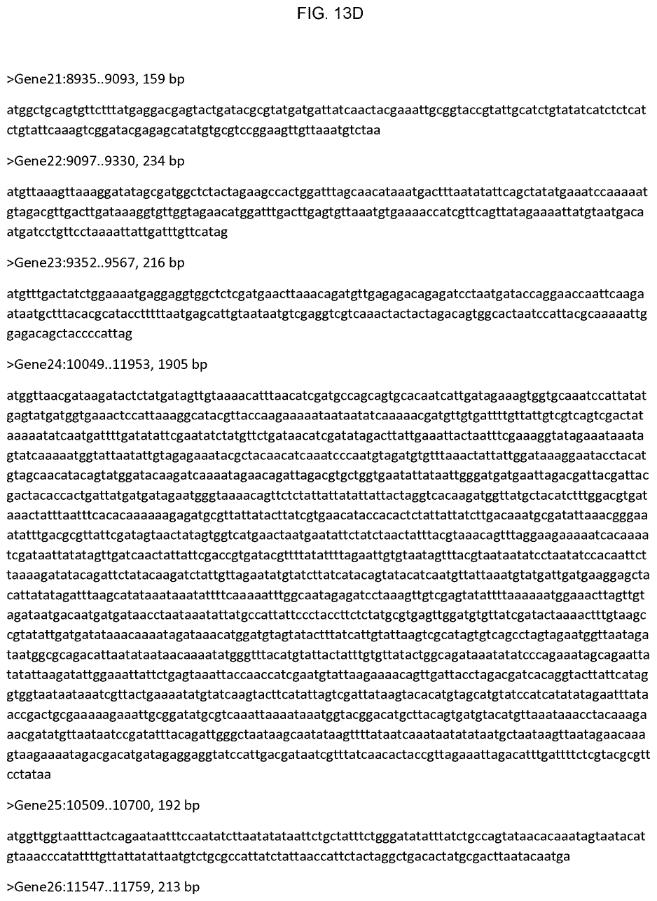

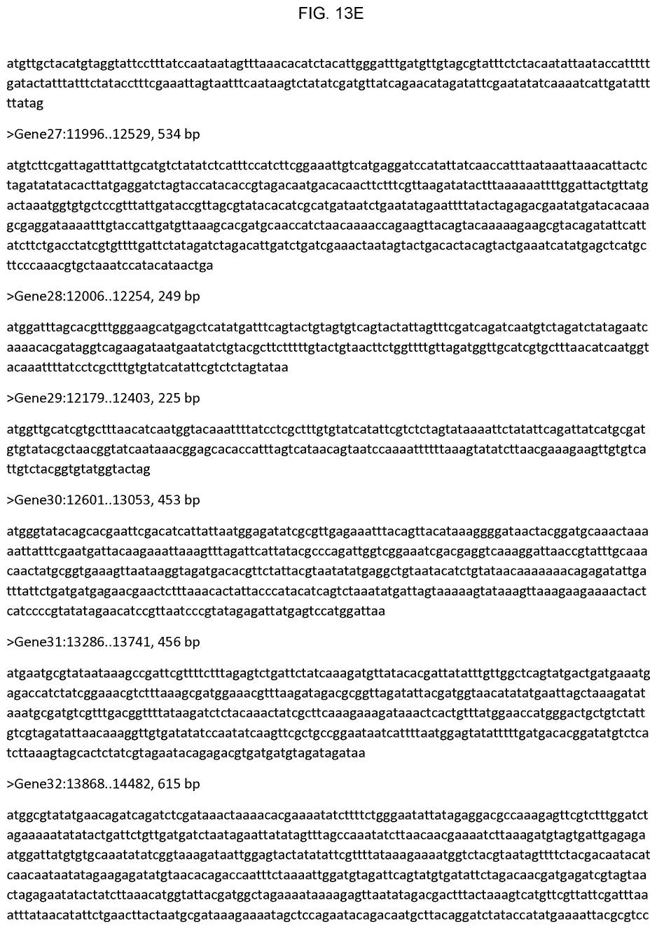

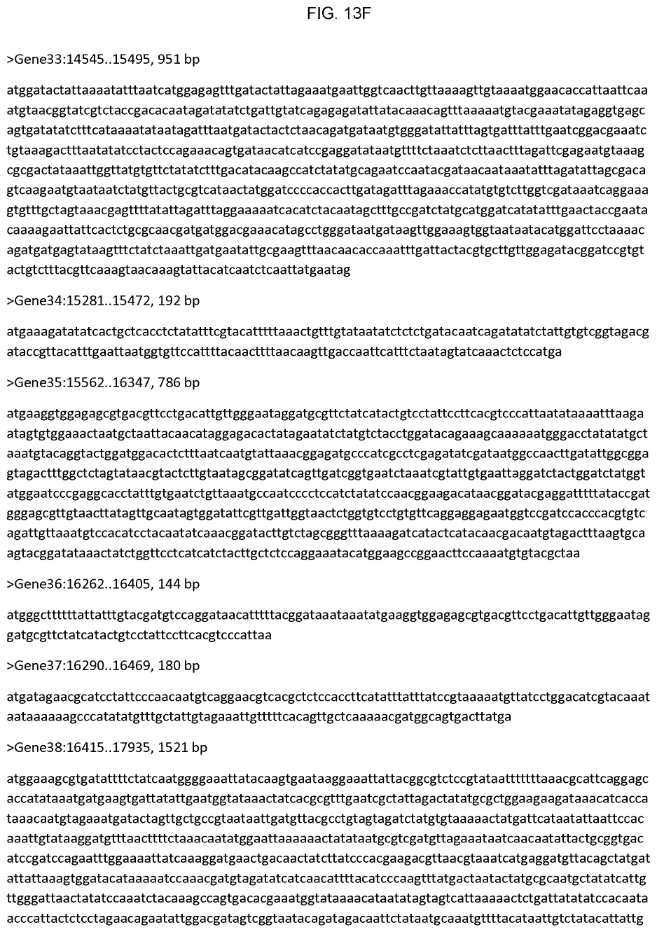

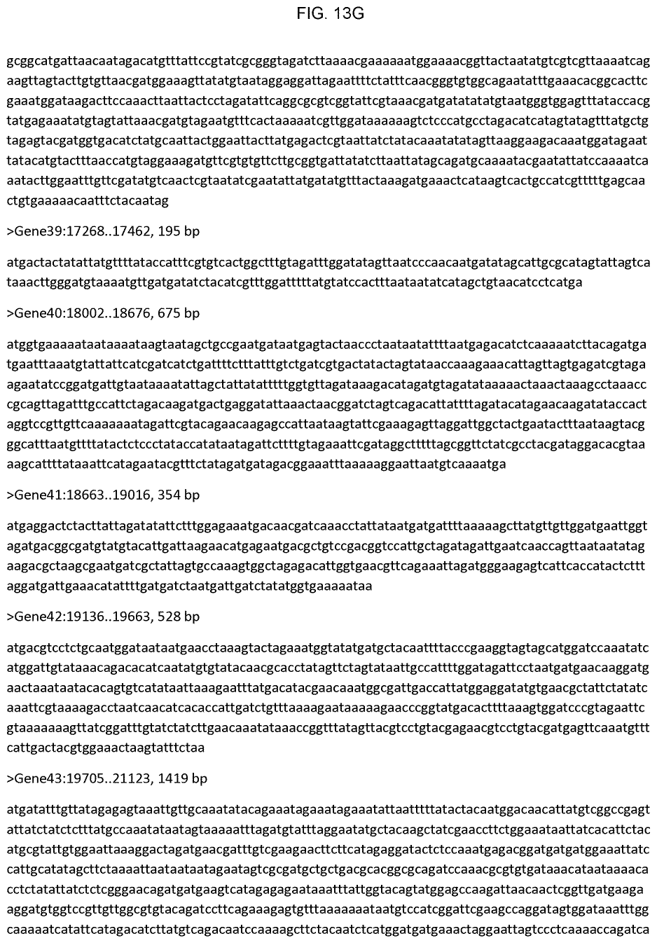

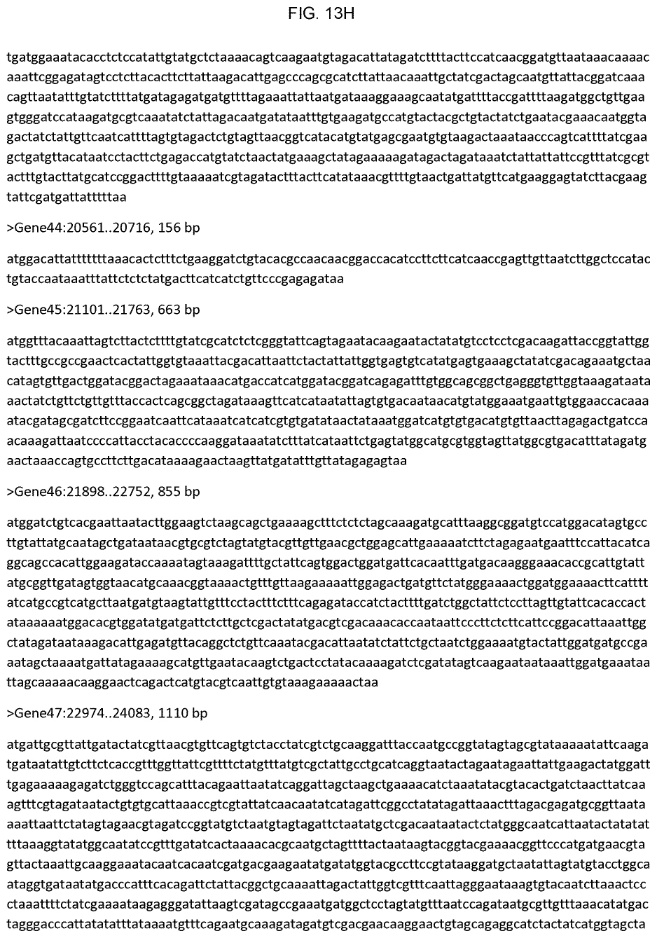

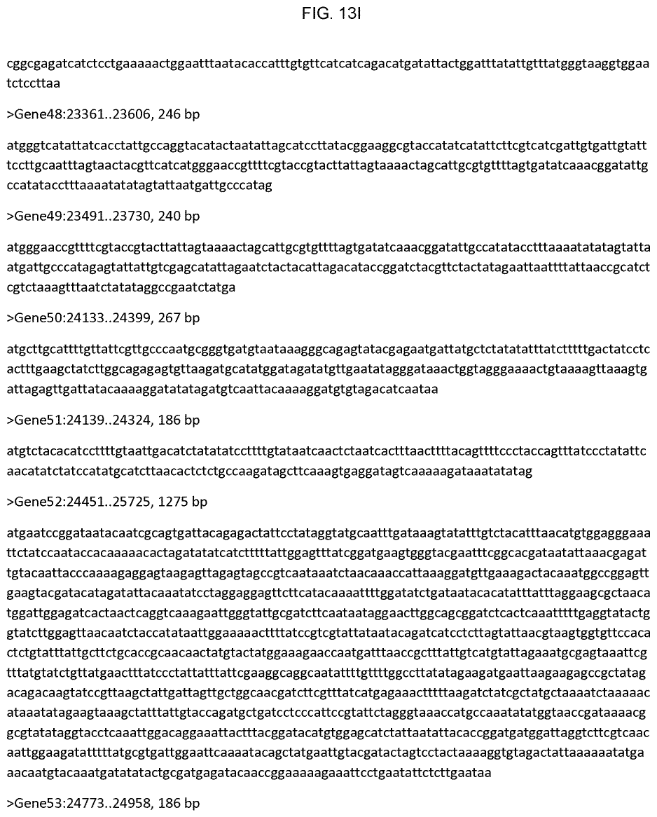

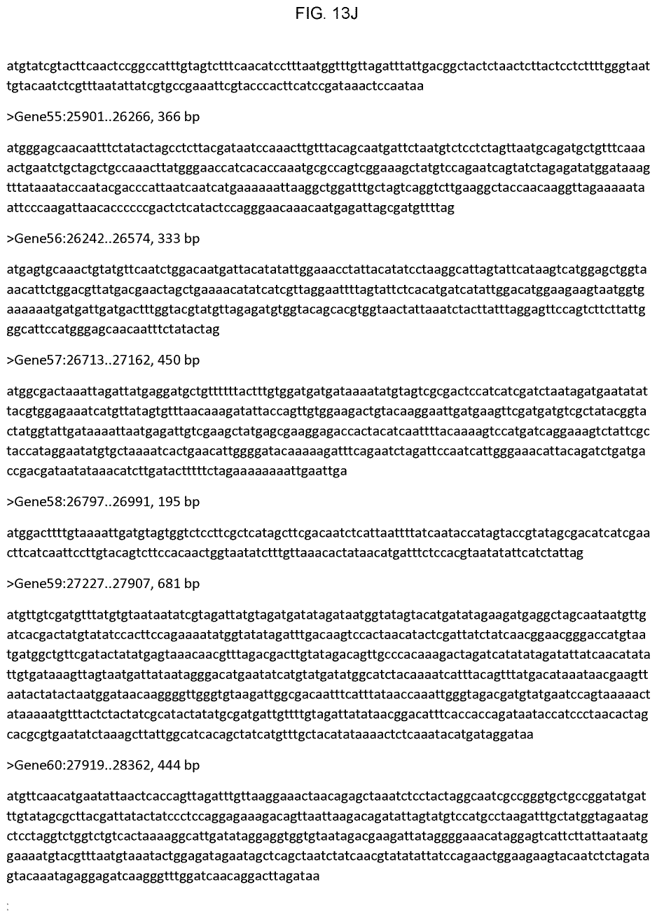

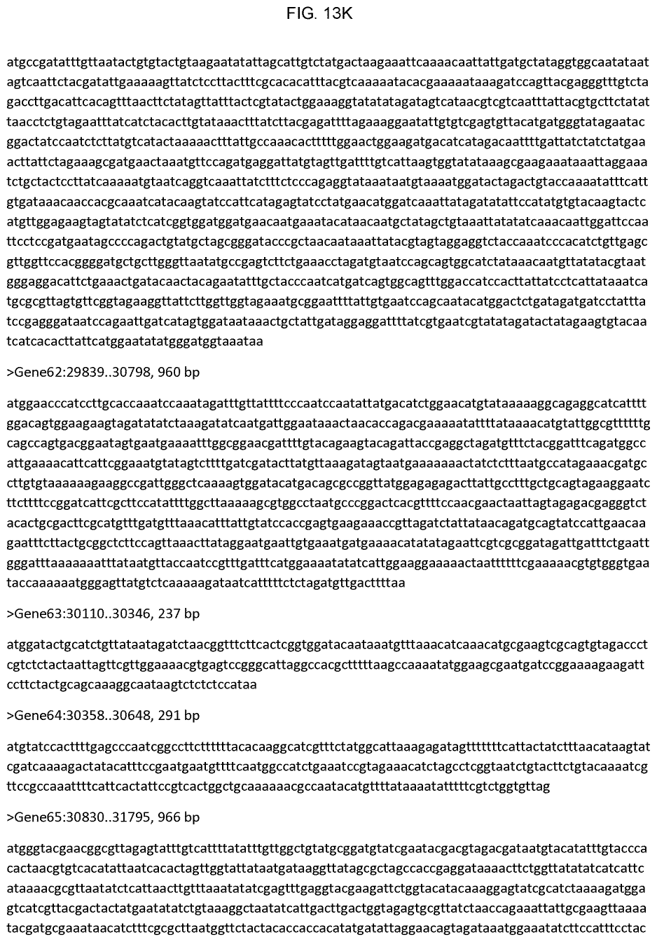

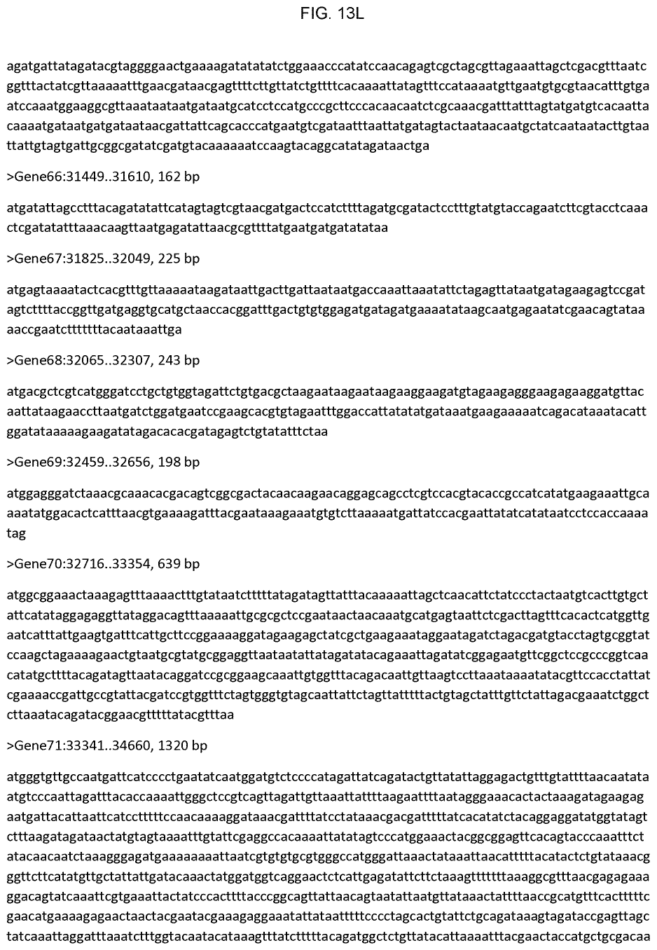

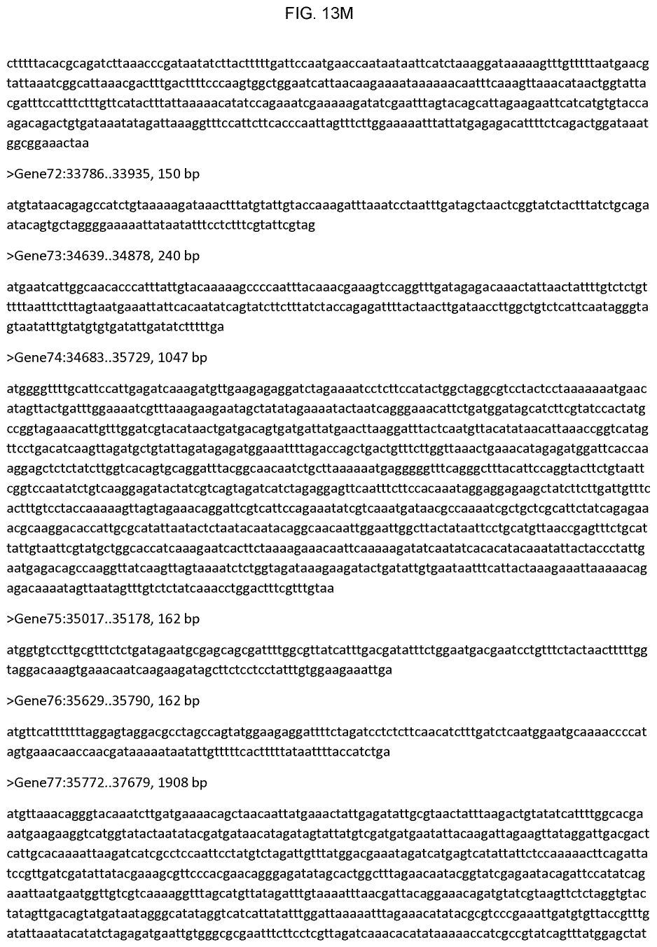

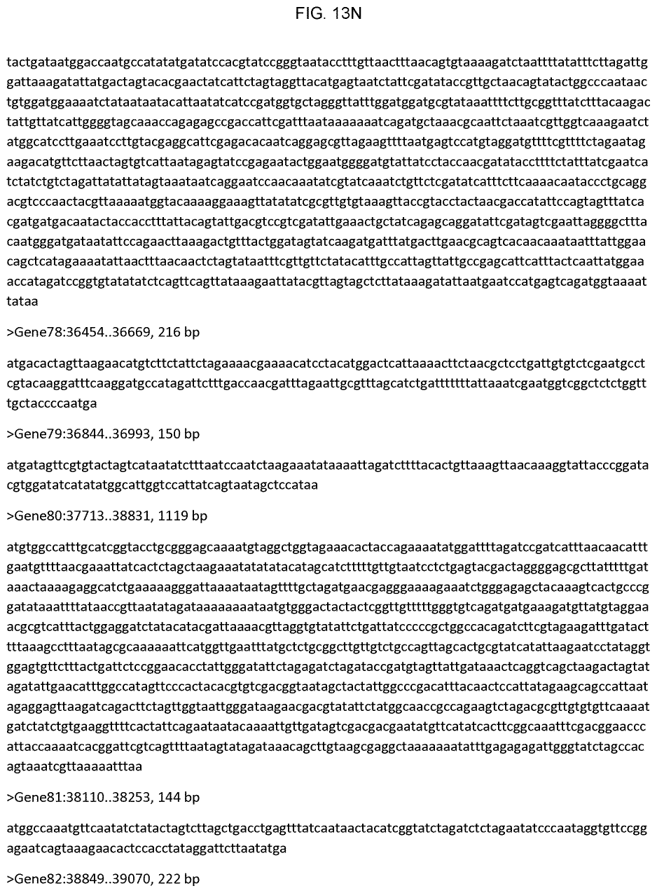

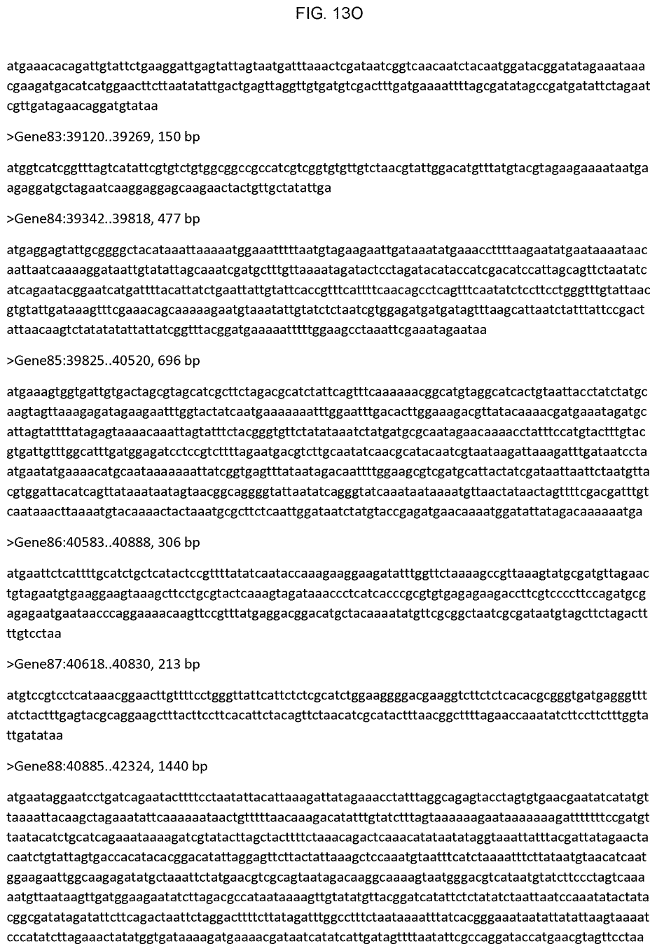

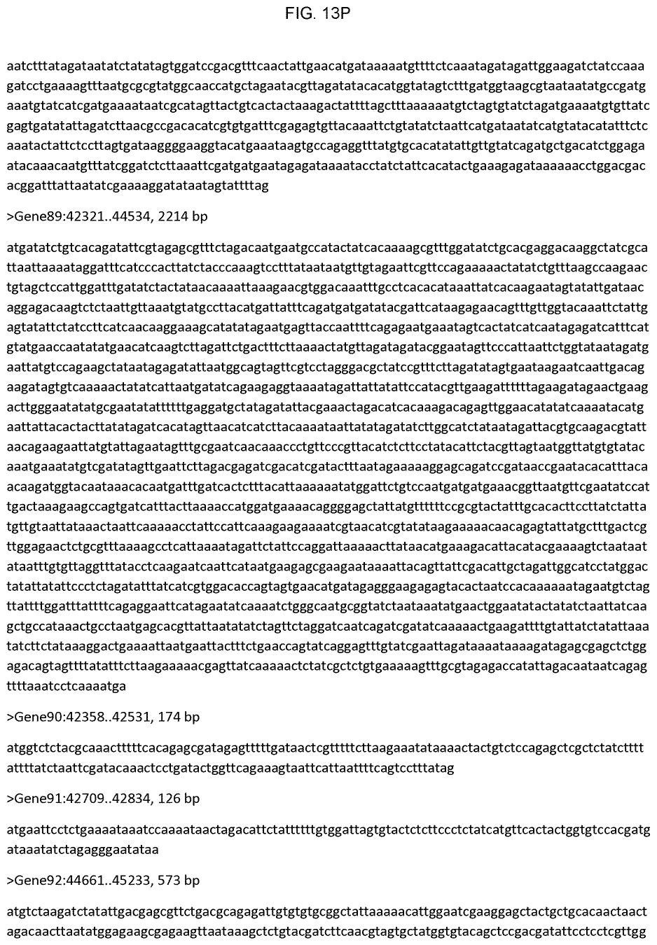

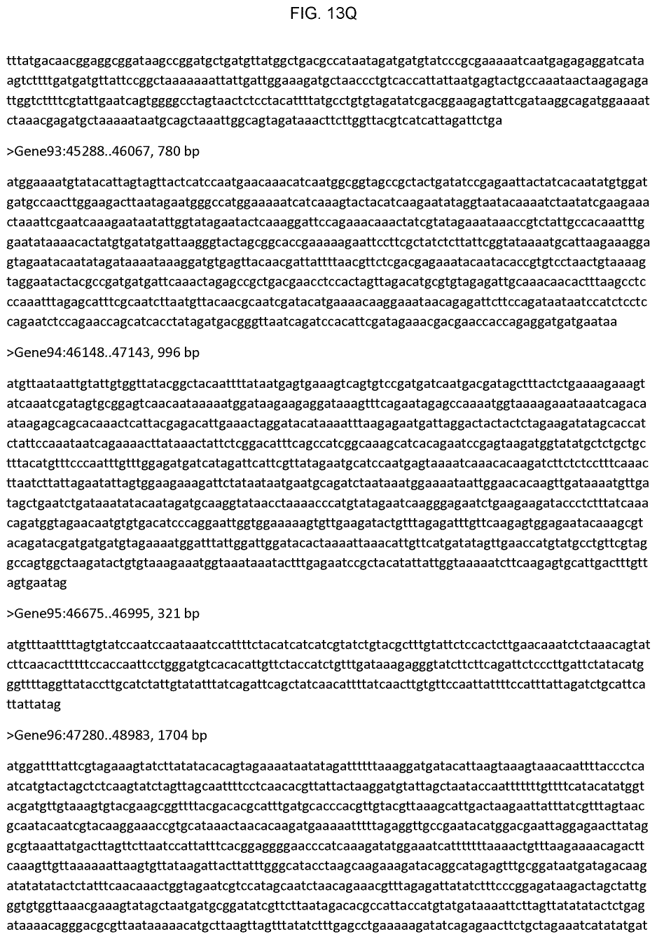

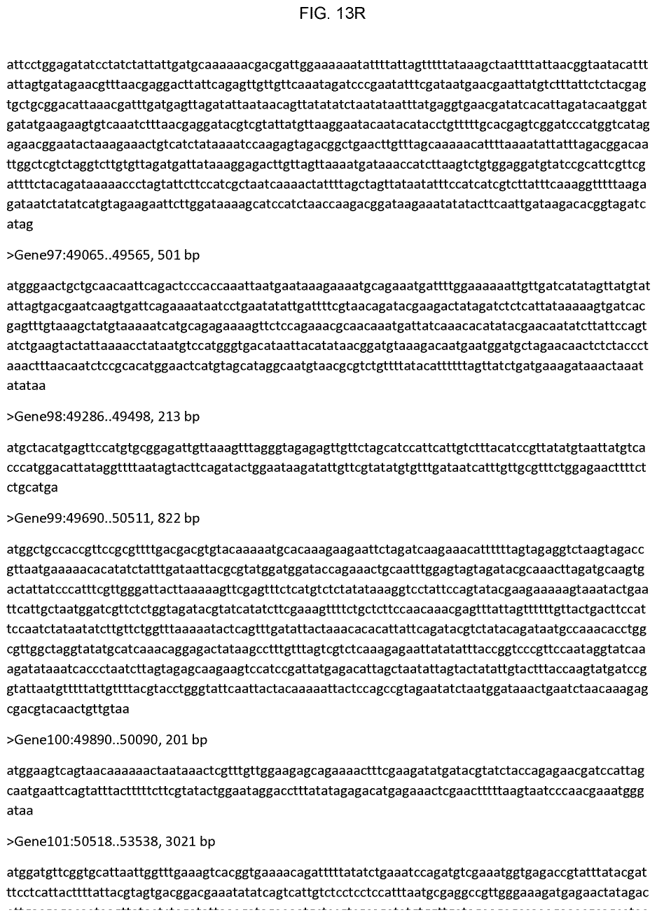

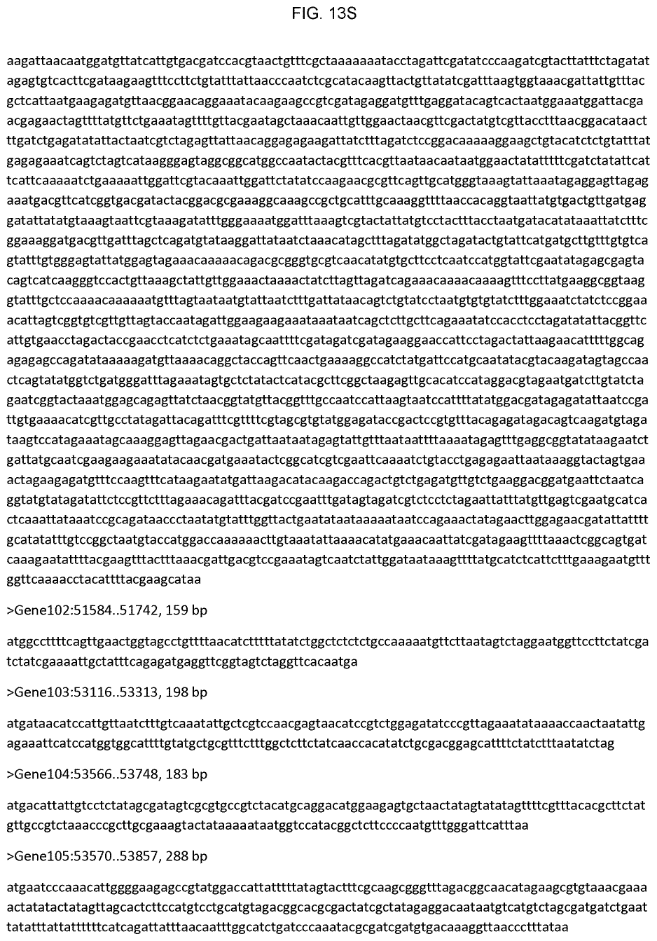

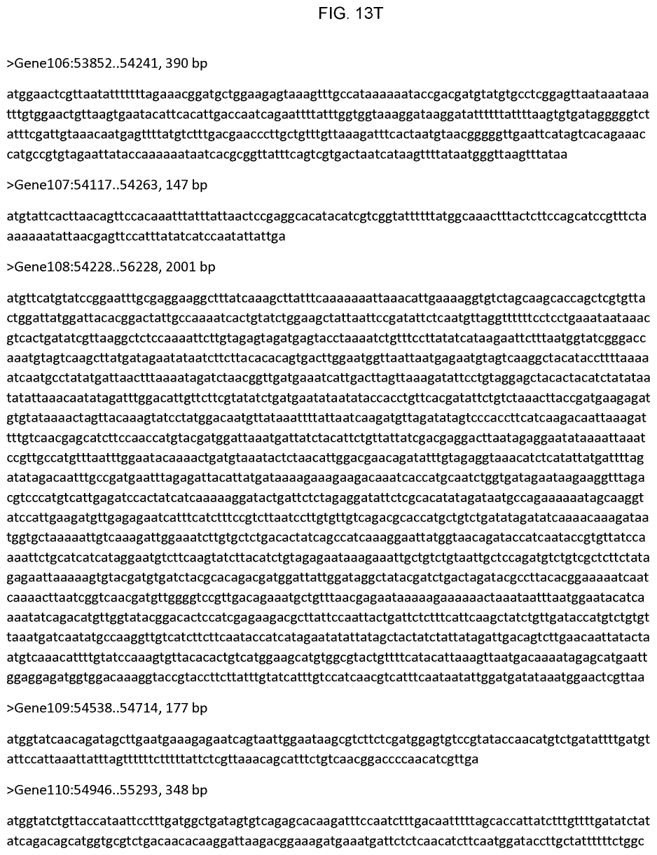

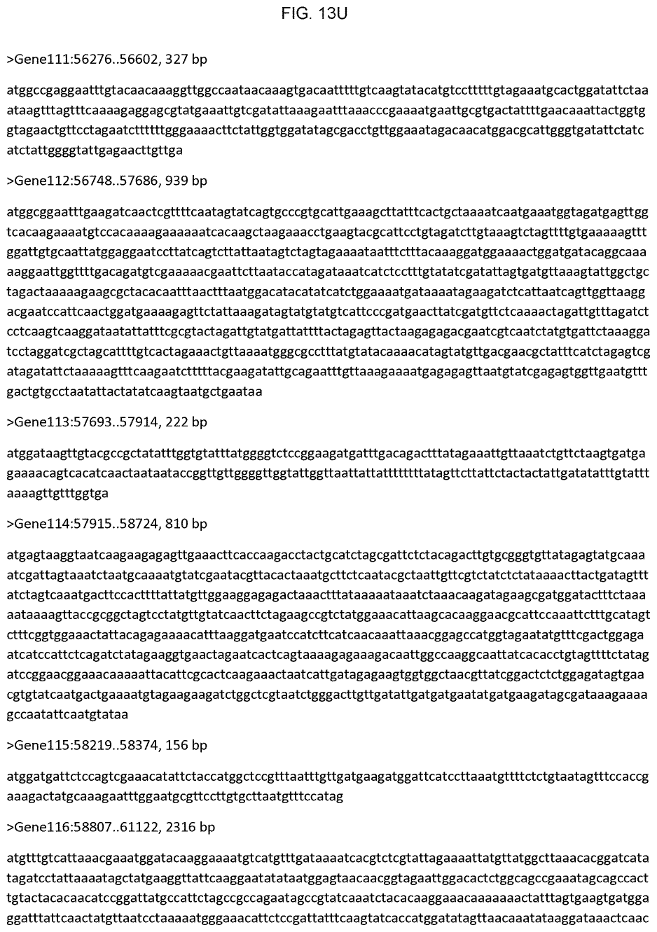

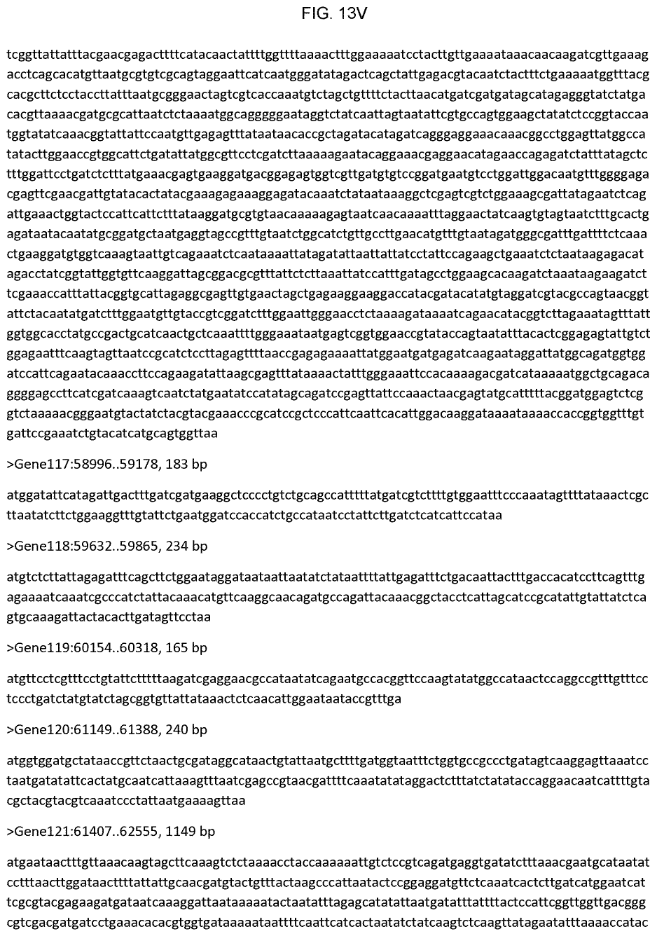

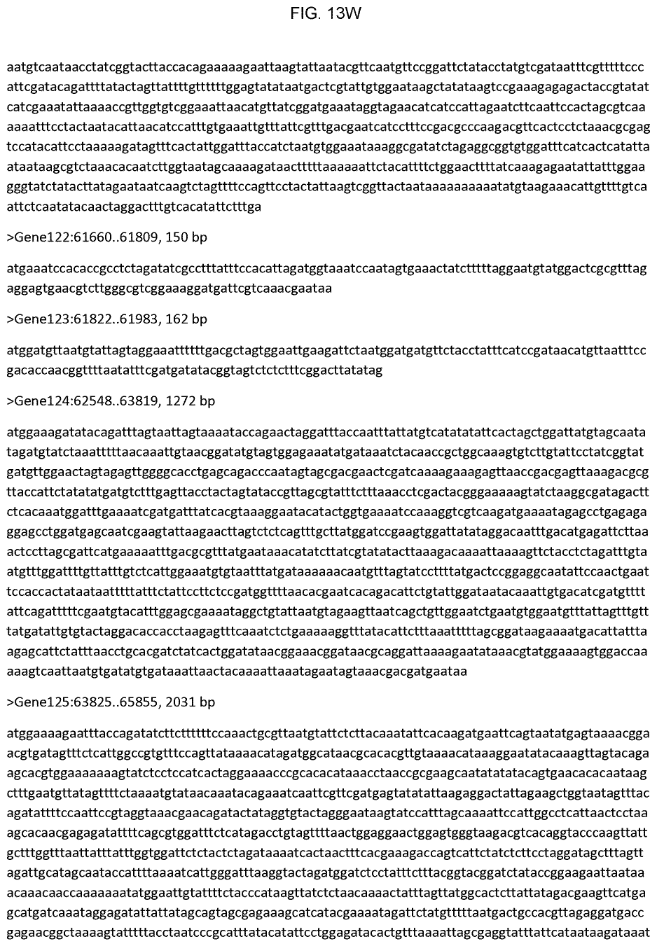

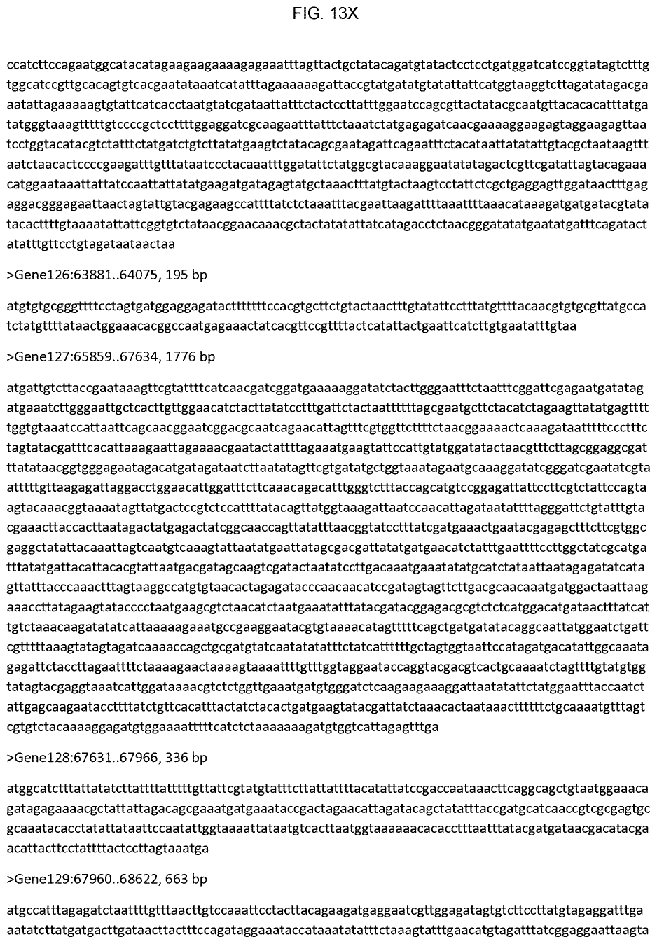

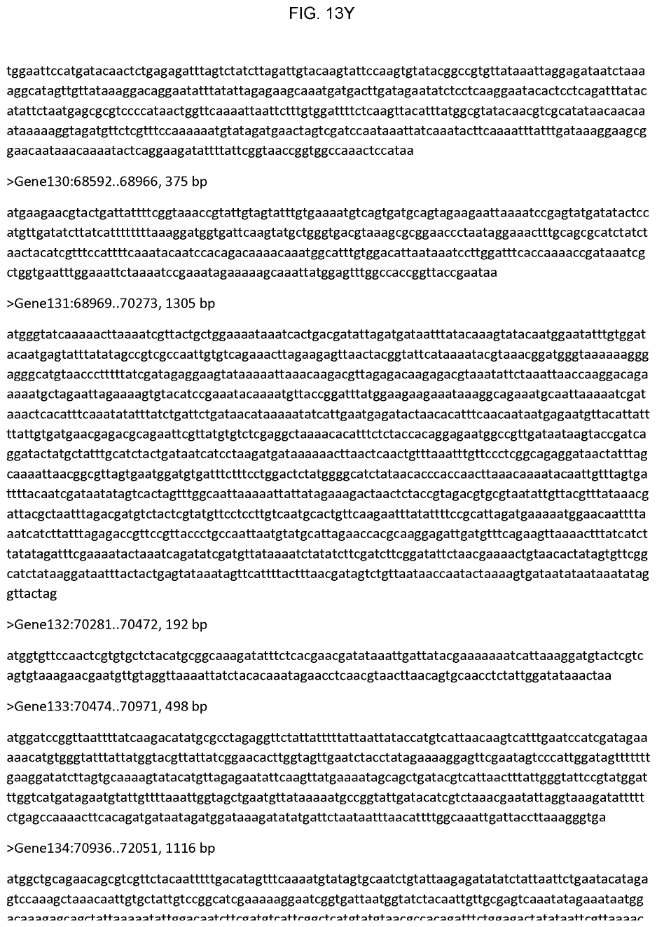

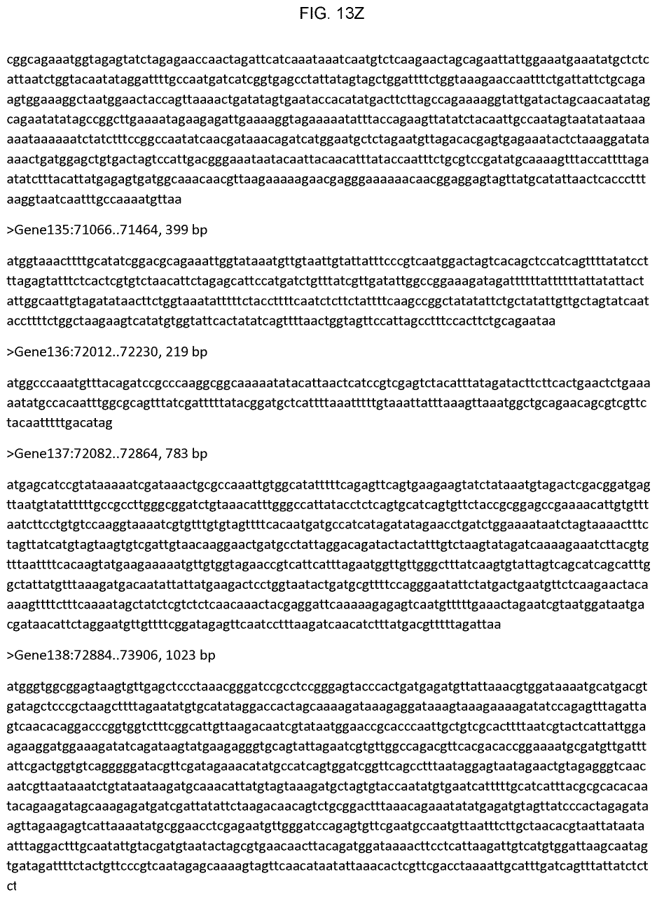

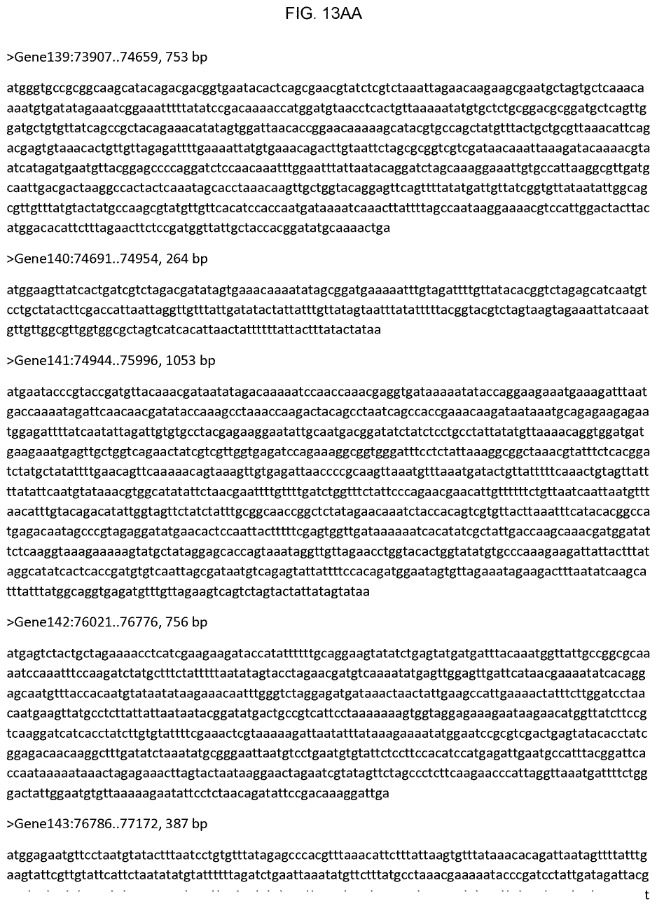

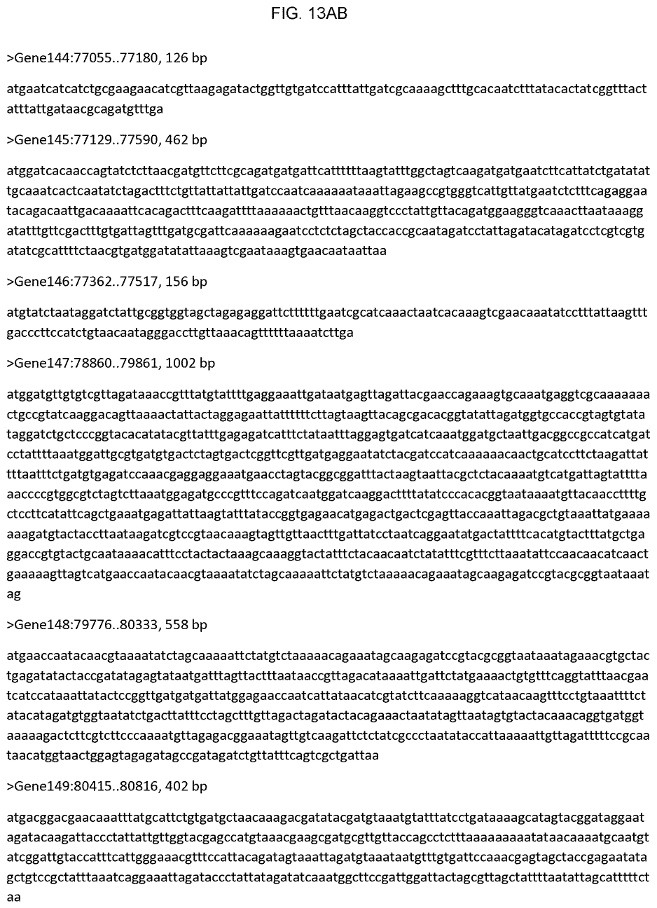

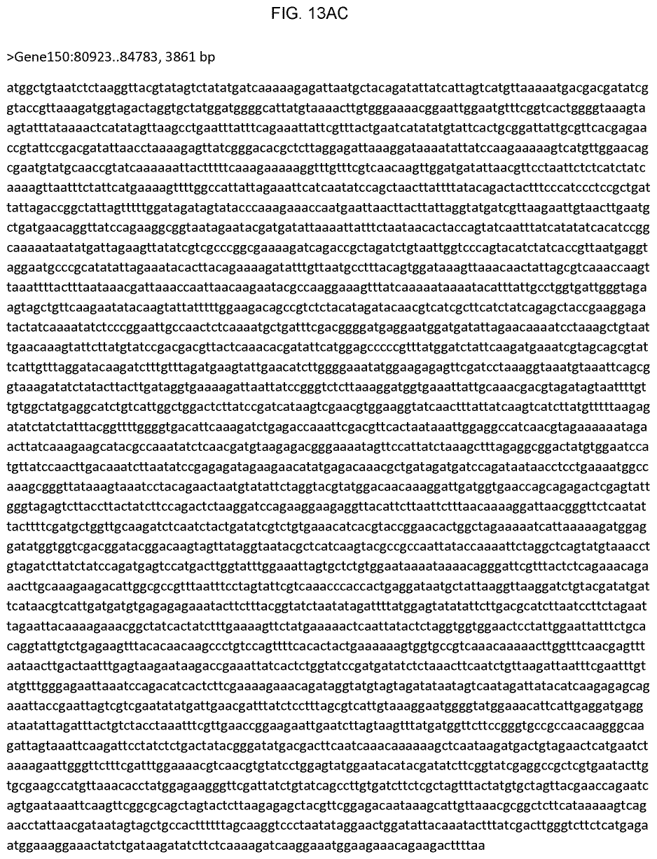

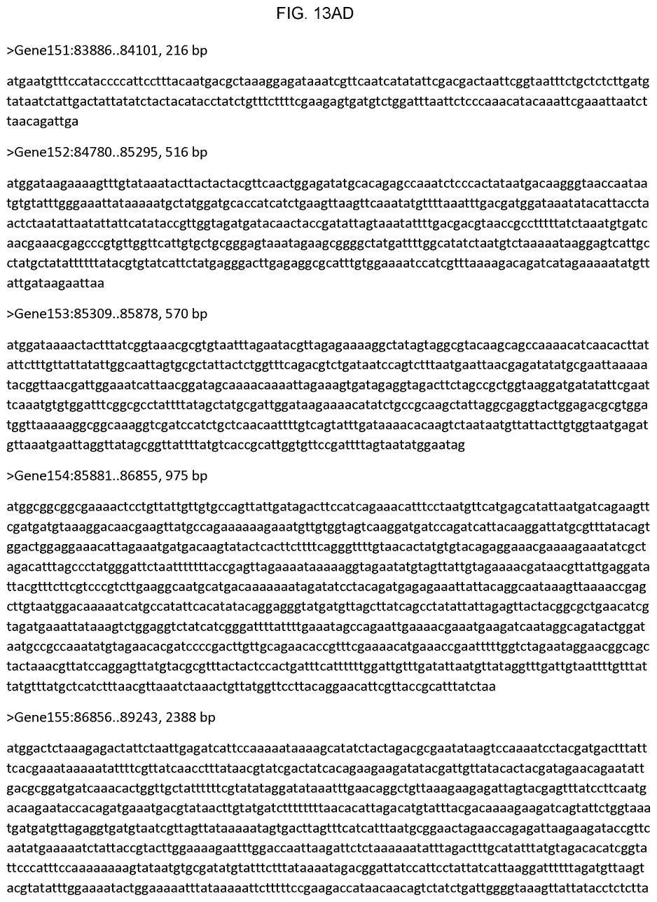

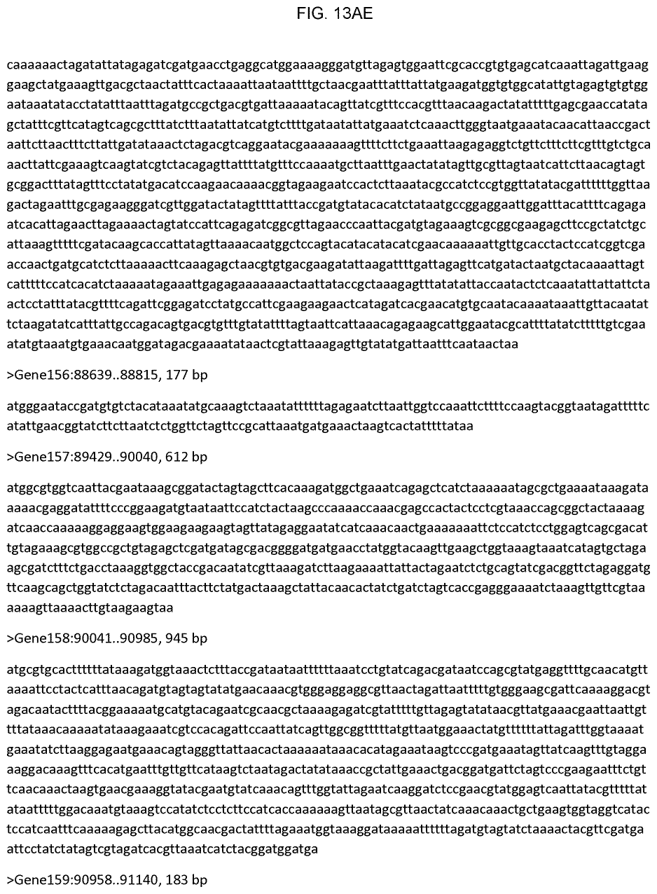

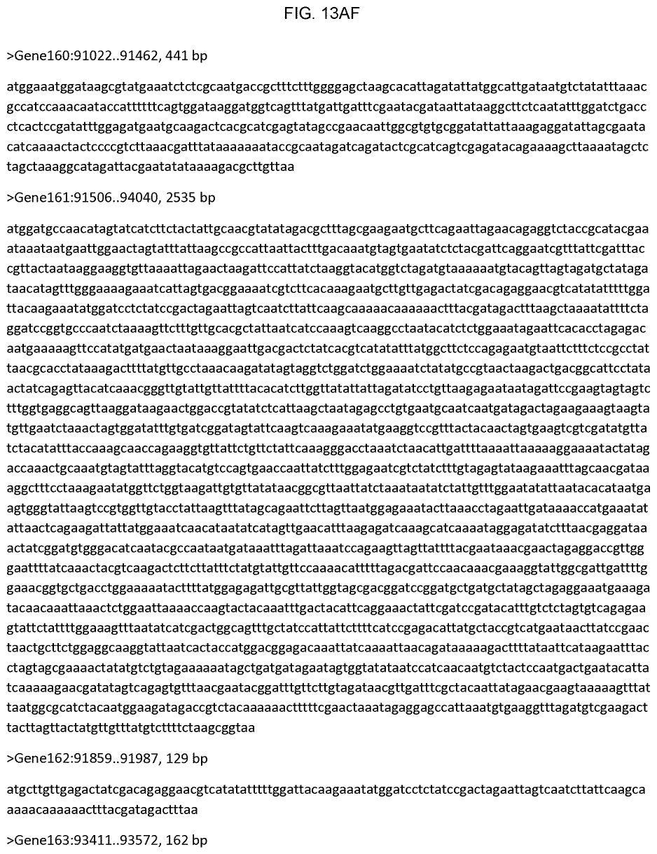

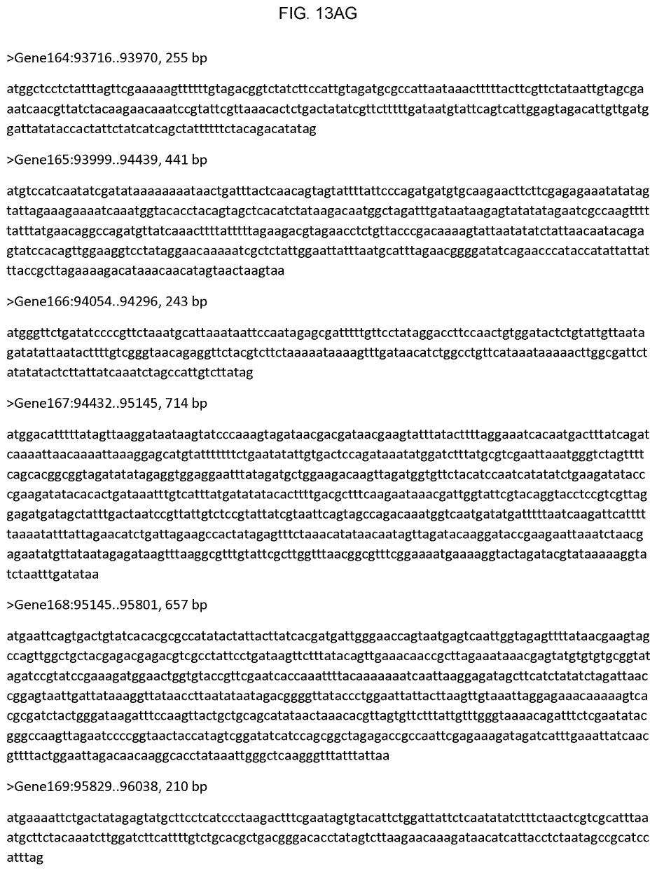

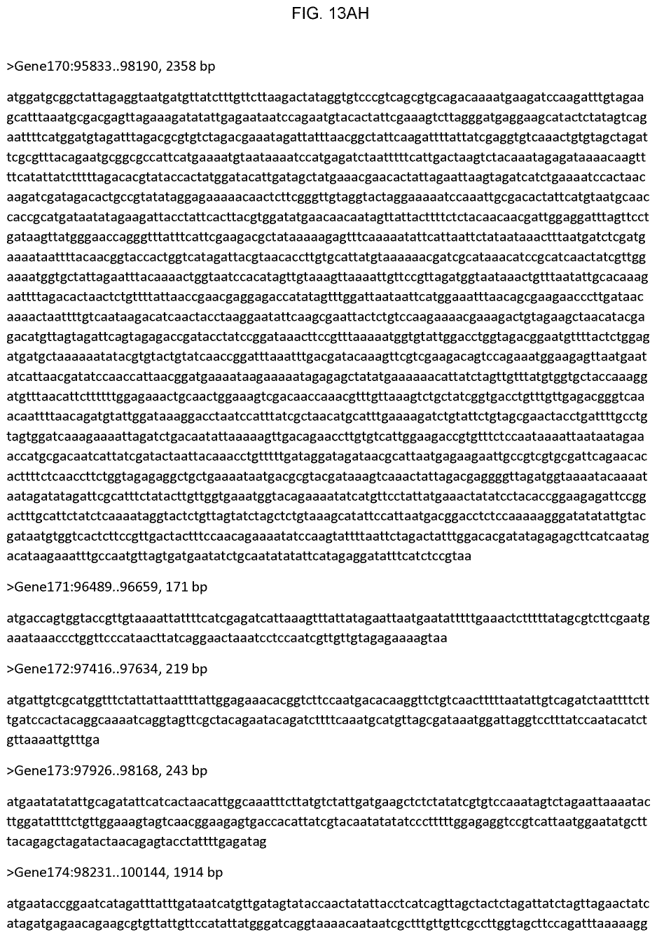

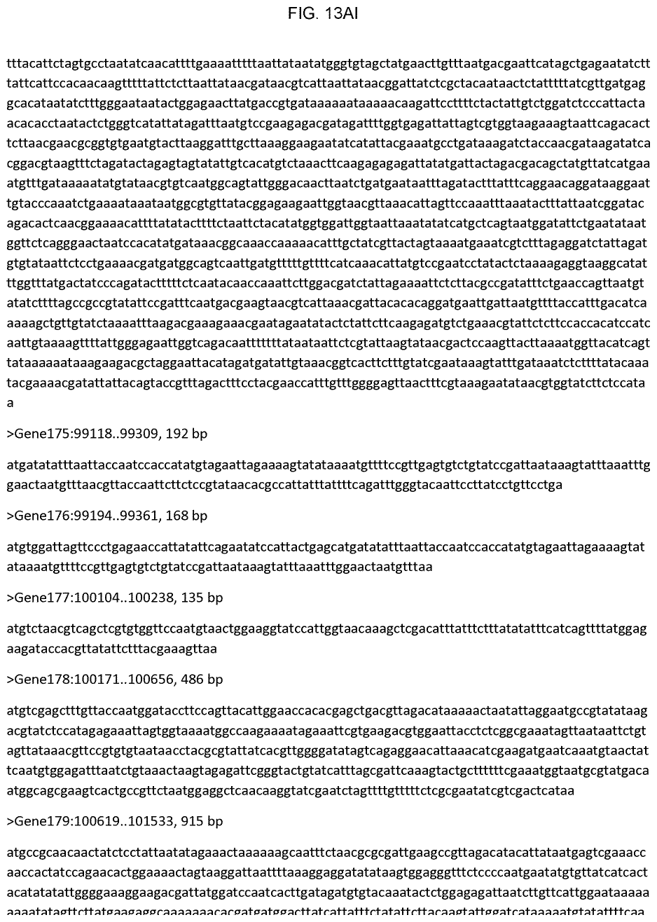

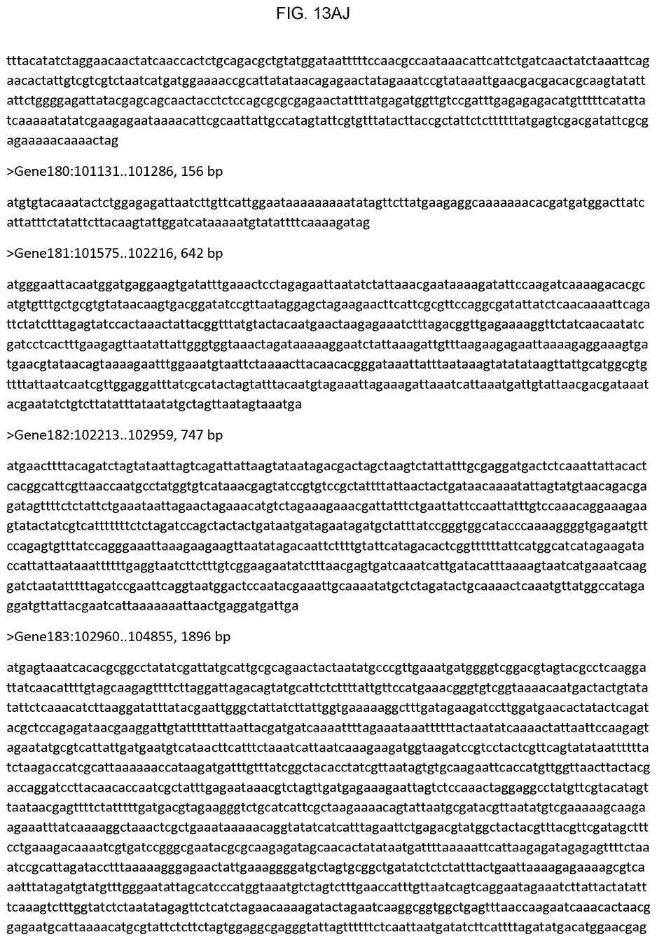

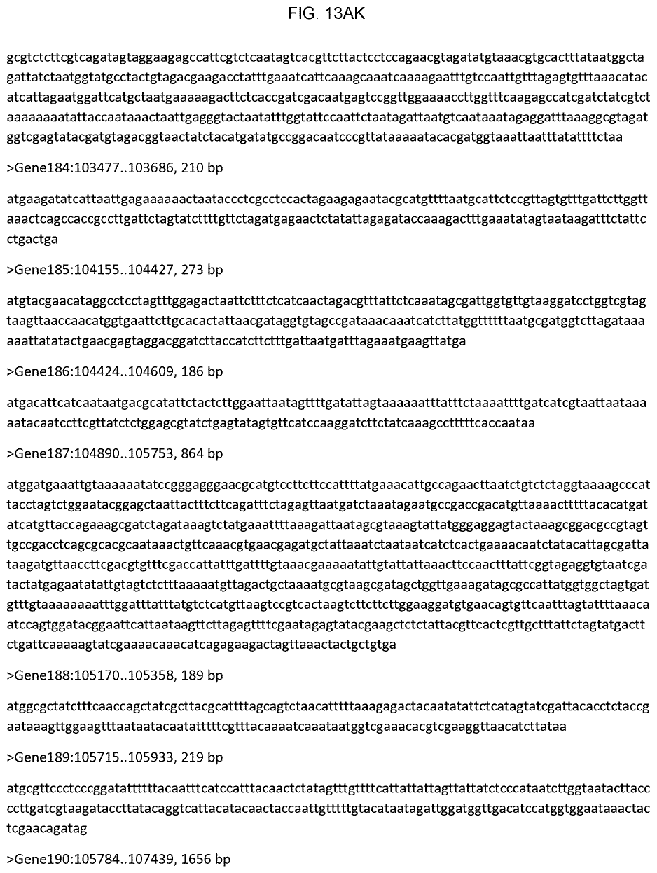

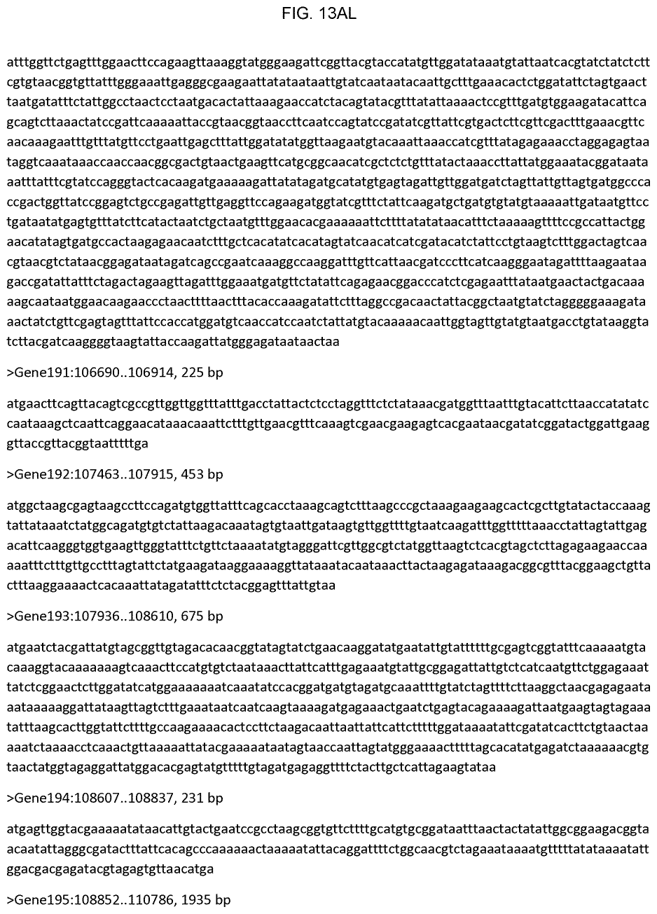

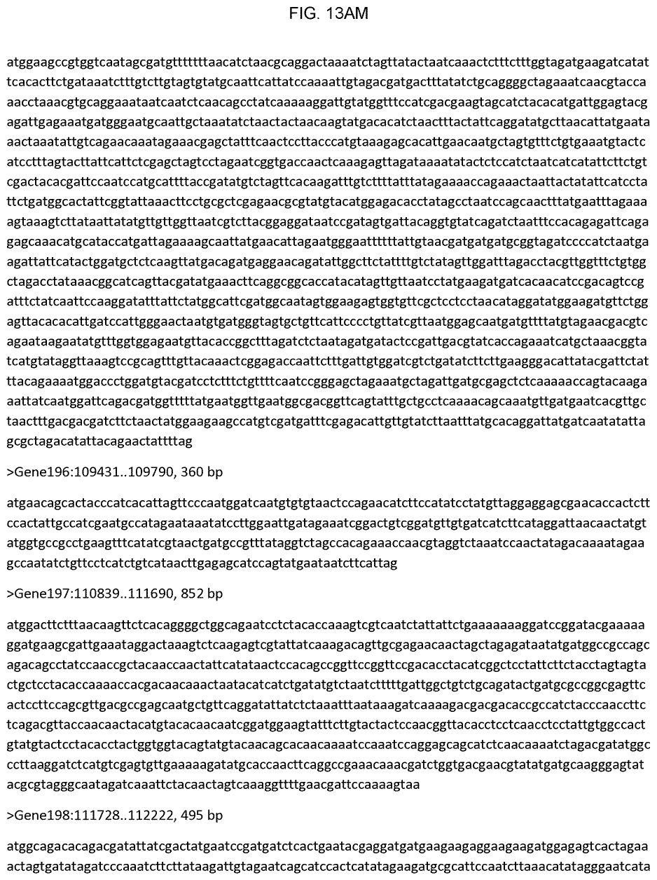

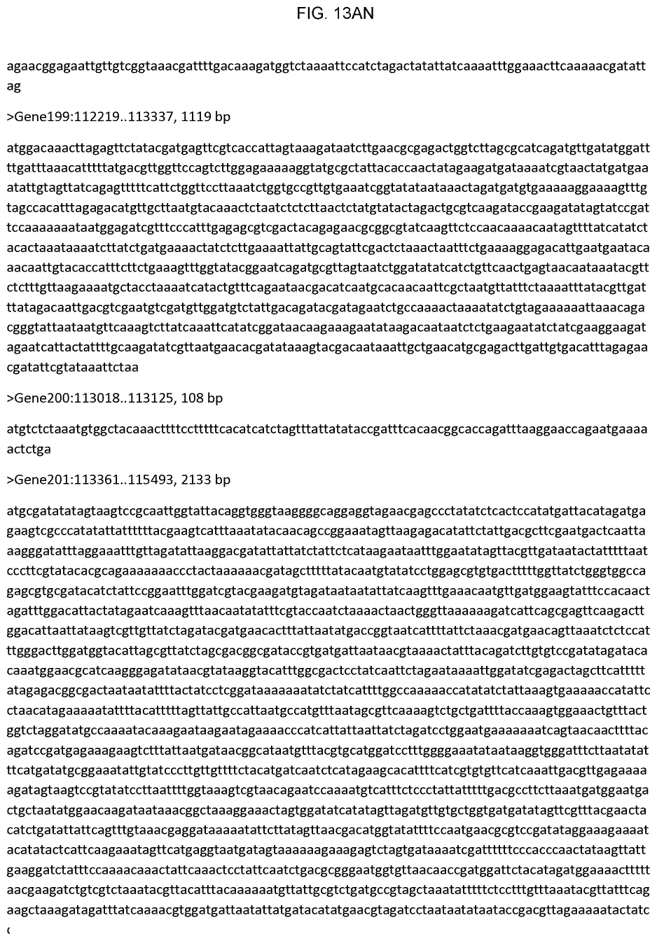

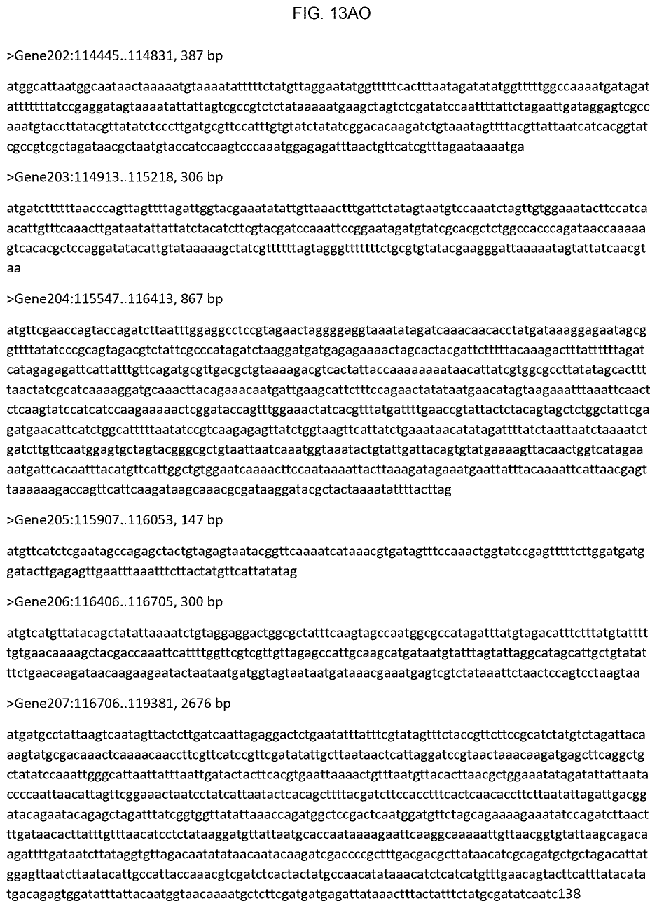

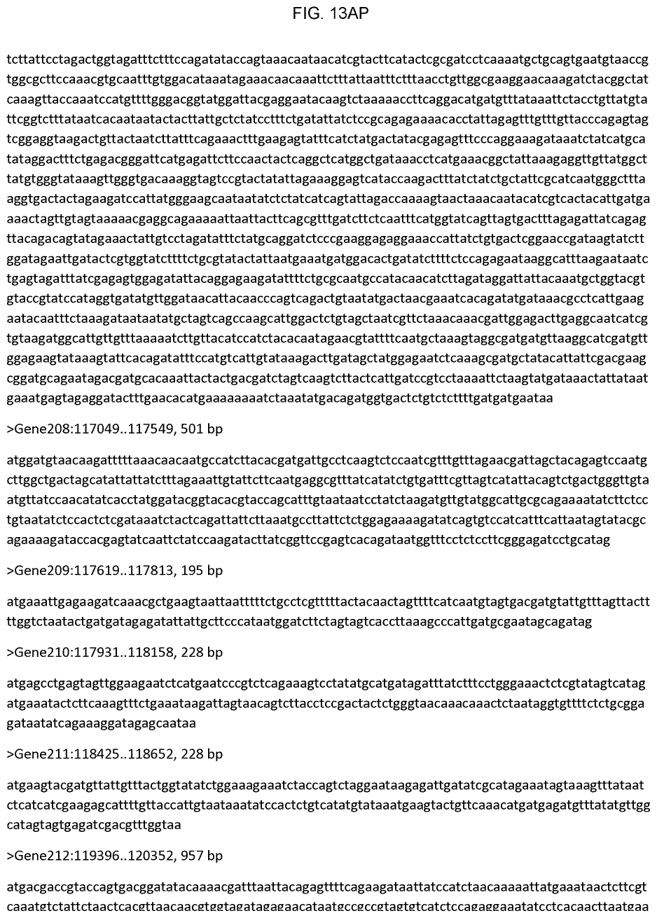

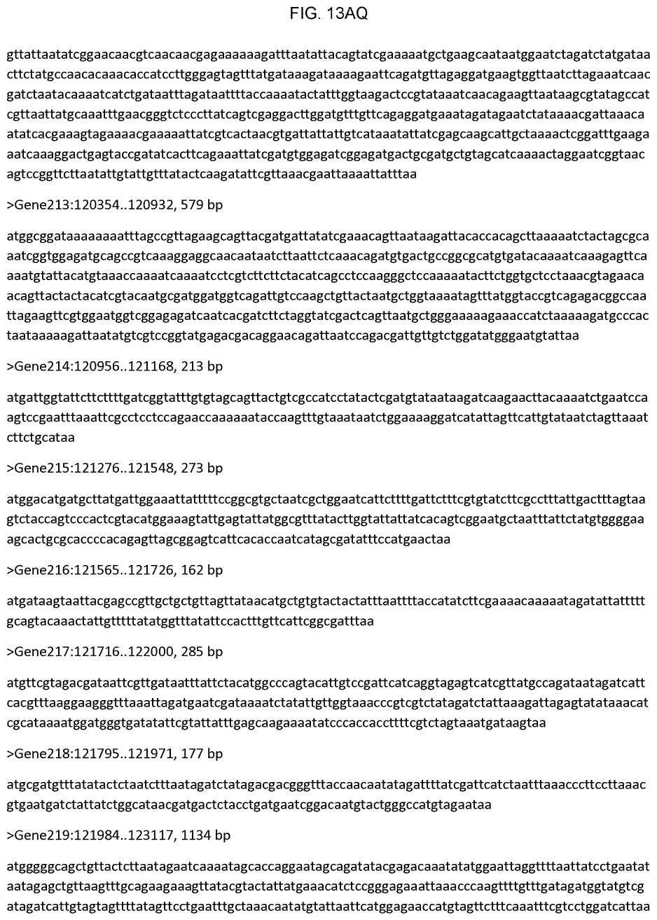

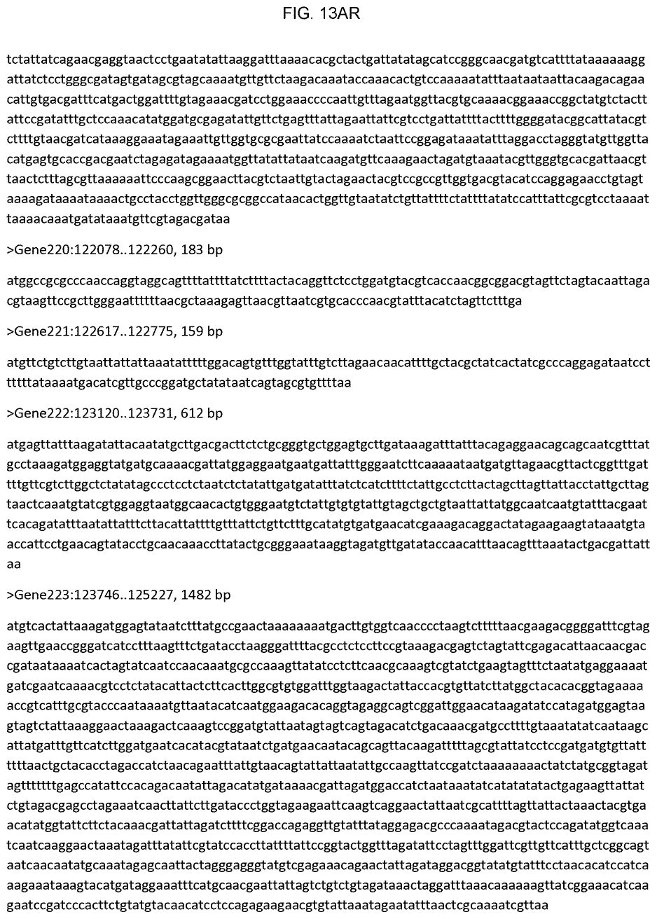

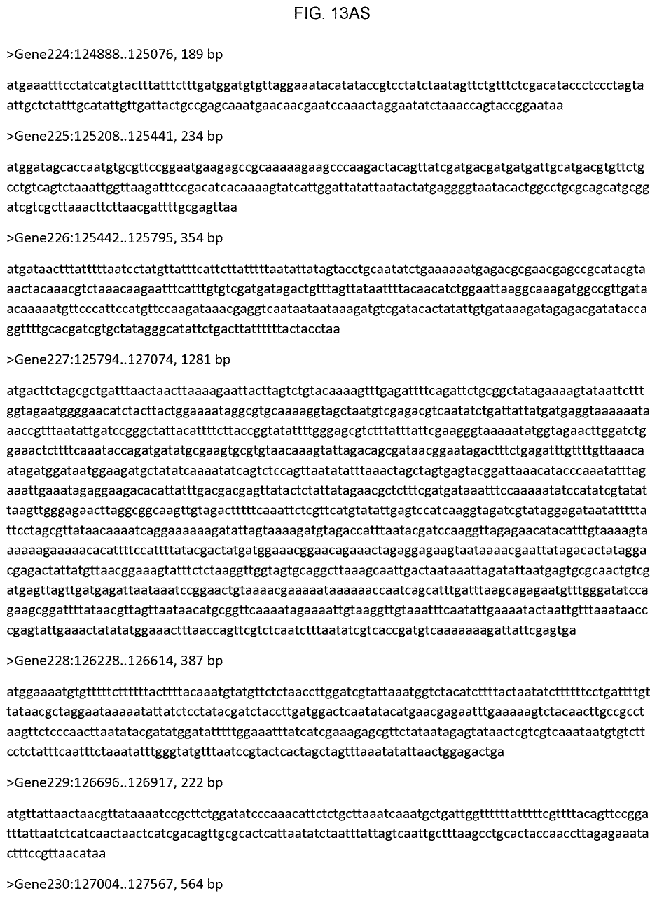

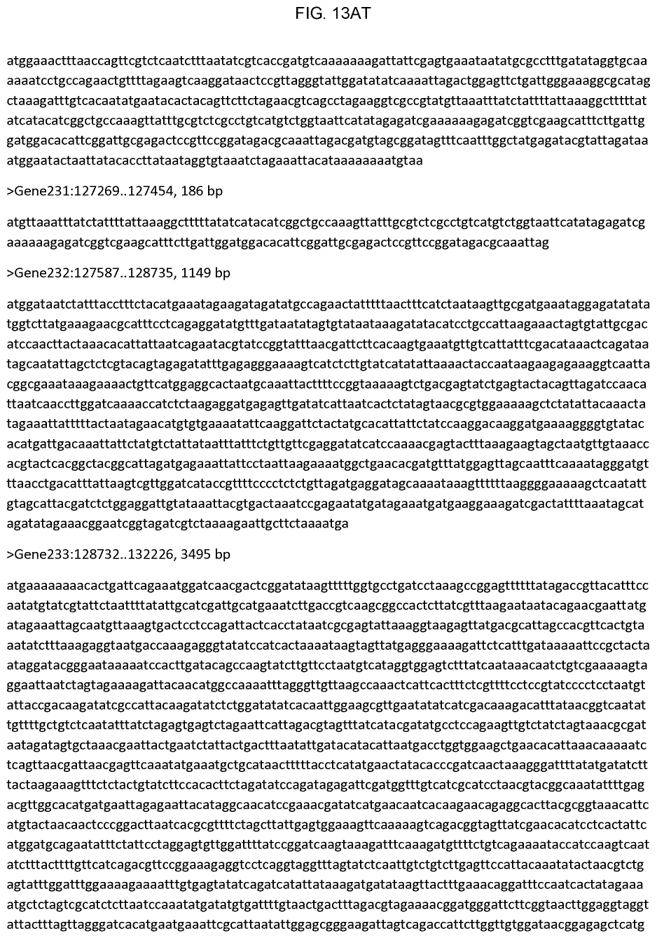

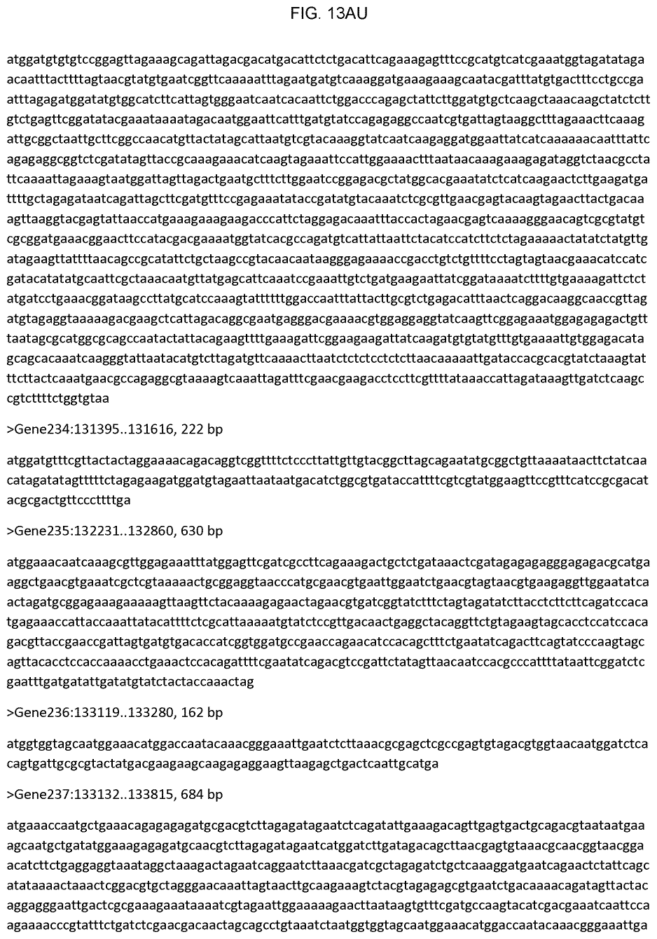

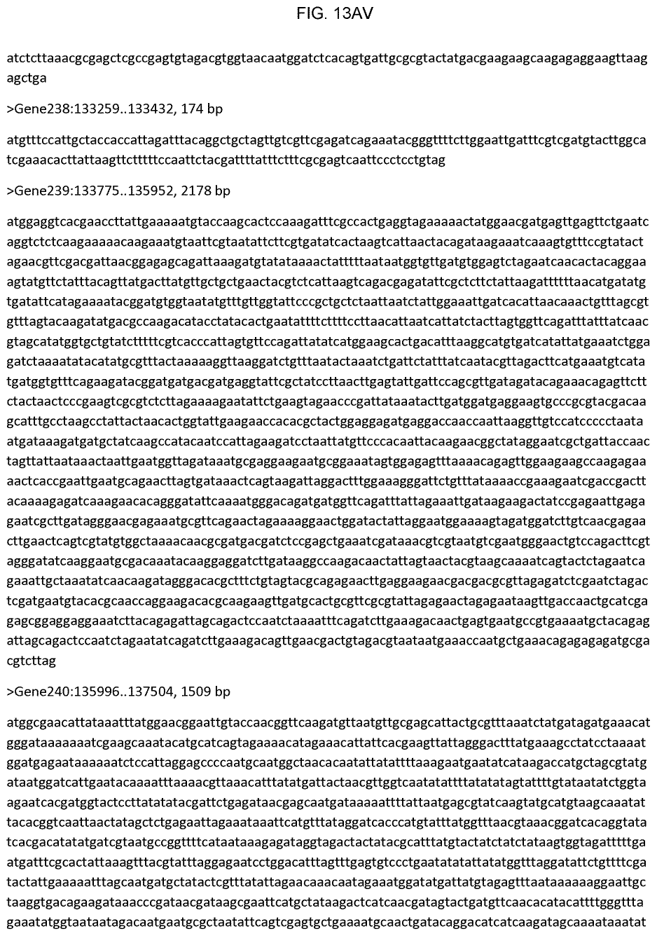

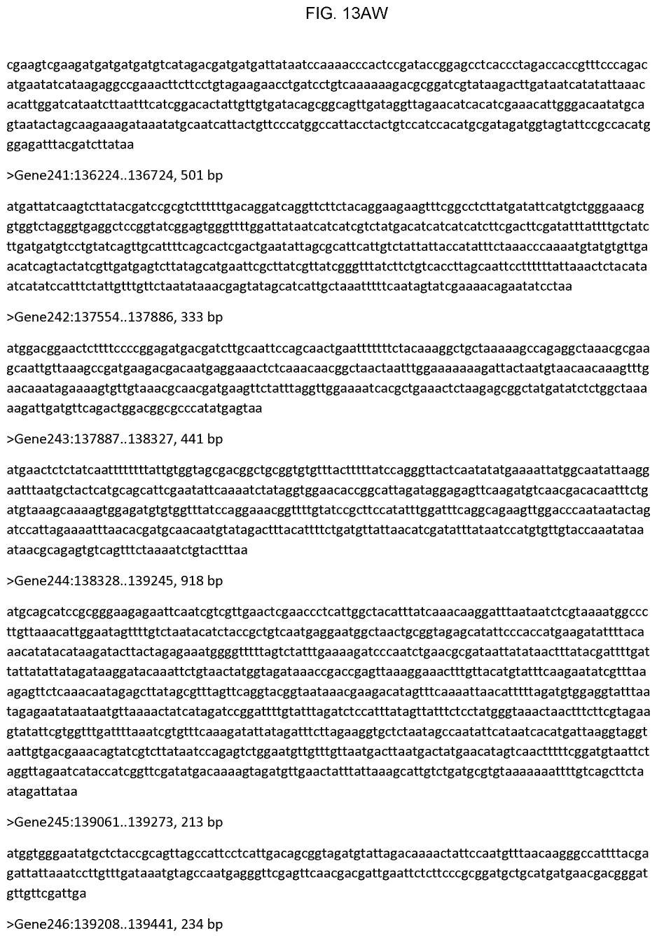

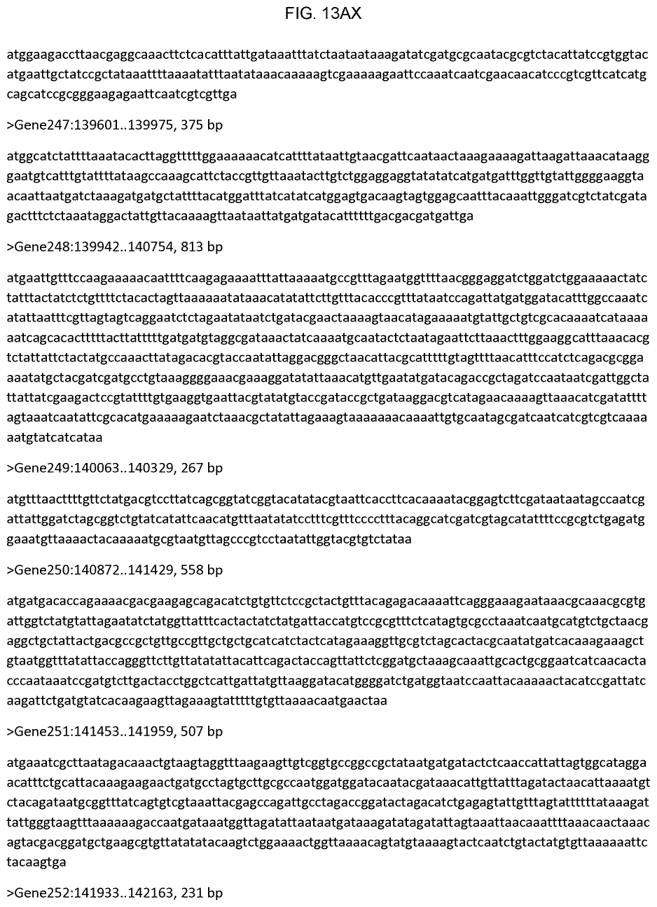

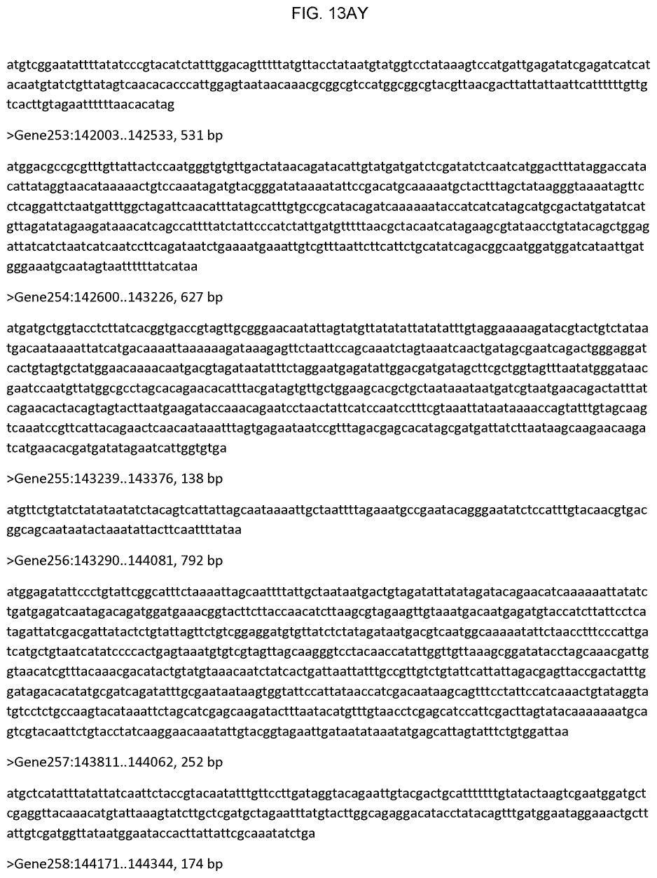

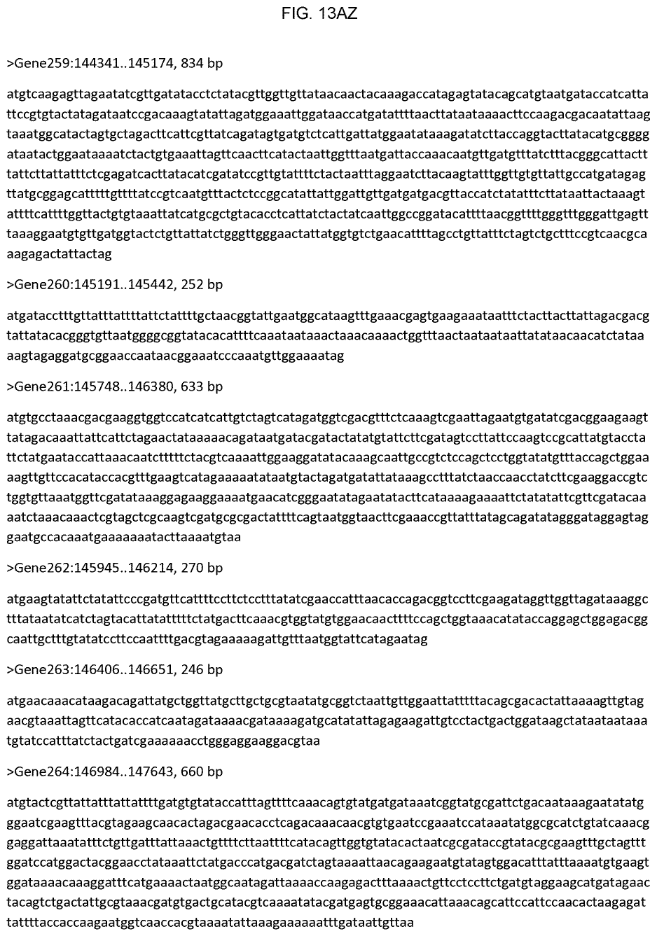

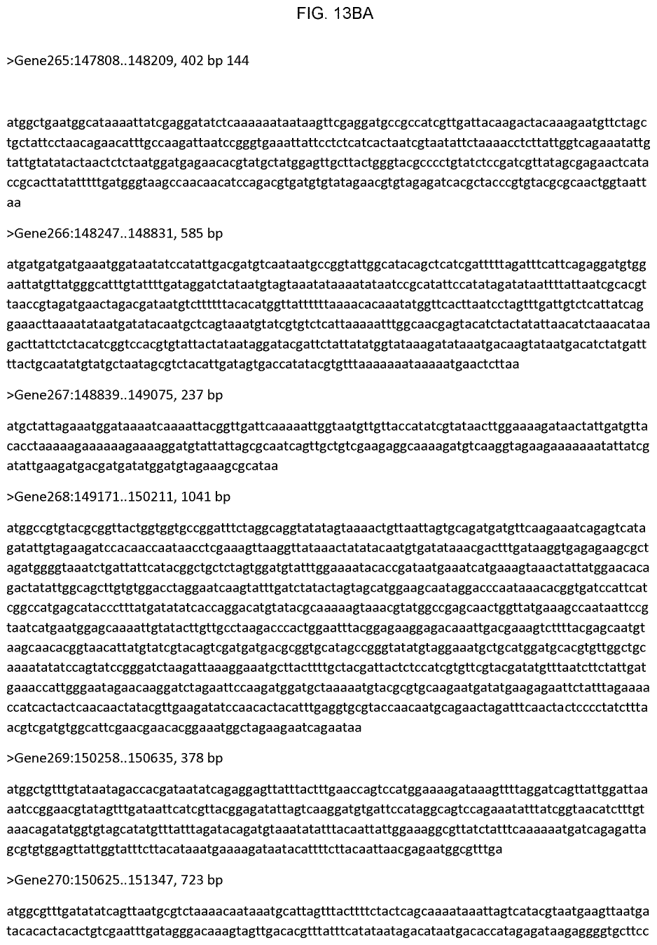

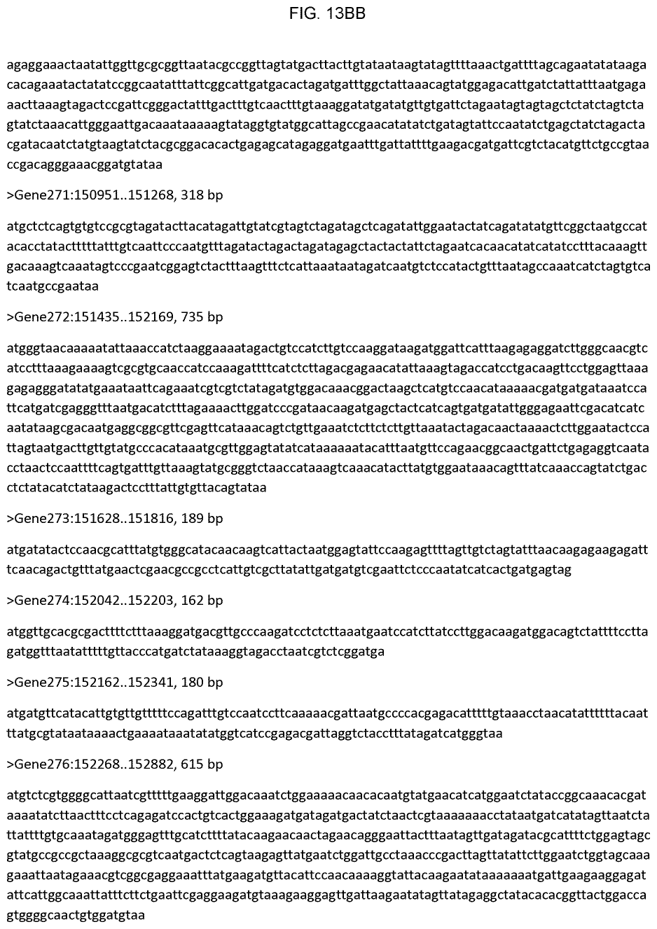

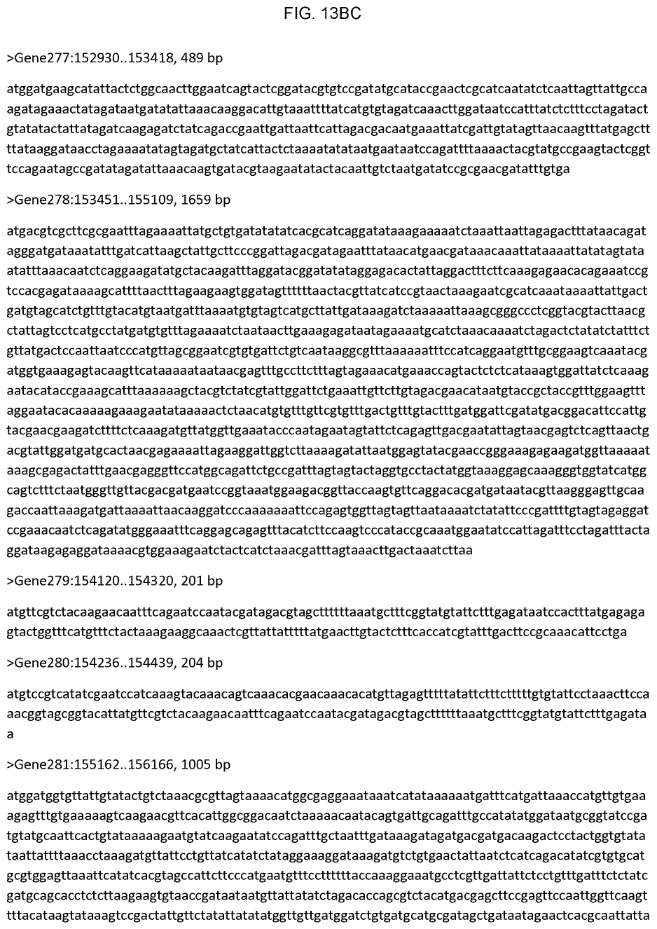

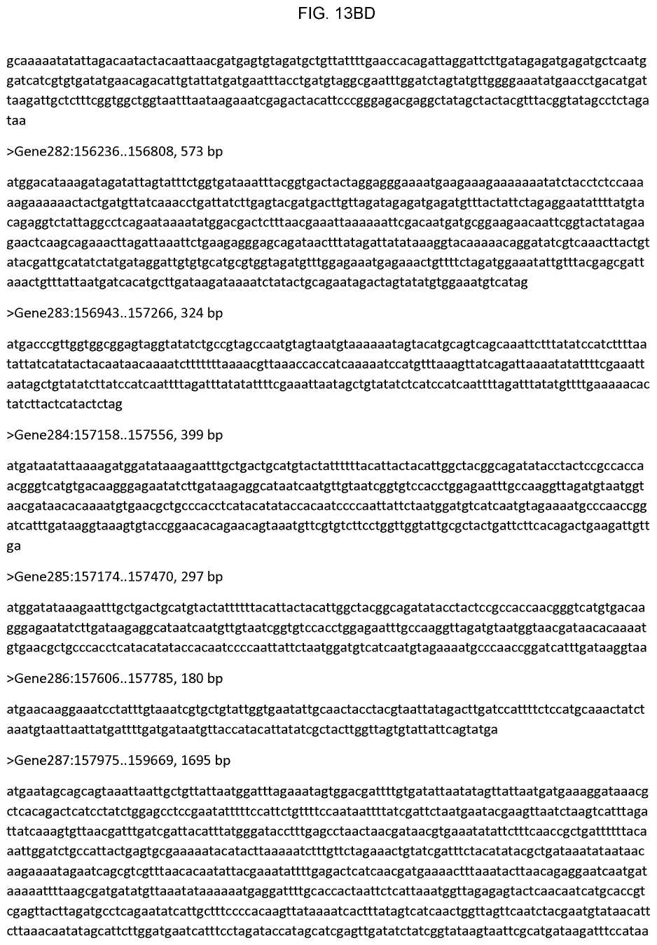

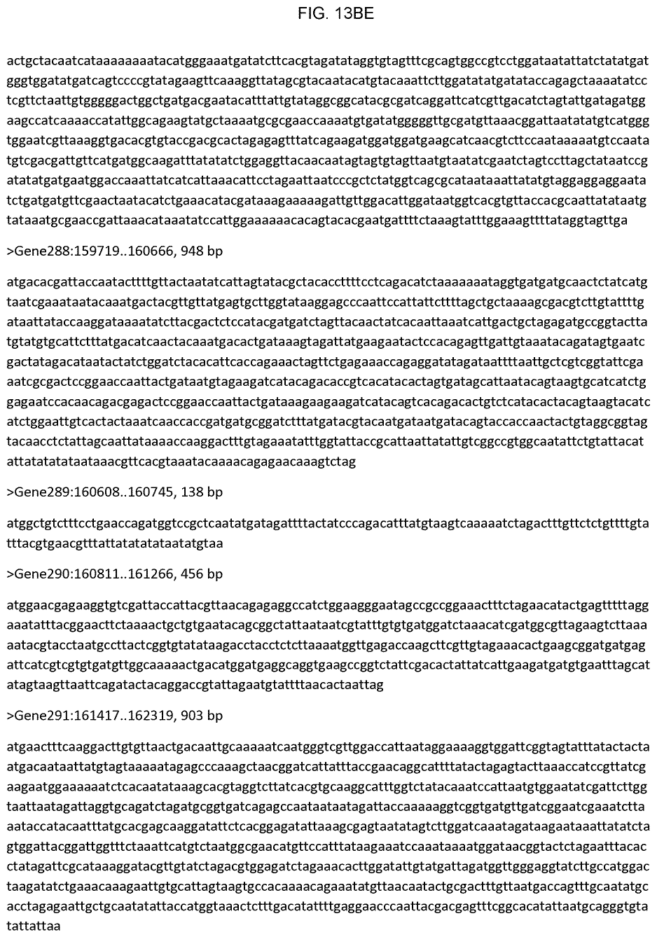

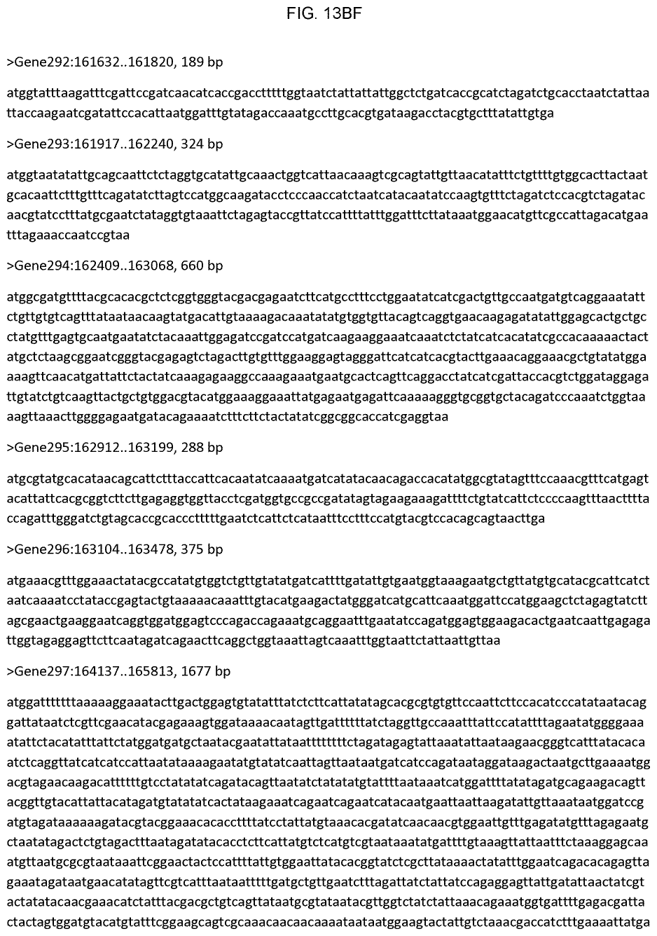

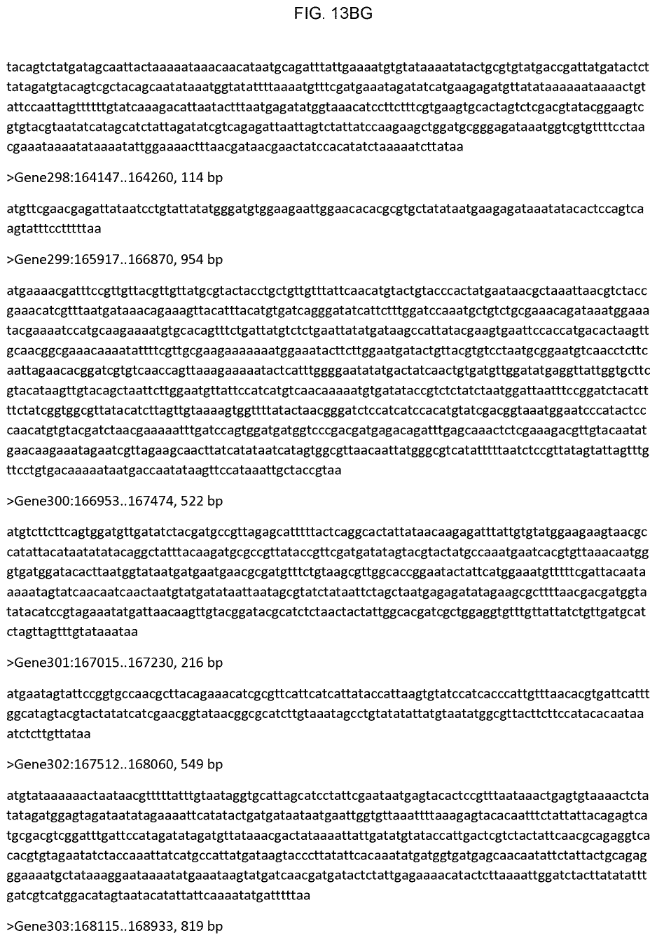

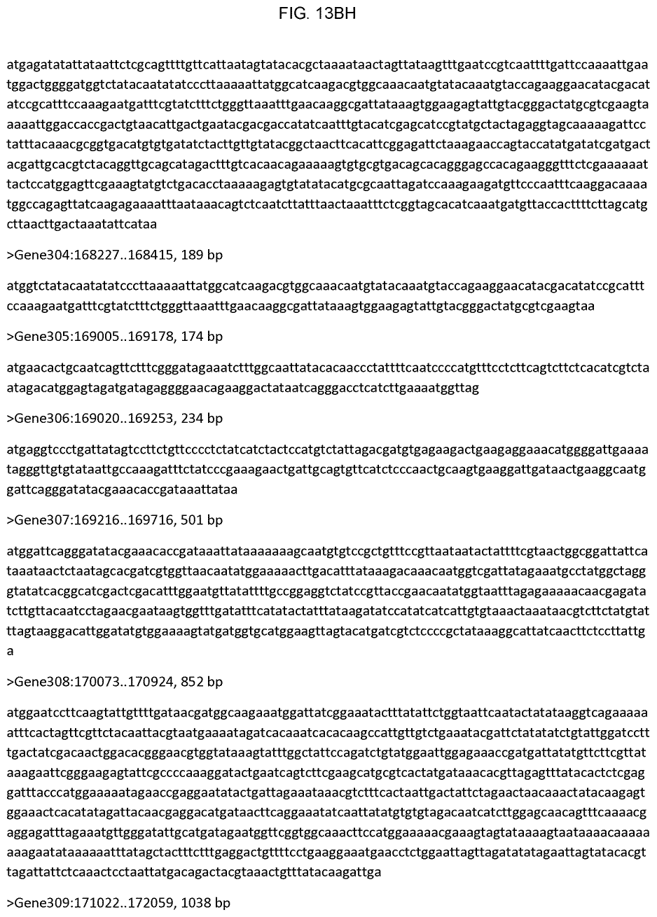

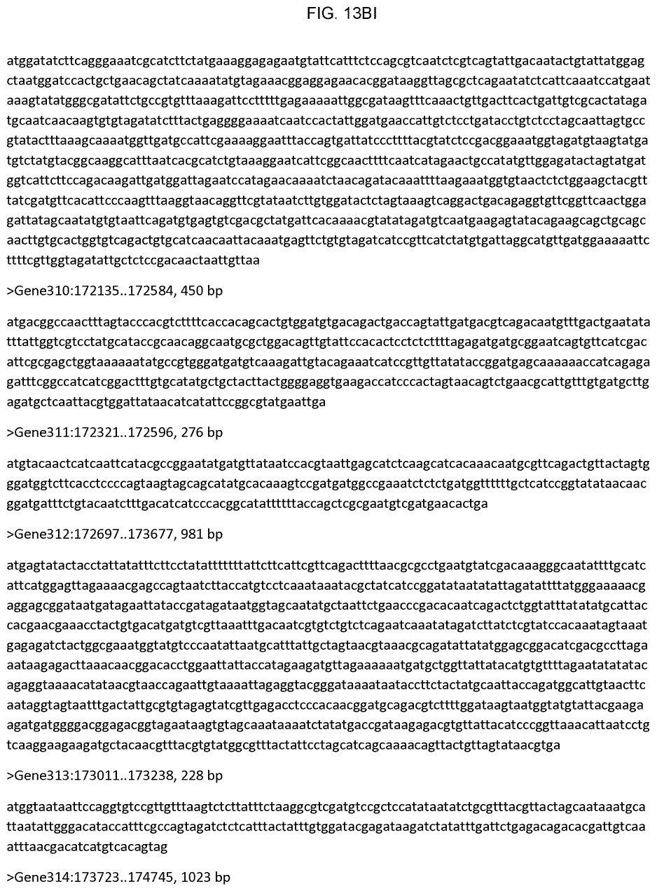

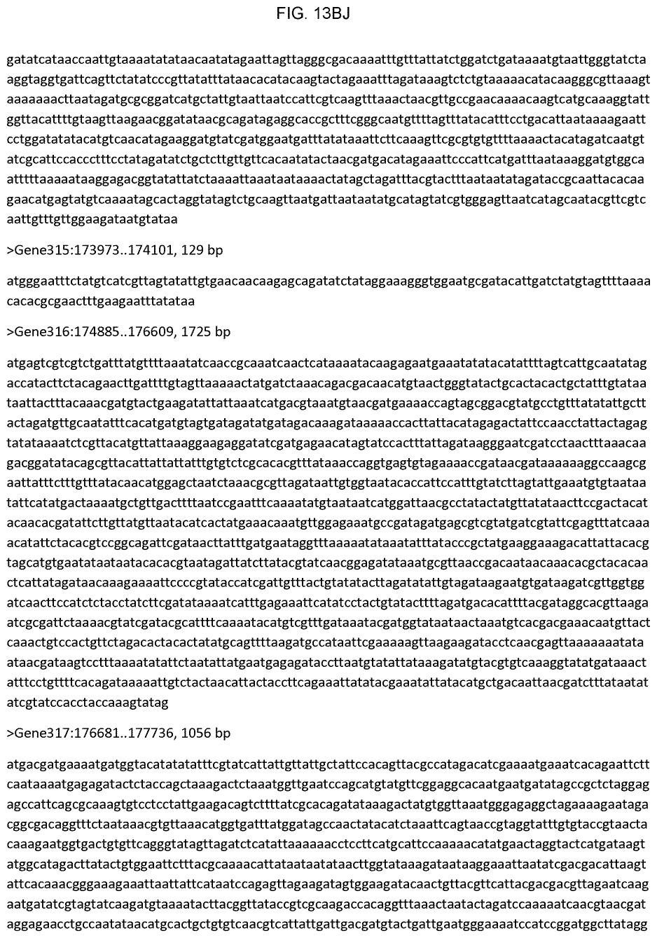

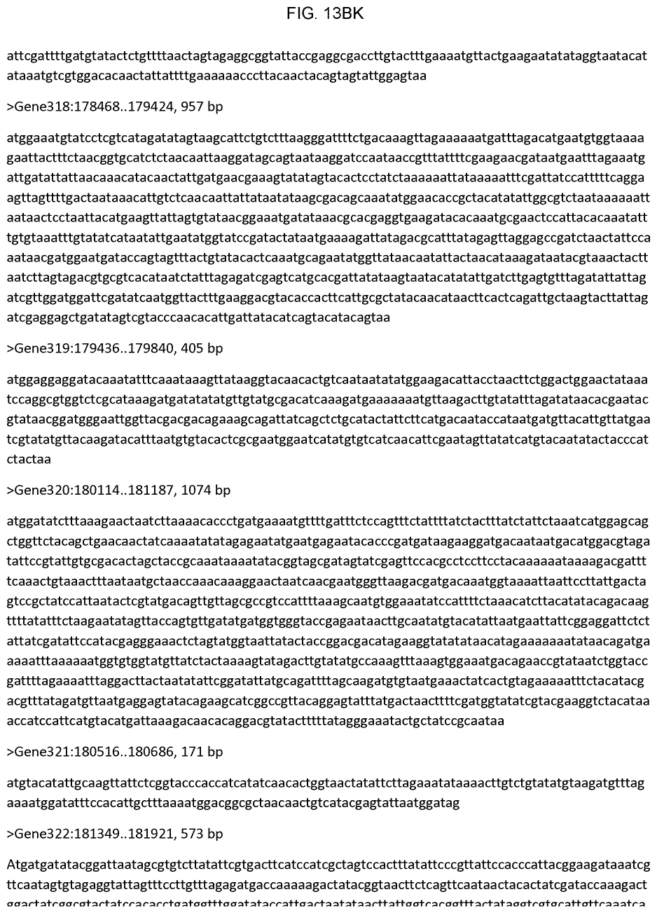

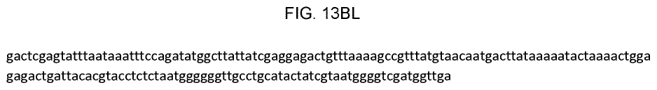

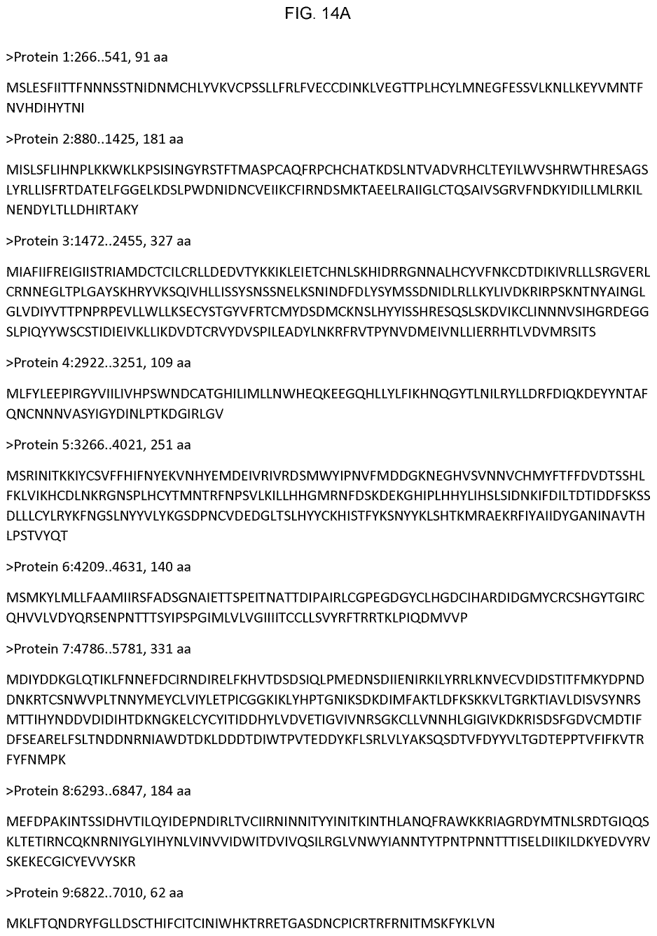

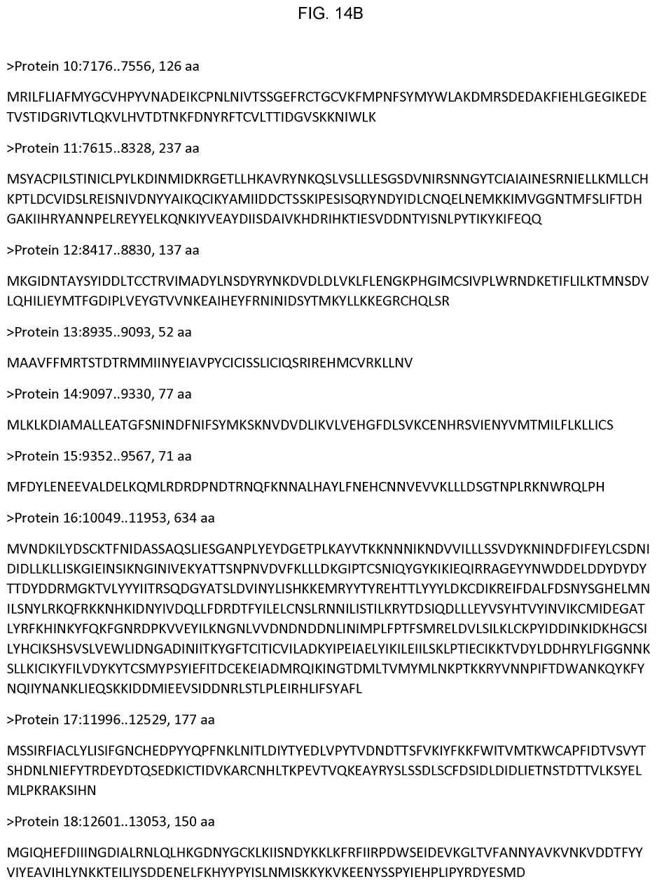

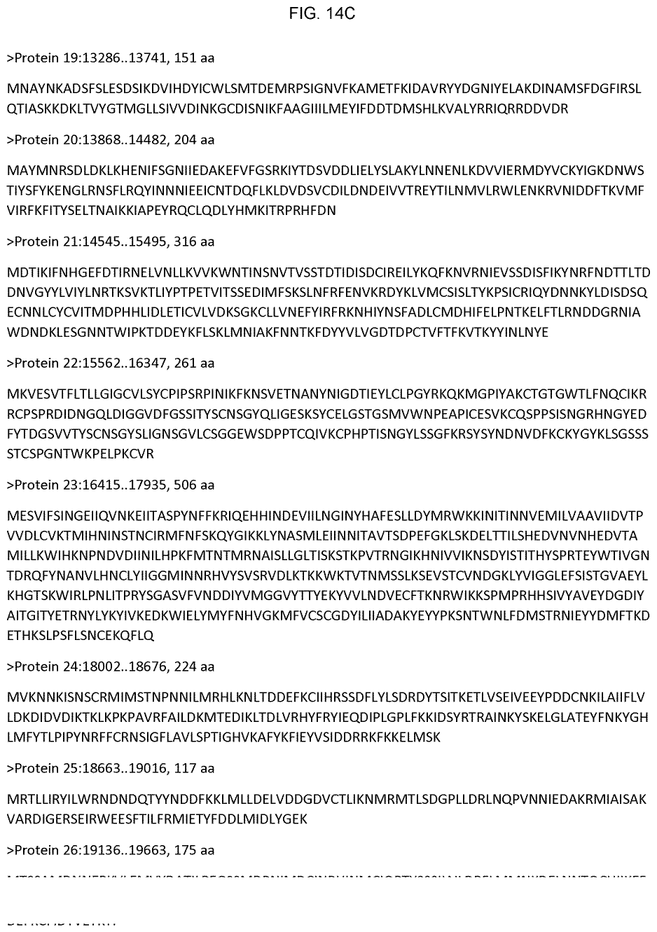

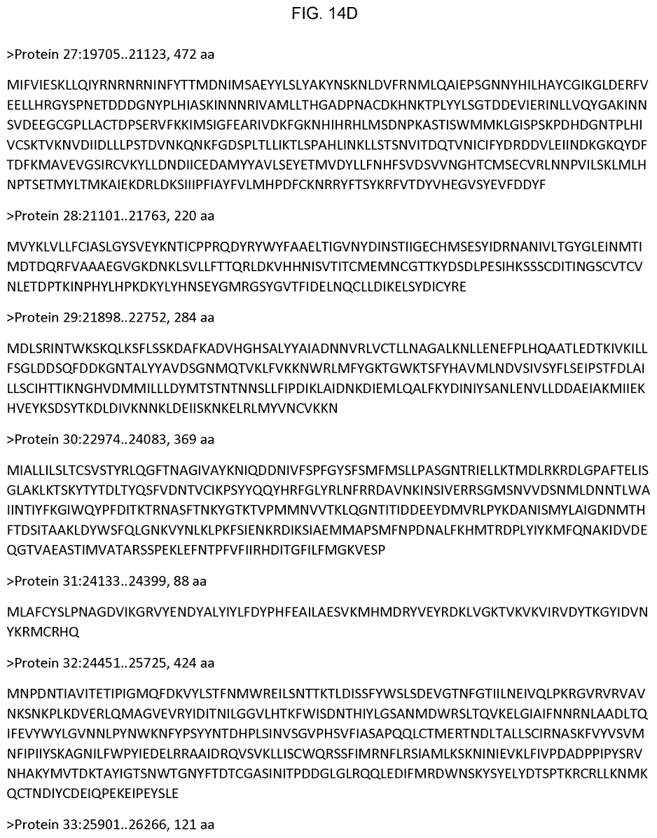

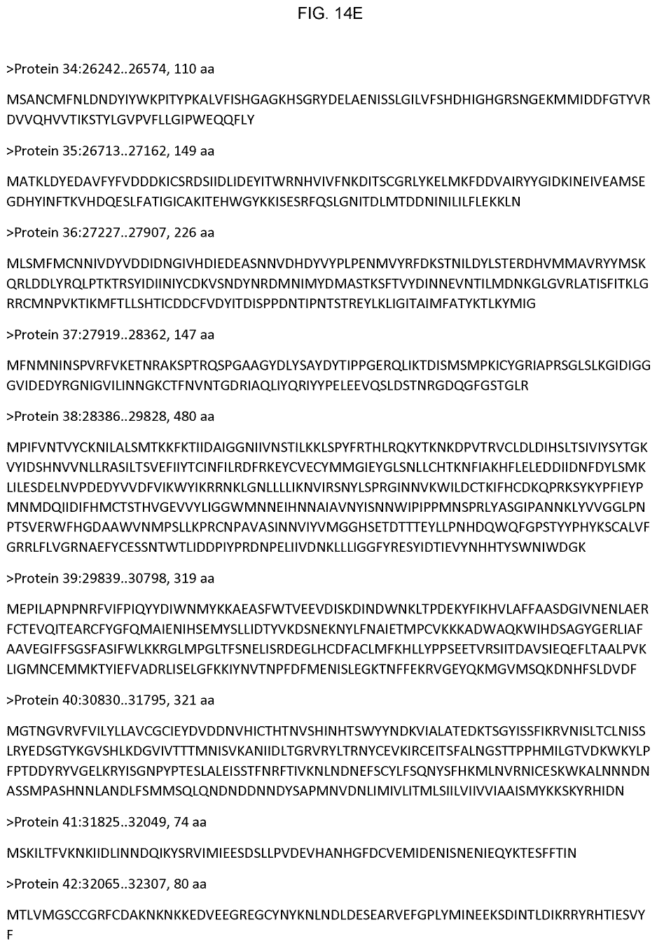

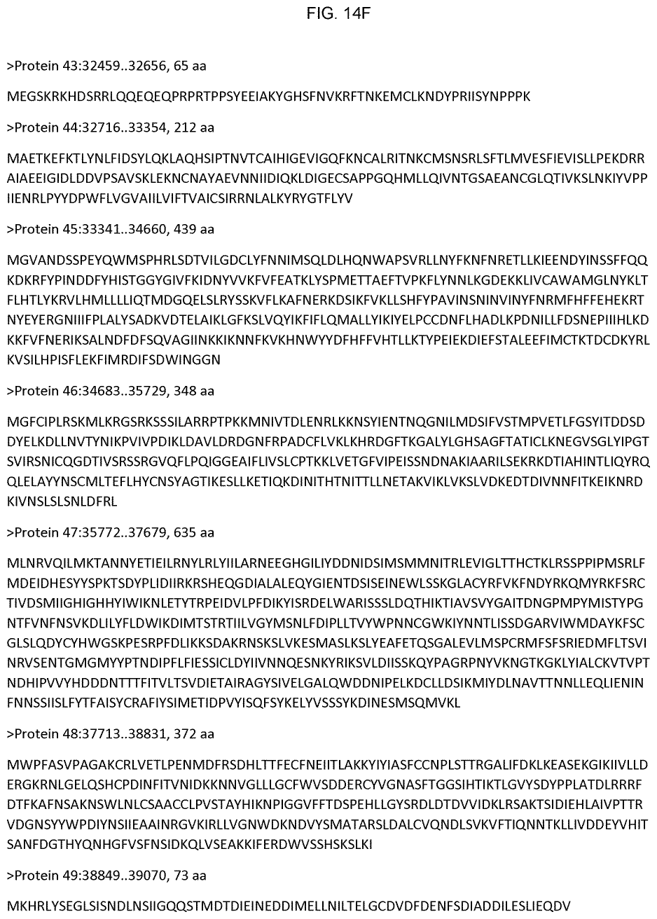

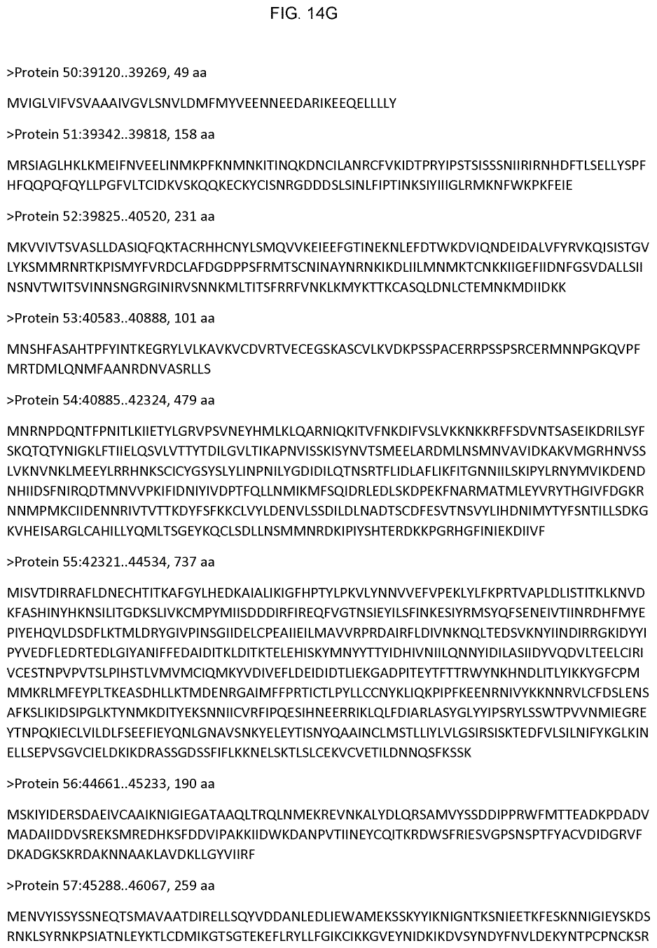

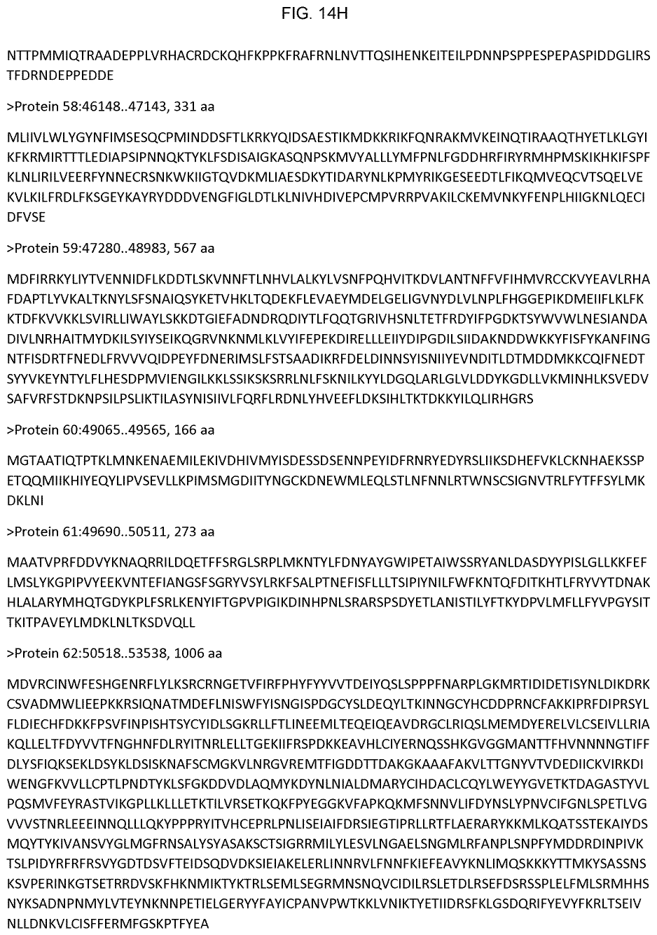

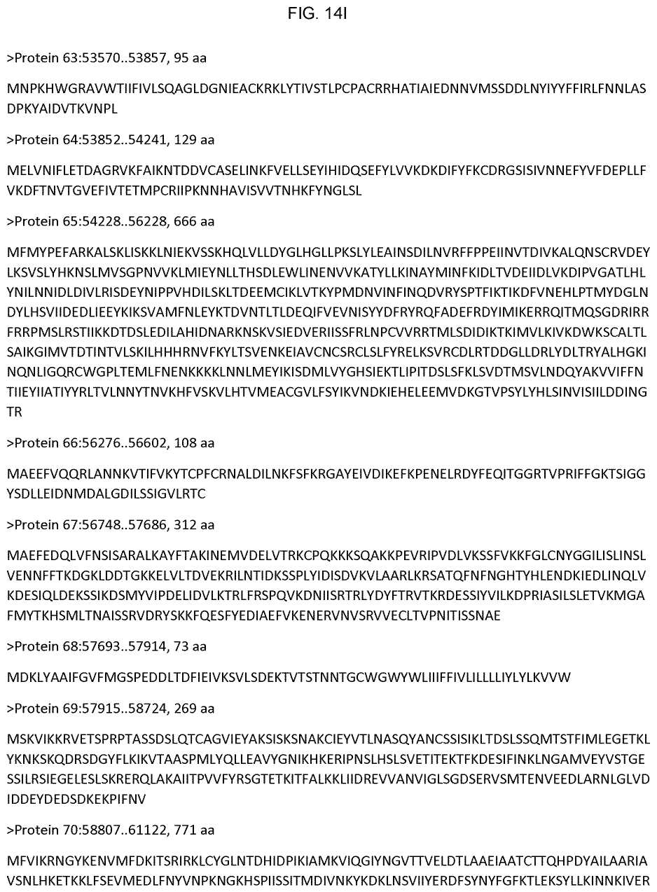

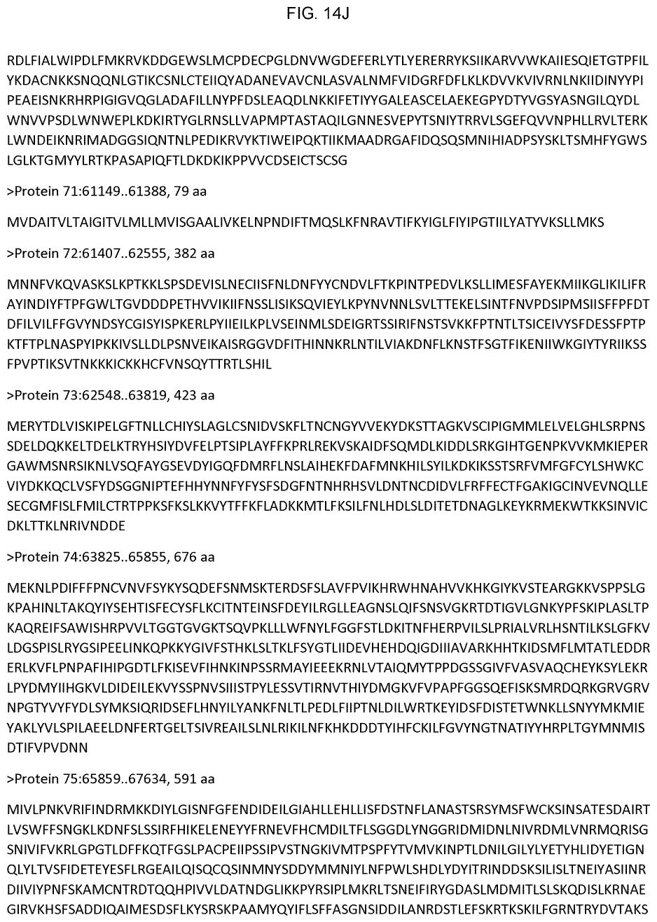

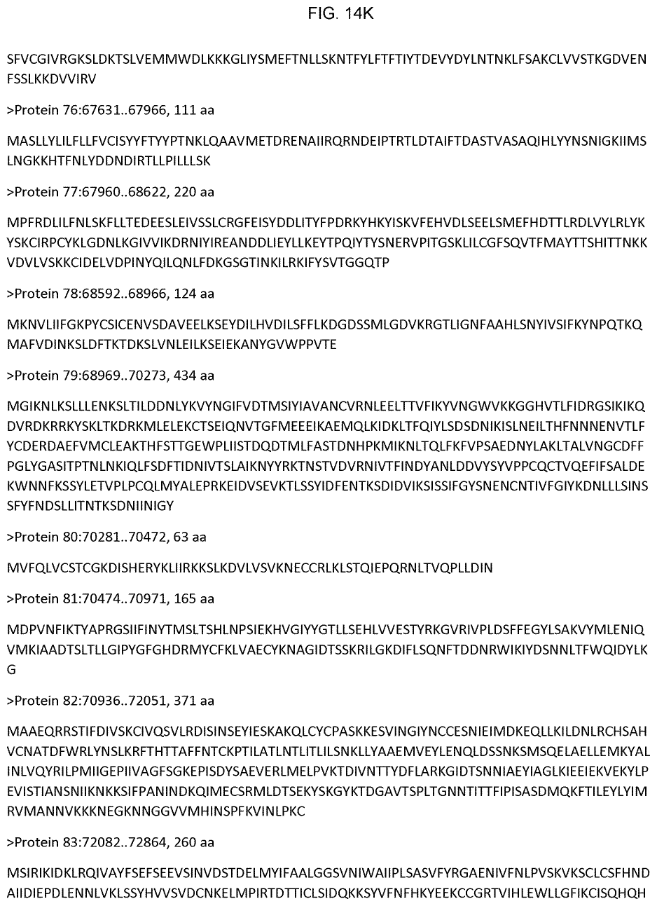

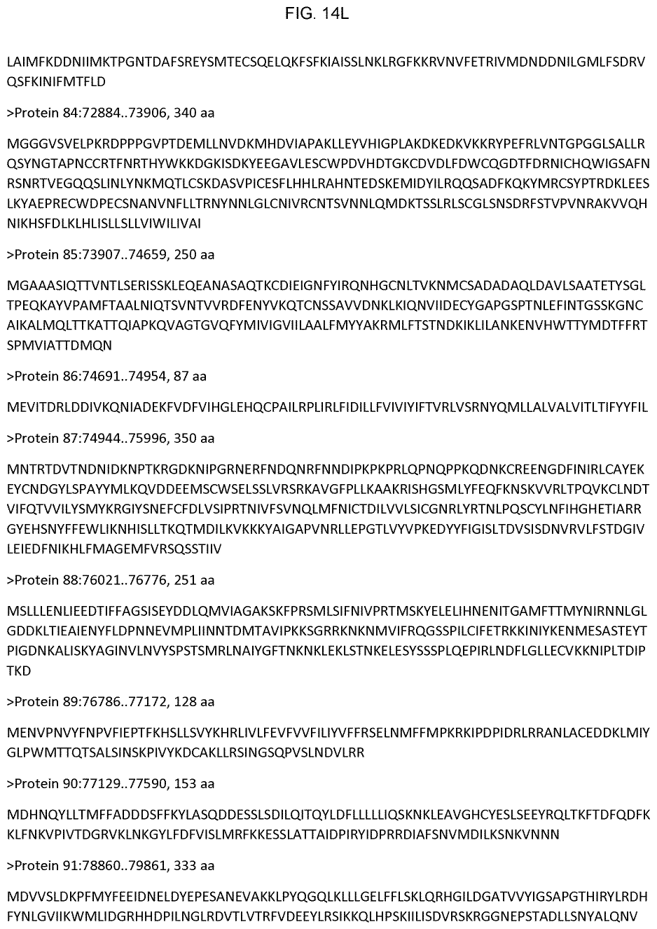

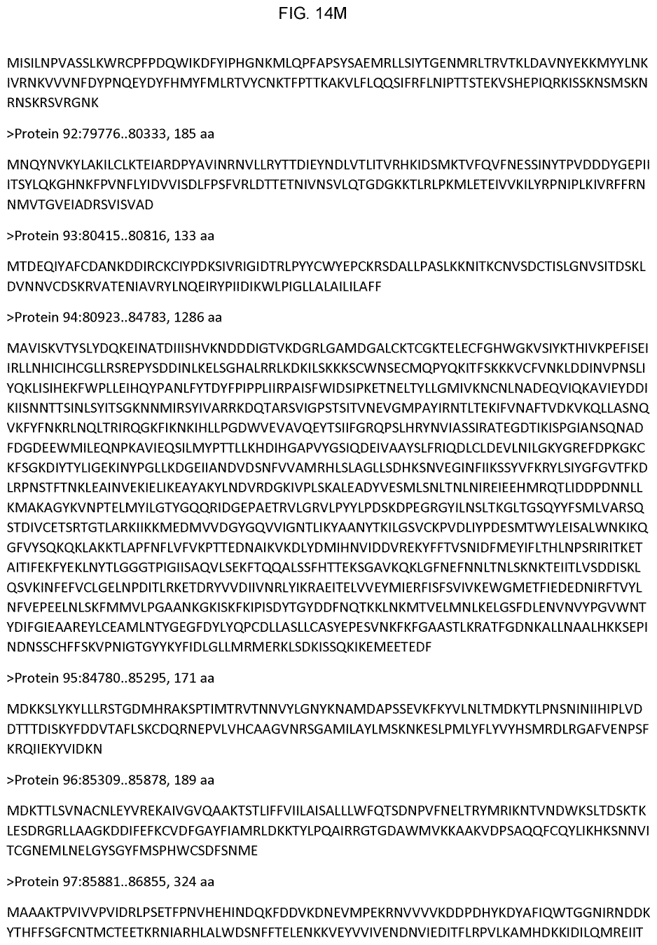

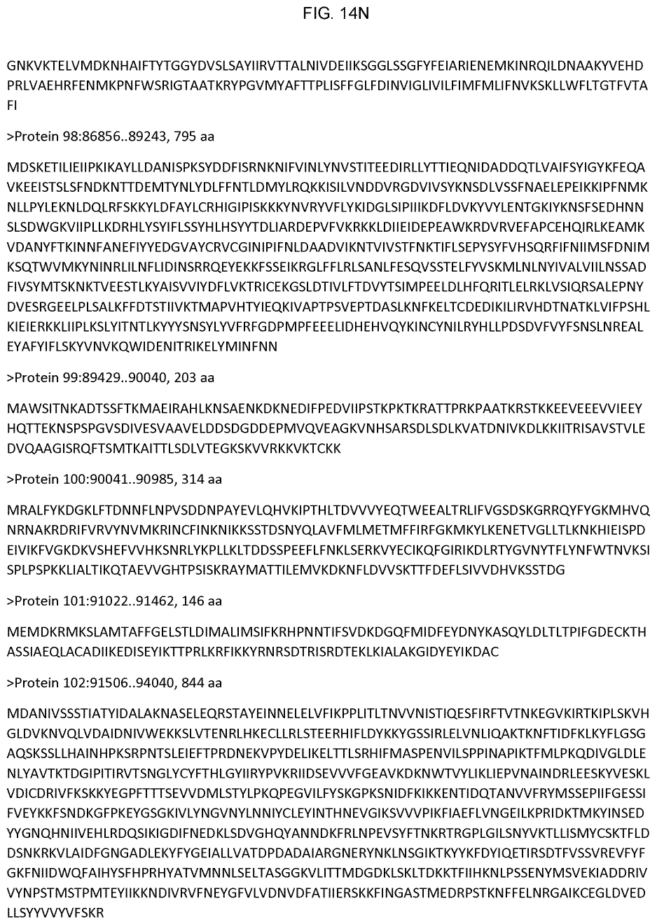

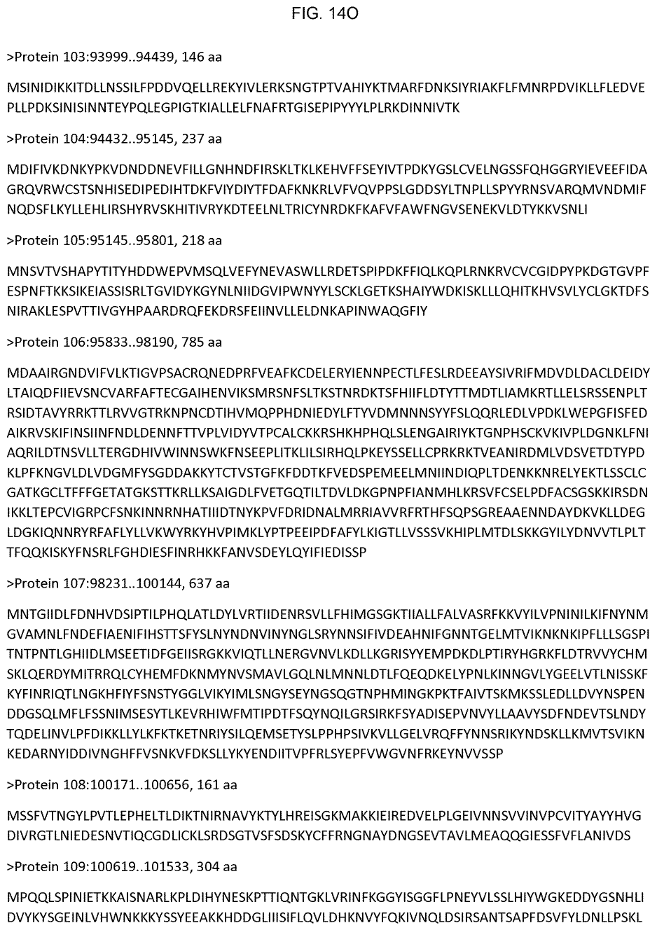

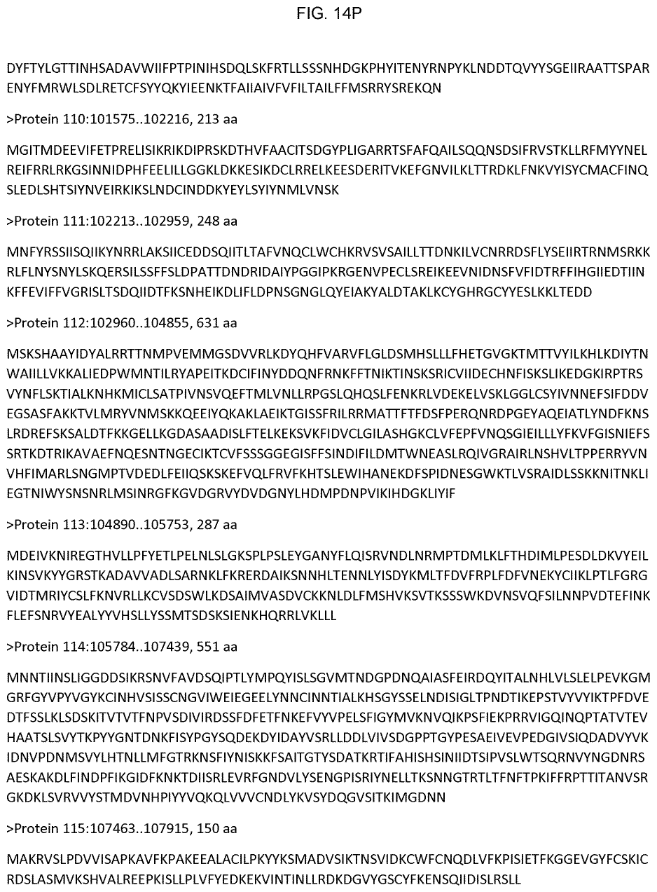

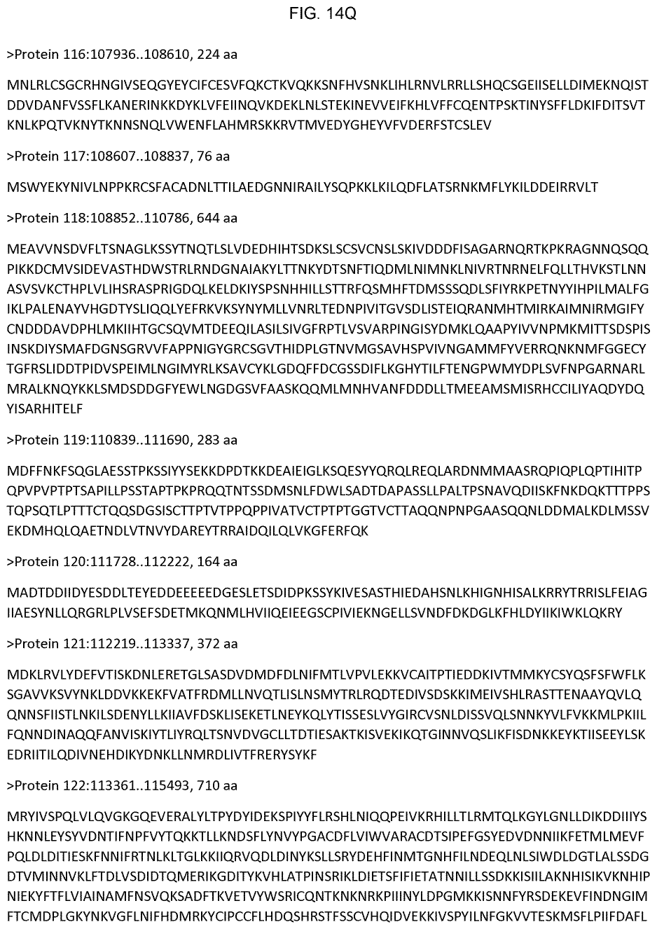

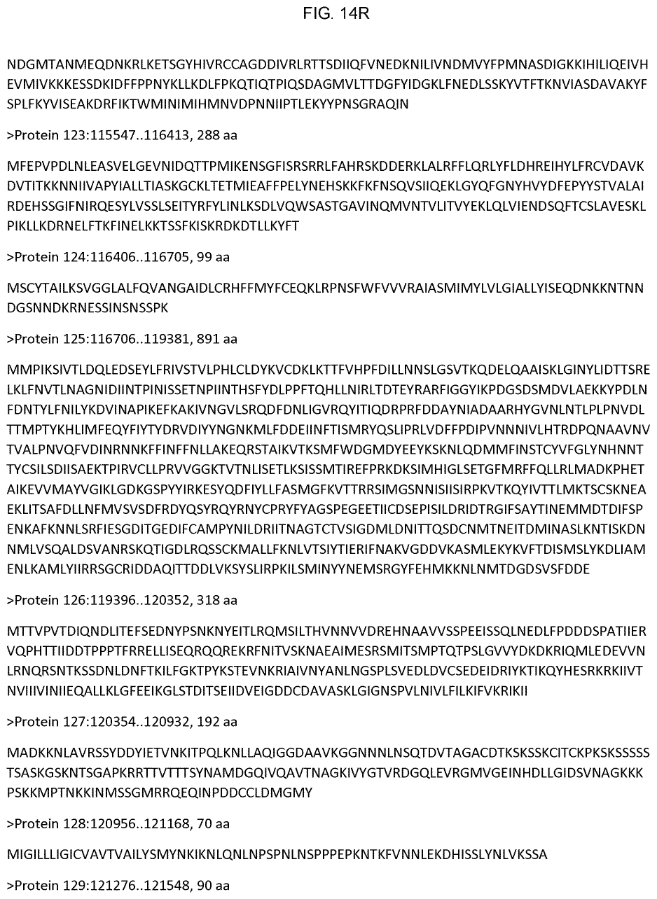

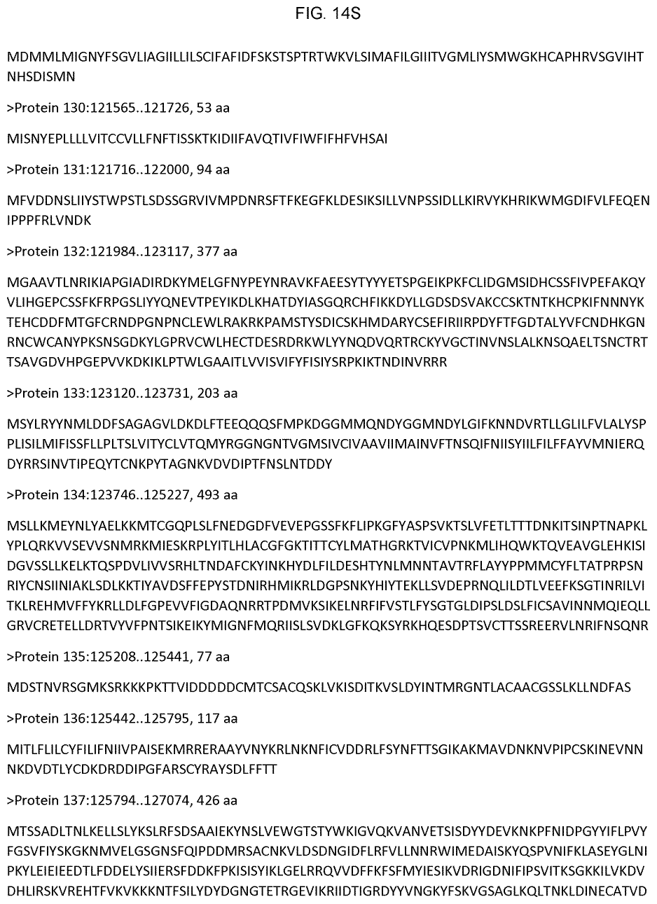

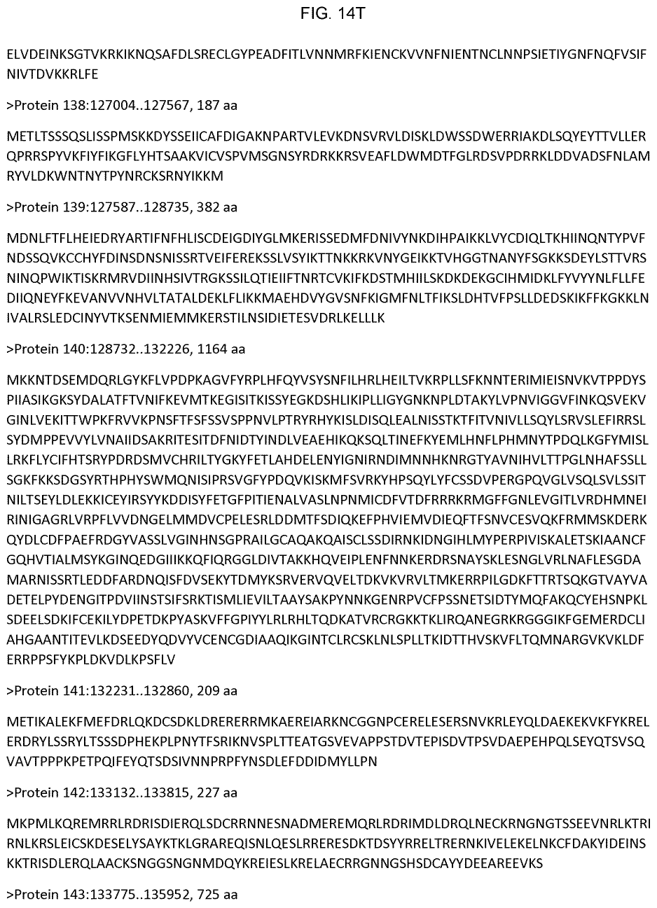

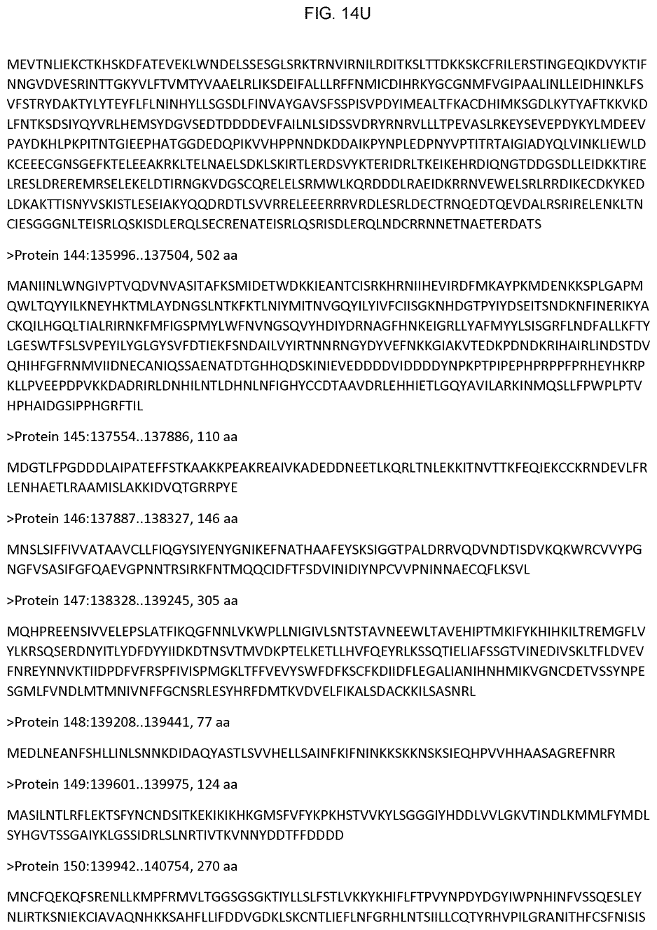

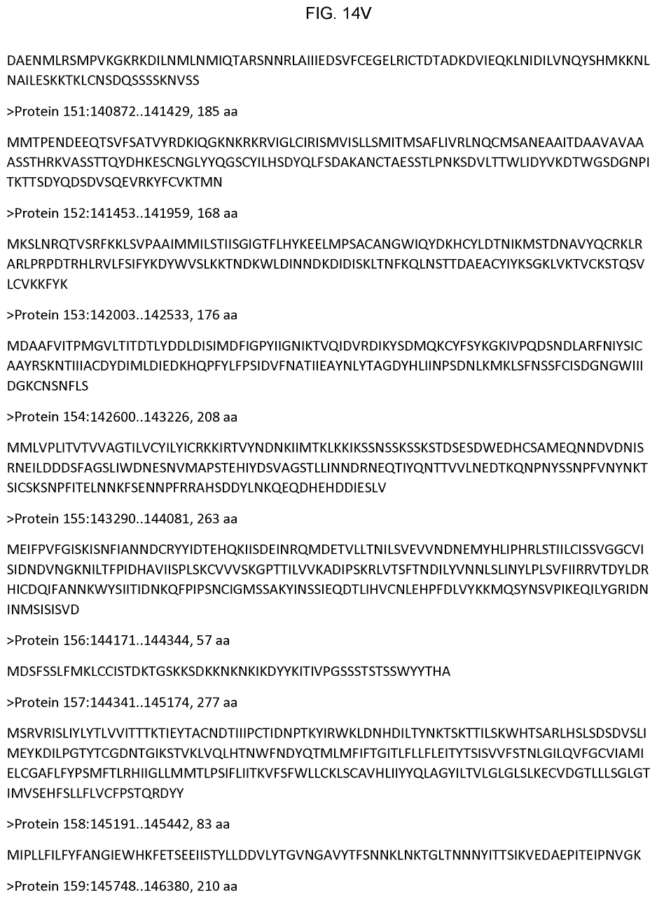

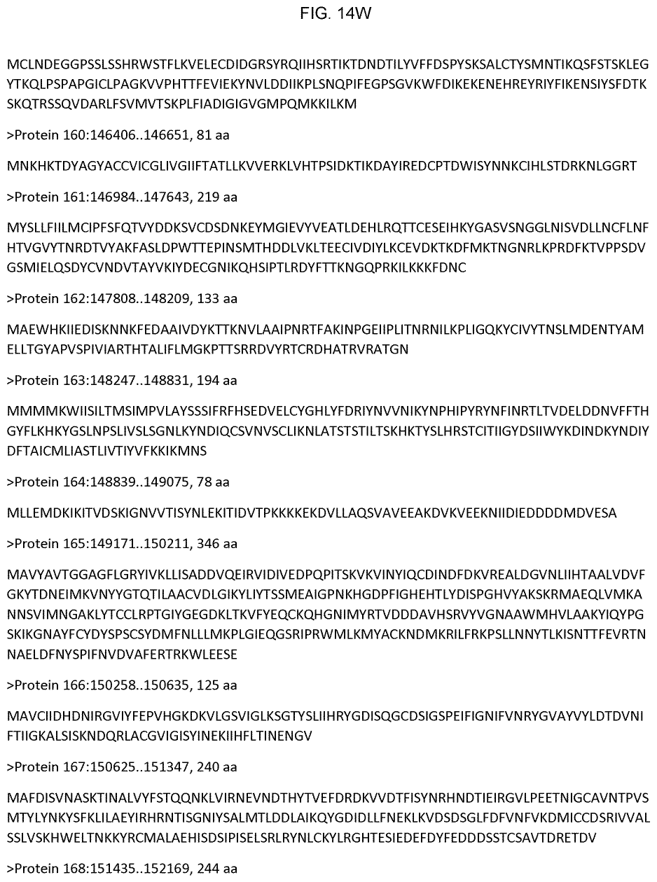

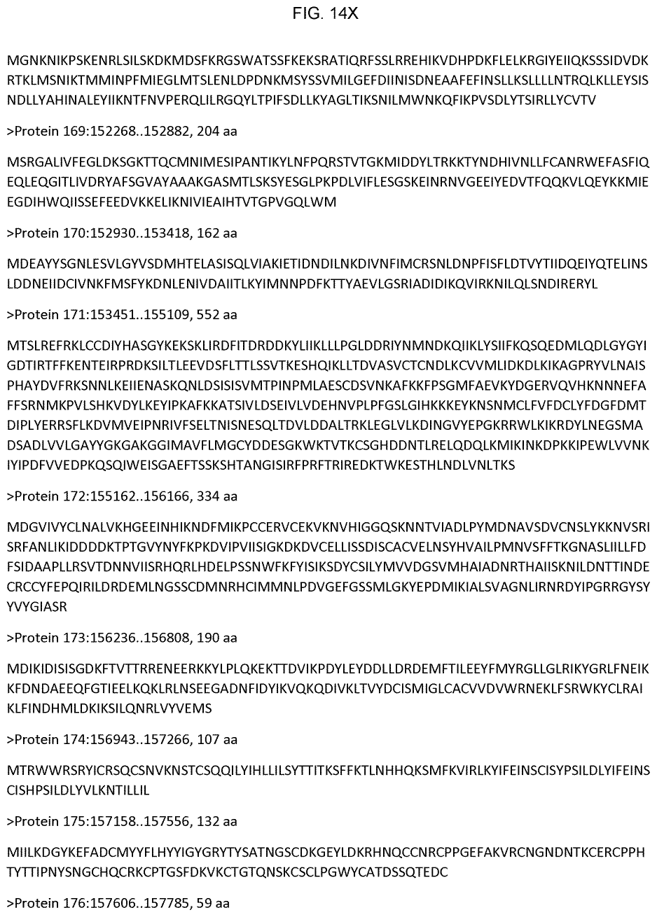

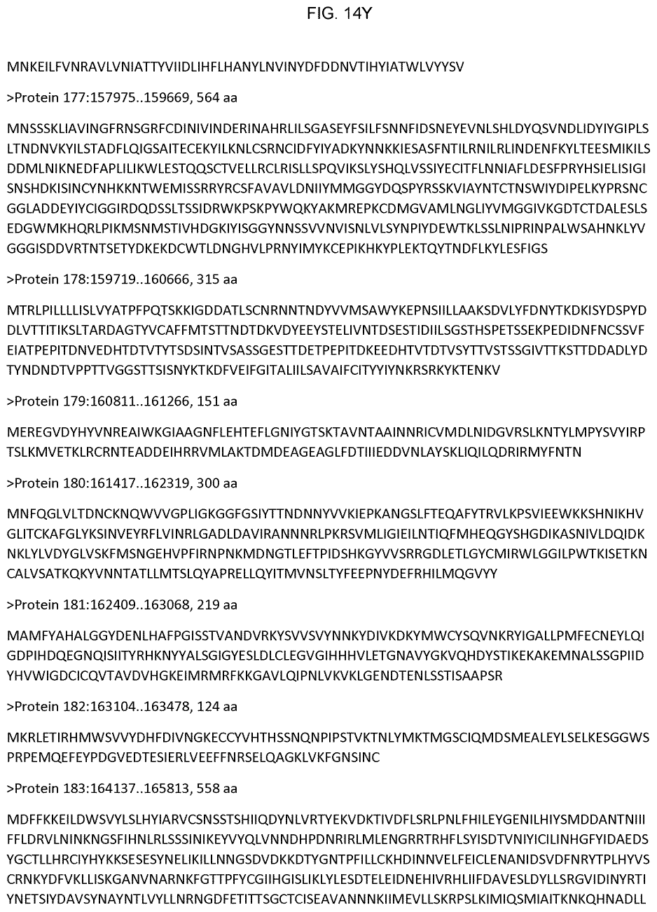

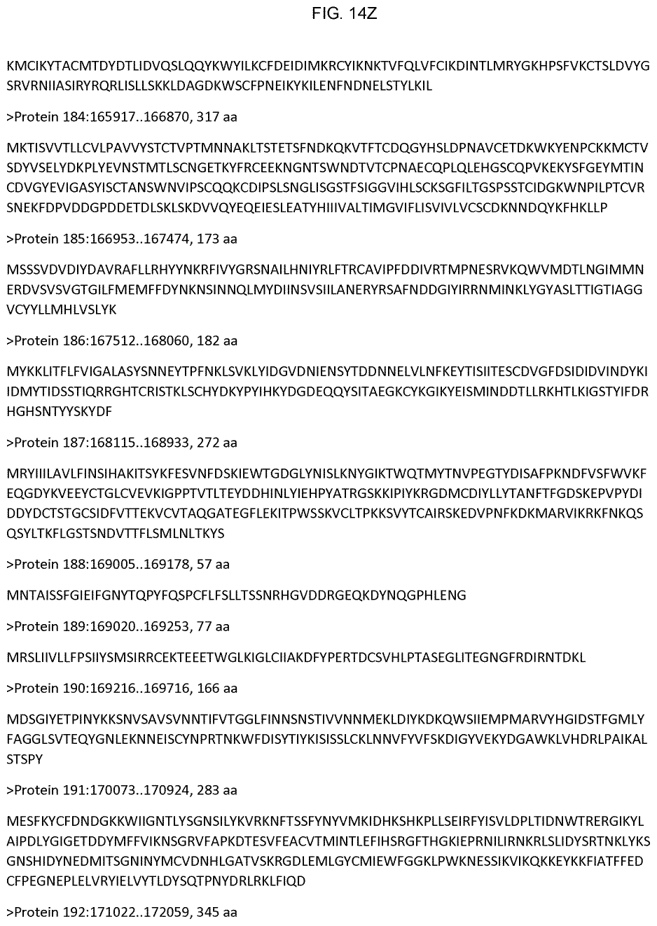

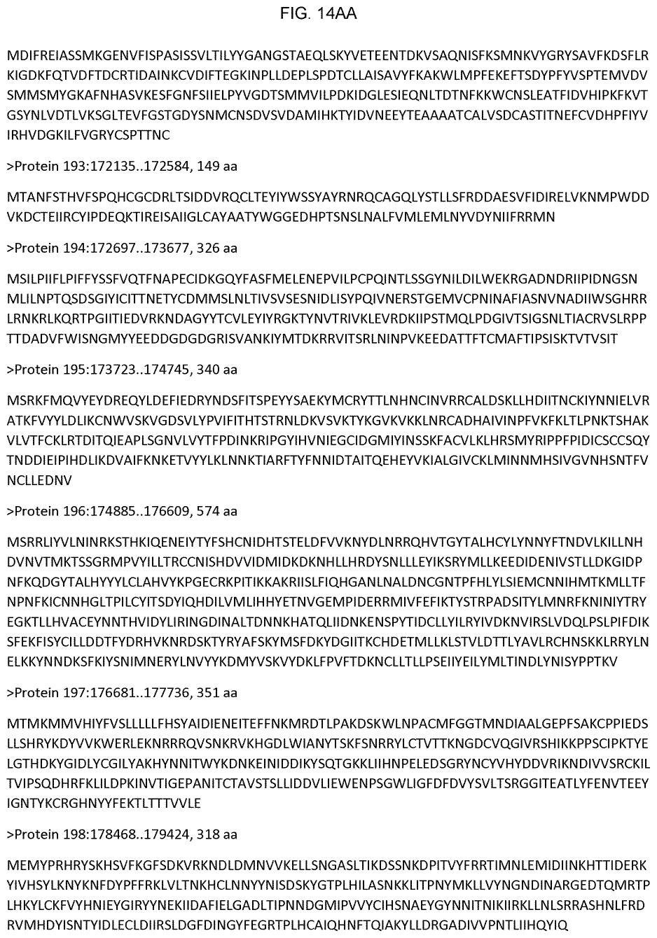

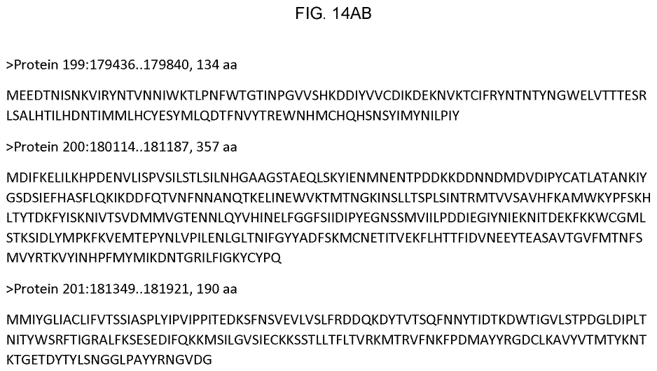

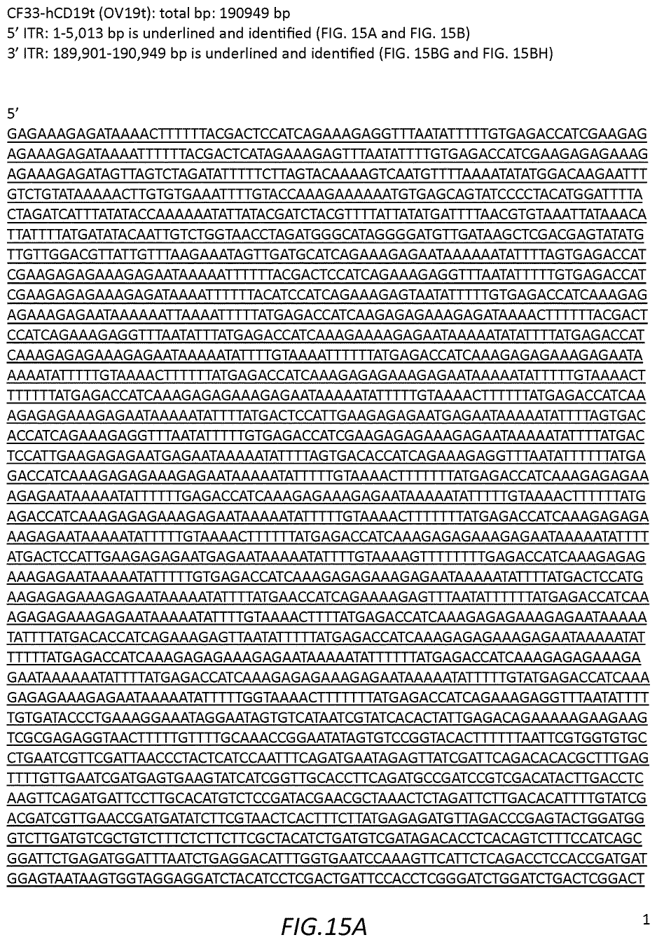

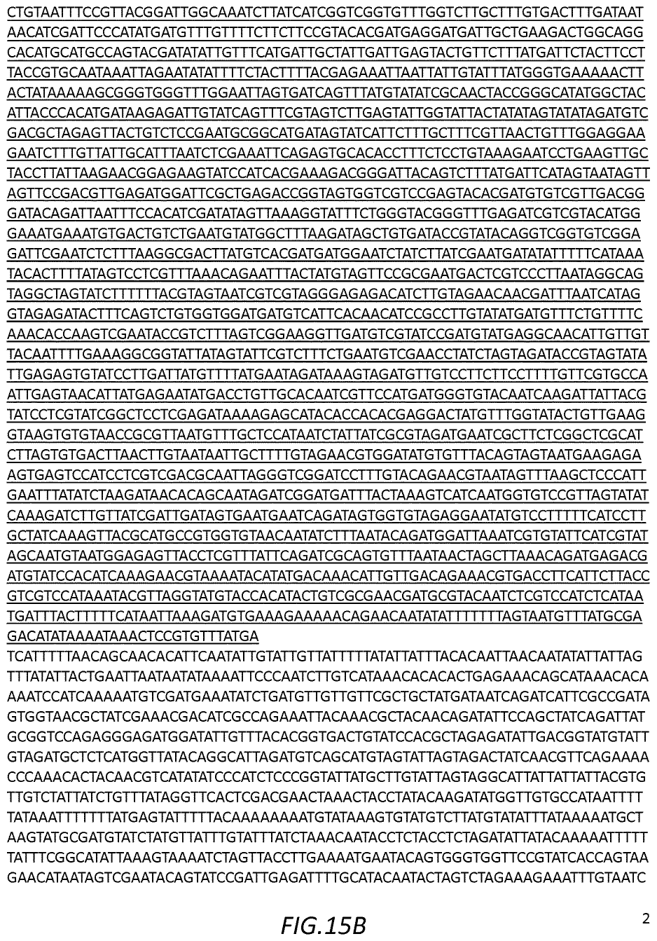

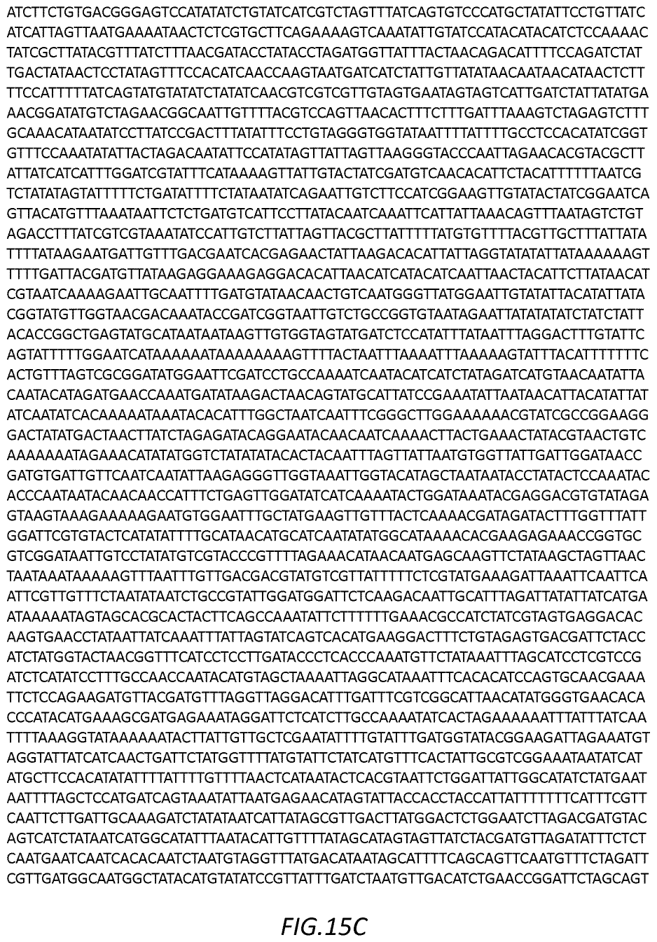

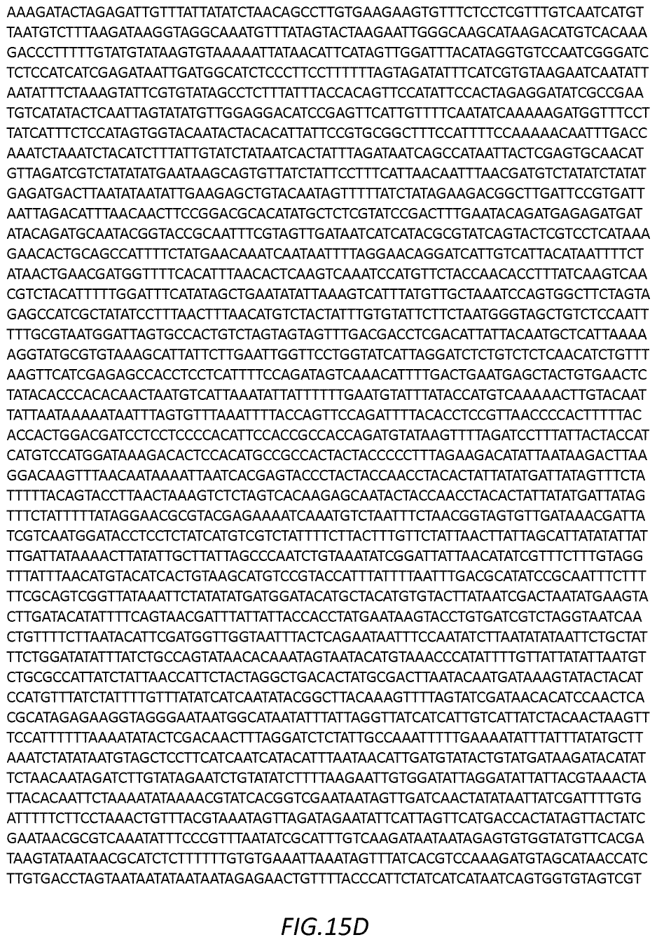

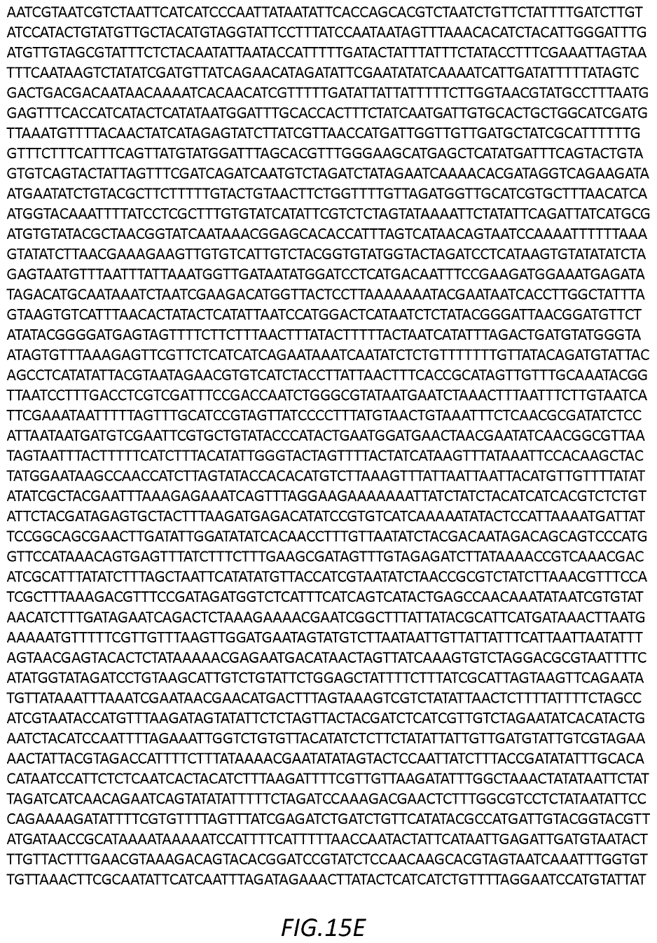

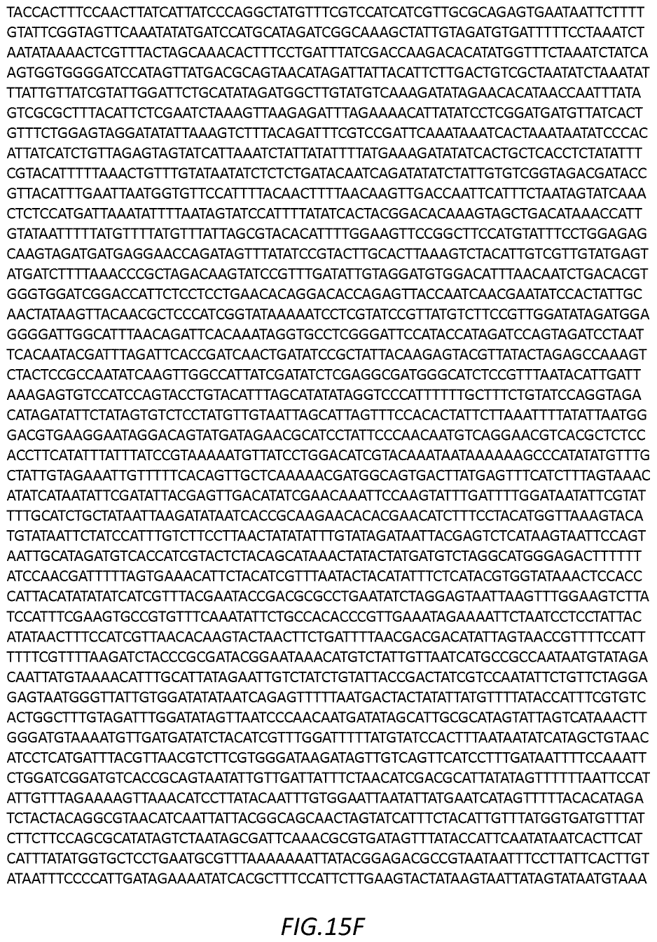

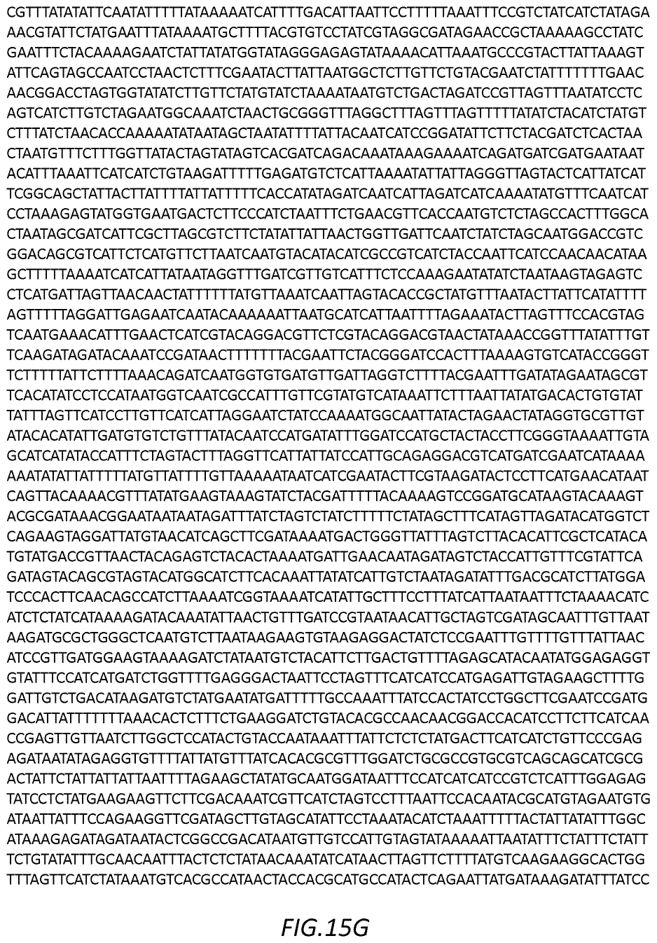

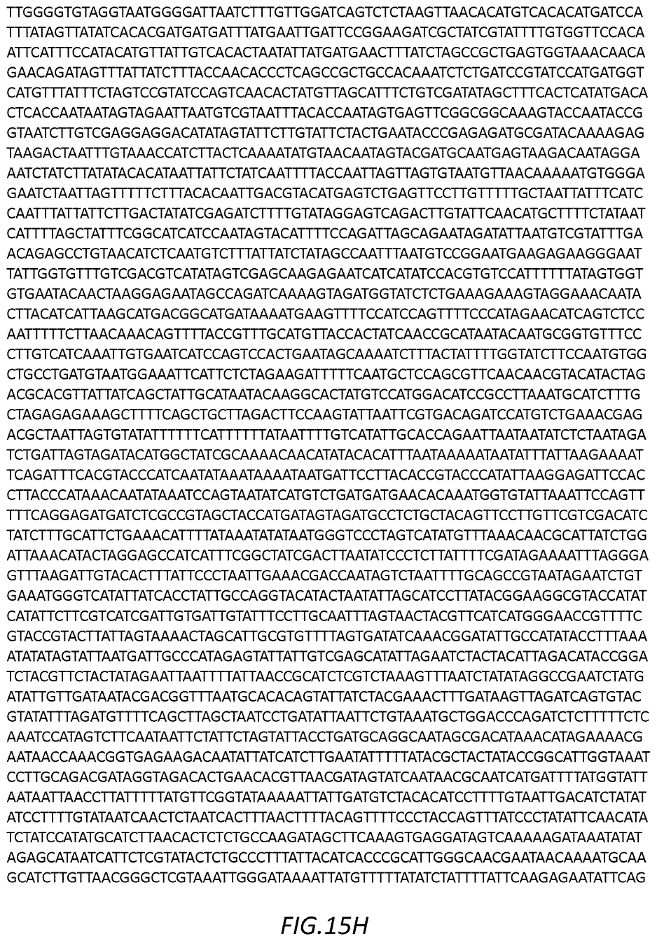

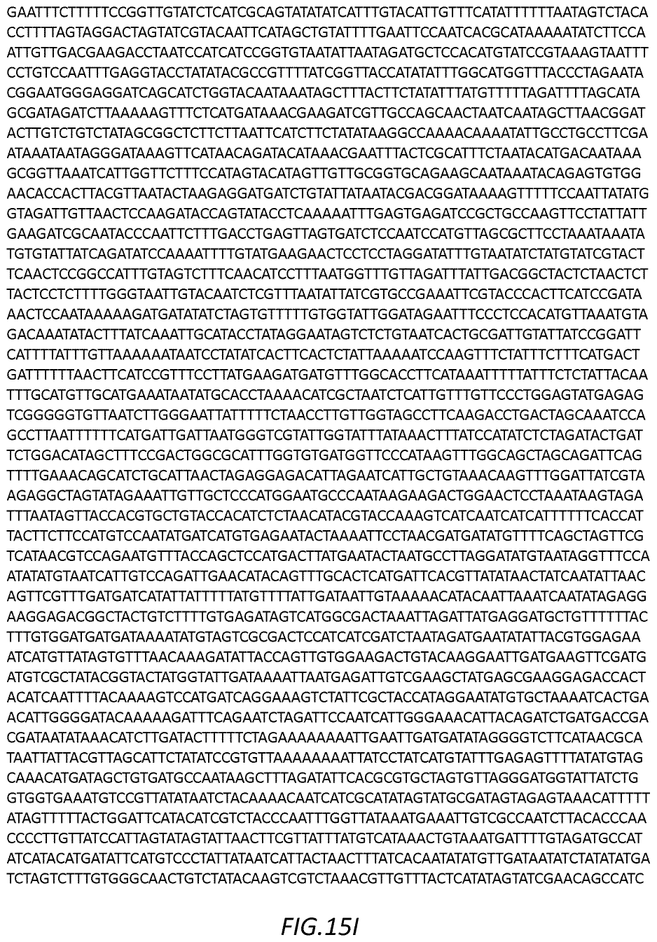

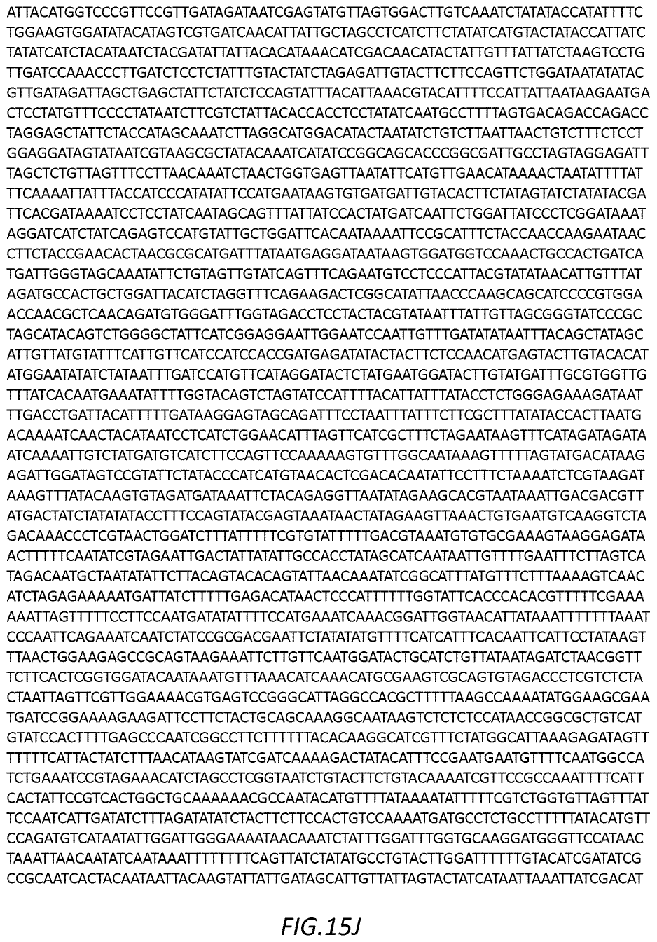

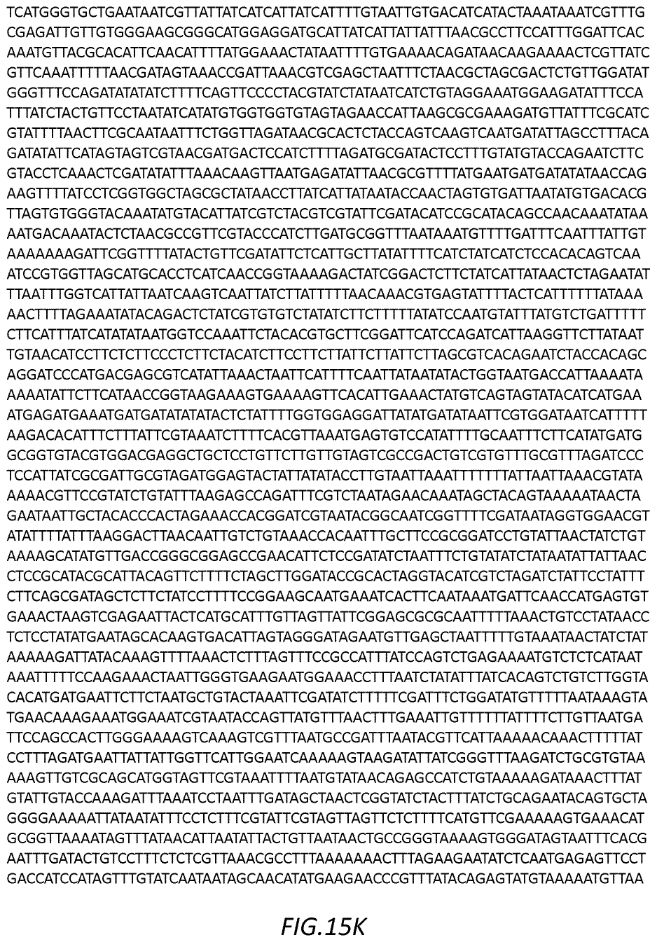

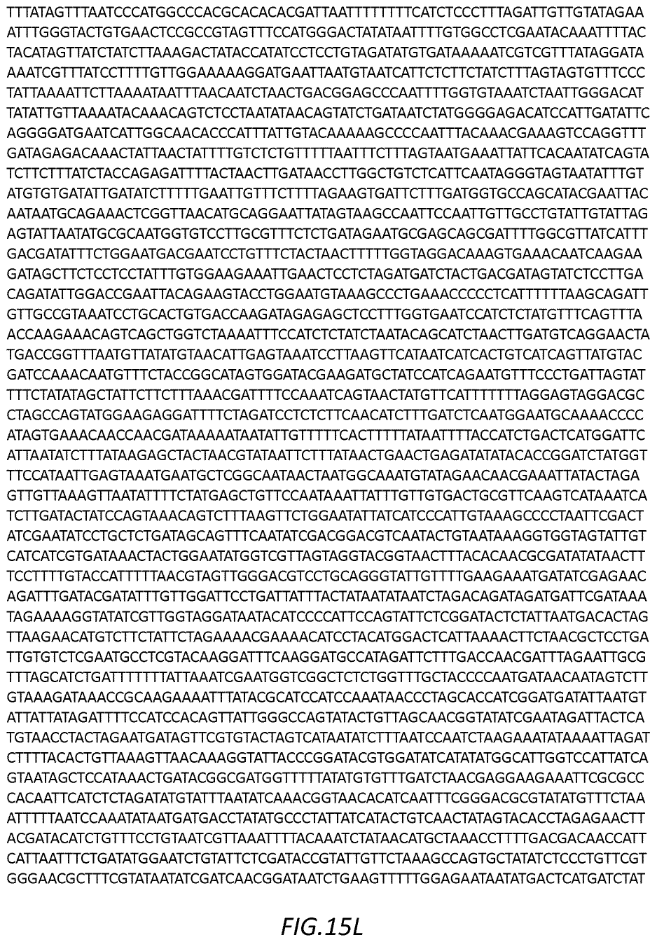

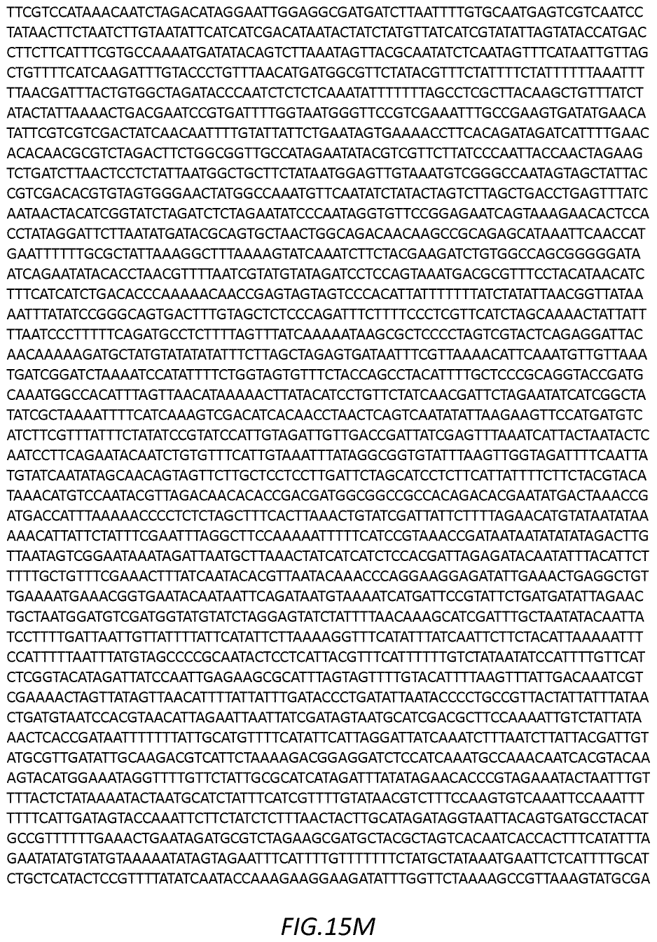

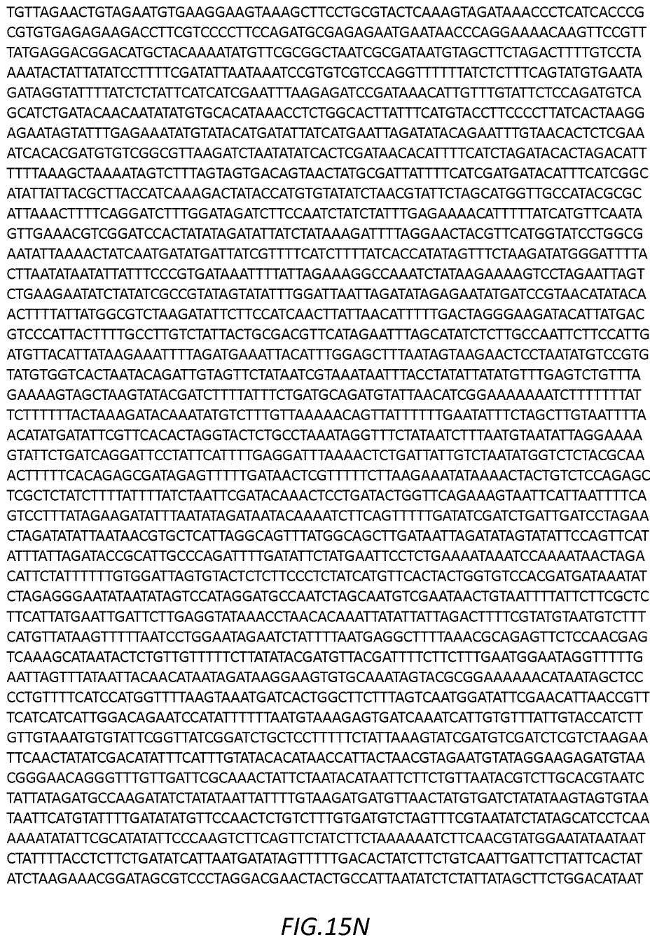

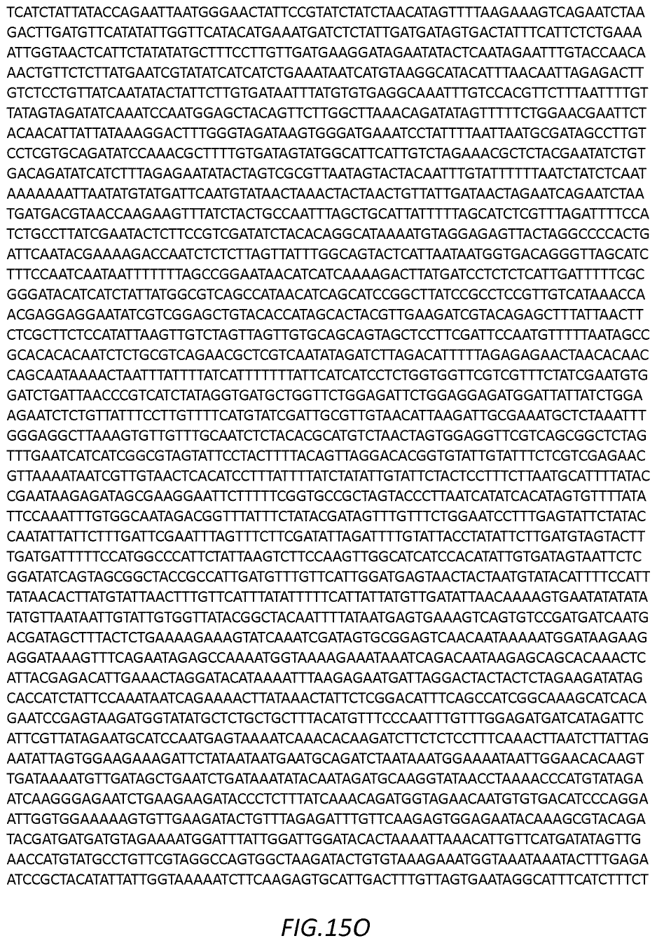

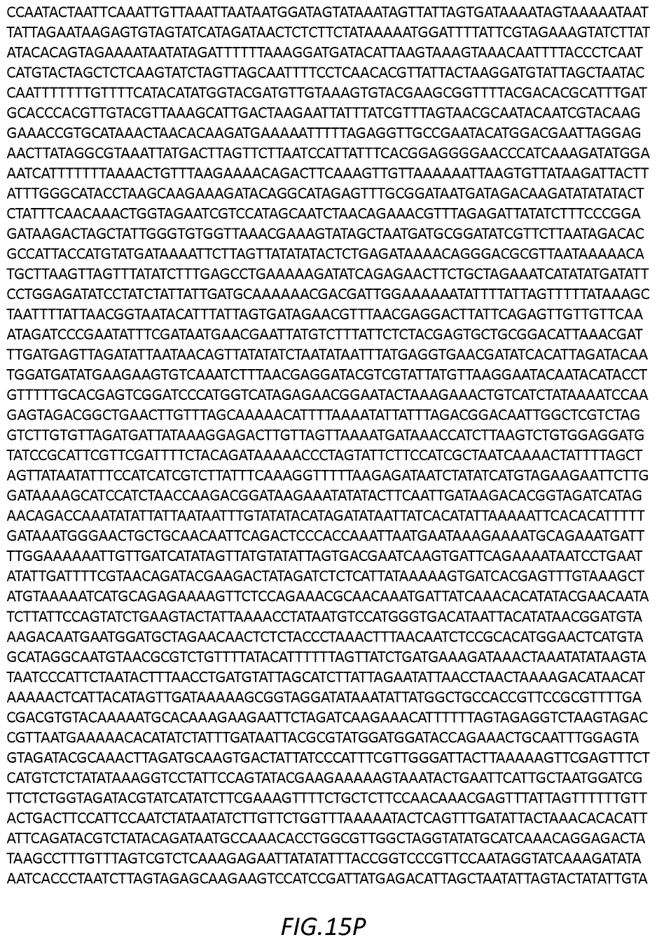

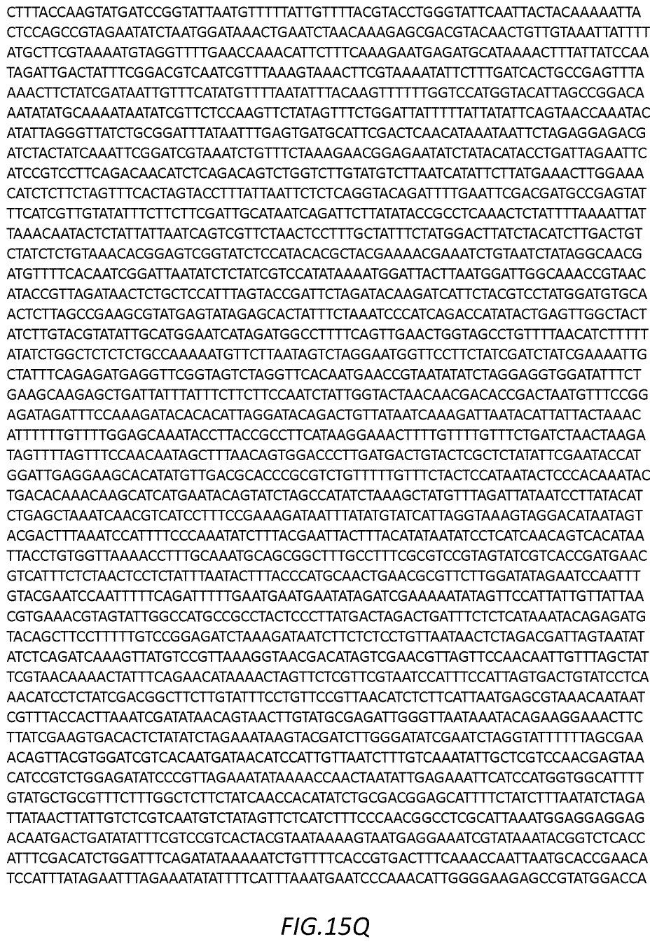

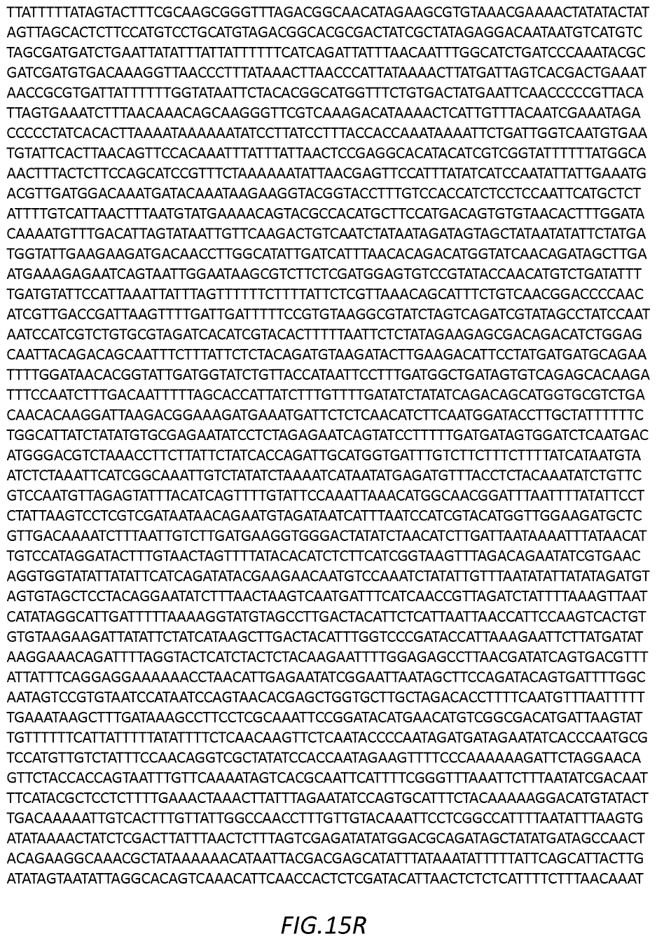

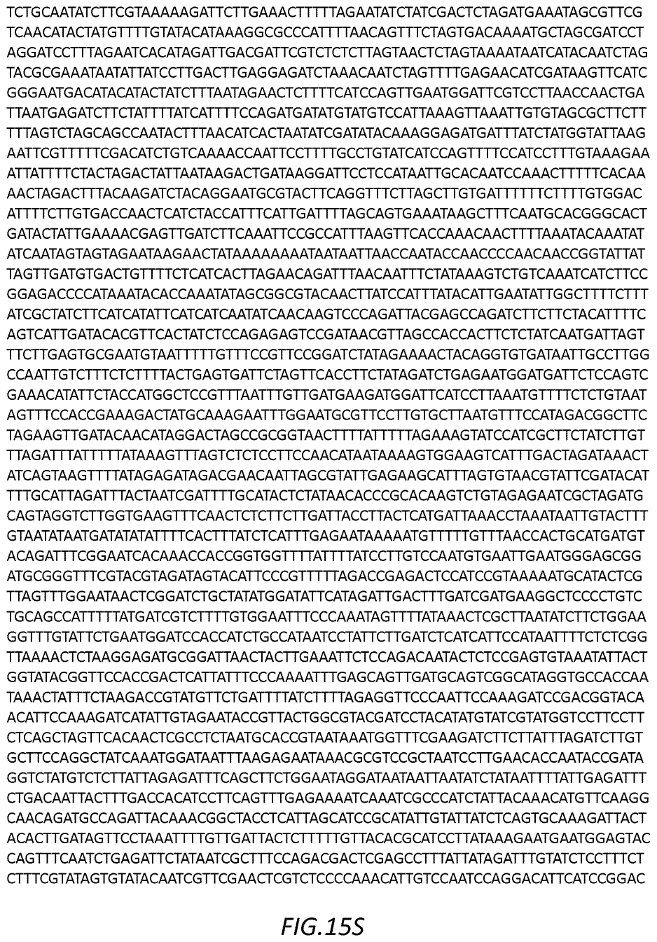

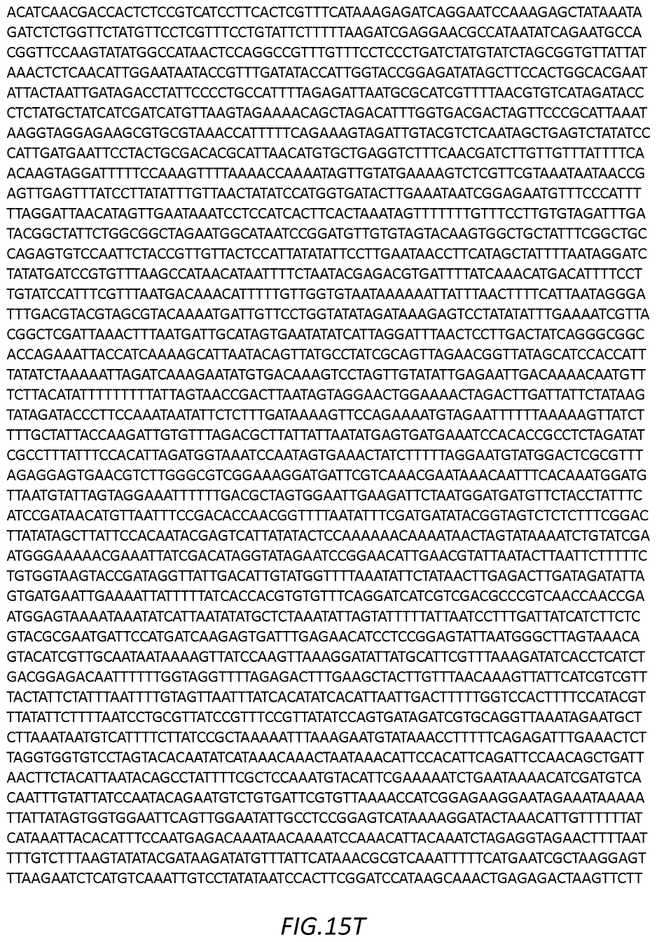

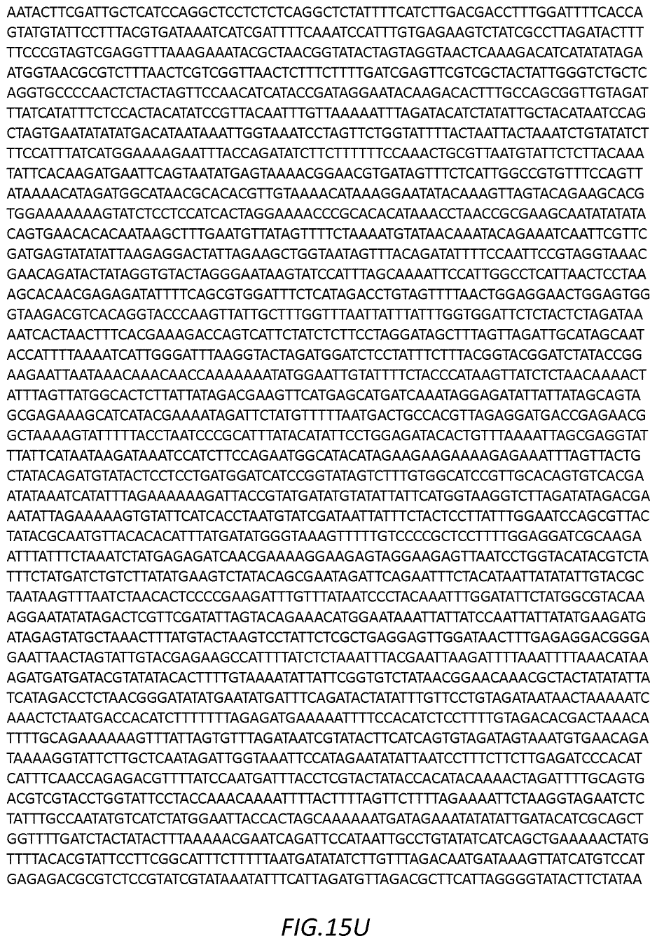

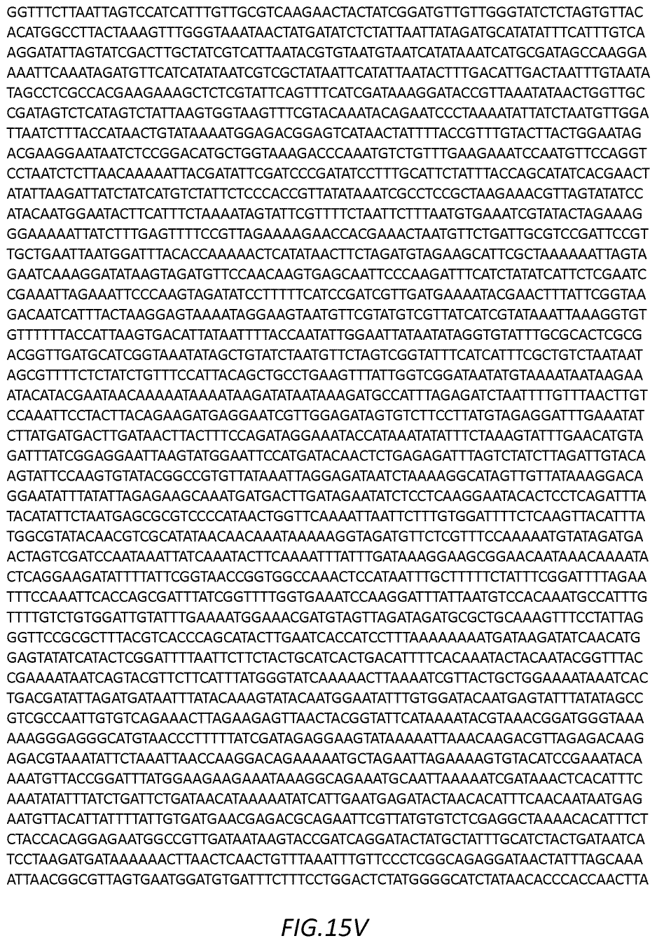

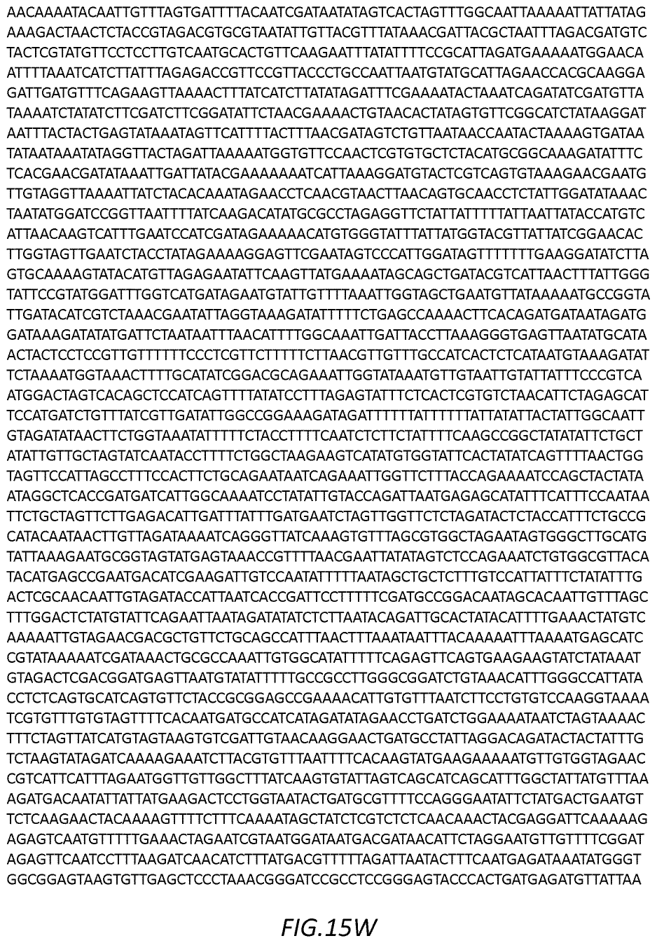

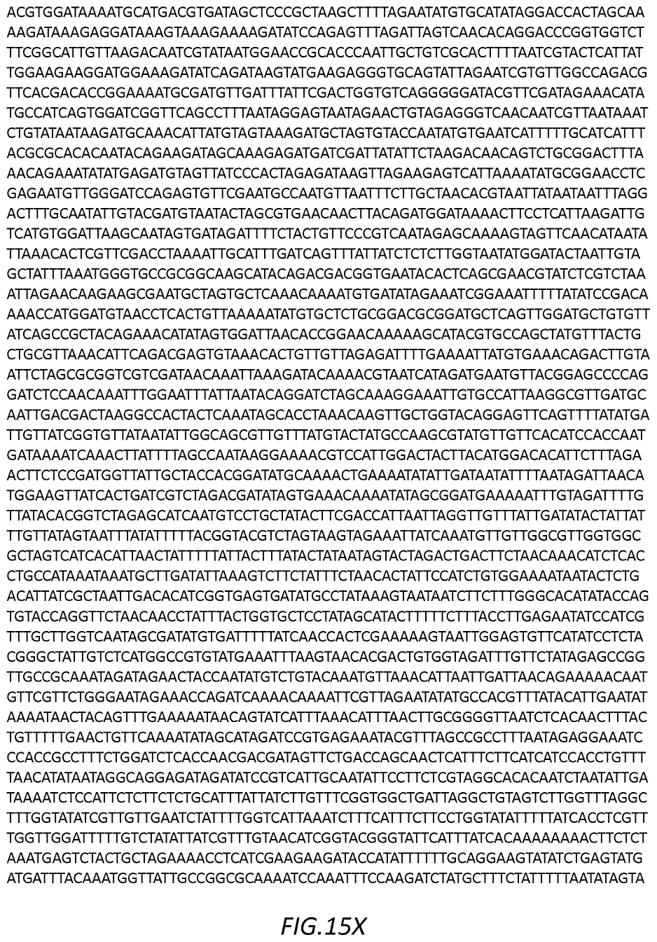

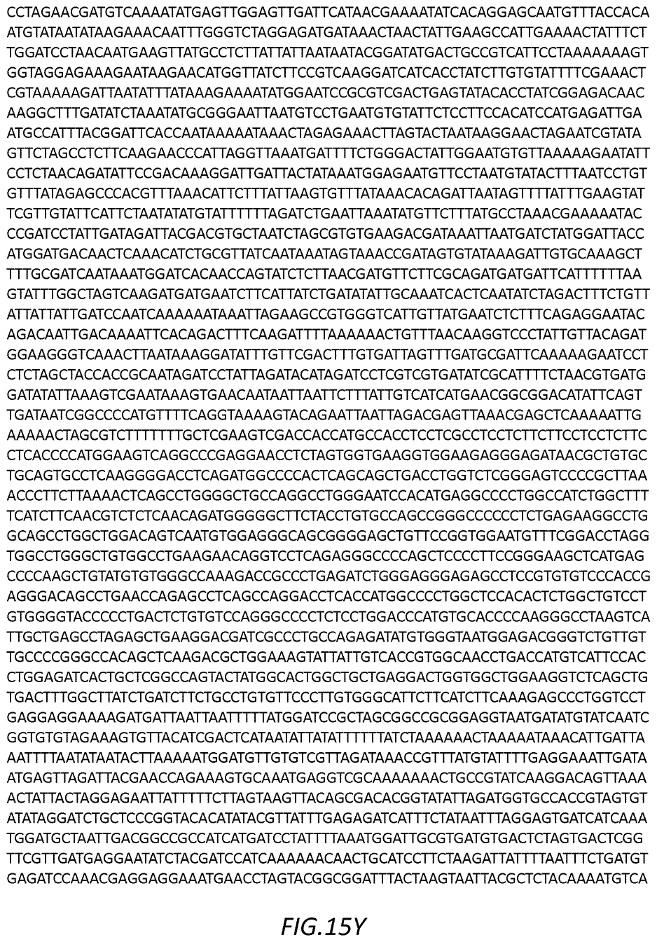

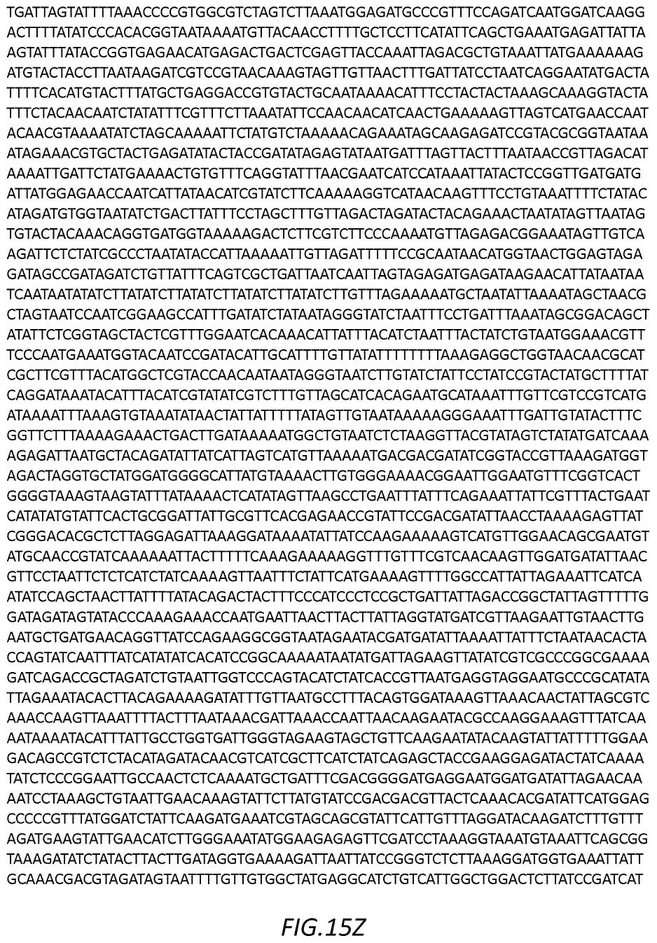

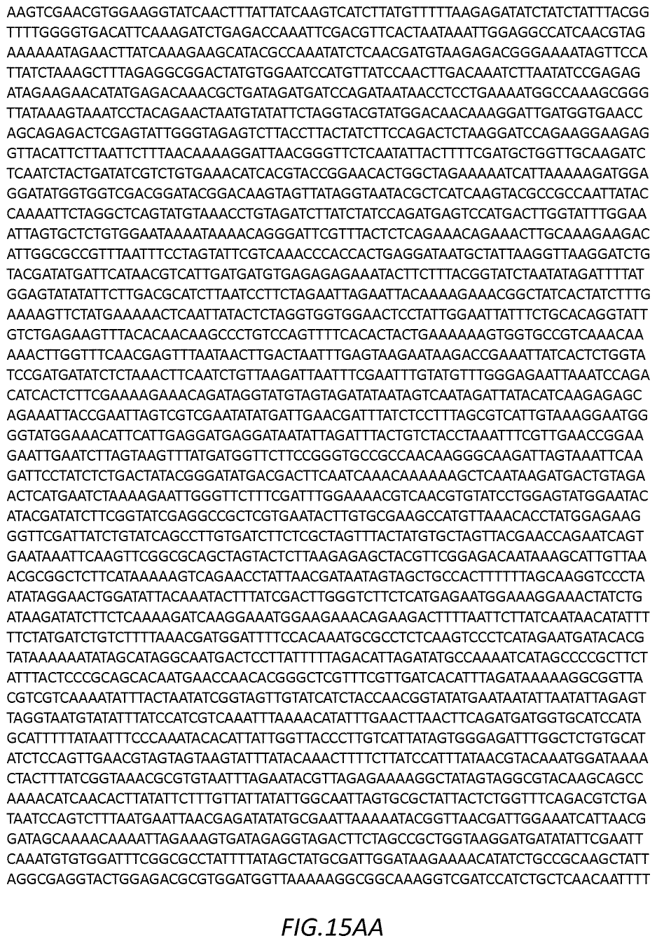

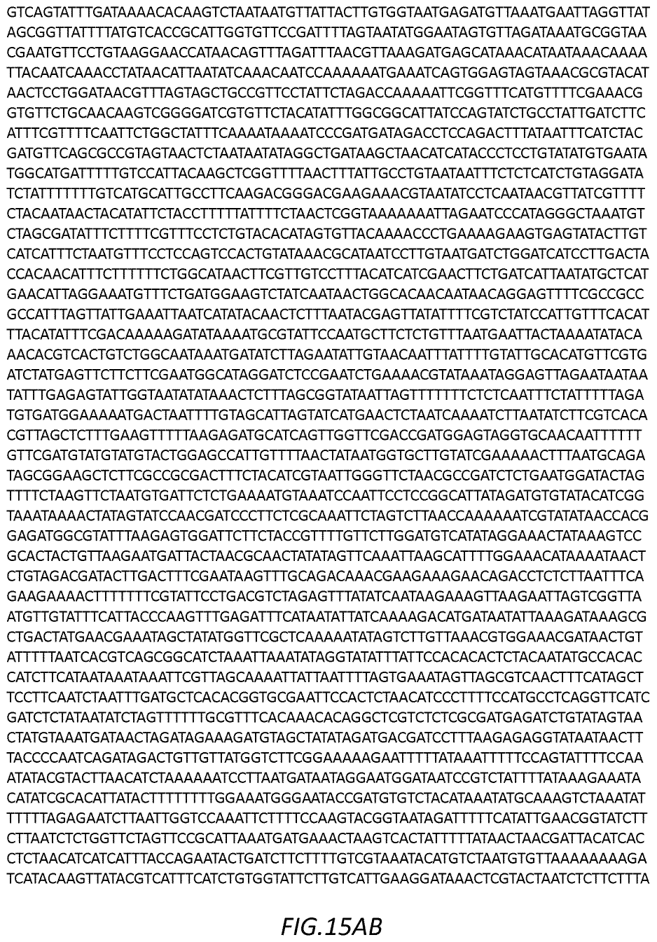

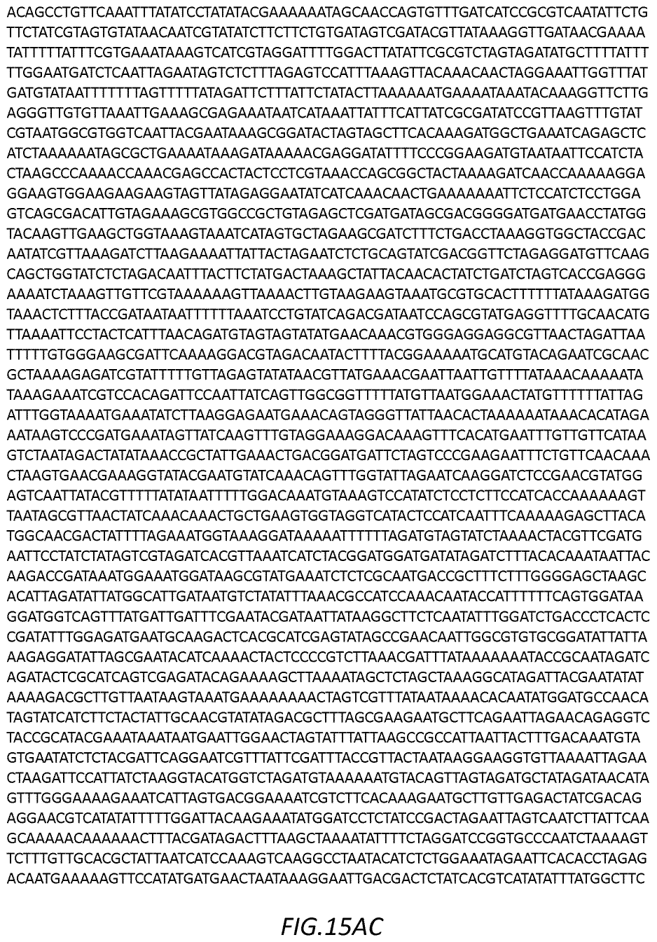

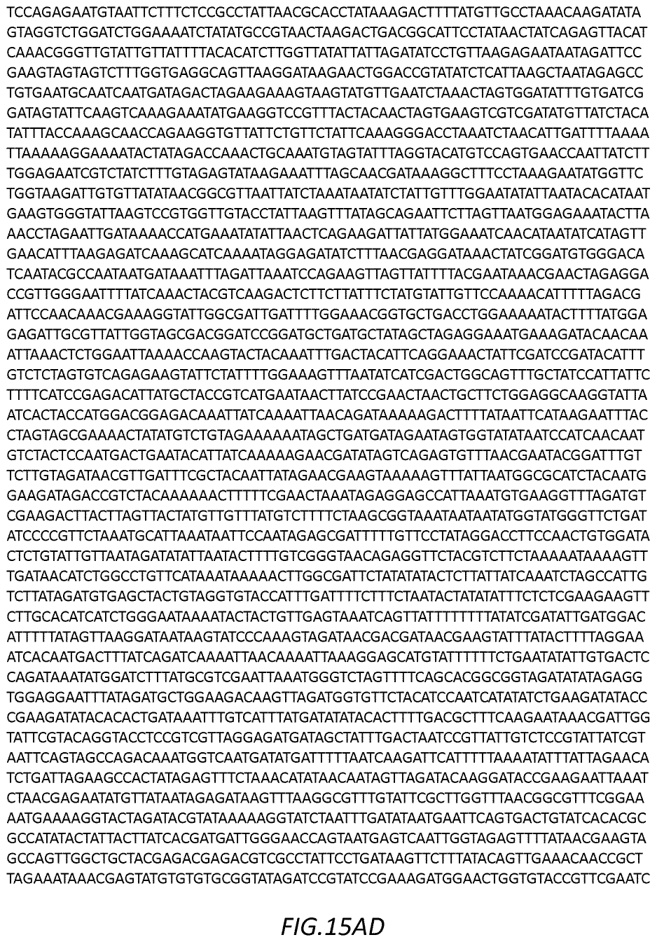

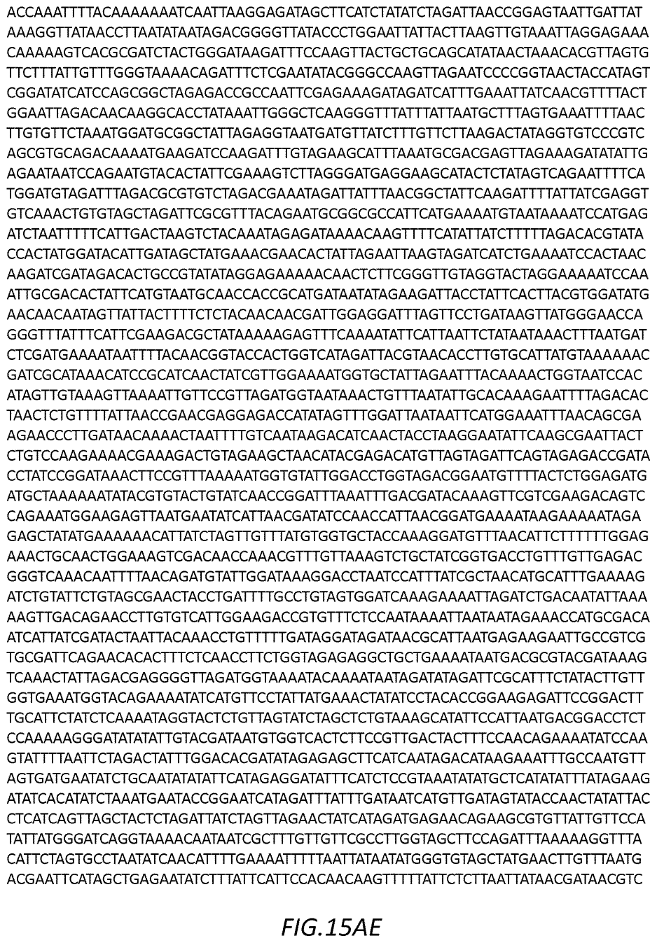

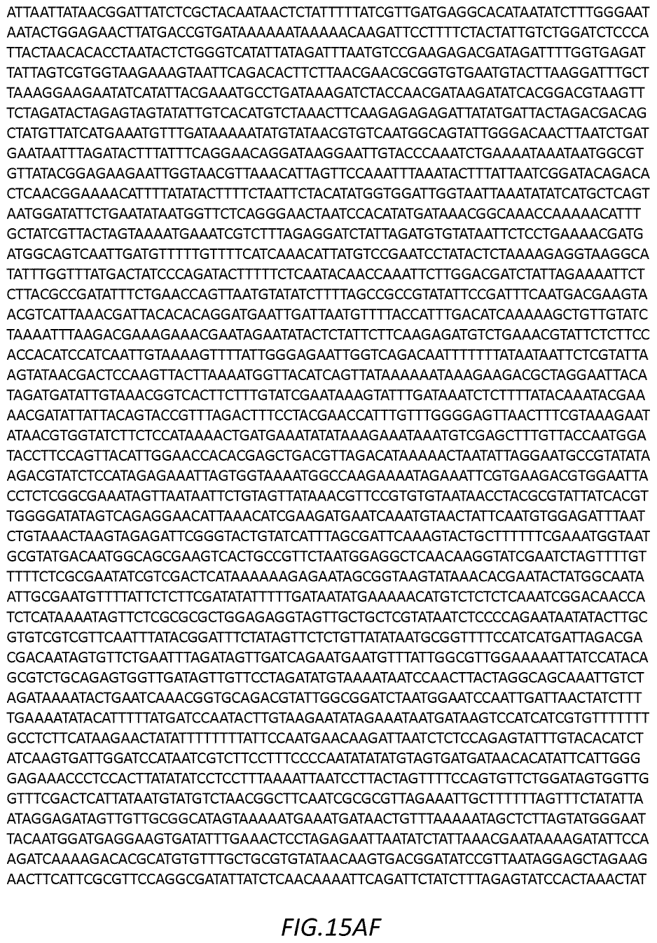

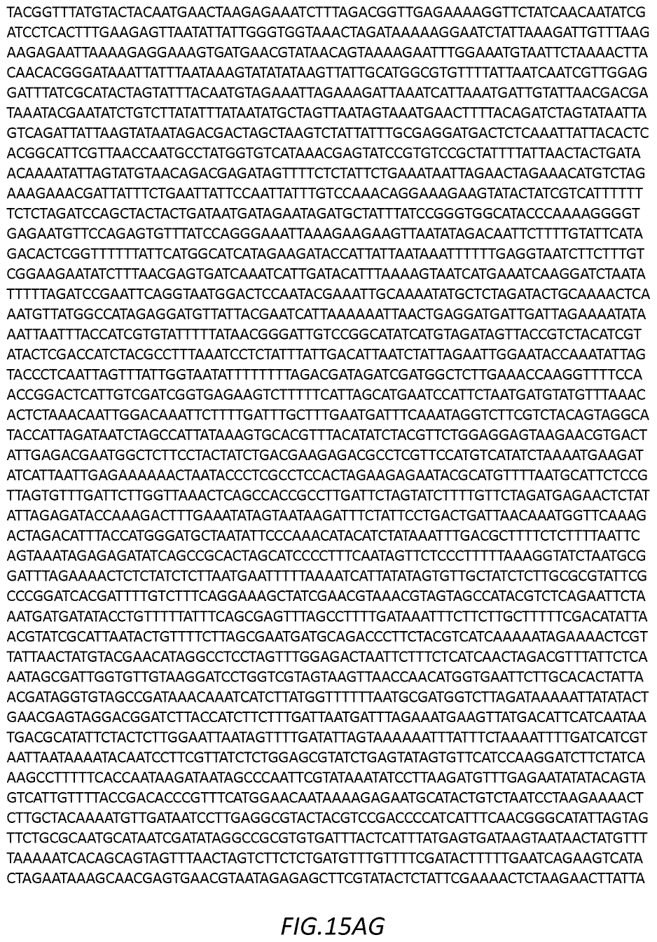

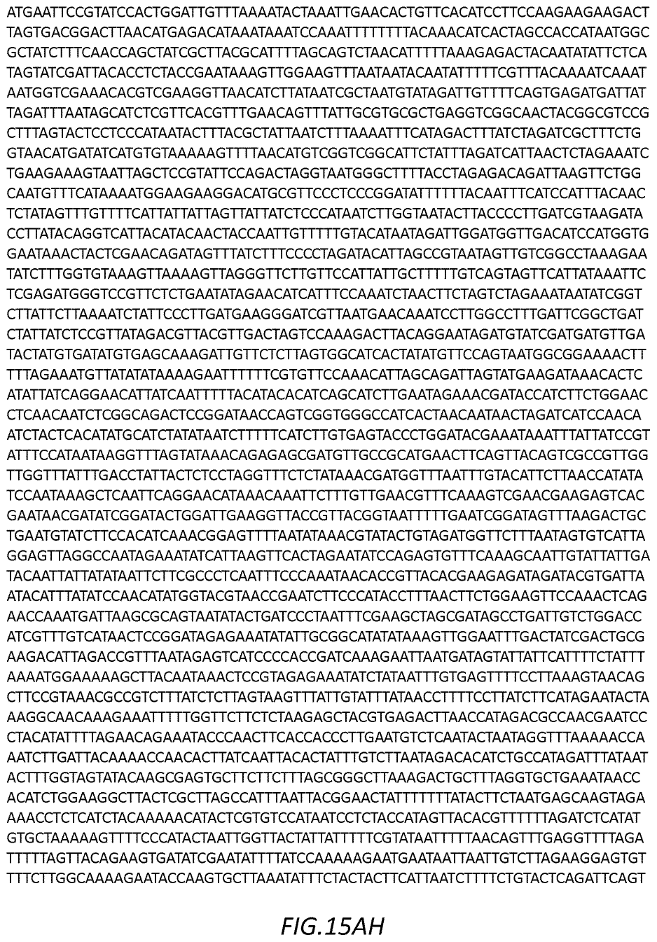

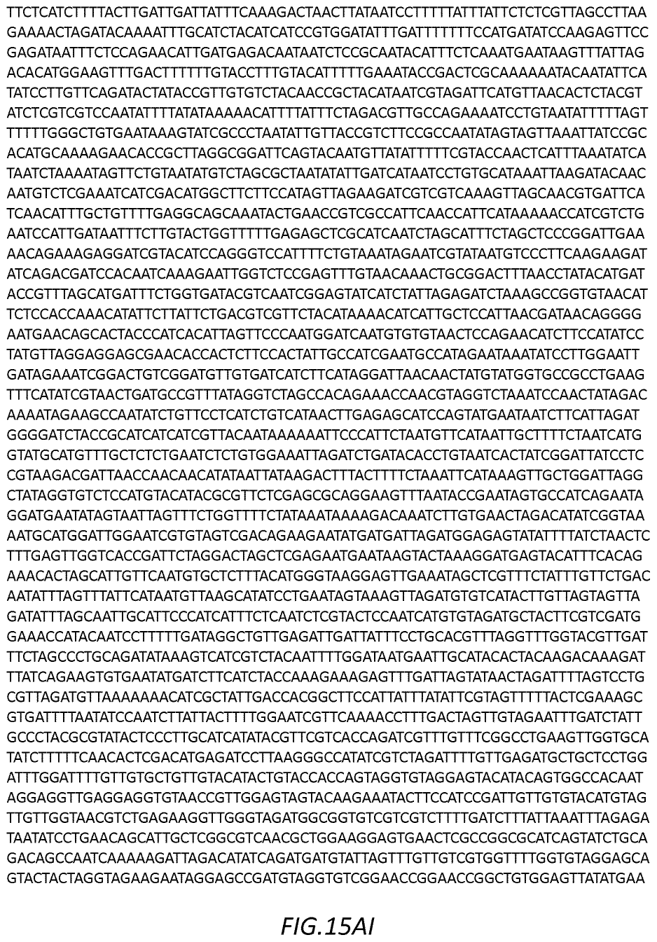

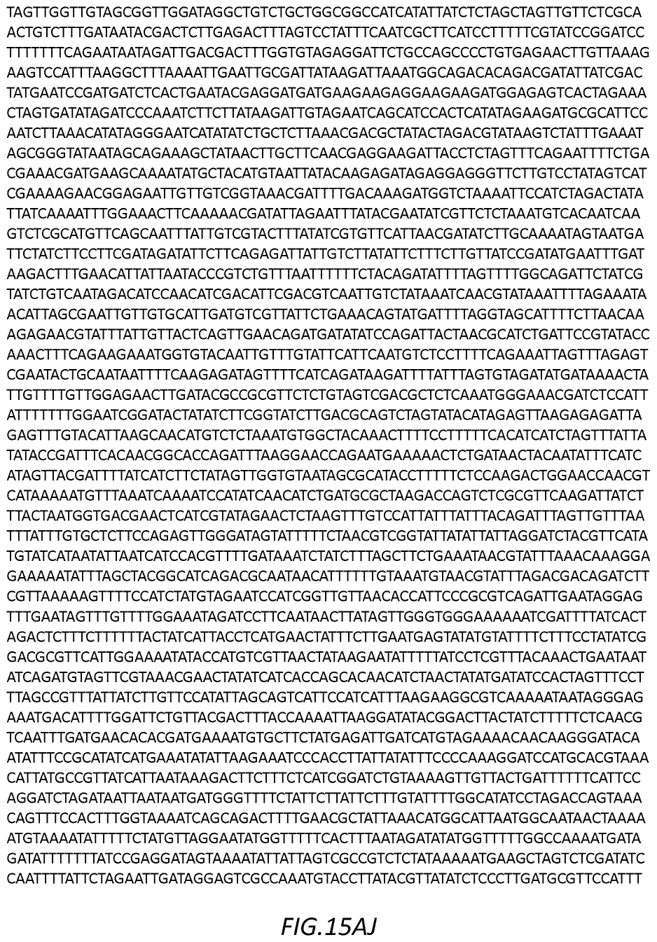

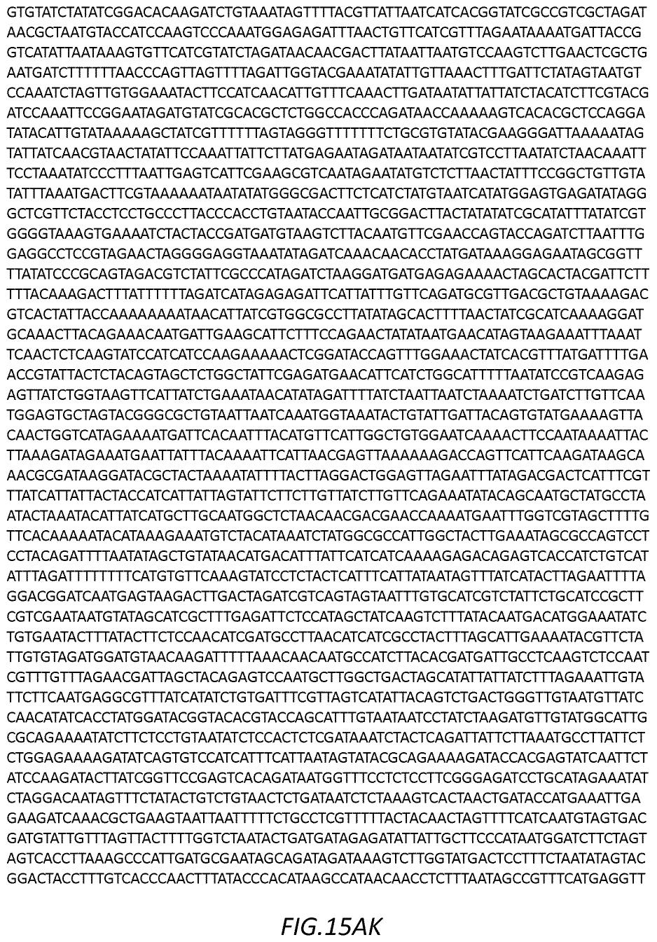

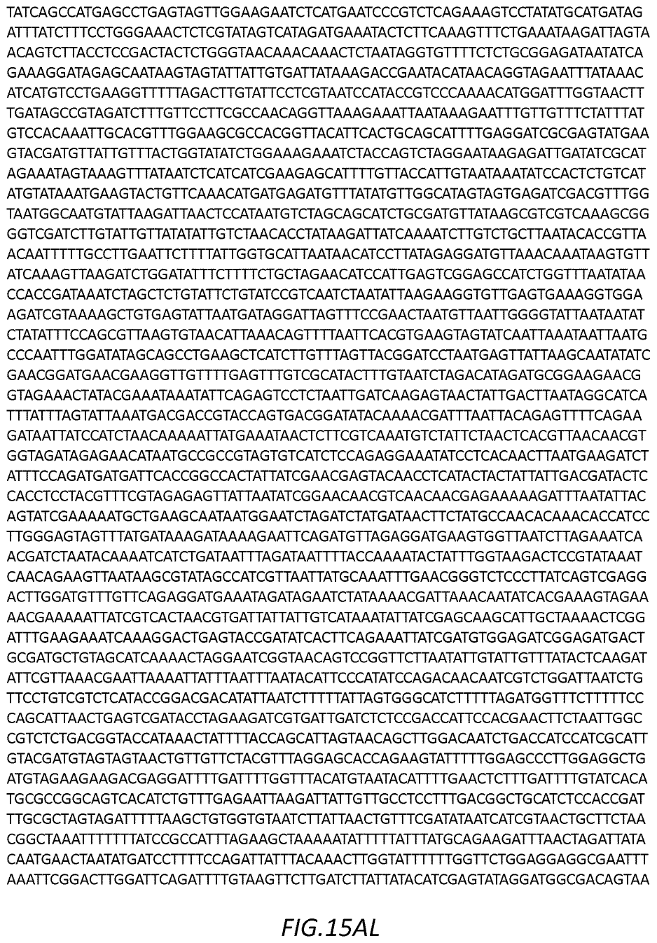

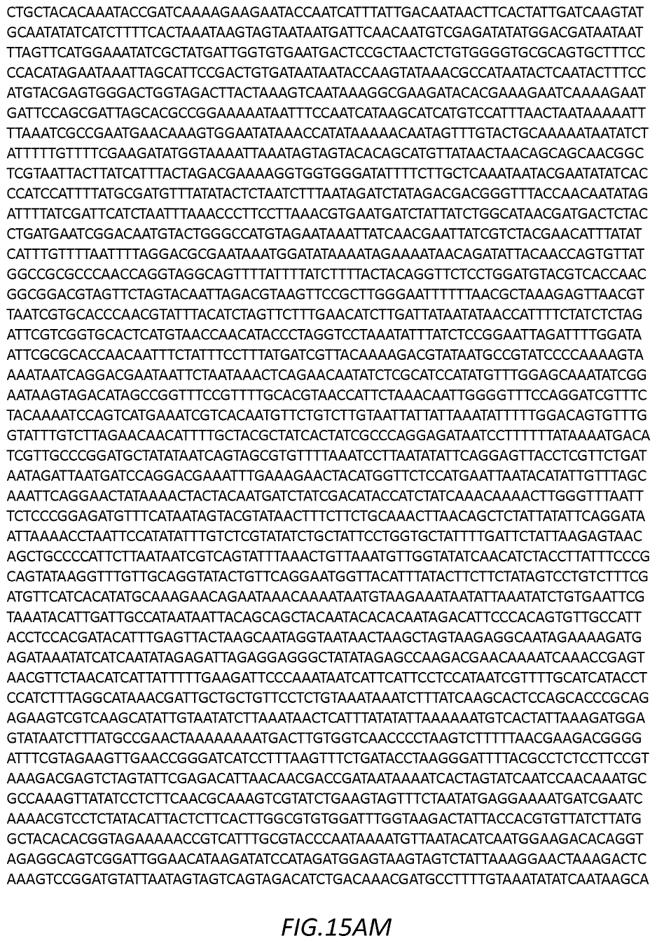

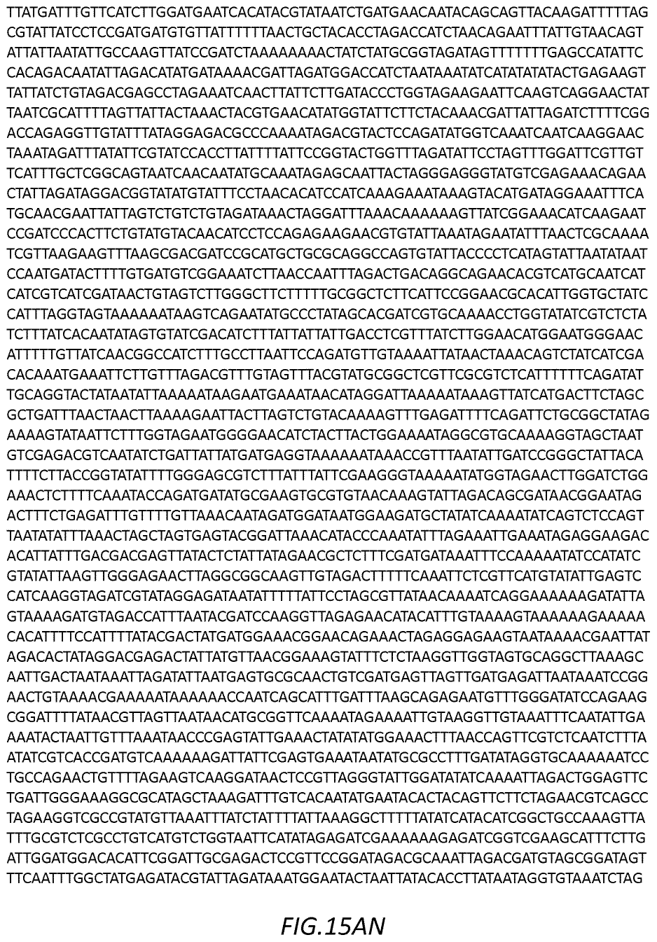

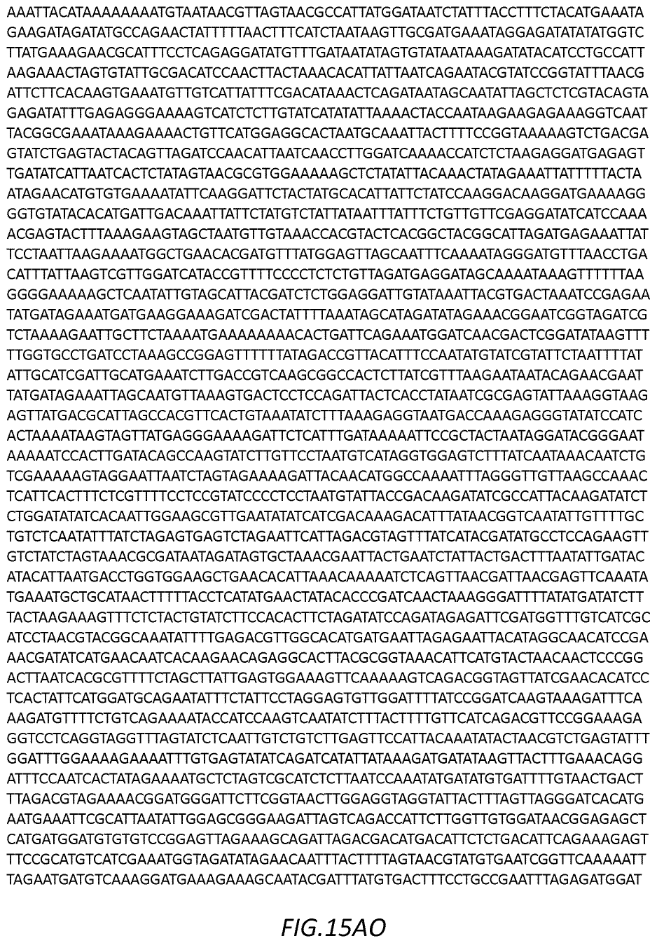

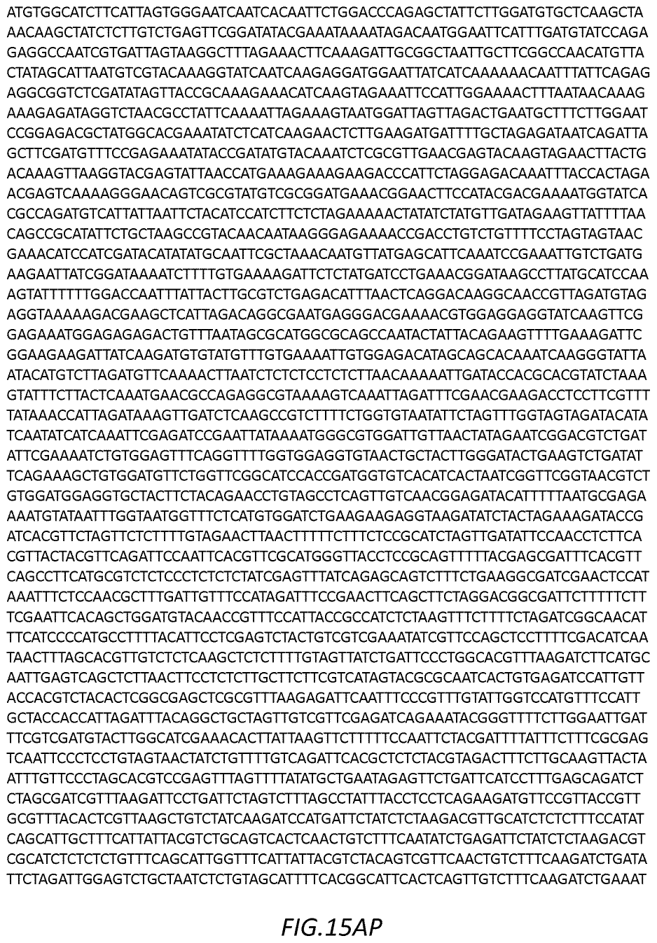

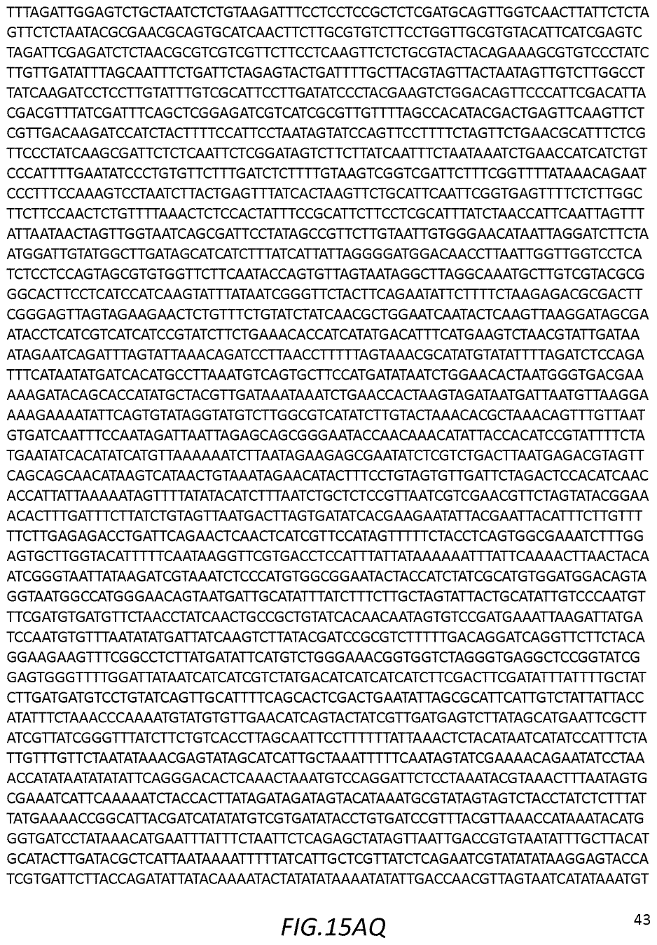

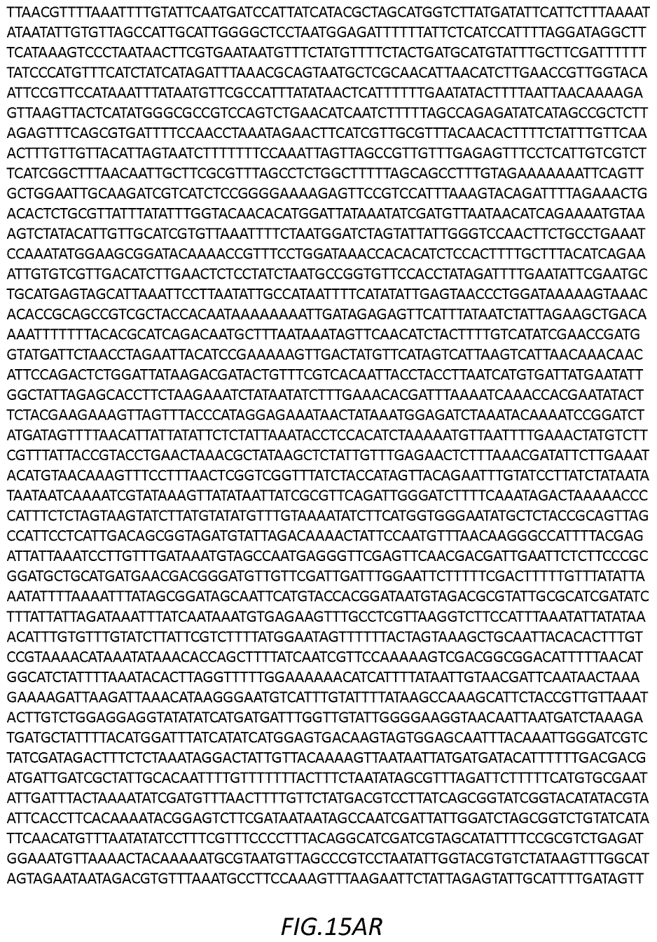

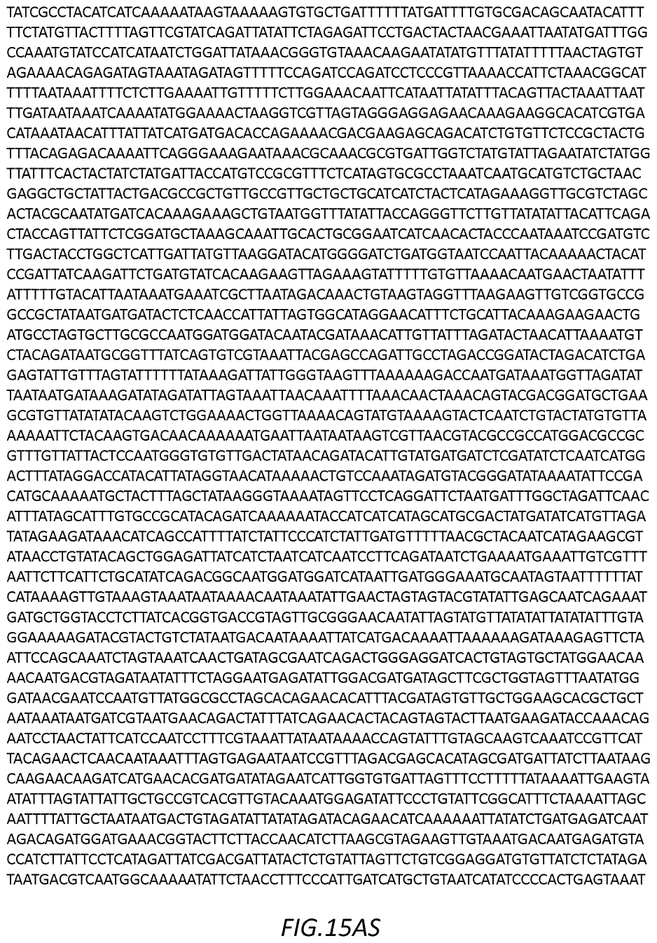

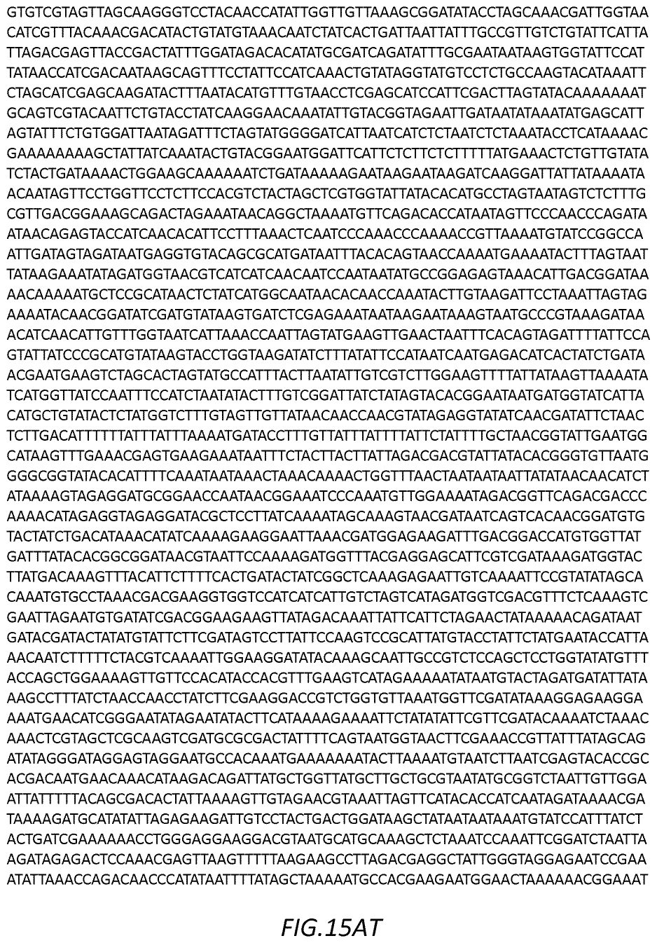

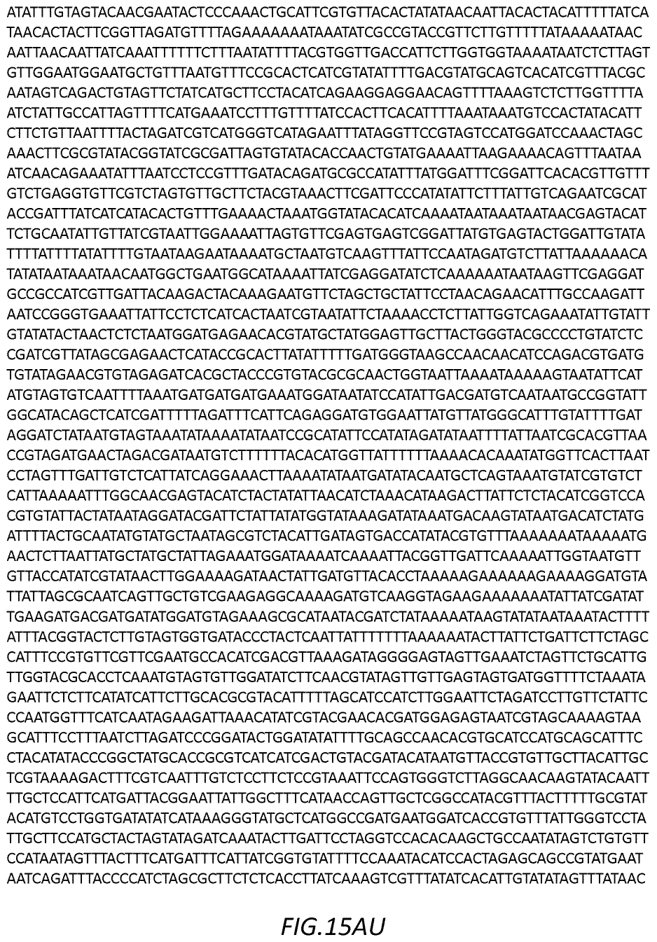

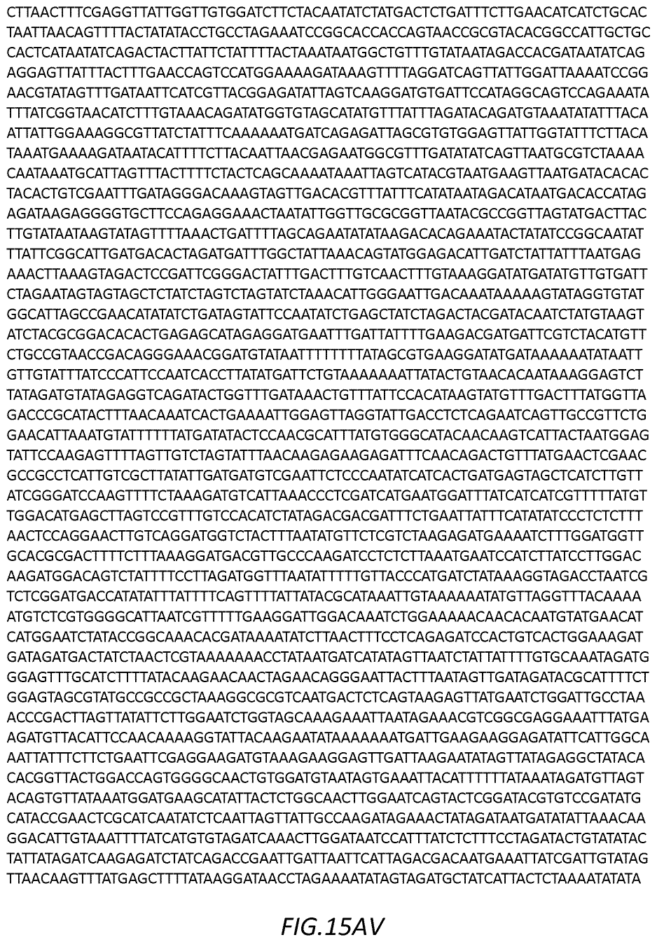

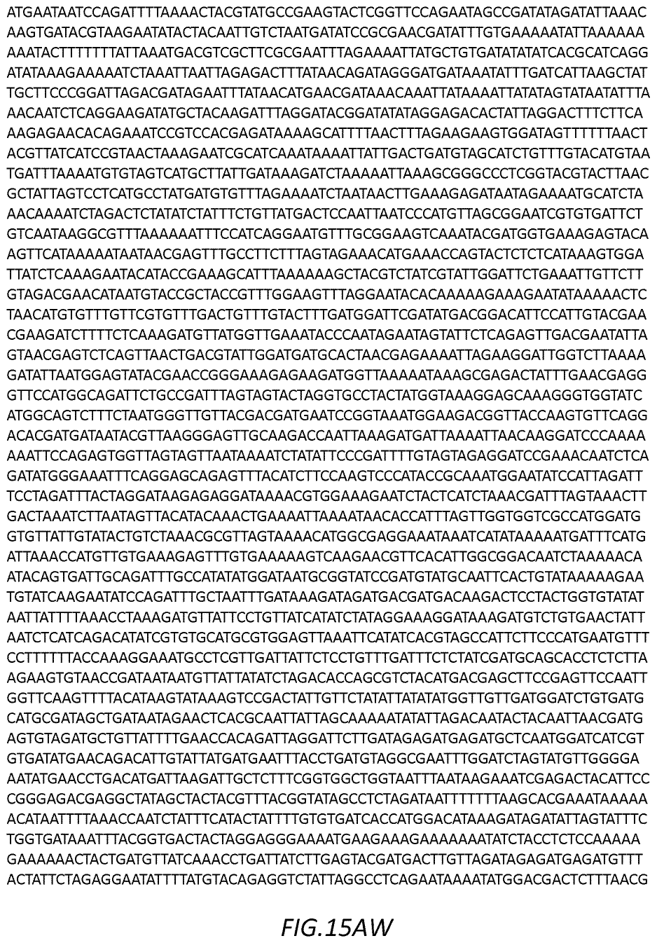

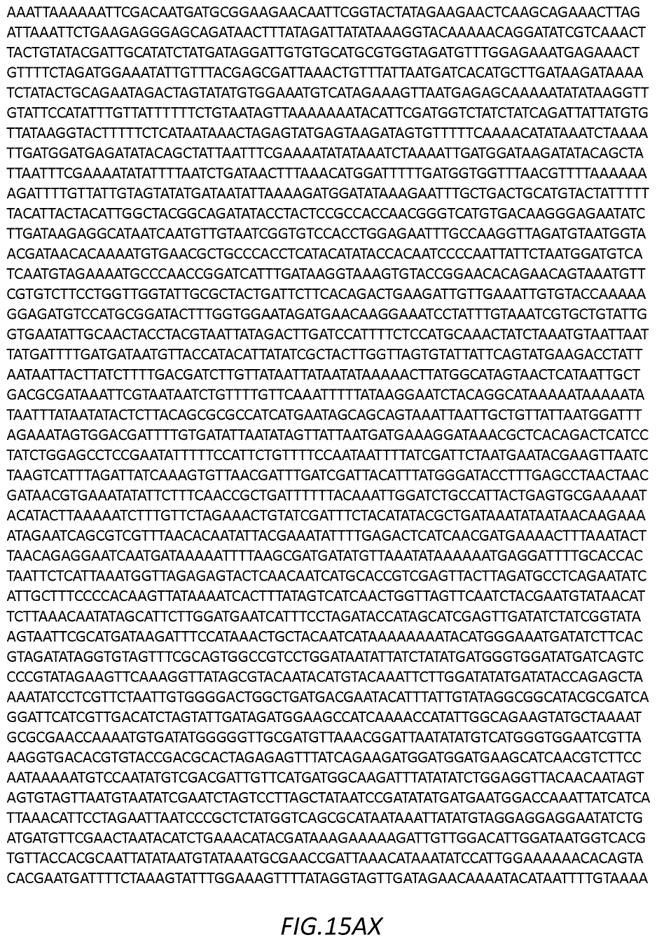

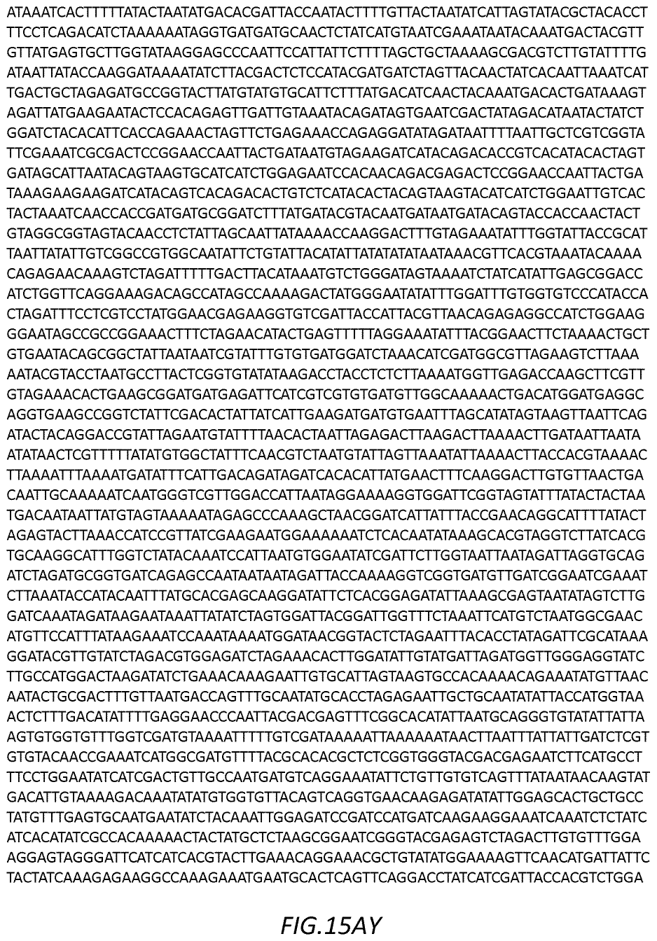

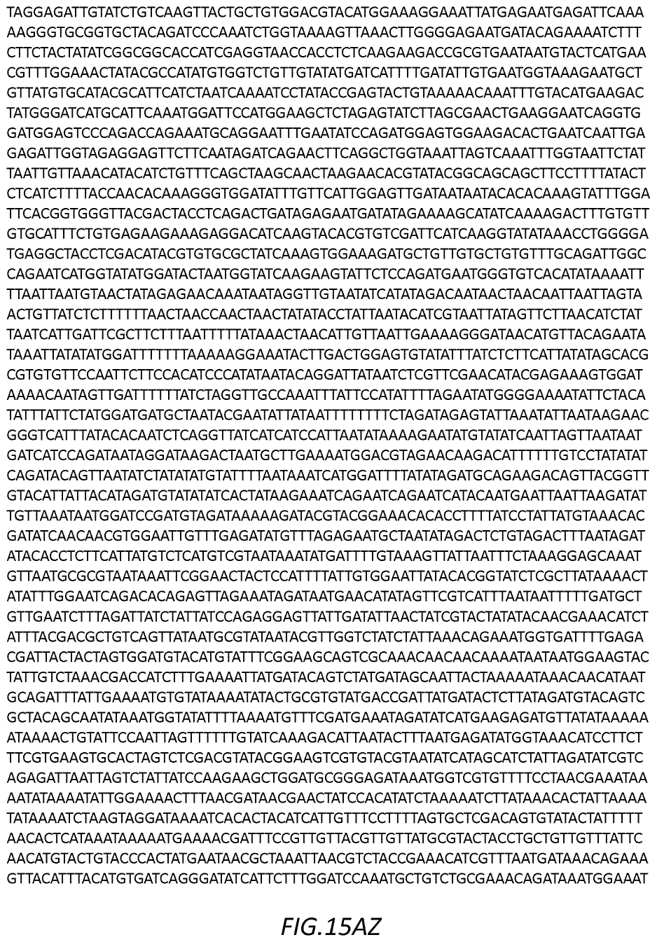

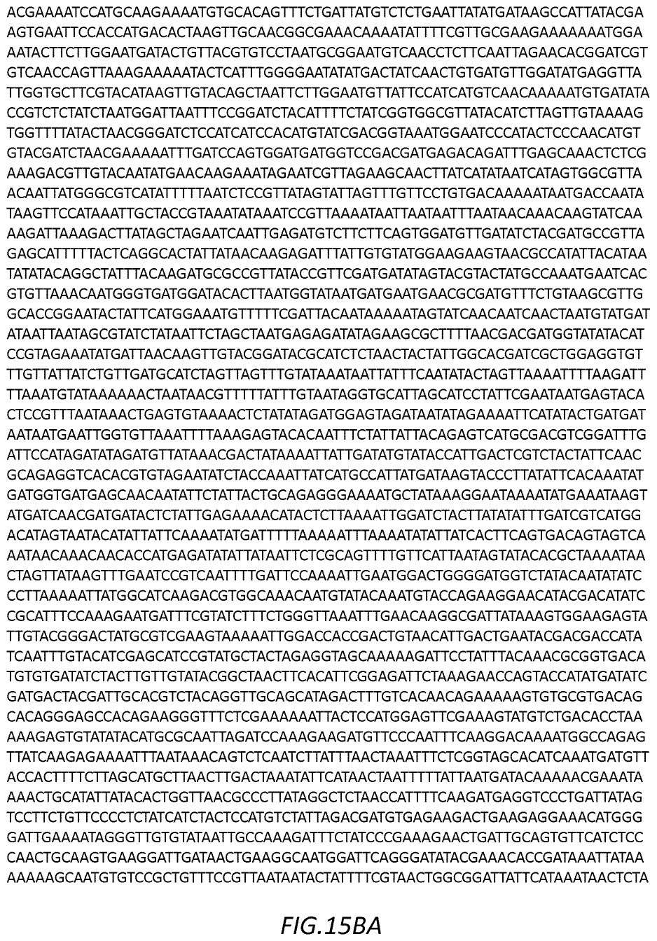

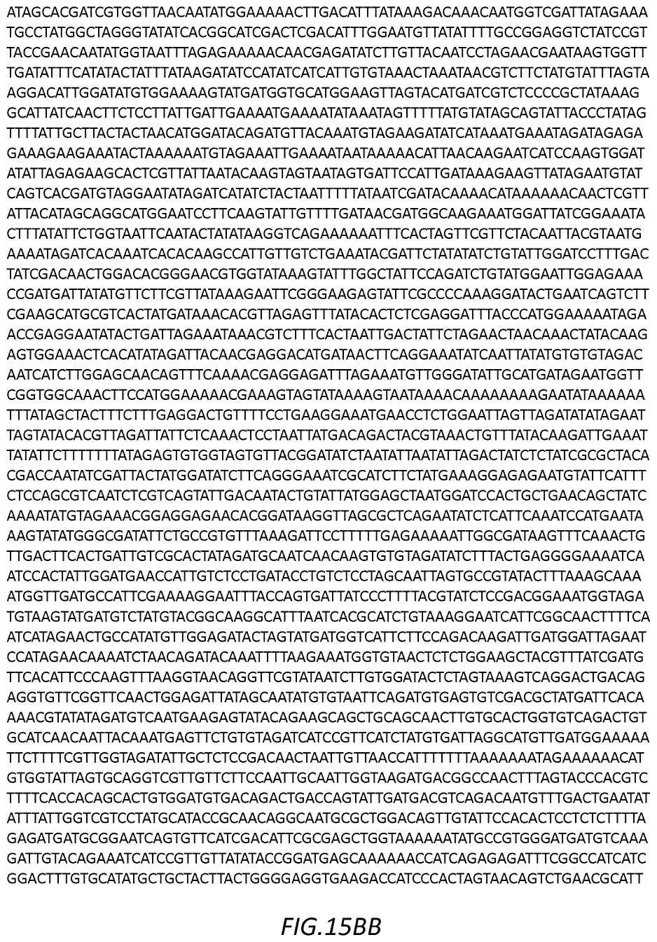

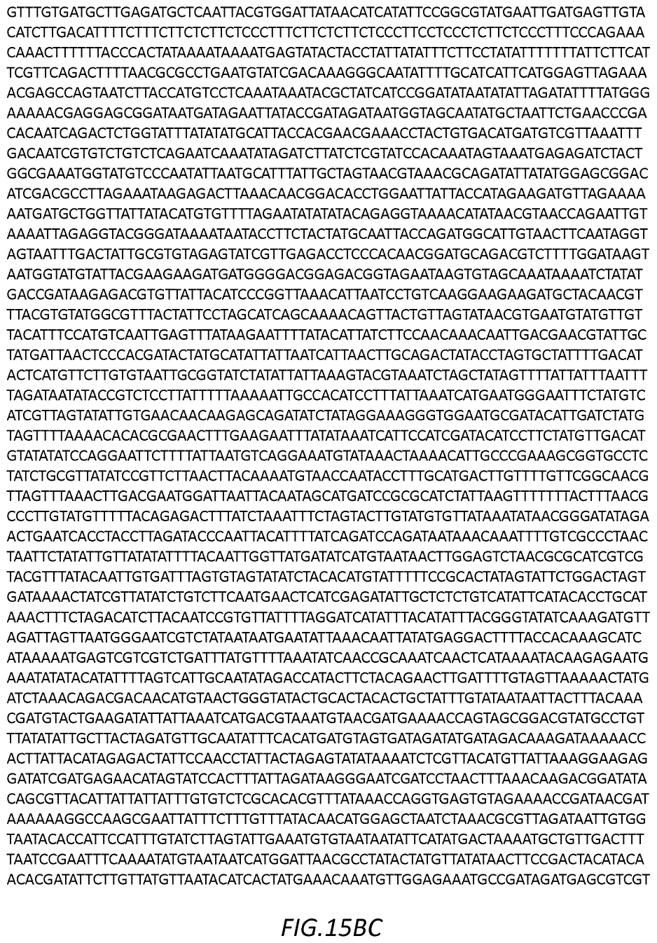

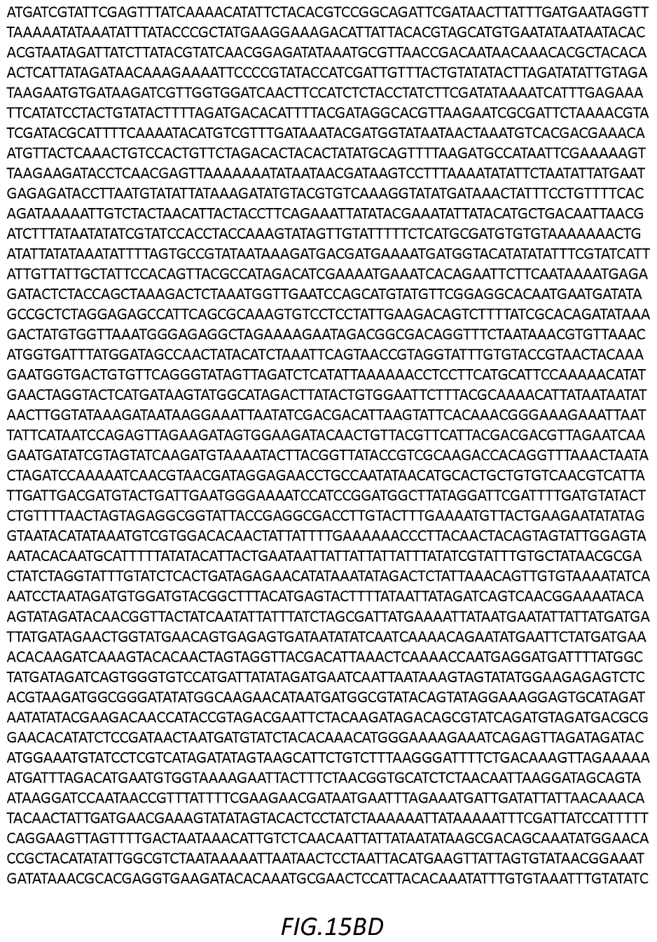

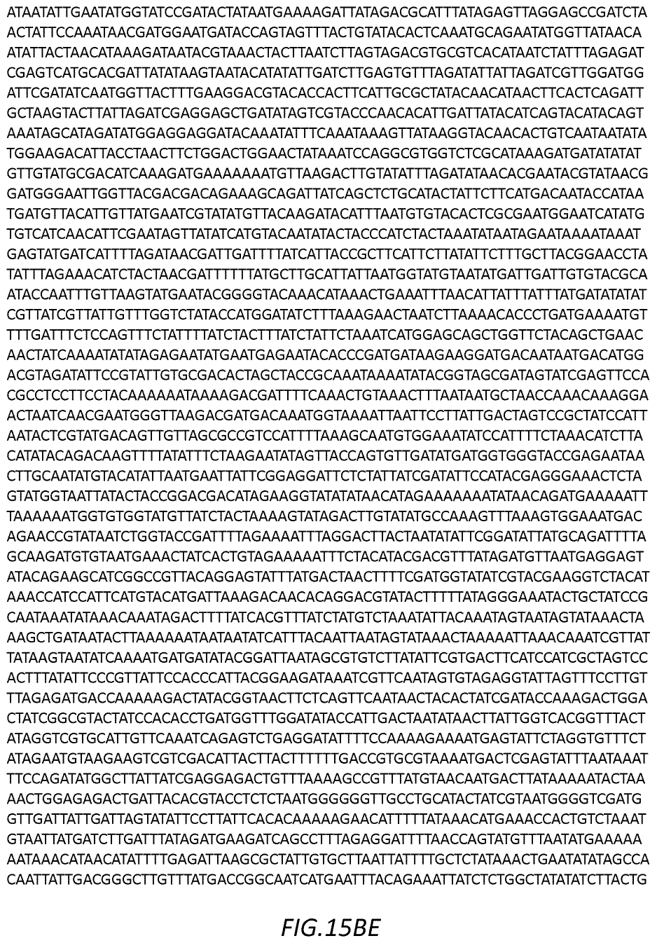

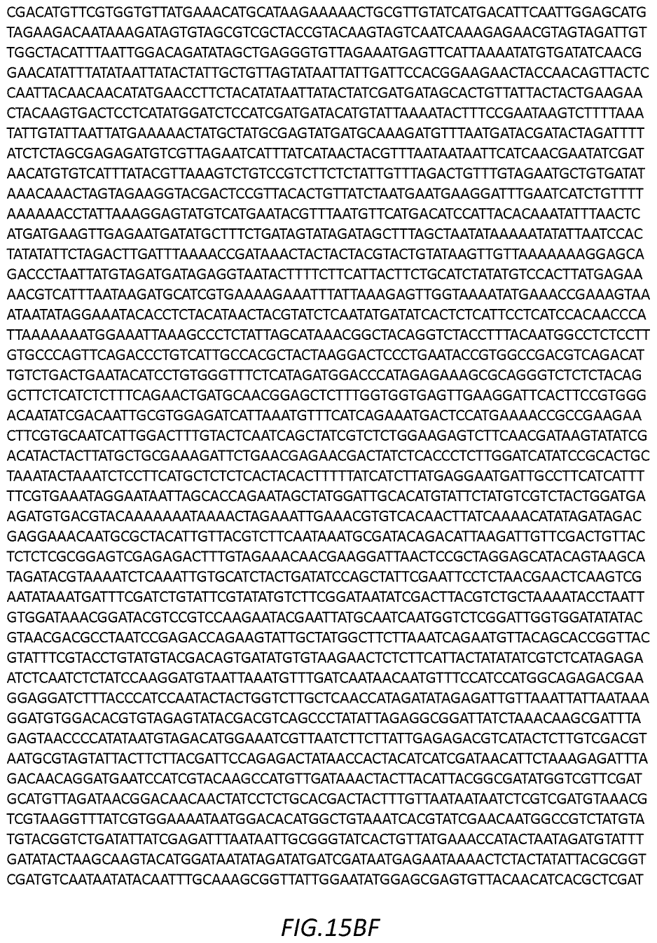

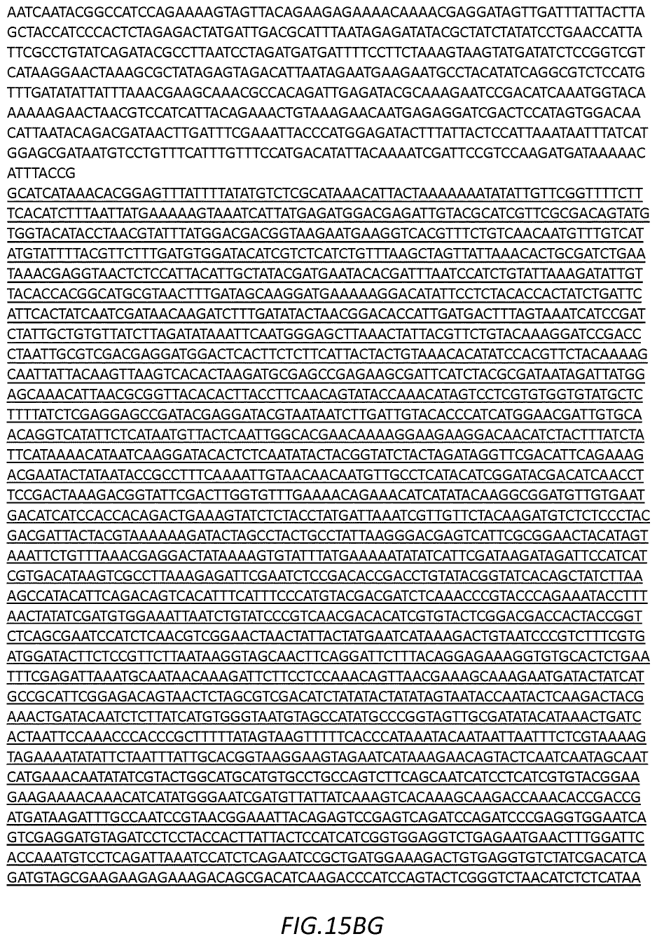

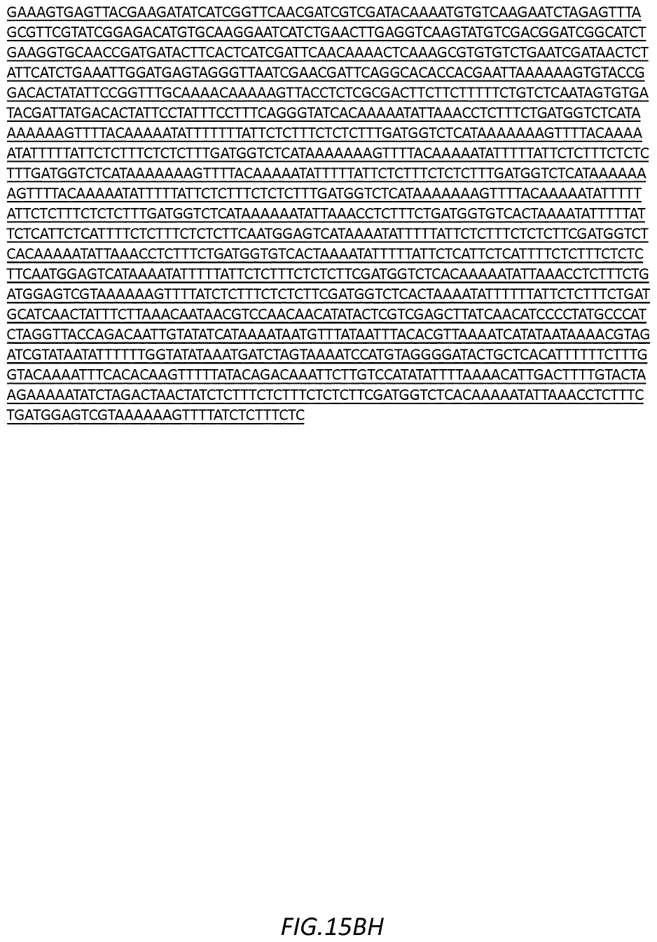

After 24 hours in culture with OV19t infected MDA-MD-468 tumor cells, we observed an increase in CD137 of preloaded non-targeting T cells when compared to non-targeting T cells with CD19-TCE added in culture and CD19-CAR T cells when accessed by flow cytometry (FIG. 4C). CD137 expression remained higher among pre-loaded T cells at 48 hours as well (FIG. 9A). We quantified T cell killing ability against MDA-MB-468 tumor cells infected with OV19t using flow cytometry. At 24 and 48 hours, we observed greater killing of non-targeting T cells with CD19-TCE in culture, CD19-TCE preloaded non-targeting T cells, and CD19-CAR T cells than OV19t with non-targeting T cells alone (FIGS. 4C and 9B). Furthermore, targeting of CD19t expression following OV19t infection by CD19-TCE was confirmed when comparing expression to co-cultured wells in the absence of CD19-TCE (FIGS. 4C and 9C). This data suggests that the presence of CD19-TCE, both pre-loaded onto T cells or suspended in culture with T cells, and CD19-CAR T cells elicit comparable anti-tumor activity against OV19t infected solid tumors in vitro.

### Example 5: Addressing Solid Tumor Antigen Heterogeneity by Combining HER2 Targeting CAR T Cells with CD19-TCE Against a Tumor Cancer Cell Line with Low Levels of HER2 Expression Following OV19t

To determine whether CD19-TCE in combination with OV19t could further improve the targeting of a CAR T cell onto a tumor with heterogenous expression patterns of an existing CAR antigen, we co-cultured MDA-MB-468 triple negative breast cancer cells with 80% of the cells belonging to the parental line and 20% of cells expressing the CAR targetable HER2 antigen (MDA-MB-468-HER2 was lentivirally transduced to stably express HER2). We then treated wells with varying MOIs of OV19t and added HER2 CAR T cells with or without CD19-TCE. These cells were co-cultured for 1-6 days, which were rechallenged as shown in FIG. 4D. At 24 h, OV19t and HER2 CAR T cells showed comparable killing to OV19t with HER2 CAR T cells and CD19-TCE (FIG. 10A). At 48h, CD19-TCE in combination with HER2 CAR T cells demonstrated slightly higher killing, but similar activation and CD19t targeting (FIG. 10B). When wells were rechallenged with antigen negative MDA-MB-468 tumor cells (not expressing HER2), the combinatorial treatment of OV19t, HER2 CAR T cells, and CD19-TCE showed additive and synergistic tumor cell killing when compared to OV19t and HER2 CAR T cells alone. Additionally, both non-targeting T cells with CD19-TCE (FIG. 4D). Wells were then rechallenged once more with antigen negative MDA-MB-468 tumor cells and again showed improved tumor cell killing when a combinatorial approach using OV19t, HER2 CAR T cells, and CD19-TCE. We demonstrated greater tumor cell killing, CD19t targeting, and T cell count at the lowest MOI (0.003125) (FIG. 4E). This data suggests that using OV19t to deliver a CD19-TCE-targetable antigen can be combined with existing CAR T cells to elicit dual-targeting of heterogenous tumors.

### REFERENCES

1. Huehls, A.M., T.A. Coupet, and C.L. Sentman, Bispecific T-cell engagers for cancer immunotherapy. Immunol Cell Biol, 2015. 93 (3): p. 290-6.
2. Goebeler, M. E. and R. C. Bargou, T cell-engaging therapies-BiTEs and beyond. Nat Rev Clin Oncol, 2020. 17 (7): p. 418-434.
3. Kantarjian, H., et al., Blinatumomab versus Chemotherapy for Advanced Acute Lymphoblastic Leukemia. N Engl J Med, 2017. 376 (9): p. 836-847.
4. Bargou, R., et al., Tumor regression in cancer patients by very low doses of a T cell-engaging antibody. Science, 2008. 321 (5891): p. 974-7.
5. Stern, L. A., V. D. Jonsson, and S. J. Priceman, CAR T Cell Therapy Progress and Challenges for Solid Tumors. Cancer Treat Res, 2020. 180: p. 297-326.
6. Hamieh, M., et al., Programming CAR T Cell Tumor Recognition: Tuned Antigen Sensing and Logic Gating. Cancer Discov, 2023. 13 (4): p. 829-843.
7. Sterner, R. C. and R. M. Sterner, CAR-T cell therapy: current limitations and potential strategies. Blood Cancer J, 2021. 11 (4): p. 69.
8. Chen, N., et al., Driving CARs on the uneven road of antigen heterogeneity in solid tumors. Curr Opin Immunol, 2018. 51: p. 103-110.
9. Murad, J. P., et al., Pre-conditioning modifies the TME to enhance solid tumor CAR T cell efficacy and endogenous protective immunity. Mol Ther, 2021. 29 (7): p. 2335-2349.
10. Bonaventura, P., et al., Cold Tumors: A Therapeutic Challenge for Immunotherapy. Front Immunol, 2019. 10: p. 168.
11. Young, R. M., et al., Next-Generation CAR T-cell Therapies. Cancer Discov, 2022. 12 (7): p. 1625-1633.
12. Kaufman, H. L., F. J. Kohlhapp, and A. Zloza, Oncolytic viruses: a new class of immunotherapy drugs. Nat Rev Drug Discov, 2015. 14 (9): p. 642-62.
13. Park, A. K., et al., Effective combination immunotherapy using oncolytic viruses to deliver CAR targets to solid tumors. Sci Transl Med, 2020. 12 (559).
14. Kim, S. I., et al., Recombinant Orthopoxvirus Primes Colon Cancer for Checkpoint Inhibitor and Cross-Primes T Cells for Antitumor and Antiviral Immunity. Mol Cancer Ther, 2021. 20 (1): p. 173-182.
15. Yang, A., et al., Development of the oncolytic virus, CF33, and its derivatives for peritoneal-directed treatment of gastric cancer peritoneal metastases. J Immunother Cancer, 2023. 11 (4).
16. Ribas, A., et al., Oncolytic Virotherapy Promotes Intratumoral T Cell Infiltration and Improves Anti-PD-1 Immunotherapy. Cell, 2017. 170 (6): p. 1109-1119 e10.
17. Papathanasiou, M. M., et al., Autologous CAR T-cell therapies supply chain: challenges and opportunities? Cancer Gene Ther, 2020. 27 (10-11): p. 799-809.
18. Goebeler, M. E. and R. Bargou, Blinatumomab: a CD19/CD3 bispecific T cell engager (BiTE) with unique anti-tumor efficacy. Leuk Lymphoma, 2016. 57 (5): p. 1021-32.
19. Moreau, P., et al., Teclistamab in Relapsed or Refractory Multiple Myeloma. N Engl J Med, 2022. 387 (6): p. 495-505.
20. Daei Sorkhabi, A., et al., The current landscape of CAR T-cell therapy for solid tumors: Mechanisms, research progress, challenges, and counterstrategies. Front Immunol, 2023. 14: p. 1113882.
21. Wang, X., et al., Phase 1 studies of central memory-derived CD19 CAR T-cell therapy following autologous HSCT in patients with B-cell NHL. Blood, 2016. 127 (24): p. 2980-90.
22. Priceman, S. J., et al., Regional Delivery of Chimeric Antigen Receptor-Engineered T Cells Effectively Targets HER2 (+) Breast Cancer Metastasis to the Brain. Clin Cancer Res, 2018. 24 (1): p. 95-105.
23. Priceman, S. J., et al., Co-stimulatory signaling determines tumor antigen sensitivity and persistence of CAR T cells targeting PSCA+ metastatic prostate cancer. Oncoimmunology, 2018. 7 (2): p. e1380764.
24. Voynov, V., et. al., Discovery Strategies to Maximize the Clinical Potential of T-Cell Engaging Antibodies for the Treatment of Solid Tumors. Antibodies (Basel), 2020 Dec. 9 (4): p. 65.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having a solid tumor comprising:
administering to the subject an effective amount of an oncolytic virus expressing a truncated CD19 (OV19t), the nucleotide sequence of OV19t comprising:
(a) an oncolytic virus nucleotide sequence; and
(b) a nucleotide sequence encoding a truncated human CD19; and administering to the subject an effective amount of a bispecific T cell engager (TCE) that binds to CD19.

2. An oncolytic virus expressing a truncated CD19 (OV19t), the nucleotide sequence of OV19t comprising:
(a) an oncolytic virus nucleotide sequence; and
(b) a nucleotide sequence encoding a truncated human CD19 (CD19t);
wherein the oncolytic virus nucleotide sequence encodes at least 25, 50, 60, 70, 80, 90, 100, 110, 120, or 121 of SEQ ID NO: Z1, Z17, Z26, Z27, Z28, Z32, Z34, Z36, Z41, Z52, Z53, Z55, Z63, Z65, Z66, Z76, Z77, Z79, Z81, Z82, Z87, Z90, Z91, Z92, Z93, Z104, Z108, Z110, Z111, Z116, Z118, Z120, Z123, Z128, Z129, Z130, Z138, Z140, Z142, Z143, Z145, Z146, Z147, Z149, Z150, Z151, Z152, Z153, Z154, Z155, Z156, Z157, Z158, Z160, Z161, Z165, Z168, Z169, Z170, Z172, Z173, Z174, Z175, Z176, Z177, Z178, Z181, Z182, Z186, Z192, Z193, Z194, Z195, Z197, Z198, Z199, Z203, Z206, Z211, Z212, Z216, Z219, Z220, Z222, Z223, Z224, Z225, Z226, Z227, Z228, Z229, Z231, Z232, Z233, Z234, Z236, Z238, Z240, Z241, Z243, Z244, Z252, Z253, Z254, Z257, Z258, Z259, Z260, Z261, Z263, Z264, Z265, Z266, Z267, Z268, Z269, Z270, Z271, Z272, Z274, and Z275; and/or
wherein the OV19t nucleotide sequence is at least 90%, 95%, 96%, 97%, 98%, 99%, 99.5%, 99.9% identical to or has no more than 300 single nucleotide changes compared to:
i) nucleotides 5,014-178,073 of SEQ ID NO: 1 over the entire length of nucleotides 5,014-178,073 SEQ ID NO: 1;
ii) nucleotides 7,000-178,000 of SEQ ID NO: 1 over the entire length of nucleotides 7,000-178,000 SEQ ID NO: 1;
iii) nucleotides 8,000-177,000 of SEQ ID NO:1 over the entire length of nucleotides 8,000-177,000 SEQ ID NO: 1;
iv) nucleotides 10,000-175,000 of SEQ ID NO: 1 over the entire length of nucleotides 10,000-175,000 SEQ ID NO: 1;
v) nucleotides 5,014-186,900 of SEQ ID NO:5 over the entire length of nucleotides 5,014-186,900 SEQ ID NO: 5;
vi) nucleotides 7,000-186,000 of SEQ ID NO:5 over the entire length of nucleotides 7,000-186,000 SEQ ID NO: 5;
vii) nucleotides 8,000-185,000 of SEQ ID NO: 1 over the entire length of nucleotides 8,000-185,000 SEQ ID NO: 5; or
viii) nucleotides 10,000-184,000 of SEQ ID NO: 1 over the entire length of nucleotides 10,000-184,000 SEQ ID NO: 5.
